(19) United States
(12) Patent Application Publication
(10) Pub. No.: US 2026/0159890 A1
(43) Pub. Date: Jun. 11, 2026

(54) SYSTEMS AND METHODS OF DETECTING SPLICE JUNCTIONS IN EXTRACELLULAR CELL-FREE MESSENGER RNA

(71) Applicant: Superfluid Dx, Inc., South San Francisco, CA (US)

(72) Inventors: John SNINSKY, Orinda, CA (US); Samantha KHOURY, New South Wales (AU); Shusuke TODEN, Palo Alto, CA (US)

(21) Appl. No.: 19/316,640

(22) Filed: Sep. 2, 2025

Related U.S. Application Data
(63) Continuation of application No. PCT/US24/18222, filed on Mar. 1, 2024.
(60) Provisional application No. 63/449,900, filed on Mar. 3, 2023.

(51) Int. Cl. C12Q 1/6883; G16B 40/00

(52) U.S. Cl. CPC C12Q 1/6883; G16B 40/00; C12Q 2600/158

(57) ABSTRACT

Disclosed herein are methods, systems, kits, and compositions for detecting a disease state of a subject by assaying cell-free messenger RNA (cf-mRNA) and detecting splice junctions in the cf-mRNA. Further disclosed herein are methods, systems, kits, and compositions for determining a risk of a disease state in a subject by assaying cf-mRNA and detecting splice junctions in the cf-mRNA.

### CROSS-REFERENCE

This application is a continuation of PCT/US24/18222, filed Mar. 1, 2024, which claims the benefit of U.S. Provisional Application No. 63/449,900, filed Mar. 3, 2023, each of which is herein incorporated by reference.

### BACKGROUND

The heterogeneous nature of Alzheimer's disease, as a complex neurodegenerative disease affecting multiple biological pathways and processes during preclinical Alzheimer's disease, clinical onset, and progression, represents one major difficulty for Alzheimer's disease drug development. An additional challenge is that in some cases, the underlying biological pathways are a mix of different types of dementias. Successful development of therapeutic agents for a heterogeneous Alzheimer's disease population may rely on the ability to appropriately enrich the trial groups for Alzheimer's disease patients likely to respond to the candidate drugs.

### SUMMARY

In one aspect, provided herein are methods for detecting a disease state of a subject, the method comprising: obtaining a biological sample from the subject; assaying cell-free messenger RNA (cf-mRNA) in the biological sample to determine a level of the cf-mRNA that contains a non-contiguous junction relative to genomic DNA, thereby detecting one or more splice junctions in the cf-mRNA; computer processing the detected one or more splice junctions; and detecting the disease state of the subject based at least in part on the computer processing. In some embodiments, the biological sample comprises a blood sample, a plasma sample, or a serum sample. In some embodiments, the biological sample comprises the plasma sample. In some embodiments, the biological sample comprises the serum sample. In some embodiments, the disease state comprises a severity of the disease state. In some embodiments, the disease state comprises a presence or an absence of Alzheimer's disease. In some embodiments, the assaying comprises one or more of sequencing, array hybridization, or nucleic acid amplification. In some embodiments, the sequencing comprises next generation sequencing (NGS). In some embodiments, the NGS comprises RNA sequencing. In some embodiments, the assaying comprises converting the cf-mRNA to complementary deoxyribonucleic acid (cDNA), thereby producing sample cDNA. In some embodiments, the assaying comprises comparing the sample cDNA to a reference sample. In some embodiments, the assaying comprises determining a relative abundance of the one or more splice junctions. In some embodiments, the methods further comprise comparing the determined relative abundance to a reference sample. In some embodiments, the method further comprises administering a treatment to the subject, thereby treating the disease state of the subject. In some embodiments, the treatment comprises a medicinal therapy, a behavioral therapy, a sleep therapy, or a combination thereof. In some embodiments, the treatment comprises the medicinal therapy. In some embodiments, the medicinal therapy comprises a cholinesterase inhibitor. In some embodiments, the medicinal therapy comprises a N-methyl-D-aspartate (NMDA) antagonist. In some embodiments, the medicinal therapy comprises an amyloid monoclonal antibody (mab) therapy. In some embodiments, the computer processing comprises use of machine learning. In some embodiments, the computer processing comprises use of prediction or classification. In some embodiments, the classification comprises use of a trained classifier. In some embodiments, the one or more splice junctions correspond to one or more genes. In some embodiments, the one or more genes are expressed in a first population of subjects with Alzheimer's disease as compared to a second population of subjects without Alzheimer's disease. In some embodiments, the one or more genes comprise a member selected from the group consisting of: ABLIM1 (ENSG00000099204.21:t:114491791-114491878;114491878-114545005), ACAA1 (ENSG00000060971.19:s:38126493-38126700;38126341-38126510), ACAP1 (ENSG00000072818.12:t:7341948-7342067;7336787-7341948), ACOT8 (ENSG00000101473.17:t:45848450-45849110;45848675-45857188), ACP1 (ENSG00000143727.16:t:272192-273155;272065-272192), ADD3 (ENSG00000148700.15:t:110100625-110100848;110008299-110100625), ADGRE5 (ENSG00000123146.20:t:14397414-14397804;14391079-14397658), ADK (ENSG00000156110.15:t:74200764-74200838;74151343-74200764), AGAP3 (ENSG00000133612.19:s:151119987-151120145;151120145-151122725), AKAP13 (ENSG00000170776.22:t:85575131-85575329;85543955-85575131), ALAD (ENSG00000148218.16:s:113393161-113393634;113392169-113393447), AMPD2 (ENSG00000116337.20:t:109625303-109625433;109621266-109625303), ANAPC11 (ENSG00000141552.18:t:81894298-81894624;81891833-81894467), AP1B1 (ENSG00000100280.17:s:29330378-29330532;29329720-29330378), AP1B1 (ENSG00000100280.17:t:29327680-29328895;29328895-29329712), APP (ENSG00000142192.21:s:26000015-26000182;25982477-26000015), APP (ENSG00000142192.21:t:25897573-25897983;25897673-25911741), ARAP1 (ENSG00000186635.15:s:72693325-72693470;72688537-72693325), ARFRP1 (ENSG00000101246.20:t:63706999-63707658;63707097-63707867), ARHGAP17 (ENSG00000140750.17:t:24935470-24936827;24935639-24941987), ARHGAP17 (ENSG00000288353.1:t:188953-190310;189122-195470), ARHGEF10 (ENSG00000274726.4:s:43270-43560;43560-47749), ARHGEF1 (ENSG00000076928.19:s:41896377-41896878;41896482-41897315), ARHGEF7 (ENSG00000102606.20:t:111217679-111217885;111210002-111217679), ARMC10 (ENSG00000170632.14:s:103074881-103075411;103075411-103075777), ARRDC2 (ENSG00000105643.11:t:18008711-18008861;18001573-18008711), ATE1 (ENSG00000107669.19:t:121924266-121924329;121924329-121928347), ATP6V1D (ENSG00000100554.12:t:67350611-67350690;67350690-67359658), ATXN2L (ENSG00000168488.19:s:28835933-28836176;28836122-28836748), ATXN2L (ENSG000000168488.19:t:28836728-28837237;28836122-28836748), ATXN2L (ENSG000000168488.19:t:28836728-28837237;28836485-28836728), AURKB (ENSG00000178999.13:t:8207738-8208225;8207840-8210530), BLOC1S6 (ENSG00000104164.12:s:45592135-45592276;45592276-45605428), C12orf76 (ENSG00000174456.16:s:110048363-110048870;110042459-110048363), CAMTA1 (ENSG00000171735.20:t:6825092-6825210;6820250-6825092), CBFA2T3 (ENSG00000129993.15:t:88892244-88892485;88892485-88898078), CBFB (ENSG00000067955.15:t:67066682-67066962;67036755-67066682), CCDC28B (ENSG00000160050.16:t:32204598-32204620;32204379-32204598), CCDC92 (ENSG00000119242.9:s:123972529-123972831;123943493-123972529), CCDC92 (ENSG00000119242.9:t:123943347-123943495;123943493-123972529), CD300H (ENSG00000284690.3:s:74563919-74564275;74560824-74563919), CD34 (ENSG00000174059.17:s:207888682-207888846;207887923-207888682), CDAN1 (ENSG00000140326.13:s:42736989-42737128;42736780-42737013), CDC14B (ENSG00000081377.17:t:96565393-96565512;96565483-96619219), CDK5RAP2 (ENSG00000136861.19:t:120402806-120403071;120403071-120403316), CELF2 (ENSG00000048740.19:t:11249153-11249201;11165682-11249153), CEP164 (ENSG00000110274.16:t:117409618-117410701;117409028-117409618), CLEC1B (ENSG00000165682.15:s:9995140-9995246;9986158-9995140), COX20 (ENSG00000203667.10:s:244835616-244835756;244835756-244842195), CPNE1 (ENSG000000214078.13:s:35627280-35627531;35626800-35627280), CYTH2 (ENSG00000105443.17:s:48474838-48476276;48474949-48477717), CYTH2 (ENSG00000105443.17:t:48478066-48478145;48477719-48478069), DAZAP1 (ENSG00000071626.17:s:1422348-1422396;1422396-1425878), DCTD (ENSG00000129187.15:s:182917288-182917477;182915575-182917311), DCTD (ENSG00000129187.15:t:182915461-182915575;182915575-182917288), DECR1 (ENSG00000104325.7:s:90001405-90001561;90001561-90017124), DECR1 (ENSG00000104325.7:s:90001405-90001561;90001561-90018909), DECR1 (ENSG00000104325.7:t:90018564-90018966;90001561-90018909), DEF8 (ENSG00000140995.17:t:89954172-89954376;89949513-89954243), DGKD (ENSG00000077044.11:s:233390403-233390483;233390483-233392070), DGKZ (ENSG00000149091.15:t:46367291-46367399;46345585-46367291), DNM2 (ENSG00000079805.19:s:10795372-10795439;10795439-10797380), DOCK8 (ENSG00000107099.18:t:271627-271729;215029-271627), DUT (ENSG00000128951.14:t:48332103-48332737;48331479-48332268), DYRK1A (ENSG00000157540.22:s:37418905-37420384;37420384-37472684), DYSF (ENSG00000135636.16:t:71480883-71480938;71454086-71480883), EIF4G1 (ENSG00000114867.22:t:184317321-184317497;184314674-184317321), EIF4G3 (ENSG00000075151.24:t:20981048-20981227;20981227-20997601), EMC8 (ENSG00000131148.9:s:85781211-85781760;85779867-85781211), EPSTI1 (ENSG00000133106.15:s:42991978-42992271;42969177-42991978), EXOSC1 (ENSG00000171311.13:s:97438663-97438703;97437750-97438663), EXOSC1 (ENSG00000171311.13:t:97437700-97437750;97437750-97438663), F2RL3 (ENSG00000127533.4:s:16888999-16889298;16889298-16889577), FAM219A (ENSG00000164970.15:s:34405865-34405964;34402807-34405916), FAM3A (ENSG00000071889.17:s:154512823-154512936;154511871-154512823), FBXO44 (ENSG00000132879.14:t:11658736-11658871;11658628-11658736), FKBP1B (ENSG00000119782.14:s:24060814-24060926;24060926-24063019), FMNL3 (ENSG00000161791.14:t:49657082-49657190;49657190-49658442), FOXP1 (ENSG00000114861.24:t:71112536-71112637;71112637-71130498), G3BP1 (ENSG00000145907.16:t:151786572-151786715;151772036-151786603), GET1 (ENSG00000182093.16:t:39390698-39390803;39380556-39390698), GLUL (ENSG00000135821.19:t:182388572-182390304;182388750-182391175), GORASP1 (ENSG00000114745.15:s:39107479-39108063;39103553-39107479), GORASP1 (ENSG00000114745.15:t:39101016-39101102;39103553-39107479), GSE1 (ENSG00000131149.19:t:85648552-85648751;85556363-85648552), GSTT1 (ENSG00000277656.3:t:270997-271173;271173-278295), GUCD1 (ENSG00000138867.17:t:24548917-24549001;24549001-24555615), HDAC7 (ENSG00000061273.18:s:47796207-47796298;47796016-47796207), HES6 (ENSG00000144485.11:t:238239487-238239568;238239568-238239825), HUS1 (ENSG00000136273.13:s:47979410-47979615;47978816-47979410), IFI27 (ENSG00000275214.4:t:1230073-1230504;1229442-1230343), IGF2BP3 (ENSG00000136231.14:t:23342064-23342189;23342189-23343718), IKBKB (ENSG00000104365.16:t:42290156-42290273;42288728-42290156), IKBKG (ENSG00000269335.7:t:154551988-154552189;154542452-154551988), IKZF3 (ENSG00000161405.17:t:39777651-39777767;39777767-39788258), IL10RA (ENSG00000110324.12:t:117988382-117988502;117986534-117988382), IL15RA (ENSG00000134470.21:t:5960367-5960567;5960567-5963743), IMPDH1 (ENSG00000106348.18:t:128405767-128405948;128405865-128409289), INO80E (ENSG00000169592.15:s:30001414-30001575;30001530-30005221), INPP5D (ENSG00000281614.3:s:67445-67595;67595-71086), IP6K2 (ENSG00000068745.15:t:48694872-48695421;48695421-48717157), IRF7 (ENSG00000276561.4:s:144369-144427;144292-144369), IRF7 (ENSG00000276561.4:s:144676-144900;144424-144690), ITSN2 (ENSG00000198399.16:s:24246129-24246320;24242206-24246129), JAML (ENSG00000160593.19:t:118212407-118212561;118212561-118214824), JOSD2 (ENSG00000161677.12:t:50506378-50506572;50506572-50507574), KANK2 (ENSG00000197256.11:s:11195556-11195881;11194590-11195556), KANK2 (ENSG00000197256.11:t:11194553-11194590;11194590-11195556), KANSL3 (ENSG00000114982.19:s:96636739-96637228;96631543-96636921), KANSL3 (ENSG00000114982.19:t:96631312-96631543;96631543-96636921), KAT5 (ENSG00000172977.13:s:65712922-65713319;65713058-65713348), KATNB1 (ENSG00000140854.13:s:57755345-57755494;57755494-57755841), KDM5C (ENSG00000126012.13:s:53217796-53217966;53217274-53217796), KIAA1671 (ENSG00000197077.14:t:25170820-25170938;25049364-25170820), KIFC3 (ENSG00000140859.16:t:57772223-57772288;57772288-57785464), KIFC3 (ENSG00000140859.16:t:57798072-57798282;57798282-57802370), KMT2B (ENSG00000272333.8:t:35719784-35720940;35719541-35719784), LDB1 (ENSG00000198728.11:s:102109029-102109177;102108323-102109029), LDB2 (ENSG00000169744.13:s:16511981-16512104;16508686-16511981), LETMD1 (ENSG00000050426.16:s:51048301-51048518;51048478-51056350), LST1 (ENSG00000204482.11:s:31587117-31587318;31587318-31587641), LST1 (ENSG00000204482.11:t:31587944-31587966;31587318-31587944), LST1 (ENSG00000206433.10:s:2842938-2843143;2843139-2844386), LST1 (ENSG00000223465.8:s:2834852-2835057;2835053-2835376), LST1 (ENSG00000223465.8:t:2835679-2835701;2835053-2835679), LST1 (ENSG00000226182.8:t:3065231-3065253;3064605-3065231), LST1 (ENSG00000230791.8:s:2886832-2887014;2887014-2887225), LST1 (ENSG00000230791.8:t:2887225-2887247;2886599-2887225), LST1 (ENSG00000231048.8:s:2892158-2892363;2892359-2892682), LST1 (ENSG00000231048.8:t:2892985-2893007;2892359-2892985), LTB (ENSG00000204487.8:s:3059543-3059714;3058917-3059543), LTB (ENSG00000223448.7:s:2887297-2887468;2886671-2887297), LTB (ENSG00000238114.7:s:2881536-2881707;2880910-2881536), LTBP4 (ENSG00000090006.18:s:40619347-40619493;40619493-40622401), LTBP4 (ENSG00000090006.18:t:40619347-40619493;40614446-40619347), LTBP4 (ENSG00000090006.18:t:40623604-40623732;40623021-40623604), MAPK9 (ENSG00000050748.18:t:180269280-180269409;180269409-180279796), MARK2 (ENSG00000072518.22:s:63904786-63905043;63905043-63908260), MAST3 (ENSG00000099308.13:s:18146881-18147044;18147017-18147443), MAX (ENSG00000125952.20:t:65077193-65077428;65077428-65077913), MBNL1 (ENSG00000152601.18:t:152414941-152415111;152269092-152414941,) METTL9 (ENSG00000197006.15:s:21624931-21625233;21625115-21655227), MGLL (ENSG00000074416.16:t:127821694-127821838;127821838-127822309), MGRN1 (ENSG00000102858.13:s:4677463-4677572;4677572-4681550), MIEF1 (ENSG00000100335.15:s:39511849-39512026;39512026-39512232), MLX (ENSG00000108788.12:s:42567619-42567655;42567655-42568837), MMAB (ENSG00000139428.12:s:109561420-109561517;109561329-109561420), MPI (ENSG00000178802.18:s:74890005-74890201;74890089-74890527), MPRIP (ENSG00000133030.22:t:17142627-17142765;17138429-17142627), MRPL33 (ENSG00000243147.8:s:27772674-27772855;27772692-27779433), MSRB3 (ENSG00000174099.12:s:65278643-65278865;65278865-65326826), MTA1 (ENSG00000182979.18:s:105445418-105445511;105445511-105450058), MTA1 (ENSG00000182979.18:t:105450058-105450184;105445511-105450058), MTHFD2 (ENSG00000065911.13:t:74207704-74207826;74205889-74207704), MTMR12 (ENSG00000150712.11:s:32312758-32312987;32274122-32312758), MTMR12 (ENSG00000150712.11:t:32273980-32274122;32274122-32312758), NAP1L4 (ENSG00000273562.4:t:175479-176694;176694-182308), NCOA2 (ENSG00000140396.13:t:70141179-70141399;70141399-70148273), NCOR2 (ENSG00000196498.14:s:124330845-124330898;124326370-124330845), NCOR2 (ENSG00000196498.14:s:124378237-124378384;124372610-124378237), NCOR2 (ENSG00000196498.14:t:124372022-124372610;124372610-124378237), NDRG2 (ENSG00000165795.25:t:21022392-21022820;21022497-21022864), NDUFV3 (ENSG00000160194.18:t:42908864-42913304;42897047-42908864), NFE2L1 (ENSG00000082641.16:s:48056386-48056598;48056598-48057032), NFIA (ENSG00000162599.18:t:61455303-61462788;61406727-61455303), NKIRAS2 (ENSG00000168256.18:t:42023654-42025644;42022640-42023654), NOC2L (ENSG00000188976.11:s:945042-945146;944800-945057), NTAN1 (ENSG00000275779.4:s:613600-613788;613788-621580), NTPCR (ENSG00000135778.12:t:232956347-232956443;232950744-232956347), NUDT22 (ENSG00000149761.9:t:64229131-64229344;64227132-64229247), NUP214 (ENSG00000126883.19:s:131146129-131146304;131146304-131147490), NXT2 (ENSG00000101888.12:t:109538045-109538131;109537270-109538045), OCEL1 (ENSG00000099330.9:s:17226213-17226353;17226353-17226693), P2RX4 (ENSG00000135124.16:s:121210065-121210298;121210298-121217134), PABIR2 (ENSG00000156504.17:s:134781821-134781917;134772283-134781821), PALM2AKAP2 (ENSG00000157654.19:s:110138171-110138539;110138539-110168399), PANK4 (ENSG00000157881.16:t:2521101-2521315;2521315-2526464), PAPOLA (ENSG00000090060.19:s:96556175-96556413;96556413-96560649), PARL (ENSG00000175193.14:s:183862607-183862801;183844326-183862697), PARL (ENSG00000175193.14:t:183844231-183844774;183844326-183862753), PARVB (ENSG00000188677.15:t:44093928-44094017;44069162-44093928), PCYT1B (ENSG00000102230.14:t:24618985-24619084;24619084-24646989), PDE7A (ENSG00000205268.11:t:65782783-65782843;65782843-65841371), PEX26 (ENSG00000215193.14:s:18083437-18083732;18083732-18087972), PFKFB3 (ENSG00000170525.21:t:6213623-6213748;6203336-6213623), PKN1 (ENSG00000123143.13:t:14441143-14441443;14433542-14441143), PLEKHM1 (ENSG00000225190.12:s:45481603-45482612;45478147-45482433), PLS3 (ENSG00000102024.19:t:115610243-115610323;115561260-115610243), PML (ENSG00000140464.20:s:74034478-74034558;74034530-74042989), PML (ENSG00000140464.20:t:74033156-74033422;74032715-74033156), POLR2J3 (ENSG00000168255.20:t:102566997-102567083;102567083-102568010), POLR2J3 (ENSG00000285437.2:t:102566966-102567083;102567083-102568010), PPIE (ENSG00000084072.17:s:39752910-39753266;39753052-39753287), PPM1N (ENSG00000213889.11:t:45499949-45500066;45497343-45499949), PPP6R2 (ENSG00000100239.16:s:50436367-50436452;50436452-50436988), PPP6R2 (ENSG00000100239.16:t:50437506-50437603;50437068-50437506), PRKAR1B (ENSG00000188191.16:t:596146-596304;596304-602553), PRKCD (ENSG00000163932.16:t:53178404-53178537;53165215-53178404), PSEN1 (ENSG00000080815.20:t:73170797-73171923;73148094-73170797), PSMB8 (ENSG00000230034.8:t:4086512-4086659;4086659-4087849), PSMG4 (ENSG00000180822.12:s:3263684-3263759;3263759-3264209), PTK2B (ENSG00000120899.18:t:27450749-27450895;27445919-27450749), PTPN12 (ENSG00000127947.16:s:77600664-77600965;77600806-77607235), PTPN18 (ENSG00000072135.13:t:130368897-130369201;130356200-130369133), PUF60 (ENSG00000179950.15:s:143821118-143821743;143818534-143821597), PUF60 (ENSG00000179950.15:s:143821118-143821743;143820716-143821597), PUM1 (ENSG00000134644.16:s:30967099-30967310;30966278-30967167), PYM1 (ENSG00000170473.17:t:55903387-55903480;55903480-55927076), RAB11FIP1 (ENSG00000156675.16:t:37870387-37870528;37870528-37871278), RAB11FIP5 (ENSG00000135631.17:t:73075993-73076182;73076182-73088050), RAB4A (ENSG00000168118.12:t:229295848-229295910;229271370-229295848), RABGAP1L (ENSG00000152061.24:t:174969277-174969387;174957549-174969277), RAP1B (ENSG00000127314.18:t:68650244-68650882;68648781-68650400), RARA (ENSG00000131759.18:t:40348316-40348464;40331396-40348316), RBCK1 (ENSG00000125826.22:s:409830-410025;410025-417526), RBM10 (ENSG00000182872.16:t:47173128-47173197;47169498-47173128), RBM39 (ENSG00000131051.24:s:35713019-35713203;35709256-35713019), RCOR3 (ENSG00000117625.14:t:211313424-211316385;211312961-211313424), REPS2 (ENSG00000169891.18:s:17022123-17022371;17022271-17025059), RFFL (ENSG00000092871.17:s:35026374-35026568;35021781-35026374), RHD (ENSG00000187010.21:s:25303322-25303459;25303459-25328898), RMND5B (ENSG00000145916.19:t:178138108-178138396;178131054-178138108), RPUSD1 (ENSG00000007376.8:t:786826-786928;786928-787077), SEC16A (ENSG00000148396.19:s:136447227-136447364;136446949-136447227), SIGLEC10 (ENSG00000142512.15:t:51414422-51414515;51414515-51415181), SIRT3 (ENSG00000142082.15:s:218778-219041;216718-218778), SLA2 (ENSG00000101082.14:s:36615225-36615374;36614387-36615225), SLC66A2 (ENSG00000122490.19:t:79903446-79904183;79904183-79919184), SMARCA4 (ENSG00000127616.21:s:11034914-11035132;11035132-11041298), SMARCA4 (ENSG00000127616.21:t:11040634-11041560;11035132-11041298), SMARCC2 (ENSG000000139613.12:s:56173696-56173849;56173029-56173696), SNX20 (ENSG000000167208.16:s:50675770-50675921;50669148-50675770), SPIB (ENSG00000269404.7:s:50423605-50423755;50423751-50428038), SRSF6 (ENSG00000124193.16:s:43458361-43458509;43458509-43459153), SSX2IP (ENSG00000117155.17:t:84670646-84670815;84670815-84690371), STAMBP (ENSG00000124356.17:t:73830845-73831059;73829070-73830845), STAT3 (ENSG00000168610.16:t:42317182-42317224;42317224-42319856), SWI5 (ENSG00000175854.15:t:128276587-128276755;128276402-128276707), SYNE1 (ENSG00000131018.25:s:152301869-152302269;152300781-152301869), TANGO2 (ENSG00000183597.16:t:20061530-20061802;20056013-20061530), TCF3 (ENSG00000071564.19:s:1615686-1615804;1612433-1615686), TCF7L2 (ENSG00000148737.18:s:113146011-113146097;113146097-113150998), TECR (ENSG00000099797.15:s:14562356-14562575;14562575-14563174), TJP2 (ENSG00000119139.21:t:69212548-69212601;69151771-69212548), TLE4 (ENSG00000106829.21:s:79627374-79627752;79627448-79652593), TLE4 (ENSG00000106829.21:t:79652590-79652629;79627448-79652593), TMBIM1 (ENSG00000135926.15:s:218292466-218292586;218282181-218292466), TMBIM4 (ENSG000000155957.19:s:66169855-66170027;66161172-66169855), TMEM11 (ENSG00000178307.10:s:21214091-21214176;21211227-21214091), TMEM126B (ENSG00000171204.13:t:85631687-85631808;85628688-85631687), TMEM219 (ENSG00000149932.17:t:29962677-29963308;29962132-29963107), TNFSF12 (ENSG00000239697.12:t:7549466-7549618;7549312-7549474), TNK2 (ENSG00000061938.21:t:195888426-195888606;195888606-195908485), TNRC18 (ENSG00000182095.15:t:5325096-5325574;5325248-5329937), TRAPPC2 (ENSG00000196459.15:s:13734525-13734635;13719982-13734525), TRAPPC6A (ENSG00000007255.10:t:45164725-45164970;45164970-45165127), TSC22D3 (ENSG00000157514.18:t:107715773-107715950;107715950-107775100), TSPAN32 (ENSG00000064201.16:s:2314485-2314666;2314571-2316229), UBAC2 (ENSG00000134882.16:t:99238427-99238554;99200939-99238427), UFD1 (ENSG00000070010.19:t:19471687-19471808;19471808-19479083), URI1 (ENSG00000105176.18:s:29942239-29942664;29942664-29985223), URI1 (ENSG00000105176.18:t:29985223-29985301;29942664-29985223), USP15 (ENSG00000135655.16:s:62325872-62325933;62325933-62349221), USP22 (ENSG00000124422.12:t:21028542-21028674;21028674-21043321), VGLL4 (ENSG00000144560.16:t:11601833-11602022;11602022-11702971), WWP2 (ENSG00000198373.13:t:69786996-69787080;69762391-69786996), YAF2 (ENSG00000015153.15:s:42237599-42237800;42199235-42237599), YIPF6 (ENSG00000181704.12:s:68498562-68499157;68499123-68511849), YIPF6 (ENSG00000181704.12:t:68513327-68515340;68511977-68513327), ZBTB7B (ENSG00000160685.14:t:155014448-155015814;155002943-155014655), ZEB2 (ENSG00000169554.23:t:144517278-144517557;144517419-144517612), ZFAND1 (ENSG00000104231.11:s:81718182-81718224;81715114-81718182), ZFAND1 (ENSG00000104231.11:t:81714987-81715114;81715114-81718182), ZFAND2B (ENSG00000158552.13:s:219207648-219207779;219207774-219207887), ZMIZ2 (ENSG00000122515.16:s:44759281-44759460;44759460-44760151), ZMYND8 (ENSG00000101040.20:t:47236326-47236516;47236516-47238758), ZNF185 (ENSG00000147394.20:s:152922720-152922809;152922809-152928575), ZNF451 (ENSG00000112200.17:t:57099061-57099141;57090274-57099061), and a combination thereof. In some embodiments, the one or more genes comprise a member selected from the group consisting of: LDB2 (ENSG00000169744.13:s:16511981-16512104;16508686-16511981), JOSD2 (ENSG00000161677.12:t:50506378-50506572;50506572-50507574), KIFC3 (ENSG00000140859.16:t:57798072-57798282;57798282-57802370), DOCK8 (ENSG00000107099.18:t:271627-271729;215029-271627), METTL9 (ENSG00000197006.15:s:21624931-21625233;21625115-21655227), CCDC28B (ENSG00000160050.16:t:32204598-32204620;32204379-32204598), EXOSC1 (ENSG00000171311.13:s:97438663-97438703;97437750-97438663), CBFA2T3 (ENSG00000129993.15:t:88892244-88892485;88892485-88898078), PUF60 (ENSG00000179950.15:s:143821118-143821743;143818534-143821597), NOC2L (ENSG00000188976.11:s:945042-945146;944800-945057), AP1B1 (ENSG00000100280.17:s:29330378-29330532;29329720-29330378), PPIE (ENSG00000084072.17:s:39752910-39753266;39753052-39753287), ARAP1 (ENSG00000186635.15:s:72693325-72693470;72688537-72693325), POLR2J3 (ENSG00000285437.2:t:102566966-102567083;102567083-102568010), DAZAP1 (ENSG00000071626.17:s:1422348-1422396;1422396-1425878), and ARHGEF1 (ENSG00000076928.19:s:41896377-41896878;41896482-41897315). In some embodiments, the one or more genes comprise a member selected from the group consisting of: SPIB (ENSG00000269404.7:s:50423605-50423755;50423751-50428038), KIFC3 (ENSG00000140859.16:t:57798072-57798282;57798282-57802370), LDB2 (ENSG00000169744.13:s:16511981-16512104;16508686-16511981), JOSD2 (ENSG00000161677.12:t:50506378-50506572;50506572-50507574), LST1 (ENSG00000230791.8:t:2887225-2887247;2886599-2887225), CCDC28B (ENSG00000160050.16:t:32204598-32204620;32204379-32204598), METTL9 (ENSG00000197006.15:s:21624931-21625233;21625115-21655227), PPIE (ENSG00000084072.17:s:39752910-39753266;39753052-39753287), CBFA2T3 (ENSG00000129993.15:t:88892244-88892485;88892485-88898078), TCF3 (ENSG00000071564.19:s:1615686-1615804;1612433-1615686), DCTD (ENSG00000129187.15:s:182917288-182917477;182915575-182917311), IL10RA (ENSG00000110324.12:t:117988382-117988502;117986534-117988382), ZNF451 (ENSG00000112200.17:t:57099061-57099141;57090274-57099061), AP1B1 (ENSG00000100280.17:s:29330378-29330532;29329720-29330378), EXOSC1 (ENSG00000171311.13:s:97438663-97438703;97437750-97438663), PUF60 (ENSG00000179950.15:s:143821118-143821743;143818534-143821597), POLR2J3 (ENSG00000285437.2:t:102566966-102567083;102567083-102568010), DOCK8 (ENSG00000107099.18:t:271627-271729;215029-271627), NOC2L (ENSG00000188976.11:s:945042-945146;944800-945057), NAP1L4 (ENSG00000273562.4:t:175479-176694;176694-182308), DAZAP1 (ENSG00000071626.17:s:1422348-1422396;1422396-1425878), ARAP1 (ENSG00000186635.15:s:72693325-72693470;72688537-72693325), RBM39 (ENSG00000131051.24:s:35713019-35713203;35709256-35713019), MSRB3 (ENSG00000174099.12:s:65278643-65278865;65278865-65326826), ZBTB7B (ENSG00000160685.14:t:155014448-155015814;155002943-155014655), NUP214 (ENSG00000126883.19:s:131146129-131146304;131146304-131147490), and ARHGEF1 (ENSG00000076928.19:s:41896377-41896878;41896482-41897315). In some embodiments, the one or more genes comprise a member selected from the group consisting of: ACAP1 (ENSG00000072818.12:t:7341948-7342067;7336787-7341948), ADGRE5 (ENSG00000123146.20:t:14397414-14397804;14391079-14397658), ADK (ENSG00000156110.15:t:74200764-74200838;74151343-74200764), ARHGEF1 (ENSG00000076928.19:s:41896377-41896878;41896482-41897315), ATXN2L (ENSG00000168488.19:t:28836728-28837237;28836485-28836728), CBFB (ENSG00000067955.15:t:67066682-67066962;67036755-67066682), CCDC92 (ENSG00000119242.9:s:123972529-123972831;123943493-123972529), DCTD (ENSG00000129187.15:s:182917288-182917477;182915575-182917311), DECR1 (ENSG00000104325.7:s:90001405-90001561;90001561-90017124), DOCK8 (ENSG00000107099.18:t:271627-271729;215029-271627), DYRK1A (ENSG00000157540.22:s:37418905-37420384;37420384-37472684), EXOSC1 (ENSG00000171311.13:s:97438663-97438703;97437750-97438663), FAM219A (ENSG00000164970.15:s:34405865-34405964;34402807-34405916), GLUL (ENSG00000135821.19:t:182388572-182390304;182388750-182391175), GSE1 (ENSG00000131149.19:t:85648552-85648751;85556363-85648552), GUCD1 (ENSG00000138867.17:t:24548917-24549001;24549001-24555615), LST1 (ENSG00000230791.8:t:2887225-2887247;2886599-2887225), MAST3 (ENSG00000099308.13:s:18146881-18147044;18147017-18147443), METTL9 (ENSG00000197006.15:s:21624931-21625233;21625115-21655227), MPRIP (ENSG00000133030.22:t:17142627-17142765;17138429-17142627), MRPL33 (ENSG00000243147.8:s:27772674-27772855;27772692-27779433), NAP1L4 (ENSG00000273562.4:t:175479-176694;176694-182308), NCOR2 (ENSG00000196498.14:s:124330845-124330898;124326370-124330845), NDRG2 (ENSG00000165795.25:t:21022392-21022820;21022497-21022864), NKIRAS2 (ENSG00000168256.18:t:42023654-42025644;42022640-42023654), PDE7A (ENSG00000205268.11:t:65782783-65782843;65782843-65841371), PKN1 (ENSG00000123143.13:t:14441143-14441443;14433542-14441143), PLEKHM1 (ENSG00000225190.12:s:45481603-45482612;45478147-45482433), PTPN12 (ENSG00000127947.16:s:77600664-77600965;77600806-77607235), RAB11FIP1 (ENSG00000156675.16:t:37870387-37870528;37870528-37871278), RCOR3 (ENSG00000117625.14:t:211313424-211316385;211312961-211313424), RFFL (ENSG00000092871.17:s:35026374-35026568;35021781-35026374), SPIB (ENSG00000269404.7:s:50423605-50423755;50423751-50428038), SSX2IP (ENSG00000117155.17:t:84670646-84670815;84670815-84690371), TMEM219 (ENSG00000149932.17:t:29962677-29963308;29962132-29963107), TNFSF12 (ENSG00000239697.12:t:7549466-7549618;7549312-7549474), TRAPPC2 (ENSG00000196459.15:s:13734525-13734635;13719982-13734525), USP15 (ENSG00000135655.16:s:62325872-62325933;62325933-62349221), YIPF6 (ENSG00000181704.12:t:68513327-68515340;68511977-68513327), ZFAND2B (ENSG00000158552.13:s:219207648-219207779;219207774-219207887), ZMYND8 (ENSG00000101040.20:t:47236326-47236516;47236516-47238758), and ZNF451 (ENSG00000112200.17:t:57099061-57099141;57090274-57099061. In some embodiments, the one or more splice junctions comprise one or more isoforms. In some embodiments, the one or more splice junctions comprise exon-exon junctions, exon-intron junctions, or intron-intron junctions.

In another aspect, provided herein are methods of determining a risk of a disease state in a subject, the method comprising: obtaining a biological sample from the subject; assaying cell-free messenger RNA (cf-mRNA) in the biological sample to detect one or more splice junctions in the cf-mRNA, wherein the one or more splice junctions correspond to one or more genes. In some embodiments, the biological sample comprises a blood sample, a plasma sample, or a serum sample. In some embodiments, the biological sample comprises the plasma sample. In some embodiments, the biological sample comprises the serum sample. In some embodiments, the disease state comprises a severity of the disease state. In some embodiments, the disease state comprises a presence or an absence of Alzheimer's disease. In some embodiments, the assaying comprises one or more of sequencing, array hybridization, or nucleic acid amplification. In some embodiments, the sequencing comprising next generation sequencing (NGS). In some embodiments, the NGS comprises RNA sequencing. In some embodiments, the assaying comprises converting the cf-mRNA to complementary deoxyribonucleic acid (cDNA), thereby producing sample cDNA. In some embodiments, the assaying comprises comparing the sample cDNA to a reference sample. In some embodiments, the assaying comprises determining a relative abundance of the one or more splice junctions. In some embodiments, the methods further comprise comparing the determined relative abundance to a reference sample. In some embodiments, the method further comprises administering a treatment to the subject, thereby treating the disease state of the subject. In some embodiments, the treatment comprises a medicinal therapy, a behavioral therapy, a sleep therapy, or a combination thereof. In some embodiments, the treatment comprises the medicinal therapy. In some embodiments, the medicinal therapy comprises a cholinesterase inhibitor. In some embodiments, the medicinal therapy comprises a N-methyl-D-aspartate (NMDA) antagonist. In some embodiments, the medicinal therapy comprises an amyloid monoclonal antibody (mab) therapy. In some embodiments, the method further comprises computer processing the detected one or more splice junctions. In some embodiments, the computer processing comprises use of machine learning. In some embodiments, the computer processing comprises use of prediction or classification. In some embodiments, the classification comprises use of a trained classifier. In some embodiments, the one or more genes are expressed in a first population of subjects with Alzheimer's disease as compared to a second population of subjects without Alzheimer's disease. In some embodiments, the one or more genes comprise a member selected from the group consisting of: ABLIM1 (ENSG00000099204.21:t:114491791-114491878;114491878-114545005), ACAA1 (ENSG00000060971.19:s:38126493-38126700;38126341-38126510), ACAP1 (ENSG00000072818.12:t:7341948-7342067;7336787-7341948), ACOT8 (ENSG00000101473.17:t:45848450-45849110;45848675-45857188), ACP1 (ENSG00000143727.16:t:272192-273155;272065-272192), ADD3 (ENSG00000148700.15:t:110100625-110100848;110008299-110100625), ADGRE5 (ENSG00000123146.20:t:14397414-14397804;14391079-14397658), ADK (ENSG00000156110.15:t:74200764-74200838;74151343-74200764), AGAP3 (ENSG00000133612.19:s:151119987-151120145;151120145-151122725), AKAP13 (ENSG00000170776.22:t:85575131-85575329;85543955-85575131), ALAD (ENSG00000148218.16:s:113393161-113393634;113392169-113393447), AMPD2 (ENSG00000116337.20:t:109625303-109625433;109621266-109625303), ANAPC11 (ENSG00000141552.18:t:81894298-81894624;81891833-81894467), AP1B1 (ENSG00000100280.17:s:29330378-29330532;29329720-29330378), AP1B1 (ENSG00000100280.17:t:29327680-29328895;29328895-29329712), APP (ENSG00000142192.21:s:26000015-26000182;25982477-26000015), APP (ENSG00000142192.21:t:25897573-25897983;25897673-25911741), ARAP1 (ENSG00000186635.15:s:72693325-72693470;72688537-72693325), ARFRP1 (ENSG00000101246.20:t:63706999-63707658;63707097-63707867), ARHGAP17 (ENSG00000140750.17:t:24935470-24936827;24935639-24941987), ARHGAP17 (ENSG00000288353.1:t:188953-190310;189122-195470), ARHGEF10 (ENSG00000274726.4:s:43270-43560;43560-47749), ARHGEF1 (ENSG00000076928.19:s:41896377-41896878;41896482-41897315), ARHGEF7 (ENSG00000102606.20:t:111217679-111217885;111210002-111217679), ARMC10 (ENSG00000170632.14:s:103074881-103075411;103075411-103075777), ARRDC2 (ENSG00000105643.11:t:18008711-18008861;18001573-18008711), ATE1 (ENSG00000107669.19:t:121924266-121924329;121924329-121928347), ATP6V1D (ENSG00000100554.12:t:67350611-67350690;67350690-67359658), ATXN2L (ENSG00000168488.19:s:28835933-28836176;28836122-28836748), ATXN2L (ENSG00000168488.19:t:28836728-28837237;28836122-28836748), ATXN2L (ENSG00000168488.19:t:28836728-28837237;28836485-28836728), AURKB (ENSG00000178999.13:t:8207738-8208225;8207840-8210530), BLOC1S6 (ENSG00000104164.12:s:45592135-45592276;45592276-45605428), C12orf76 (ENSG00000174456.16:s:110048363-110048870;110042459-110048363), CAMTA1 (ENSG00000171735.20:t:6825092-6825210;6820250-6825092), CBFA2T3 (ENSG00000129993.15:t:88892244-88892485;88892485-88898078), CBFB (ENSG00000067955.15:t:67066682-67066962;67036755-67066682), CCDC28B (ENSG00000160050.16:t:32204598-32204620;32204379-32204598), CCDC92 (ENSG00000119242.9:s:123972529-123972831;123943493-123972529), CCDC92 (ENSG00000119242.9:t:123943347-123943495;123943493-123972529), CD300H (ENSG00000284690.3:s:74563919-74564275;74560824-74563919), CD34 (ENSG00000174059.17:s:207888682-207888846;207887923-207888682), CDAN1 (ENSG00000140326.13:s:42736989-42737128;42736780-42737013), CDC14B (ENSG00000081377.17:t:96565393-96565512;96565483-96619219), CDK5RAP2 (ENSG00000136861.19:t:120402806-120403071;120403071-120403316), CELF2 (ENSG00000048740.19:t:11249153-11249201;11165682-11249153), CEP164 (ENSG00000110274.16:t:117409618-117410701;117409028-117409618), CLEC1B (ENSG00000165682.15:s:9995140-9995246;9986158-9995140), COX20 (ENSG00000203667.10:s:244835616-244835756;244835756-244842195), CPNE1 (ENSG00000214078.13:s:35627280-35627531;35626800-35627280), CYTH2 (ENSG00000105443.17:s:48474838-48476276;48474949-48477717), CYTH2 (ENSG00000105443.17:t:48478066-48478145;48477719-48478069), DAZAP1 (ENSG00000071626.17:s:1422348-1422396;1422396-1425878), DCTD (ENSG00000129187.15:s:182917288-182917477;182915575-182917311), DCTD (ENSG00000129187.15:t:182915461-182915575;182915575-182917288), DECR1 (ENSG00000104325.7:s:90001405-90001561;90001561-90017124), DECR1 (ENSG00000104325.7:s:90001405-90001561;90001561-90018909), DECR1 (ENSG00000104325.7:t:90018564-90018966;90001561-90018909), DEF8 (ENSG00000140995.17:t:89954172-89954376;89949513-89954243), DGKD (ENSG00000077044.11:s:233390403-233390483;233390483-233392070), DGKZ (ENSG00000149091.15:t:46367291-46367399;46345585-46367291), DNM2 (ENSG00000079805.19:s:10795372-10795439;10795439-10797380), DOCK8 (ENSG00000107099.18:t:271627-271729;215029-271627), DUT (ENSG00000128951.14:t:48332103-48332737;48331479-48332268), DYRK1A (ENSG00000157540.22:s:37418905-37420384;37420384-37472684), DYSF (ENSG00000135636.16:t:71480883-71480938;71454086-71480883), EIF4G1 (ENSG00000114867.22:t:184317321-184317497;184314674-184317321), EIF4G3 (ENSG00000075151.24:t:20981048-20981227;20981227-20997601), EMC8 (ENSG00000131148.9:s:85781211-85781760;85779867-85781211), EPSTI1 (ENSG00000133106.15:s:42991978-42992271;42969177-42991978), EXOSC1 (ENSG00000171311.13:s:97438663-97438703;97437750-97438663), EXOSC1 (ENSG00000171311.13:t:97437700-97437750;97437750-97438663), F2RL3 (ENSG00000127533.4:s:16888999-16889298;16889298-16889577), FAM219A (ENSG00000164970.15:s:34405865-34405964;34402807-34405916), FAM3A (ENSG00000071889.17:s:154512823-154512936;154511871-154512823), FBXO44 (ENSG00000132879.14:t:11658736-11658871;11658628-11658736), FKBP1B (ENSG00000119782.14:s:24060814-24060926;24060926-24063019), FMNL3 (ENSG00000161791.14:t:49657082-49657190;49657190-49658442), FOXP1 (ENSG00000114861.24:t:71112536-71112637;71112637-71130498), G3BP1 (ENSG00000145907.16:t:151786572-151786715;151772036-151786603), GET (ENSG00000182093.16:t:39390698-39390803;39380556-39390698), GLUL (ENSG00000135821.19:t:182388572-182390304;182388750-182391175), GORASP1 (ENSG00000114745.15:s:39107479-39108063;39103553-39107479), GORASP1 (ENSG00000114745.15:t:39101016-39101102;39103553-39107479), GSE1 (ENSG00000131149.19:t:85648552-85648751;85556363-85648552), GSTT1 (ENSG00000277656.3:t:270997-271173;271173-278295), GUCD1 (ENSG00000138867.17:t:24548917-24549001;24549001-24555615), HDAC7 (ENSG00000061273.18:s:47796207-47796298;47796016-47796207), HES6 (ENSG00000144485.11:t:238239487-238239568;238239568-238239825), HUS1 (ENSG00000136273.13:s:47979410-47979615;47978816-47979410), IFI27 (ENSG00000275214.4:t:1230073-1230504;1229442-1230343), IGF2BP3 (ENSG00000136231.14:t:23342064-23342189;23342189-23343718), IKBKB (ENSG00000104365.16:t:42290156-42290273;42288728-42290156), IKBKG (ENSG00000269335.7:t:154551988-154552189;154542452-154551988), IKZF3 (ENSG00000161405.17:t:39777651-39777767;39777767-39788258), IL10RA (ENSG0000010324.12:t:117988382-117988502;117986534-117988382), IL15RA (ENSG00000134470.21:t:5960367-5960567;5960567-5963743), IMPDH1 (ENSG00000106348.18:t:128405767-128405948;128405865-128409289), INO80E (ENSG00000169592.15:s:30001414-30001575;30001530-30005221), INPP5D (ENSG00000281614.3:s:67445-67595;67595-71086), IP6K2 (ENSG00000068745.15:t:48694872-48695421;48695421-48717157), IRF7 (ENSG00000276561.4:s:144369-144427;144292-144369), IRF7 (ENSG00000276561.4:s:144676-144900;144424-144690), ITSN2 (ENSG00000198399.16:s:24246129-24246320;24242206-24246129), JAML (ENSG00000160593.19:t:118212407-118212561;118212561-118214824), JOSD2 (ENSG00000161677.12:t:50506378-50506572;50506572-50507574), KANK2 (ENSG00000197256.11:s:11195556-11195881;11194590-11195556), KANK2 (ENSG00000197256.11:t:11194553-11194590;11194590-11195556), KANSL3 (ENSG00000114982.19:s:96636739-96637228;96631543-96636921), KANSL3 (ENSG00000114982.19:t:96631312-96631543;96631543-96636921), KAT5 (ENSG00000172977.13:s:65712922-65713319;65713058-65713348), KATNB1 (ENSG00000140854.13:s:57755345-57755494;57755494-57755841), KDM5C (ENSG00000126012.13:s:53217796-53217966;53217274-53217796), KIAA1671 (ENSG00000197077.14:t:25170820-25170938;25049364-25170820), KIFC3 (ENSG00000140859.16:t:57772223-57772288;57772288-57785464), KIFC3 (ENSG00000140859.16:t:57798072-57798282;57798282-57802370), KMT2B (ENSG00000272333.8:t:35719784-35720940;35719541-35719784), LDB1 (ENSG00000198728.11:s:102109029-102109177;102108323-102109029), LDB2 (ENSG00000169744.13:s:16511981-16512104;16508686-16511981), LETMD1 (ENSG00000050426.16:s:51048301-51048518;51048478-51056350), LST1 (ENSG00000204482.11:s:31587117-31587318;31587318-31587641), LST1 (ENSG00000204482.11:t:31587944-31587966;31587318-31587944), LST1 (ENSG00000206433.10:s:2842938-2843143;2843139-2844386), LST1 (ENSG00000223465.8:s:2834852-2835057;2835053-2835376), LST1 (ENSG00000223465.8:t:2835679-2835701;2835053-2835679), LST1 (ENSG00000226182.8:t:3065231-3065253;3064605-3065231), LST1 (ENSG00000230791.8:s:2886832-2887014;2887014-2887225), LST1 (ENSG00000230791.8:t:2887225-2887247;2886599-2887225), LST1 (ENSG00000231048.8:s:2892158-2892363;2892359-2892682), LST1 (ENSG00000231048.8:t:2892985-2893007;2892359-2892985), LTB (ENSG00000204487.8:s:3059543-3059714;3058917-3059543), LTB (ENSG00000223448.7:s:2887297-2887468;2886671-2887297), LTB (ENSG00000238114.7:s:2881536-2881707;2880910-2881536), LTBP4 (ENSG00000090006.18:s:40619347-40619493;40619493-40622401), LTBP4 (ENSG00000090006.18:t:40619347-40619493;40614446-40619347), LTBP4 (ENSG00000090006.18:t:40623604-40623732;40623021-40623604), MAPK9 (ENSG00000050748.18:t:180269280-180269409;180269409-180279796), MARK2 (ENSG00000072518.22:s:63904786-63905043;63905043-63908260), MAST3 (ENSG00000099308.13:s:18146881-18147044;18147017-18147443), MAX (ENSG000000125952.20:t:65077193-65077428;65077428-65077913), MBNL1 (ENSG00000152601.18:t:152414941-152415111;152269092-152414941,) METTL9 (ENSG00000197006.15:s:21624931-21625233;21625115-21655227), MGLL (ENSG00000074416.16:t:127821694-127821838;127821838-127822309), MGRN1 (ENSG00000102858.13:s:4677463-4677572;4677572-4681550), MIEF1 (ENSG00000100335.15:s:39511849-39512026;39512026-39512232), MLX (ENSG00000108788.12:s:42567619-42567655;42567655-42568837), MMAB (ENSG00000139428.12:s:109561420-109561517;109561329-109561420), MPI (ENSG00000178802.18:s:74890005-74890201;74890089-74890527), MPRIP (ENSG00000133030.22:t:17142627-17142765;17138429-17142627), MRPL33 (ENSG00000243147.8:s:27772674-27772855;27772692-27779433), MSRB3 (ENSG00000174099.12:s:65278643-65278865;65278865-65326826), MTA1 (ENSG00000182979.18:s:105445418-105445511;105445511-105450058), MTA1 (ENSG00000182979.18:t:105450058-105450184;105445511-105450058), MTHFD2 (ENSG00000065911.13:t:74207704-74207826;74205889-74207704), MTMR12 (ENSG00000150712.11:s:32312758-32312987;32274122-32312758), MTMR12 (ENSG00000150712.11:t:32273980-32274122;32274122-32312758), NAP1 L4 (ENSG00000273562.4:t:175479-176694;176694-182308), NCOA2 (ENSG00000140396.13:t:70141179-70141399;70141399-70148273), NCOR2 (ENSG00000196498.14:s:124330845-124330898;124326370-124330845), NCOR2 (ENSG00000196498.14:s:124378237-124378384;124372610-124378237), NCOR2 (ENSG0000196498.14:t:124372022-124372610;124372610-124378237), NDRG2 (ENSG00000165795.25:t:21022392-21022820;21022497-21022864), NDUFV3 (ENSG00000160194.18:t:42908864-42913304;42897047-42908864), NFE2L1 (ENSG00000082641.16:s:48056386-48056598;48056598-48057032), NFIA (ENSG00000162599.18:t:61455303-61462788;61406727-61455303), NKIRAS2 (ENSG00000168256.18:t:42023654-42025644;42022640-42023654), NOC2L (ENSG00000188976.11:s:945042-945146;944800-945057), NTAN1 (ENSG00000275779.4:s:613600-613788;613788-621580), NTPCR (ENSG00000135778.12:t:232956347-232956443;232950744-232956347), NUDT22 (ENSG00000149761.9:t:64229131-64229344;64227132-64229247), NUP214 (ENSG00000126883.19:s:131146129-131146304;131146304-131147490), NXT2 (ENSG00000101888.12:t:109538045-109538131;109537270-109538045), OCEL1 (ENSG00000099330.9:s:17226213-17226353;17226353-17226693), P2RX4 (ENSG00000135124.16:s:121210065-121210298;121210298-121217134), PABIR2 (ENSG0000156504.17:s:134781821-134781917;134772283-134781821), PALM2AKAP2 (ENSG00000157654.19:s:110138171-110138539;110138539-110168399), PANK4 (ENSG00000157881.16:t:2521101-2521315;2521315-2526464), PAPOLA (ENSG00000090060.19:s:96556175-96556413;96556413-96560649), PARL (ENSG00000175193.14:s:183862607-183862801;183844326-183862697), PARL (ENSG00000175193.14:t:183844231-183844774;183844326-183862753), PARVB (ENSG00000188677.15:t:44093928-44094017;44069162-44093928), PCYT1B (ENSG00000102230.14:t:24618985-24619084;24619084-24646989), PDE7A (ENSG00000205268.11:t:65782783-65782843;65782843-65841371), PEX26 (ENSG00000215193.14:s:18083437-18083732;18083732-18087972), PFKFB3 (ENSG00000170525.21:t:6213623-6213748;6203336-6213623), PKN1 (ENSG00000123143.13:t:14441143-14441443;14433542-14441143), PLEKHM1 (ENSG00000225190.12:s:45481603-45482612;45478147-45482433), PLS3 (ENSG00000102024.19:t:115610243-115610323;115561260-115610243), PML (ENSG00000140464.20:s:74034478-74034558;74034530-74042989), PML (ENSG00000140464.20:t:74033156-74033422;74032715-74033156), POLR2J3 (ENSG00000168255.20:t:102566997-102567083;102567083-102568010), POLR2J3 (ENSG00000285437.2:t:102566966-102567083;102567083-102568010), PPIE (ENSG00000084072.17:s:39752910-39753266;39753052-39753287), PPM1N (ENSG00000213889.11:t:45499949-45500066;45497343-45499949), PPP6R2 (ENSG00000100239.16:s:50436367-50436452;50436452-50436988), PPP6R2 (ENSG00000100239.16:t:50437506-50437603;50437068-50437506), PRKAR1B (ENSG00000188191.16:t:596146-596304;596304-602553), PRKCD (ENSG00000163932.16:t:53178404-53178537;53165215-53178404), PSEN1 (ENSG00000080815.20:t:73170797-73171923;73148094-73170797), PSMB8 (ENSG00000230034.8:t:4086512-4086659;4086659-4087849), PSMG4 (ENSG00000180822.12:s:3263684-3263759;3263759-3264209), PTK2B (ENSG00000120899.18:t:27450749-27450895;27445919-27450749), PTPN12 (ENSG00000127947.16:s:77600664-77600965;77600806-77607235), PTPN18 (ENSG00000072135.13:t:130368897-130369201;130356200-130369133), PUF60 (ENSG00000179950.15:s:143821118-143821743;143818534-143821597), PUF60 (ENSG00000179950.15:s:143821118-143821743;143820716-143821597), PUM1 (ENSG00000134644.16:s:30967099-30967310;30966278-30967167), PYM1 (ENSG00000170473.17:t:55903387-55903480;55903480-55927076), RAB11FIP1 (ENSG00000156675.16:t:37870387-37870528;37870528-37871278), RAB11FIP5 (ENSG00000135631.17:t:73075993-73076182;73076182-73088050), RAB4A (ENSG00000168118.12:t:229295848-229295910;229271370-229295848), RABGAP1L (ENSG00000152061.24:t:174969277-174969387;174957549-174969277), RAP1B (ENSG00000127314.18:t:68650244-68650882;68648781-68650400), RARA (ENSG00000131759.18:t:40348316-40348464;40331396-40348316), RBCK1 (ENSG00000125826.22:s:409830-410025;410025-417526), RBM10 (ENSG00000182872.16:t:47173128-47173197;47169498-47173128), RBM39 (ENSG00000131051.24:s:35713019-35713203;35709256-35713019), RCOR3 (ENSG00000117625.14:t:211313424-211316385;211312961-211313424), REPS2 (ENSG0000169891.18:s:17022123-17022371;17022271-17025059), RFFL (ENSG0000092871.17:s:35026374-35026568;35021781-35026374), RHD (ENSG00000187010.21:s:25303322-25303459;25303459-25328898), RMND5B (ENSG00000145916.19:t:178138108-178138396;178131054-178138108), RPUSD1 (ENSG00000007376.8:t:786826-786928;786928-787077), SEC16A (ENSG00000148396.19:s:136447227-136447364;136446949-136447227), SIGLEC10 (ENSG00000142512.15:t:51414422-51414515;51414515-51415181), SIRT3 (ENSG00000142082.15:s:218778-219041;216718-218778), SLA2 (ENSG00000101082.14:s:36615225-36615374;36614387-36615225), SLC66A2 (ENSG00000122490.19:t:79903446-79904183;79904183-79919184), SMARCA4 (ENSG00000127616.21:s:11034914-11035132;11035132-11041298), SMARCA4 (ENSG00000127616.21:t:11040634-11041560;11035132-11041298), SMARCC2 (ENSG00000139613.12:s:56173696-56173849;56173029-56173696), SNX20 (ENSG00000167208.16:s:50675770-50675921;50669148-50675770), SPIB (ENSG00000269404.7:s:50423605-50423755;50423751-50428038), SRSF6 (ENSG00000124193.16:s:43458361-43458509;43458509-43459153), SSX2IP (ENSG00000117155.17:t:84670646-84670815;84670815-84690371), STAMBP (ENSG00000124356.17:t:73830845-73831059;73829070-73830845), STAT3 (ENSG00000168610.16:t:42317182-42317224;42317224-42319856), SWI5 (ENSG00000175854.15:t:128276587-128276755;128276402-128276707), SYNE1 (ENSG00000131018.25:s:152301869-152302269;152300781-152301869), TANGO2 (ENSG00000183597.16:t:20061530-20061802;20056013-20061530), TCF3 (ENSG00000071564.19:s:1615686-1615804;1612433-1615686), TCF7L2 (ENSG00000148737.18:s:113146011-113146097;113146097-113150998), TECR (ENSG00000099797.15:s:14562356-14562575;14562575-14563174), TJP2 (ENSG00000119139.21:t:69212548-69212601;69151771-69212548), TLE4 (ENSG00000106829.21:s:79627374-79627752;79627448-79652593), TLE4 (ENSG00000106829.21:t:79652590-79652629;79627448-79652593), TMBIM1 (ENSG00000135926.15:s:218292466-218292586;218282181-218292466), TMBIM4 (ENSG00000155957.19:s:66169855-66170027;66161172-66169855), TMEM11 (ENSG00000178307.10:s:21214091-21214176;21211227-21214091), TMEM126B (ENSG00000171204.13:t:85631687-85631808;85628688-85631687), TMEM219 (ENSG00000149932.17:t:29962677-29963308;29962132-29963107), TNFSF12 (ENSG00000239697.12:t:7549466-7549618;7549312-7549474), TNK2 (ENSG00000061938.21:t:195888426-195888606;195888606-195908485), TNRC18 (ENSG00000182095.15:t:5325096-5325574;5325248-5329937), TRAPPC2 (ENSG00000196459.15:s:13734525-13734635;13719982-13734525), TRAPPC6A (ENSG00000007255.10:t:45164725-45164970;45164970-45165127), TSC22D3 (ENSG00000157514.18:t:107715773-107715950;107715950-107775100), TSPAN32 (ENSG00000064201.16:s:2314485-2314666;2314571-2316229), UBAC2 (ENSG00000134882.16:t:99238427-99238554;99200939-99238427), UFD1 (ENSG00000070010.19:t:19471687-19471808;19471808-19479083), URI1 (ENSG00000105176.18:s:29942239-29942664;29942664-29985223), URI1 (ENSG00000105176.18:t:29985223-29985301;29942664-29985223), USP15 (ENSG00000135655.16:s:62325872-62325933;62325933-62349221), USP22 (ENSG00000124422.12:t:21028542-21028674;21028674-21043321), VGLL4 (ENSG00000144560.16:t:11601833-11602022;11602022-11702971), WWP2 (ENSG00000198373.13:t:69786996-69787080;69762391-69786996), YAF2 (ENSG00000015153.15:s:42237599-42237800;42199235-42237599), YIPF6 (ENSG00000181704.12:s:68498562-68499157;68499123-68511849), YIPF6 (ENSG00000181704.12:t:68513327-68515340;68511977-68513327), ZBTB7B (ENSG00000160685.14:t:155014448-155015814;155002943-155014655), ZEB2 (ENSG00000169554.23:t:144517278-144517557;144517419-144517612), ZFAND1 (ENSG00000104231.11:s:81718182-81718224;81715114-81718182), ZFAND1 (ENSG00000104231.11:t:81714987-81715114;81715114-81718182), ZFAND2B (ENSG00000158552.13:s:219207648-219207779;219207774-219207887), ZMIZ2 (ENSG00000122515.16:s:44759281-44759460;44759460-44760151), ZMYND8 (ENSG00000101040.20:t:47236326-47236516;47236516-47238758), ZNF185 (ENSG00000147394.20:s:152922720-152922809;152922809-152928575), and ZNF451 (ENSG00000112200.17:t:57099061-57099141;57090274-57099061). In some embodiments, the one or more genes comprise a member selected from the group consisting of: LDB2 (ENSG00000169744.13:s:16511981-16512104;16508686-16511981), JOSD2 (ENSG00000161677.12:t:50506378-50506572;50506572-50507574), KIFC3 (ENSG00000140859.16:t:57798072-57798282;57798282-57802370), DOCK8 (ENSG00000107099.18:t:271627-271729;215029-271627), METTL9 (ENSG00000197006.15:s:21624931-21625233;21625115-21655227), CCDC28B (ENSG00000160050.16:t:32204598-32204620;32204379-32204598), EXOSC1 (ENSG00000171311.13:s:97438663-97438703;97437750-97438663), CBFA2T3 (ENSG00000129993.15:t:88892244-88892485;88892485-88898078), PUF60 (ENSG00000179950.15:s:143821118-143821743;143818534-143821597), NOC2L (ENSG00000188976.11:s:945042-945146;944800-945057), AP1B1 (ENSG00000100280.17:s:29330378-29330532;29329720-29330378), PPIE (ENSG00000084072.17:s:39752910-39753266;39753052-39753287), ARAP1 (ENSG00000186635.15:s:72693325-72693470;72688537-72693325), POLR2J3 (ENSG00000285437.2:t:102566966-102567083;102567083-102568010), DAZAP1 (ENSG00000071626.17:s:1422348-1422396;1422396-1425878), and ARHGEF1 (ENSG00000076928.19:s:41896377-41896878;41896482-41897315). In some embodiments, the one or more genes comprise a member selected from the group consisting of: SPIB (ENSG00000269404.7:s:50423605-50423755;50423751-50428038), KIFC3 (ENSG00000140859.16:t:57798072-57798282;57798282-57802370), LDB2 (ENSG00000169744.13:s:16511981-16512104;16508686-16511981), JOSD2 (ENSG00000161677.12:t:50506378-50506572;50506572-50507574), LST1 (ENSG00000230791.8:t:2887225-2887247;2886599-2887225), CCDC28B (ENSG00000160050.16:t:32204598-32204620;32204379-32204598), METTL9 (ENSG00000197006.15:s:21624931-21625233;21625115-21655227), PPIE (ENSG00000084072.17:s:39752910-39753266;39753052-39753287), CBFA2T3 (ENSG00000129993.15:t:88892244-88892485;88892485-88898078), TCF3 (ENSG00000071564.19:s:1615686-1615804;1612433-1615686), DCTD (ENSG00000129187.15:s:182917288-182917477;182915575-182917311), IL10RA (ENSG00000110324.12:t:117988382-117988502;117986534-117988382), ZNF451 (ENSG00000112200.17:t:57099061-57099141;57090274-57099061), AP1B1 (ENSG00000100280.17:s:29330378-29330532;29329720-29330378), EXOSC1 (ENSG00000171311.13:s:97438663-97438703;97437750-97438663), PUF60 (ENSG00000179950.15:s:143821118-143821743;143818534-143821597), POLR2J3 (ENSG00000285437.2:t:102566966-102567083;102567083-102568010), DOCK8 (ENSG00000107099.18:t:271627-271729;215029-271627), NOC2L (ENSG00000188976.11:s:945042-945146;944800-945057), NAP1L4 (ENSG00000273562.4:t:175479-176694;176694-182308), DAZAP1 (ENSG00000071626.17:s:1422348-1422396;1422396-1425878), ARAP1 (ENSG00000186635.15:s:72693325-72693470;72688537-72693325), RBM39 (ENSG00000131051.24:s:35713019-35713203;35709256-35713019), MSRB3 (ENSG00000174099.12:s:65278643-65278865;65278865-65326826), ZBTB7B (ENSG00000160685.14:t:155014448-155015814;155002943-155014655), NUP214 (ENSG00000126883.19:s:131146129-131146304;131146304-131147490), and ARHGEF1 (ENSG00000076928.19:s:41896377-41896878;41896482-41897315). In some embodiments, the one or more genes comprise a member selected from the group consisting of: ACAP1 (ENSG00000072818.12:t:7341948-7342067;7336787-7341948), ADGRE5 (ENSG00000123146.20:t:14397414-14397804;14391079-14397658), ADK (ENSG00000156110.15:t:74200764-74200838;74151343-74200764), ARHGEF1 (ENSG00000076928.19:s:41896377-41896878;41896482-41897315), ATXN2L (ENSG00000168488.19:t:28836728-28837237;28836485-28836728), CBFB (ENSG00000067955.15:t:67066682-67066962;67036755-67066682), CCDC92 (ENSG00000119242.9:s:123972529-123972831;123943493-123972529), DCTD (ENSG00000129187.15:s:182917288-182917477;182915575-182917311), DECR1 (ENSG00000104325.7:s:90001405-90001561;90001561-90017124), DOCK8 (ENSG00000107099.18:t:271627-271729;215029-271627), DYRK1A (ENSG00000157540.22:s:37418905-37420384;37420384-37472684), EXOSC1 (ENSG00000171311.13:s:97438663-97438703;97437750-97438663), FAM219A (ENSG00000164970.15:s:34405865-34405964;34402807-34405916), GLUL (ENSG00000135821.19:t:182388572-182390304;182388750-182391175), GSE1 (ENSG00000131149.19:t:85648552-85648751;85556363-85648552), GUCD1 (ENSG00000138867.17:t:24548917-24549001;24549001-24555615), LST1 (ENSG00000230791.8:t:2887225-2887247;2886599-2887225), MAST3 (ENSG00000099308.13:s:18146881-18147044;18147017-18147443), METTL9 (ENSG00000197006.15:s:21624931-21625233;21625115-21655227), MPRIP (ENSG00000133030.22:t:17142627-17142765;17138429-17142627), MRPL33 (ENSG00000243147.8:s:27772674-27772855;27772692-27779433), NAP1L4 (ENSG00000273562.4:t:175479-176694;176694-182308), NCOR2 (ENSG00000196498.14:s:124330845-124330898;124326370-124330845), NDRG2 (ENSG00000165795.25:t:21022392-21022820;21022497-21022864), NKIRAS2 (ENSG00000168256.18:t:42023654-42025644;42022640-42023654), PDE7A (ENSG00000205268.11:t:65782783-65782843;65782843-65841371), PKN1 (ENSG00000123143.13:t:14441143-14441443;14433542-14441143), PLEKHM1 (ENSG00000225190.12:s:45481603-45482612;45478147-45482433), PTPN12 (ENSG00000127947.16:s:77600664-77600965;77600806-77607235), RAB11FIP1 (ENSG00000156675.16:t:37870387-37870528;37870528-37871278), RCOR3 (ENSG00000117625.14:t:211313424-211316385;211312961-211313424), RFFL (ENSG00000092871.17:s:35026374-35026568;35021781-35026374), SPIB (ENSG00000269404.7:s:50423605-50423755;50423751-50428038), SSX2IP (ENSG00000117155.17:t:84670646-84670815;84670815-84690371), TMEM219 (ENSG00000149932.17:t:29962677-29963308;29962132-29963107), TNFSF12 (ENSG00000239697.12:t:7549466-7549618;7549312-7549474), TRAPPC2 (ENSG00000196459.15:s:13734525-13734635;13719982-13734525), USP15 (ENSG00000135655.16:s:62325872-62325933;62325933-62349221), YIPF6 (ENSG00000181704.12:t:68513327-68515340;68511977-68513327), ZFAND2B (ENSG00000158552.13:s:219207648-219207779;219207774-219207887), ZMYND8 (ENSG00000101040.20:t:47236326-47236516;47236516-47238758), and ZNF451 (ENSG00000112200.17:t:57099061-57099141;57090274-57099061. In some embodiments, the one or more splice junctions comprise one or more isoforms. In some embodiments, the one or more splice junctions comprise exon-exon junctions, exon-intron junctions, or intron-intron junctions. In some embodiments, the method of determining the risk of the disease comprises an accuracy of 70% or more. In some embodiments, the method of determining the risk of the disease comprises an accuracy of 80% or more. In some embodiments, the method of determining the risk of the disease comprises an accuracy of 90% or more. In some embodiments, the method of determining the risk of the disease comprises a sensitivity of 70% or more. In some embodiments, the method of determining the risk of the disease comprises a sensitivity of 75% or more. In some embodiments, the method of determining the risk of the disease comprises a sensitivity of 80% or more.

In another aspect, provided herein are methods of assessing an effect of a compound, the method comprising: assaying a first expression profile of a first cell-free biological sample obtained or derived from a subject at a first time point, to detect a first set of splice junctions; administering the compound to the subject; assaying a second expression profile of a second cell-free biological sample obtained or derived from the subject at a second time point subsequent to the administering, to detect a second set of splice junctions; computer processing the detected first and second sets of splice junctions; and assessing the effect of the compound based at least in part on the computer processing. In some embodiments, the assaying the first expression profile comprises one or more of sequencing, array hybridization, or nucleic acid amplification. In some embodiments, the assaying the second expression profile comprises one or more of sequencing, array hybridization, or nucleic acid amplification. In some embodiments, the sequencing comprises next generation sequencing (NGS). In some embodiments, the NGS comprises RNA sequencing. In some embodiments, the method further comprises comparing the detected first and second sets of splice junctions. In some embodiments, the method further comprises determining a difference between the detected first and second sets of splice junctions. In some embodiments, the difference indicates the effect of the compound. In some embodiments, the difference comprises one or more expressed splice junctions. In some embodiments, the one or more expressed splice junctions comprises a member selected from the group consisting of: ABLIM1 (ENSG00000099204.21:t:114491791-114491878;114491878-114545005), ACAA1 (ENSG00000060971.19:s:38126493-38126700;38126341-38126510), ACAP1 (ENSG00000072818.12:t:7341948-7342067;7336787-7341948), ACOT8 (ENSG00000101473.17:t:45848450-45849110;45848675-45857188), ACP1 (ENSG00000143727.16:t:272192-273155;272065-272192), ADD3 (ENSG00000148700.15:t:110100625-110100848;110008299-110100625), ADGRE5 (ENSG00000123146.20:t:14397414-14397804;14391079-14397658), ADK (ENSG00000156110.15:t:74200764-74200838;74151343-74200764), AGAP3 (ENSG00000133612.19:s:151119987-151120145;151120145-151122725), AKAP13 (ENSG00000170776.22:t:85575131-85575329;85543955-85575131), ALAD (ENSG00000148218.16:s:113393161-113393634;113392169-113393447), AMPD2 (ENSG00000116337.20:t:109625303-109625433;109621266-109625303), ANAPC11 (ENSG00000141552.18:t:81894298-81894624;81891833-81894467), AP1B1 (ENSG00000100280.17:s:29330378-29330532;29329720-29330378), AP1B1 (ENSG00000100280.17:t:29327680-29328895;29328895-29329712), APP (ENSG00000142192.21:s:26000015-26000182;25982477-26000015), APP (ENSG00000142192.21:t:25897573-25897983;25897673-25911741), ARAP1 (ENSG00000186635.15:s:72693325-72693470;72688537-72693325), ARFRP1 (ENSG00000101246.20:t:63706999-63707658;63707097-63707867), ARHGAP17 (ENSG00000140750.17:t:24935470-24936827;24935639-24941987), ARHGAP17 (ENSG00000288353.1:t:188953-190310;189122-195470), ARHGEF10 (ENSG00000274726.4:s:43270-43560;43560-47749), ARHGEF1 (ENSG00000076928.19:s:41896377-41896878;41896482-41897315), ARHGEF7 (ENSG00000102606.20:t:111217679-111217885;111210002-111217679), ARMC10 (ENSG00000170632.14:s:103074881-103075411;103075411-103075777), ARRDC2 (ENSG00000105643.11:t:18008711-18008861;18001573-18008711), ATE1 (ENSG00000107669.19:t:121924266-121924329;121924329-121928347), ATP6V1D (ENSG00000100554.12:t:67350611-67350690;67350690-67359658), ATXN2L (ENSG00000168488.19:s:28835933-28836176;28836122-28836748), ATXN2L (ENSG00000168488.19:t:28836728-28837237;28836122-28836748), ATXN2L (ENSG00000168488.19:t:28836728-28837237;28836485-28836728), AURKB (ENSG00000178999.13:t:8207738-8208225;8207840-8210530), BLOC1S6 (ENSG00000104164.12:s:45592135-45592276;45592276-45605428), C12orf76 (ENSG00000174456.16:s:110048363-110048870;110042459-110048363), CAMTA1 (ENSG00000171735.20:t:6825092-6825210;6820250-6825092), CBFA2T3 (ENSG00000129993.15:t:88892244-88892485;88892485-88898078), CBFB (ENSG00000067955.15:t:67066682-67066962;67036755-67066682), CCDC28B (ENSG00000160050.16:t:32204598-32204620;32204379-32204598), CCDC92 (ENSG00000119242.9:s:123972529-123972831;123943493-123972529), CCDC92 (ENSG00000119242.9:t:123943347-123943495;123943493-123972529), CD300H (ENSG00000284690.3:s:74563919-74564275;74560824-74563919), CD34 (ENSG00000174059.17:s:207888682-207888846;207887923-207888682), CDAN1 (ENSG00000140326.13:s:42736989-42737128;42736780-42737013), CDC14B (ENSG00000081377.17:t:96565393-96565512;96565483-96619219), CDK5RAP2 (ENSG00000136861.19:t:120402806-120403071;120403071-120403316), CELF2 (ENSG00000048740.19:t:11249153-11249201;11165682-11249153), CEP164 (ENSG00000110274.16:t:117409618-117410701;117409028-117409618), CLEC1B (ENSG00000165682.15:s:9995140-9995246;9986158-9995140), COX20 (ENSG00000203667.10:s:244835616-244835756;244835756-244842195), CPNE1 (ENSG00000214078.13:s:35627280-35627531;35626800-35627280), CYTH2 (ENSG00000105443.17:s:48474838-48476276;48474949-48477717), CYTH2 (ENSG00000105443.17:t:48478066-48478145;48477719-48478069), DAZAP1 (ENSG00000071626.17:s:1422348-1422396;1422396-1425878), DCTD (ENSG00000129187.15:s:182917288-182917477;182915575-182917311), DCTD (ENSG00000129187.15:t:182915461-182915575;182915575-182917288), DECR1 (ENSG00000104325.7:s:90001405-90001561;90001561-90017124), DECR1 (ENSG00000104325.7:s:90001405-90001561;90001561-90018909), DECR1 (ENSG00000104325.7:t:90018564-90018966;90001561-90018909), DEF8 (ENSG00000140995.17:t:89954172-89954376;89949513-89954243), DGKD (ENSG00000077044.11:s:233390403-233390483;233390483-233392070), DGKZ (ENSG00000149091.15:t:46367291-46367399;46345585-46367291), DNM2 (ENSG00000079805.19:s:10795372-10795439;10795439-10797380), DOCK8 (ENSG00000107099.18:t:271627-271729;215029-271627), DUT (ENSG00000128951.14:t:48332103-48332737;48331479-48332268), DYRK1A (ENSG00000157540.22:s:37418905-37420384;37420384-37472684), DYSF (ENSG00000135636.16:t:71480883-71480938;71454086-71480883), EIF4G1 (ENSG00000114867.22:t:184317321-184317497;184314674-184317321), EIF4G3 (ENSG00000075151.24:t:20981048-20981227;20981227-20997601), EMC8 (ENSG00000131148.9:s:85781211-85781760;85779867-85781211), EPSTI1 (ENSG00000133106.15:s:42991978-42992271;42969177-42991978), EXOSC1 (ENSG00000171311.13:s:97438663-97438703;97437750-97438663), EXOSC1 (ENSG00000171311.13:t:97437700-97437750;97437750-97438663), F2RL3 (ENSG00000127533.4:s:16888999-16889298;16889298-16889577), FAM219A (ENSG00000164970.15:s:34405865-34405964;34402807-34405916), FAM3A (ENSG00000071889.17:s:154512823-154512936;154511871-154512823), FBXO44 (ENSG00000132879.14:t:11658736-11658871;11658628-11658736), FKBP1B (ENSG00000119782.14:s:24060814-24060926;24060926-24063019), FMNL3 (ENSG00000161791.14:t:49657082-49657190;49657190-49658442), FOXP1 (ENSG00000114861.24:t:71112536-71112637;71112637-71130498), G3BP1 (ENSG00000145907.16:t:151786572-151786715;151772036-151786603), GET (ENSG00000182093.16:t:39390698-39390803;39380556-39390698), GLUL (ENSG00000135821.19:t:182388572-182390304;182388750-182391175), GORASP1 (ENSG00000114745.15:s:39107479-39108063;39103553-39107479), GORASP1 (ENSG00000114745.15:t:39101016-39101102;39103553-39107479), GSE1 (ENSG00000131149.19:t:85648552-85648751;85556363-85648552), GSTT1 (ENSG00000277656.3:t:270997-271173;271173-278295), GUCD1 (ENSG00000138867.17:t:24548917-24549001;24549001-24555615), HDAC7 (ENSG00000061273.18:s:47796207-47796298;47796016-47796207), HES6 (ENSG00000144485.11:t:238239487-238239568;238239568-238239825), HUS1 (ENSG00000136273.13:s:47979410-47979615;47978816-47979410), IFI27 (ENSG00000275214.4:t:1230073-1230504;1229442-1230343), IGF2BP3 (ENSG00000136231.14:t:23342064-23342189;23342189-23343718), IKBKB (ENSG00000104365.16:t:42290156-42290273;42288728-42290156), IKBKG (ENSG00000269335.7:t:154551988-154552189;154542452-154551988), IKZF3 (ENSG00000161405.17:t:39777651-39777767;39777767-39788258), IL10RA (ENSG0000010324.12:t:117988382-117988502;117986534-117988382), IL15RA (ENSG00000134470.21:t:5960367-5960567;5960567-5963743), IMPDH1 (ENSG00000106348.18:t:128405767-128405948;128405865-128409289), INO80E (ENSG00000169592.15:s:30001414-30001575;30001530-30005221), INPP5D (ENSG00000281614.3:s:67445-67595;67595-71086), IP6K2 (ENSG00000068745.15:t:48694872-48695421;48695421-48717157), IRF7 (ENSG00000276561.4:s:144369-144427;144292-144369), IRF7 (ENSG00000276561.4:s:144676-144900;144424-144690), ITSN2 (ENSG00000198399.16:s:24246129-24246320;24242206-24246129), JAML (ENSG00000160593.19:t:118212407-118212561;118212561-118214824), JOSD2 (ENSG00000161677.12:t:50506378-50506572;50506572-50507574), KANK2 (ENSG00000197256.11:s:11195556-11195881;11194590-11195556), KANK2 (ENSG00000197256.11:t:11194553-11194590;11194590-11195556), KANSL3 (ENSG00000114982.19:s:96636739-96637228;96631543-96636921), KANSL3 (ENSG00000114982.19:t:96631312-96631543;96631543-96636921), KAT5 (ENSG00000172977.13:s:65712922-65713319;65713058-65713348), KATNB1 (ENSG00000140854.13:s:57755345-57755494;57755494-57755841), KDM5C (ENSG00000126012.13:s:53217796-53217966;53217274-53217796), KIAA1671 (ENSG00000197077.14:t:25170820-25170938;25049364-25170820), KIFC3 (ENSG00000140859.16:t:57772223-57772288;57772288-57785464), KIFC3 (ENSG00000140859.16:t:57798072-57798282;57798282-57802370), KMT2B (ENSG00000272333.8:t:35719784-35720940;35719541-35719784), LDB1 (ENSG00000198728.11:s:102109029-102109177;102108323-102109029), LDB2 (ENSG00000169744.13:s:16511981-16512104;16508686-16511981), LETMD1 (ENSG00000050426.16:s:51048301-51048518;51048478-51056350), LST1 (ENSG00000204482.11:s:31587117-31587318;31587318-31587641), LST1 (ENSG00000204482.11:t:31587944-31587966;31587318-31587944), LST1 (ENSG00000206433.10:s:2842938-2843143;2843139-2844386), LST1 (ENSG00000223465.8:s:2834852-2835057;2835053-2835376), LST1 (ENSG00000223465.8:t:2835679-2835701;2835053-2835679), LST1 (ENSG00000226182.8:t:3065231-3065253;3064605-3065231), LST1 (ENSG00000230791.8:s:2886832-2887014;2887014-2887225), LST1 (ENSG00000230791.8:t:2887225-2887247;2886599-2887225), LST1 (ENSG00000231048.8:s:2892158-2892363;2892359-2892682), LST1 (ENSG00000231048.8:t:2892985-2893007;2892359-2892985), LTB (ENSG00000204487.8:s:3059543-3059714;3058917-3059543), LTB (ENSG00000223448.7:s:2887297-2887468;2886671-2887297), LTB (ENSG00000238114.7:s:2881536-2881707;2880910-2881536), LTBP4 (ENSG00000090006.18:s:40619347-40619493;40619493-40622401), LTBP4 (ENSG00000090006.18:t:40619347-40619493;40614446-40619347), LTBP4 (ENSG00000090006.18:t:40623604-40623732;40623021-40623604), MAPK9 (ENSG00000050748.18:t:180269280-180269409;180269409-180279796), MARK2 (ENSG00000072518.22:s:63904786-63905043;63905043-63908260), MAST3 (ENSG00000099308.13:s:18146881-18147044;18147017-18147443), MAX (ENSG00000125952.20:t:65077193-65077428;65077428-65077913), MBNL1 (ENSG00000152601.18:t:152414941-152415111;152269092-152414941,) METTL9 (ENSG00000197006.15:s:21624931-21625233;21625115-21655227), MGLL (ENSG00000074416.16:t:127821694-127821838;127821838-127822309), MGRN1 (ENSG00000102858.13:s:4677463-4677572;4677572-4681550), MIEF1 (ENSG00000100335.15:s:39511849-39512026;39512026-39512232), MLX (ENSG00000108788.12:s:42567619-42567655;42567655-42568837), MMAB (ENSG00000139428.12:s:109561420-109561517;109561329-109561420), MPI (ENSG00000178802.18:s:74890005-74890201;74890089-74890527), MPRIP (ENSG00000133030.22:t:17142627-17142765;17138429-17142627), MRPL33 (ENSG00000243147.8:s:27772674-27772855;27772692-27779433), MSRB3 (ENSG00000174099.12:s:65278643-65278865;65278865-65326826), MTA1 (ENSG00000182979.18:s:105445418-105445511;105445511-105450058), MTA1 (ENSG00000182979.18:t:105450058-105450184;105445511-105450058), MTHFD2 (ENSG00000065911.13:t:74207704-74207826;74205889-74207704), MTMR12 (ENSG00000150712.11:s:32312758-32312987;32274122-32312758), MTMR12 (ENSG00000150712.11:t:32273980-32274122;32274122-32312758), NAP1 L4 (ENSG00000273562.4:t:175479-176694;176694-182308), NCOA2 (ENSG00000140396.13:t:70141179-70141399;70141399-70148273), NCOR2 (ENSG00000196498.14:s:124330845-124330898;124326370-124330845), NCOR2 (ENSG00000196498.14:s:124378237-124378384;124372610-124378237), NCOR2 (ENSG00000196498.14:t:124372022-124372610;124372610-124378237), NDRG2 (ENSG00000165795.25:t:21022392-21022820;21022497-21022864), NDUFV3 (ENSG00000160194.18:t:42908864-42913304;42897047-42908864), NFE2L1 (ENSG00000082641.16:s:48056386-48056598;48056598-48057032), NFIA (ENSG00000162599.18:t:61455303-61462788;61406727-61455303), NKIRAS2 (ENSG00000168256.18:t:42023654-42025644;42022640-42023654), NOC2L (ENSG00000188976.11:s:945042-945146;944800-945057), NTAN1 (ENSG00000275779.4:s:613600-613788;613788-621580), NTPCR (ENSG00000135778.12:t:232956347-232956443;232950744-232956347), NUDT22 (ENSG00000149761.9:t:64229131-64229344;64227132-64229247), NUP214 (ENSG00000126883.19:s:131146129-131146304;131146304-131147490), NXT2 (ENSG00000101888.12:t:109538045-109538131;109537270-109538045), OCEL1 (ENSG00000099330.9:s:17226213-17226353;17226353-17226693), P2RX4 (ENSG00000135124.16:s:121210065-121210298;121210298-121217134), PABIR2 (ENSG0000156504.17:s:134781821-134781917;134772283-134781821), PALM2AKAP2 (ENSG00000157654.19:s:110138171-110138539;110138539-110168399), PANK4 (ENSG00000157881.16:t:2521101-2521315;2521315-2526464), PAPOLA (ENSG00000090060.19:s:96556175-96556413;96556413-96560649), PARL (ENSG00000175193.14:s:183862607-183862801;183844326-183862697), PARL (ENSG00000175193.14:t:183844231-183844774;183844326-183862753), PARVB (ENSG00000188677.15:t:44093928-44094017;44069162-44093928), PCYT1B (ENSG00000102230.14:t:24618985-24619084;24619084-24646989), PDE7A (ENSG00000205268.11:t:65782783-65782843;65782843-65841371), PEX26 (ENSG00000215193.14:s:18083437-18083732;18083732-18087972), PFKFB3 (ENSG00000170525.21:t:6213623-6213748;6203336-6213623), PKN1 (ENSG00000123143.13:t:14441143-14441443;14433542-14441143), PLEKHM1 (ENSG00000225190.12:s:45481603-45482612;45478147-45482433), PLS3 (ENSG00000102024.19:t:115610243-115610323;115561260-115610243), PML (ENSG00000140464.20:s:74034478-74034558;74034530-74042989), PML (ENSG00000140464.20:t:74033156-74033422;74032715-74033156), POLR2J3 (ENSG00000168255.20:t:102566997-102567083;102567083-102568010), POLR2J3 (ENSG00000285437.2:t:102566966-102567083;102567083-102568010), PPIE (ENSG00000084072.17:s:39752910-39753266;39753052-39753287), PPM1N (ENSG00000213889.11:t:45499949-45500066;45497343-45499949), PPP6R2 (ENSG00000100239.16:s:50436367-50436452;50436452-50436988), PPP6R2 (ENSG00000100239.16:t:50437506-50437603;50437068-50437506), PRKAR1B (ENSG00000188191.16:t:596146-596304;596304-602553), PRKCD (ENSG00000163932.16:t:53178404-53178537;53165215-53178404), PSEN1 (ENSG00000080815.20:t:73170797-73171923;73148094-73170797), PSMB8 (ENSG00000230034.8:t:4086512-4086659;4086659-4087849), PSMG4 (ENSG00000180822.12:s:3263684-3263759;3263759-3264209), PTK2B (ENSG00000120899.18:t:27450749-27450895;27445919-27450749), PTPN12 (ENSG00000127947.16:s:77600664-77600965;77600806-77607235), PTPN18 (ENSG00000072135.13:t:130368897-130369201;130356200-130369133), PUF60 (ENSG00000179950.15:s:143821118-143821743;143818534-143821597), PUF60 (ENSG00000179950.15:s:143821118-143821743;143820716-143821597), PUM1 (ENSG00000134644.16:s:30967099-30967310;30966278-30967167), PYM1 (ENSG00000170473.17:t:55903387-55903480;55903480-55927076), RAB11FIP1 (ENSG00000156675.16:t:37870387-37870528;37870528-37871278), RAB11FIP5 (ENSG00000135631.17:t:73075993-73076182;73076182-73088050), RAB4A (ENSG00000168118.12:t:229295848-229295910;229271370-229295848), RABGAP1L (ENSG00000152061.24:t:174969277-174969387;174957549-174969277), RAP1B (ENSG00000127314.18:t:68650244-68650882;68648781-68650400), RARA (ENSG00000131759.18:t:40348316-40348464;40331396-40348316), RBCK1 (ENSG00000125826.22:s:409830-410025;410025-417526), RBM10 (ENSG00000182872.16:t:47173128-47173197;47169498-47173128), RBM39 (ENSG00000131051.24:s:35713019-35713203;35709256-35713019), RCOR3 (ENSG00000117625.14:t:211313424-211316385;211312961-211313424), REPS2 (ENSG0000169891.18:s:17022123-17022371;17022271-17025059), RFFL (ENSG0000092871.17:s:35026374-35026568;35021781-35026374), RHD (ENSG00000187010.21:s:25303322-25303459;25303459-25328898), RMND5B (ENSG00000145916.19:t:178138108-178138396;178131054-178138108), RPUSD1 (ENSG00000007376.8:t:786826-786928;786928-787077), SEC16A (ENSG00000148396.19:s:136447227-136447364;136446949-136447227), SIGLEC10 (ENSG00000142512.15:t:51414422-51414515;51414515-51415181), SIRT3 (ENSG00000142082.15:s:218778-219041;216718-218778), SLA2 (ENSG00000101082.14:s:36615225-36615374;36614387-36615225), SLC66A2 (ENSG00000122490.19:t:79903446-79904183;79904183-79919184), SMARCA4 (ENSG00000127616.21:s:11034914-11035132;11035132-11041298), SMARCA4 (ENSG00000127616.21:t:11040634-11041560;11035132-11041298), SMARCC2 (ENSG00000139613.12:s:56173696-56173849;56173029-56173696), SNX20 (ENSG00000167208.16:s:50675770-50675921;50669148-50675770), SPIB (ENSG00000269404.7:s:50423605-50423755;50423751-50428038), SRSF6 (ENSG00000124193.16:s:43458361-43458509;43458509-43459153), SSX2IP (ENSG00000117155.17:t:84670646-84670815;84670815-84690371), STAMBP (ENSG00000124356.17:t:73830845-73831059;73829070-73830845), STAT3 (ENSG00000168610.16:t:42317182-42317224;42317224-42319856), SWI5 (ENSG00000175854.15:t:128276587-128276755;128276402-128276707), SYNE1 (ENSG00000131018.25:s:152301869-152302269;152300781-152301869), TANGO2 (ENSG00000183597.16:t:20061530-20061802;20056013-20061530), TCF3 (ENSG00000071564.19:s:1615686-1615804;1612433-1615686), TCF7L2 (ENSG00000148737.18:s:113146011-113146097;113146097-113150998), TECR (ENSG00000099797.15:s:14562356-14562575;14562575-14563174), TJP2 (ENSG00000119139.21:t:69212548-69212601;69151771-69212548), TLE4 (ENSG00000106829.21:s:79627374-79627752;79627448-79652593), TLE4 (ENSG00000106829.21:t:79652590-79652629;79627448-79652593), TMBIM1 (ENSG00000135926.15:s:218292466-218292586;218282181-218292466), TMBIM4 (ENSG00000155957.19:s:66169855-66170027;66161172-66169855), TMEM11 (ENSG00000178307.10:s:21214091-21214176;21211227-21214091), TMEM126B (ENSG00000171204.13:t:85631687-85631808;85628688-85631687), TMEM219 (ENSG00000149932.17:t:29962677-29963308;29962132-29963107), TNFSF12 (ENSG00000239697.12:t:7549466-7549618;7549312-7549474), TNK2 (ENSG00000061938.21:t:195888426-195888606;195888606-195908485), TNRC18 (ENSG00000182095.15:t:5325096-5325574;5325248-5329937), TRAPPC2 (ENSG00000196459.15:s:13734525-13734635;13719982-13734525), TRAPPC6A (ENSG00000007255.10:t:45164725-45164970;45164970-45165127), TSC22D3 (ENSG00000157514.18:t:107715773-107715950;107715950-107775100), TSPAN32 (ENSG00000064201.16:s:2314485-2314666;2314571-2316229), UBAC2 (ENSG00000134882.16:t:99238427-99238554;99200939-99238427), UFD1 (ENSG00000070010.19:t:19471687-19471808;19471808-19479083), URI1 (ENSG00000105176.18:s:29942239-29942664;29942664-29985223), URI1 (ENSG00000105176.18:t:29985223-29985301;29942664-29985223), USP15 (ENSG00000135655.16:s:62325872-62325933;62325933-62349221), USP22 (ENSG00000124422.12:t:21028542-21028674;21028674-21043321), VGLL4 (ENSG00000144560.16:t:11601833-11602022;11602022-11702971), WWP2 (ENSG00000198373.13:t:69786996-69787080;69762391-69786996), YAF2 (ENSG00000015153.15:s:42237599-42237800;42199235-42237599), YIPF6 (ENSG00000181704.12:s:68498562-68499157;68499123-68511849), YIPF6 (ENSG00000181704.12:t:68513327-68515340;68511977-68513327), ZBTB7B (ENSG00000160685.14:t:155014448-155015814;155002943-155014655), ZEB2 (ENSG00000169554.23:t:144517278-144517557;144517419-144517612), ZFAND1 (ENSG00000104231.11:s:81718182-81718224;81715114-81718182), ZFAND1 (ENSG00000104231.11:t:81714987-81715114;81715114-81718182), ZFAND2B (ENSG00000158552.13:s:219207648-219207779;219207774-219207887), ZMIZ2 (ENSG00000122515.16:s:44759281-44759460;44759460-44760151), ZMYND8 (ENSG00000101040.20:t:47236326-47236516;47236516-47238758), ZNF185 (ENSG00000147394.20:s:152922720-152922809;152922809-152928575), and ZNF451 (ENSG00000112200.17:t:57099061-57099141;57090274-57099061). In some embodiments, the one or more expressed splice junctions comprise a member selected from the group consisting of: LDB2 (ENSG00000169744.13:s:16511981-16512104;16508686-16511981), JOSD2 (ENSG00000161677.12:t:50506378-50506572;50506572-50507574), KIFC3 (ENSG00000140859.16:t:57798072-57798282;57798282-57802370), DOCK8 (ENSG00000107099.18:t:271627-271729;215029-271627), METTL9 (ENSG00000197006.15:s:21624931-21625233;21625115-21655227), CCDC28B (ENSG00000160050.16:t:32204598-32204620;32204379-32204598), EXOSC1 (ENSG00000171311.13:s:97438663-97438703;97437750-97438663), CBFA2T3 (ENSG00000129993.15:t:88892244-88892485;88892485-88898078), PUF60 (ENSG00000179950.15:s:143821118-143821743;143818534-143821597), NOC2L (ENSG00000188976.11:s:945042-945146;944800-945057), AP1B1 (ENSG00000100280.17:s:29330378-29330532;29329720-29330378), PPIE (ENSG00000084072.17:s:39752910-39753266;39753052-39753287), ARAP1 (ENSG00000186635.15:s:72693325-72693470;72688537-72693325), POLR2J3 (ENSG00000285437.2:t:102566966-102567083;102567083-102568010), DAZAP1 (ENSG00000071626.17:s:1422348-1422396;1422396-1425878), and ARHGEF1 (ENSG00000076928.19:s:41896377-41896878;41896482-41897315). In some embodiments, the one or more expressed splice junctions comprise a member selected from the group consisting of: SPIB (ENSG00000269404.7:s:50423605-50423755;50423751-50428038), KIFC3 (ENSG00000140859.16:t:57798072-57798282;57798282-57802370), LDB2 (ENSG00000169744.13:s:16511981-16512104;16508686-16511981), JOSD2 (ENSG00000161677.12:t:50506378-50506572;50506572-50507574), LST1 (ENSG00000230791.8:t:2887225-2887247;2886599-2887225), CCDC28B (ENSG00000160050.16:t:32204598-32204620;32204379-32204598), METTL9 (ENSG00000197006.15:s:21624931-21625233;21625115-21655227), PPIE (ENSG00000084072.17:s:39752910-39753266;39753052-39753287), CBFA2T3 (ENSG00000129993.15:t:88892244-88892485;88892485-88898078), TCF3 (ENSG00000071564.19:s:1615686-1615804;1612433-1615686), DCTD (ENSG00000129187.15:s:182917288-182917477;182915575-182917311), IL10RA (ENSG00000110324.12:t:117988382-117988502;117986534-117988382), ZNF451 (ENSG00000112200.17:t:57099061-57099141;57090274-57099061), AP1B1 (ENSG00000100280.17:s:29330378-29330532;29329720-29330378), EXOSC1 (ENSG00000171311.13:s:97438663-97438703;97437750-97438663), PUF60 (ENSG00000179950.15:s:143821118-143821743;143818534-143821597), POLR2J3 (ENSG00000285437.2:t:102566966-102567083;102567083-102568010), DOCK8 (ENSG00000107099.18:t:271627-271729;215029-271627), NOC2L (ENSG00000188976.11:s:945042-945146;944800-945057), NAP1L4 (ENSG00000273562.4:t:175479-176694;176694-182308), DAZAP1 (ENSG00000071626.17:s:1422348-1422396;1422396-1425878), ARAP1 (ENSG00000186635.15:s:72693325-72693470;72688537-72693325), RBM39 (ENSG00000131051.24:s:35713019-35713203;35709256-35713019), MSRB3 (ENSG00000174099.12:s:65278643-65278865;65278865-65326826), ZBTB7B (ENSG00000160685.14:t:155014448-155015814;155002943-155014655), NUP214 (ENSG00000126883.19:s:131146129-131146304;131146304-131147490), and ARHGEF1 (ENSG00000076928.19:s:41896377-41896878;41896482-41897315). In some embodiments, the one or more expressed splice junctions comprise a member selected from the group consisting of: ACAP1 (ENSG00000072818.12:t:7341948-7342067;7336787-7341948), ADGRE5 (ENSG00000123146.20:t:14397414-14397804;14391079-14397658), ADK (ENSG00000156110.15:t:74200764-74200838;74151343-74200764), ARHGEF1 (ENSG00000076928.19:s:41896377-41896878;41896482-41897315), ATXN2L (ENSG00000168488.19:t:28836728-28837237;28836485-28836728), CBFB (ENSG00000067955.15:t:67066682-67066962;67036755-67066682), CCDC92 (ENSG00000119242.9:s:123972529-123972831;123943493-123972529), DCTD (ENSG00000129187.15:s:182917288-182917477;182915575-182917311), DECR1 (ENSG00000104325.7:s:90001405-90001561;90001561-90017124), DOCK8 (ENSG00000107099.18:t:271627-271729;215029-271627), DYRK1A (ENSG00000157540.22:s:37418905-37420384;37420384-37472684), EXOSC1 (ENSG00000171311.13:s:97438663-97438703;97437750-97438663), FAM219A (ENSG00000164970.15:s:34405865-34405964;34402807-34405916), GLUL (ENSG00000135821.19:t:182388572-182390304;182388750-182391175), GSE1 (ENSG00000131149.19:t:85648552-85648751;85556363-85648552), GUCD1 (ENSG00000138867.17:t:24548917-24549001;24549001-24555615), LST1 (ENSG00000230791.8:t:2887225-2887247;2886599-2887225), MAST3 (ENSG00000099308.13:s:18146881-18147044;18147017-18147443), METTL9 (ENSG00000197006.15:s:21624931-21625233;21625115-21655227), MPRIP (ENSG00000133030.22:t:17142627-17142765;17138429-17142627), MRPL33 (ENSG00000243147.8:s:27772674-27772855;27772692-27779433), NAP1L4 (ENSG00000273562.4:t:175479-176694;176694-182308), NCOR2 (ENSG00000196498.14:s:124330845-124330898;124326370-124330845), NDRG2 (ENSG00000165795.25:t:21022392-21022820;21022497-21022864), NKIRAS2 (ENSG00000168256.18:t:42023654-42025644;42022640-42023654), PDE7A (ENSG00000205268.11:t:65782783-65782843;65782843-65841371), PKN1 (ENSG00000123143.13:t:14441143-14441443;14433542-14441143), PLEKHM1 (ENSG00000225190.12:s:45481603-45482612;45478147-45482433), PTPN12 (ENSG00000127947.16:s:77600664-77600965;77600806-77607235), RAB11FIP1 (ENSG00000156675.16:t:37870387-37870528;37870528-37871278), RCOR3 (ENSG00000117625.14:t:211313424-211316385;211312961-211313424), RFFL (ENSG00000092871.17:s:35026374-35026568;35021781-35026374), SPIB (ENSG00000269404.7:s:50423605-50423755;50423751-50428038), SSX2IP (ENSG00000117155.17:t:84670646-84670815;84670815-84690371), TMEM219 (ENSG00000149932.17:t:29962677-29963308;29962132-29963107), TNFSF12 (ENSG00000239697.12:t:7549466-7549618;7549312-7549474), TRAPPC2 (ENSG00000196459.15:s:13734525-13734635;13719982-13734525), USP15 (ENSG00000135655.16:s:62325872-62325933;62325933-62349221), YIPF6 (ENSG00000181704.12:t:68513327-68515340;68511977-68513327), ZFAND2B (ENSG00000158552.13:s:219207648-219207779;219207774-219207887), ZMYND8 (ENSG00000101040.20:t:47236326-47236516;47236516-47238758), and ZNF451 (ENSG00000112200.17:t:57099061-57099141;57090274-57099061). In some embodiments, the compound comprises a treatment for a disease state. In some embodiments, the disease state comprises a severity of the disease state. In some embodiments, the disease state comprises a presence or an absence of Alzheimer's disease. In some embodiments, the subject is suspected of having the Alzheimer's disease. In some embodiments, the treatment comprises a medicinal therapy. In some embodiments, the medicinal therapy comprises a cholinesterase inhibitor. In some embodiments, the medicinal therapy comprises a N-methyl-D-aspartate (NMDA) antagonist. In some embodiments, the medicinal therapy comprises an amyloid monoclonal antibody (mab) therapy.

In one aspect, provided herein are compositions of any one of the methods disclosed herein.

In another aspect, provided herein are kit comprising any of the compositions disclosed herein and instructions for use of the composition according to any of the methods disclosed herein.

In another aspect, provided herein are computer systems for detecting a disease state in a subject, the system comprising: a non-transitory memory; and a processor in communication with the non-transitory memory, the processor configured to execute the following operations in order to effectuate a method comprising the operations of: assaying cell-free messenger RNA (cf-mRNA) in a biological sample to determine a level of the cf-mRNA that contains a non-contiguous junction relative to genomic DNA, thereby detecting one or more splice junctions; processing the detected one or more splice junctions; and detecting the disease state of the subject based at least in part on the processing.

In another aspect, provided herein are non-transitory computer-readable memories storing one or more instructions executable by one or more processors, that when executed by the one or more processors cause the one or more processors to perform processing comprising: assaying cell-free messenger RNA (cf-mRNA) in a biological sample to determine a level of the cf-mRNA that contains a non-contiguous junction relative to genomic DNA, thereby detecting one or more splice junctions; processing the detected one or more splice junctions; and detecting the disease state of the subject based at least in part on the processing.

In another aspect, provided herein are computer systems for determining a risk of a disease state in a subject, the system comprising: a non-transitory memory; and a processor in communication with the non-transitory memory, the processor configured to execute the following operations in order to effectuate a method comprising the operations of: assaying cell-free messenger RNA (cf-mRNA) in a biological sample to detect one or more splice junctions in the cf-mRNA, wherein the one or more splice junctions correspond to one or more genes.

In another aspect, provided herein are non-transitory computer-readable memories storing one or more instructions executable by one or more processors, that when executed by the one or more processors cause the one or more processors to perform processing comprising: assaying cell-free messenger RNA (cf-mRNA) in a biological sample to detect one or more splice junctions in the cf-mRNA, wherein the one or more splice junctions correspond to one or more genes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of the features and advantages of the present inventive concepts will be obtained by reference to the following detailed description that sets forth illustrative embodiments, in which the principles of the inventive concepts are utilized, and the accompanying drawings of which:

FIG. 1A shows an analysis of splice junctions in cell-free mRNA (cf-mRNA) between a cohort of subjects with Alzheimer's disease and a cohort of non-cognitively impaired subjects;

FIG. 1B shows an analysis of splice junctions in cell-free mRNA (cf-mRNA) between a cohort of subjects with Alzheimer's disease and a cohort of non-cognitively impaired subjects;

FIG. 2 shows a receiver operating characteristic (ROC) curve with an area under the curve (AUC) value for a 16 splice junction classifier;

FIG. 3 shows a sigmoid curve for a 16 splice junction classifier;

FIG. 4 shows a receiver operating characteristic (ROC) curve with an area under the curve (AUC) value for a 42 splice junction classifier;

FIG. 5 shows a sigmoid curve for a 42 splice junction classifier;

FIG. 6 shows a receiver operating characteristic (ROC) curve with an area under the curve (AUC) value for a 27 splice junction classifier;

FIG. 7 shows a sigmoid curve for a 27 splice junction classifier; and

FIG. 8 shows a computer system that is programmed or otherwise configured to implement methods provided herein.

### DETAILED DESCRIPTION

Methods, systems, kits, and compositions disclosed herein relate to the rapid, noninvasive detection of disorders and cell-free messenger RNA (cf-mRNA) to identify mRNA splice junctions so as to concurrently determine both a likely disorder and a likely tissue under duress. Methods disclosed herein can take into account changes in gene expression brought about by clinical factors such as age, gender, and the like. In some embodiments, a gene panel comprised of gene splice junctions known to be differentially expressed in individuals in a cohort based on clinical factors is applied to a cf-mRNA expression profile of a subject. Through practice of the methods disclosed herein, one can make predictions as to a disease's identity and stage, as well as the extent of its impact on one or more tissues without invasive investigation of the tissue or tissues suspected of being impacted by the disease.

There is a need to develop a reliable and non-invasive test to accurately identify the molecular pathology or health state of a tissue, for example, a brain tissue, bone marrow tissue, liver tissue, kidney tissue, liver tissue, and heart tissue. There is further a need to identify early stages of neurodegenerative diseases such as Alzheimer's disease. Physicians often use a numeric scale, Clinical Dementia Rating (CDR), to quantify the severity of a neurodegenerative disorder. Further, the Folstein Mini-Mental State Exam (MMSE) may be used in clinical and research settings to measure cognitive impairment of subjects.

Alzheimer's disease is a neurodegenerative disorder marked by cognitive and behavioral impairment that significantly interferes with a subject's normal day to day function. Alzheimer's disease is the most common cause of dementia and affects a large portion of the elderly population globally. Further, Alzheimer's disease is a neurodegenerative condition generally characterized by the accumulation of amyloid-O peptide, deposition of tau proteins and neurofibrillary tangles, onset of synaptic and neuronal dysfunction, lipid metabolism disturbances, activation of inflammatory response caused by microglia, and mitochondria dysfunction.

The heterogeneous nature of Alzheimer's disease, as a complex neurodegenerative disease affecting multiple biological pathways and processes during preclinical Alzheimer's disease, clinical onset, and progression, represents one major difficulty for Alzheimer's disease drug development. An additional challenge is that in some cases, the underlying biological pathways are a mix of different types of dementias. So far, therapeutic drugs targeting O-amyloids and tau proteins have shown modest results. Therefore, multiple compounds targeting commonly affected pathways in Alzheimer's disease, such as inflammation, mitochondrial dysfunction, and neuroprotective compounds are currently being developed and tested as alternatives for Alzheimer's disease treatment.

Successful development of therapeutic agents for a heterogeneous Alzheimer's disease population may rely on the ability to appropriately enrich the trial groups for Alzheimer's disease patients likely to respond to the candidate drugs. Since molecular assessment of patients based on brain biopsy is generally not feasible, non-invasive tools enabling pre-selection of patients best suited for each therapy can be useful for clinical trials. The present disclosure indicates that the molecular information revealed by the circulating transcriptome including the expression of gene splice variants may pave the way to personalized and more precise characterization of disease-related processes, thus enabling more efficient patient management and improving the probabilities of success of the interventions.

Further, given that extracellular cell-free mRNA (cf-mRNA) can enable “real time” monitoring of organ health or organ molecular pathology and organ system response to therapeutic interventions, for example the repertoire of Alzheimer's disease-related processes identified in circulation, an integration of cf-mRNA sequencing and clinical information may also allow monitoring therapy response in Alzheimer's disease patients. Cf-mRNA is bound in a different biological compartment distinct from intracellular RNA and has been proposed to be involved in intercellular communication and as indicator of cellular stress.

One potential application that would benefit from a better understanding of the molecular mechanisms involved in Alzheimer's disease is the development of new therapeutic strategies to treat Alzheimer's disease. Cf-mRNA sequencing can provide a granular characterization of Alzheimer's disease patients' circulating transcriptome, including many of genes either dysregulated in Alzheimer's disease patients or correlated with Alzheimer's disease severity. Evidence points to a role of splice variants or combinations of splice variants in disease progression.

Bayesian analysis and Machine learning can be used to identify splice variants in cell-free mRNA to provide an approach to improve patient management in clinical practice. In addition, a better understanding of the heterogeneous etiology of Alzheimer's disease may aid in the identification of new molecular entities with therapeutic potential and increase their probability of technical success in pre-clinical and clinical stages.

Diagnosis of disease often requires invasive procedures to access organs and tissues. Protein coding cell-free messenger ribonucleic acid (cf-mRNA) is released from circulating cells, organs, and tissues. Aggregated reads of protein coding cf-mRNA demonstrate differential read numbers between cohorts of subjects with different diseases or stages of disease. Protein coding cf-mRNA can be carried not only by exosomes but also by multiple types of extracellular RNA carriers including lipid bilayer vesicles (e.g., exosomes, microvesicles, apoptotic bodies, and the like), membraneless particles or granules (e.g., exomeres, supermeres, and the like), retrosomal particles (e.g., Arc, and the like) and ribonucleoprotein complexes.

The detection of protein coding cf-mRNA has several advantages over the detection of other extracellular non-coding RNAs or liquid biopsy analytes such as proteins and metabolites. First, the non-coding extracellular RNAs interact with hundreds of different messenger RNA (mRNA), and each RNA can be bound by numerous non-coding RNAs, therefore identification of biological pathways that are involved is not possible. Second, the hypothesis-independent RNA-Seq machine learning combined with bioinformatics tools permits detection of underlying biology disruptions not previously characterized. Third, the number of mRNA far exceeds the number of analytes of proteins or metabolites on a single platform. Detection of approximately 10,000 cf-mRNA gene transcripts of the approximately 20,000 human genes encoded by the genome has been performed, of which about 1,600 are differentially expressed when comparing two disease states using this platform. Prior studies indicated the mRNA in cells (e.g., intracellular) varies in quantity and in genes represented from the mRNAs released from cells (e.g., extracellular).

While some genes are single exon genes whose coding sequence (CDS) is not interrupted by noncoding introns, most genes contain multiple exons. There are an estimated 180,000 exons in the human genome. The primary gene transcripts are spliced to remove non-coding regions or introns and retain different combinations of the exons of the gene. Approximately 80% of genes are expressed as multiple isoforms or splice variants, of which approximately half encode different proteins with altered functions. Splicing may occur in both the coding and non-coding regions of cf-mRNA. Messenger RNA isoforms are discrete species with different combinations of splice variants. Alternative splice variants vary by cell types that release them as well as by health or disease state. The complexity of splice variants reflects the cellular differentiation and diversity of cellular biology. An improved understanding is expected to lead to more informed disease pathology assessment, diagnosis, and intervention. Identification of junctions may identify discrete subtypes of underlying disease that may present with a similar phenotype. Published studies have reported some intracellular mRNA isoforms are differentially regulated without a change to the overall gene transcript level.

Disclosed herein are methods of treatment comprising antisense compositions and methods of use targeted against a splice junction region of an mRNA sequence coding for a selected protein. The antisense compound can be RNase-inactive. The antisense compound can be a phosphorodiamidate-linked morpholino oligonucleotide. Compounds disclosed herein can be effective to alter expression of a protein encoded by a splice junction region of an mRNA sequence disclosed herein.

Splice variants of cf-mRNA provide a different dimension of the cf-mRNA feature space. Disclosed herein is an aggregated gene transcript analysis of cf-mRNA splice junction reads.

Disclosed herein are methods for detecting a disease state of a subject, comprising: obtaining a biological sample from the subject; assaying cell-free messenger RNA (cf-mRNA) in the biological sample to determine a level of the cf-mRNA that contains a non-contiguous junction relative to genomic DNA, thereby detecting one or more splice junctions in the cf-mRNA; computer processing the detected one or more splice junctions; and detecting the disease state of the subject based at least in part on the computer processing.

Further disclosed herein are methods of determining a risk of a disease state in a subject, comprising: obtaining a biological sample from the subject; assaying cell-free messenger RNA (cf-mRNA) in the biological sample to detect one or more splice junctions in the cf-mRNA, wherein the one or more splice junctions correspond to one or more genes.

Further disclosed herein are methods of assessing an effect of a compound, comprising: assaying a first expression profile of a first cell-free biological sample obtained or derived from a subject at a first time point, to detect a first set of splice junctions; administering the compound to the subject; assaying a second expression profile of a second cell-free biological sample obtained or derived from the subject at a second time point subsequent to the administering, to detect a second set of splice junctions; computer processing the detected first and second sets of splice junctions; and assessing the effect of the compound based at least in part on the computer processing.

### Methods

### Detecting a Disease State

Disclosed herein are methods for detecting a disease state of a subject. In some cases, the methods comprise obtaining a biological sample from a subject. In some cases, the methods comprise assaying RNA in the biological sample. The RNA may be cell-free RNA. The cell-free RNA may be cell-free messenger RNA (cf-mRNA). In some cases, the methods comprise determining a level of the cf-mRNA that contains a non-contiguous junction. The non-contiguous junction may be relative to genomic DNA. In some cases, the methods comprise detecting one or more splice junctions. The splice junctions may correspond to the non-contiguous junctions. The splice junctions may be detected in cf-mRNA. In some cases, the methods comprise computer processing the detected one or more splice junctions. The computer processing may involve machine learning. The computer processing may involve use of a classifier or model. The computer processing may comprise use of prediction or classification. The classifier or the classification may be a trained classifier. In some cases, the methods comprise detecting a disease state of a subject based at least in part on the computer processing.

The methods may comprise detecting a disease state. The disease state may comprise a presence or an absence of a disease state. The disease state may be a stage of a disease, for example an incubation stage, a prodromal stage, an illness stage, a decline stage, or a convalescence stage. The disease state may be a likelihood of having a disease. The disease state may be one or more diseases, for example, two or more, three or more, four or more, or five or more diseases. The disease state may be a combination of disease states. The disease state may be an infectious disease, a deficiency disease, a hereditary disease (e.g., genetic or non-genetic), or a physiological disease. The disease state may be a disease of a bodily region or system, for example, a vascular disease, a gastrointestinal disease, a chest disease, or the like. The disease state may be a disease of an organ or a tissue, for example, a disease state of the heart, a disease state of the liver, a disease of the lung, a disease state of the skin, a disease state of the kidney, a disease state of the brain, or the like. The disease state may originate from an organ or a tissue, for example, the heart, the liver, the lung, the skin, the brain, the kidney, or the like. The disease state may impact one or more organs or tissues, for example, one or more of the heart, the liver, the lung, the skin, the brain, the kidney, or the like. The disease state may be a disease of a bodily function, for example, a metabolic disease, or the like.

The disease state may relate to a dementia. The disease state may be Alzheimer's disease. The Alzheimer's disease may be a stage of Alzheimer's disease, such as preclinical Alzheimer's disease, mild cognitive impairment due to Alzheimer's disease, mild dementia due to Alzheimer's disease, moderate dementia due to Alzheimer's disease, or severe dementia due to Alzheimer's disease.

The disease state may relate to memory. The disease state may relate to changes in mood, personality, disorientation, or the like. The disease state may relate to problems with speech, movement, problem solving, communication, or the like. The disease state may relate to confusion. The disease state may relate to spatial awareness. The disease state may relate to judgement and decision making. The disease state may be Huntington disease, frontotemporal dementia, Lewy Body Dementia (LBD), normal pressure hydrocephalus, vascular dementia, mixed dementia, corticobasal degeneration, progressive supranuclear palsy, chronic traumatic encephalopathy, multiple sclerosis, depression, general dementia, or the like. The disease state may be major depression, dysthymia, bipolar disorder, substance-induced mood disorders, or any other mood disorders. The disease state may relate to articulation disorders, phonological disorders, disfluency, voice disorders, or the like.

The methods may comprise detecting a disease state of a subject. The subject may be an animal. The subject may be a mammal, such as a human, a non-human primate, a rodent (e.g., a rat, a mouse, a guinea pig, a hamster, or the like), a dog, a cat, a pig, a sheep, a cow, a goat, or a rabbit. The subject may be a fish, a reptile, or a bird. The subject may be a human. The subject may be an adult (e.g., 18 years of age or older). The subject may be a child (e.g., less than 18 years of age). The subject may comprise an age of greater than or equal to 1, 2, 5, 10, 15, 20, 25, 30, 35, 40, 45, 50, 55, 60, 65, 70, 75, 80, 85, 90, or 95 years of age. The subject may be from about 50 to about 85 years of age. The subject may be from about 60 to about 80 years of age. The subject may be about 70 years of age. The subject may have or be suspected of having a disease state disclosed herein. For example, the subject may have or be suspected of having a dementia, for example, Alzheimer's disease. The subject may be asymptomatic. The subject may be healthy. The subject may have one or more risk factors associated with a disease state. For example, the subject may have risk factors such as diabetes, hypertension, or the like. The subject may be predisposed to having a disease state disclosed herein. For example, the subject may be predisposed to having Alzheimer's disease. The subject may be in remission from a treatment to the disease state. The subject may have one or more symptoms of a disease state disclosed herein. For example, the subject may have symptoms such as memory loss, misplacement of items, difficulty in decision making and judging, confusion, mood swings, social withdrawal, inability to problem solve or complete tasks, or the like.

The methods may comprise obtaining a biological sample from a subject. The biological sample may be a blood sample. The biological sample may be a plasma sample. The biological sample may be a serum sample. The biological sample may be a urine sample. The biological sample may be a saliva sample. The biological sample may be a sweat sample. The biological sample may be a semen sample. The biological sample may be a vaginal discharge sample. The biological sample may be a cell-free sample. The cell-free sample may comprise cell-free RNA, such as cell-free mRNA (cf-mRNA). The biological sample may be a tissue sample. The biological sample may be a tumor biopsy sample. The biological sample may be a bone marrow sample.

The biological sample may comprise nucleic acids. The biological sample may comprise ribonucleic acids (RNAs), such as messenger RNAs (mRNAs). The RNA may be cell-free. The cell-free RNAs may be cell-free mRNAs. The RNA may be pre-mRNA. The RNA may comprise a coding region. The RNA may comprise a non-coding region. The RNA may comprise small nuclear RNAs (snRNAs), micro RNAs (miRNAs), or small interfering RNAs (siRNAs). The biological sample may comprise deoxyribonucleic acids (DNAs). The biological sample may comprise proteins.

The methods may comprise assaying the biological sample. In some cases, the methods may comprise assaying cf-mRNA in the biological sample to determine a level of the cf-mRNA that contains a non-contiguous junction relative to genomic DNA.

The methods may include sequencing. Non-limiting examples of sequencing include sequencing by synthesis (SBS), pyrosequencing, sequencing by reversible terminator chemistry, sequencing by ligation, phospholinked fluorescent nucleotide sequencing, real-time sequencing, and the like. The method may include next generation sequencing (NGS). NGS utilizes the concept of massively parallel processing to obtain high-throughput, speed, and scalability. NGS may be referred to as massive parallel sequencing, massively parallel sequencing, or second-generation sequencing. The methods may include RNA sequencing. Non-limiting examples of RNA sequencing include mRNA sequencing, total RNA sequencing, low-input RNA sequencing, ultra-low-input RNA sequencing, small RNA sequencing, single cell RNA sequencing, and the like. The methods may include DNA sequencing. Non-limiting examples of DNA sequencing include Sanger sequencing, capillary electrophoresis, sequencing by synthesis, shotgun sequencing, pyrosequencing, combinatorial probe anchor synthesis, sequencing by ligation, nanopore sequencing, single molecular real time sequencing, ion torrent sequencing, nanoball sequencing, next generation sequencing, and the like.

The methods may include array hybridization. Array hybridization may include us of a microarray. A microarray is a laboratory tool that may be used to detect the expression of multiple genes at the same time. The microarray may be an analytical microarray, an antibody microarray, a functional microarray, a spotted array, a cellular microarray, an oligonucleotide DNA microarray, or the like. The microarray may use fluorescent dyes. The microarray may use probes, such as nucleotide probes. The microarray may comprise one or more wells, such as a 16-well plate, a 24-well plate, a 96 well plate, a 384-well plate, or the like. The one or more wells may be organized in rows and columns on the microarray.

The methods may include nucleic acid amplification. Nucleic acid amplification may include polymerase chain reaction (PCR), for example, multiplex PCR, long-range PCR, single-cell PCR, fast cycling PCR, methylation specific PCR, digital PCR, hot start PCR, real-time PCR (RT-PCR), quantitative PCR (qPCR), or the like. The nucleic acid amplification may include loop mediated isothermal amplification (LAMP). The nucleic acid amplification may include nucleic acid sequence-based amplification (NASBA). The nucleic acid amplification may include a strand displacement amplification (SDA). The nucleic acid amplification may include a multiple displacement amplification (MDA). The nucleic acid amplification may include rolling circle amplification (RCA). The nucleic acid amplification may include ligase chain reaction (LCR). The nucleic acid amplification may include helicase dependent amplification (HDA). The nucleic acid amplification may include a ramification amplification method (RAM). The nucleic acid amplification may include a transcription-mediated assay (TMA).

The methods may further include identifying a tissue of a disease state. The methods may comprise analyzing the cf-mRNA in the biological sample and determining a tissue that the cf-mRNA originated from. The tissue may be identified to be under duress. The tissue may be identified to be impacted by the disease state. The tissue may be identified to be the origin of the disease state. The tissue may be nervous tissue, such as tissue of the brain, spinal cord, or nerves. The tissue may comprise circulating immune cells. The tissue may be muscle tissue, such as cardiac muscle tissue, smooth muscle tissue, or skeletal muscle tissue. The muscle tissue may originate from muscles in the body. The tissue may be epithelial tissue, such as lining of the gastrointestinal tract of organs or the skin surface (epidermis). The tissue may be connective tissue, such as tissue from fat (or other soft padding tissue), bone, or tendons. The tissue may be any tissue in the body.

The methods may further comprise identifying an organ of a disease state. One or more organs may be identified of the disease state. The methods may comprise analyzing the cf-mRNA in the biological sample and determining an organ that the cf-mRNA originated from. The organ may be identified to be under duress. The organ may be identified to be impacted by the disease state. The organ may be identified to be the origin of the disease state. The organ may be the lungs. The organ may be the liver. The organ may be the bladder. The organ may be the kidneys. The organ may be the heart. The organ may be the stomach. The organ may be the intestines, such as the small intestine or the large intestine. The organ may be the brain. The organ may be the pancreas. The organ may be the gallbladder. The organ may be any organ in the body.

The methods may further comprise identifying one or more biological pathways of the disease state. The biological pathways may be identified to be under duress. The biological pathways may be identified to be impacted by the disease state. The biological pathways may be identified to be the origin of the disease state. The biological pathways may include neurological pathways, digestive pathways, muscular pathways, respiratory pathways, endocrine pathways, reproductive pathways, skeletal pathways, lymphatic pathways, immune pathways, immunological pathways, gastrointestinal pathways, nervous system pathways, or any combination thereof. The biological pathways may relate to the disease state. For example, the biological pathways may relate to a neurodegenerative disease. In some cases, the neurodegenerative disease is Alzheimer's disease.

The methods may include producing complementary deoxyribonucleic acid (cDNA) from RNA. In some cases, the methods may include converting RNA, for example cf-mRNA, to cDNA using a reverse transcription protocol. Reverse transcription is a process that converts RNA to cDNA using, among other things, a reverse transcriptase enzyme and deoxyribonucleotide triphosphates (dNTPs). Reverse transcriptase is an enzyme that is an RNA-dependent DNA polymerase. Reverse transcription may utilize several reaction components, such as an RNA template, one or more primers, one or more reaction buffers, dNTPs, DTT, RNase inhibitor, DNA polymerase, DNA ligase, water, or a combination thereof. The reverse transcription reaction may generally follow the steps of annealing, polymerization, and deactivation.

In some cases, a sample cDNA is produced from cf-mRNA by reverse transcription. In some cases, a cDNA library may be produced from the produced cDNA sample. The cDNA library may contain DNA copies of the cf-mRNA obtained from the biological sample. The cDNA library may be compared to a reference library. The reference library may be generated from a biological sample of a subject known not to have the disease state, for example, a subject known to be non-cognitively impaired, or a subject known not to have Alzheimer's disease.

The methods may include comparing a sample cDNA to a reference sample. The reference sample may be obtained from a healthy subject known not to have the disease state. For example, the reference sample may be obtained from a non-cognitively impaired subject. The methods may comprise identifying differences between the sample cDNA and the reference sample. For example, non-contiguous junctions may be present in the sample cDNA and not present in the reference sample. For example, one or more splice junctions may be present in the sample cDNA and not present in the reference sample. Additional differences, such as differences in nucleotide sequences, may be identified between the sample cDNA and the reference sample. In some cases, the reference sample may comprise aggregated least variant gene cf-mRNAs. In some cases, the reference sample may comprise prior sampling. In some cases, the reference sample may comprise a reference interval.

In some cases, the methods may comprise detecting one or more splice junctions. Splice junctions may be referred to as the boundaries between introns and/or exons during RNA splicing in transcription. In some cases, splice junctions may comprise non-coding splice junctions, such as splice junctions in 5′ or 3′ untranslated regions. Transcription is the process by which a cell makes an RNA copy of a piece of DNA. Splicing is the process in which introns, which are the noncoding regions of genes, are excised out of the primary messenger RNA transcript, and the exons, which are the coding regions, are joined together to generate a mature messenger RNA. Non-limiting examples of splice junctions include exon-exon splice junctions (e.g., the boundary between two exons), exon-intron splice junctions (e.g., the boundary between an exon and an intron), and intron-intron splice junctions (e.g., the boundary between two introns). The identification of splice junctions involves the recognition of exon-exon, exon-intron, and intron-intron boundaries during transcription. A splice junction may comprise a boundary between two nucleotides. A splice junction may comprise more than or equal to one nucleotide, for example, more than or equal to two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

The one or more splice junctions may comprise one or more isoforms. An isoform is a specific combination of splice junctions that can result from alternative splicing. Alternative splicing, also called alternative RNA splicing or differential splicing, is a process that allows a single gene to code for multiple proteins. Alternative splicing may generate different RNAs that are translated into proteins. Alternative splicing may generate different RNAs that are translated into proteins. In alternative splicing, exons/introns from the same gene are joined together in different combinations, leading to different but related resulting mRNA transcripts during transcription. There are several modes of alternative splicing, including exon skipping alternative splicing, mutually exclusive exon alternative slicing, alternative 3′ alternative splicing, alternative 5′ alternative splicing, and intron retention alternative splicing. In exon skipping alternative splicing, an exon may be retained or spliced out of the transcript. Exon skipping alternative splicing is the most common form of alternative splicing and results in the loss of an exon in the alternatively spliced transcript. In mutually exclusive exon alternative splicing, alternative isoforms are generated by retaining only one exon of a cluster of neighboring internal exons in the mature transcript. Mutually exclusive exon alternative splicing indicates that one out of two exons (or one group out of two exon groups) is retained, while the other exon/group is spliced out. In alternative 5′ alternative splicing, an alternative 5′ splice junction is used, which changes the 3′ boundary of the upstream exon. In alternative 3′ alternative splicing, an alternative 3′ splice junction is used, which changes the 5′ boundary of the downstream exon. In intron retention alternative splicing, an intron is retained in the mature mRNA transcript. In some cases, splicing occurs in a 3′ or 5′ untranslated region.

The one or more splice junctions may correspond to one or more genes. The methods may comprise determining that more than or equal to one splice junction corresponds to more than or equal to one gene. In some cases, one splice junction corresponds to more than or equal to one, two, three, four, five six, seven, eight, nine, 10, 11, 12, 13, 14, 15, 16, 17, 18, 19, 20, 21, 22, 23, 24, or 25 genes. In some cases, two splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, three splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, four splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, five splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 10 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 15 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 20 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 25 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 50 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 100 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 250 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 500 splice junctions correspond to more than or equal to one, two, three, four, five six, seven, eight, nine, 10, 11, 12, 13, 14, 15, 16, 17, 18, 19, 20, 21, 22, 23, 24, or 25 genes.

In some cases, the methods disclosed herein may identify one or more genes that are expressed. For example, the genes may be expressed in a first population of subjects with a disease state, such as Alzheimer's disease, as compared to a second population of subjects known not to have the disease state. The second population of subjects may be non-cognitively impaired. The second population of subjects may be healthy. The second population of subjects may be known not have Alzheimer's disease.

The methods may comprise identifying one or more expressed genes. In some cases, the methods comprise identifying one or more, five or more, 10 or more, 20 or more, 30 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, 200 or more, 210 or more, 220 or more, 230 or more, 240 or more, 250 or more, 260 or more, 270 or more, 280 or more, 290 or more, 300 or more, 310 or more, 320 or more, 330 or more, 340 or more, 350 or more, 360 or more, 370 or more, 380 or more, 390 or more, 400 or more, 410 or more, 420 or more, 430 or more, 440 or more, 450 or more, 460 or more, 470 or more, 480 or more, 490 or more, or 500 or more expressed genes. In some cases, the methods comprise identifying 500 or less, 490 or less, 480 or less, 470 or less, 460 or less, 450 or less, 440 or less, 430 or less, 420 or less, 410 or less, 400 or less, 390 or less, 380 or less, 370 or less, 360 or less, 350 or less, 340 or less, 330 or less, 320 or less, 310 or less, 300 or less, 290 or less, 280 or less, 270 or less, 260 or less, 250 or less, 240 or less, 230 or less, 220 or less, 210 or less, 200 or less, 190 or less, 180 or less, 170 or less, 160 or less, 150 or less, 140 or less, 130 or less, 120 or less, 110 or less, 100 or less, 90 or less, 80 or less, 70 or less, 60 or less, 50 or less, 40 or less, 30 or less, 20 or less, 10 or less expressed genes.

The methods may comprise identifying the genes present in Tables 1-3 to be expressed between a first population of disease state subjects as compared to a second population of non-disease state subjects.

Modeling Alternative Junction Inclusion Quantification (“MAJIQ”) is a software package that can detect, quantify, and visualize local splicing variations (“LSV”) from RNA sequencing data. LSVs can include two or more splice junctions that can emanate out from a reference exon (e.g., a source LSV) or converge into a reference exon (e.g., a target LSV). LSV's can capture the classical, binary, alternative splicing events involving two alternative splice junctions. LSV's can also capture more complex (e.g., non-binary) splicing variations. A LSV ID (local splicing variations identifier), as used herein, is a unique identifier for the LSV that can be generated by the MAJIQ package. The LSV ID may be comprised of an ENSG Ensembl ID, whether it is a source (s) or target (t) LSV, exon coordinates, intron coordinates, and combinations thereof. For each gene listed herein, the LSV ID is provided in parentheses, for example Gene (LSV ID (e.g., ENSG number, source (s) or target (t), and exon/intron coordinates).

In some cases, the expressed splice junctions may comprise a member of one or more of the group consisting of: ABLIM1 (ENSG00000099204.21:t:114491791-114491878;114491878-114545005), ACAA1 (ENSG00000060971.19:s:38126493-38126700;38126341-38126510), ACAP1 (ENSG00000072818.12:t:7341948-7342067;7336787-7341948), ACOT8 (ENSG00000101473.17:t:45848450-45849110;45848675-45857188), ACP1 (ENSG00000143727.16:t:272192-273155;272065-272192), ADD3 (ENSG00000148700.15:t:110100625-110100848;110008299-110100625), ADGRE5 (ENSG00000123146.20:t:14397414-14397804;14391079-14397658), ADK (ENSG00000156110.15:t:74200764-74200838;74151343-74200764), AGAP3 (ENSG00000133612.19:s:151119987-151120145;151120145-151122725), AKAP13 (ENSG00000170776.22:t:85575131-85575329;85543955-85575131), ALAD (ENSG00000148218.16:s:113393161-113393634;113392169-113393447), AMPD2 (ENSG00000116337.20:t:109625303-109625433;109621266-109625303), ANAPC11 (ENSG00000141552.18:t:81894298-81894624;81891833-81894467), AP1B1 (ENSG00000100280.17:s:29330378-29330532;29329720-29330378), AP1B1 (ENSG00000100280.17:t:29327680-29328895;29328895-29329712), APP (ENSG00000142192.21:s:26000015-26000182;25982477-26000015), APP (ENSG00000142192.21:t:25897573-25897983;25897673-25911741), ARAP1 (ENSG00000186635.15:s:72693325-72693470;72688537-72693325), ARFRP1 (ENSG00000101246.20:t:63706999-63707658;63707097-63707867), ARHGAP17 (ENSG00000140750.17:t:24935470-24936827;24935639-24941987), ARHGAP17 (ENSG00000288353.1:t:188953-190310;189122-195470), ARHGEF10 (ENSG00000274726.4:s:43270-43560;43560-47749), ARHGEF1 (ENSG00000076928.19:s:41896377-41896878;41896482-41897315), ARHGEF7 (ENSG0000002606.20:t:111217679-111217885;111210002-111217679), ARMC10 (ENSG00000170632.14:s:103074881-103075411;103075411-103075777), ARRDC2 (ENSG00000105643.11:t:18008711-18008861;18001573-18008711), ATE1 (ENSG00000107669.19:t:121924266-121924329;121924329-121928347), ATP6V1D (ENSG00000100554.12:t:67350611-67350690;67350690-67359658), ATXN2L (ENSG00000168488.19:s:28835933-28836176;28836122-28836748), ATXN2L (ENSG00000168488.19:t:28836728-28837237;28836122-28836748), ATXN2L (ENSG00000168488.19:t:28836728-28837237;28836485-28836728), AURKB (ENSG00000178999.13:t:8207738-8208225;8207840-8210530), BLOC1S6 (ENSG00000104164.12:s:45592135-45592276;45592276-45605428), C12orf76 (ENSG00000174456.16:s:110048363-110048870;110042459-110048363), CAMTA1 (ENSG00000171735.20:t:6825092-6825210;6820250-6825092), CBFA2T3 (ENSG00000129993.15:t:88892244-88892485;88892485-88898078), CBFB (ENSG00000067955.15:t:67066682-67066962;67036755-67066682), CCDC28B (ENSG00000160050.16:t:32204598-32204620;32204379-32204598), CCDC92 (ENSG00000119242.9:s:123972529-123972831;123943493-123972529), CCDC92 (ENSG00000119242.9:t:123943347-123943495;123943493-123972529), CD300H (ENSG00000284690.3:s:74563919-74564275;74560824-74563919), CD34 (ENSG00000174059.17:s:207888682-207888846;207887923-207888682), CDAN1 (ENSG00000140326.13:s:42736989-42737128;42736780-42737013), CDC14B (ENSG00000081377.17:t:96565393-96565512;96565483-96619219), CDK5RAP2 (ENSG00000136861.19:t:120402806-120403071;120403071-120403316), CELF2 (ENSG00000048740.19:t:11249153-11249201;11165682-11249153), CEP164 (ENSG00000110274.16:t:117409618-117410701;117409028-117409618), CLEC1B (ENSG00000165682.15:s:9995140-9995246;9986158-9995140), COX20 (ENSG00000203667.10:s:244835616-244835756;244835756-244842195), CPNE1 (ENSG00000214078.13:s:35627280-35627531;35626800-35627280), CYTH2 (ENSG00000105443.17:s:48474838-48476276;48474949-48477717), CYTH2 (ENSG00000105443.17:t:48478066-48478145;48477719-48478069), DAZAP1 (ENSG00000071626.17:s:1422348-1422396;1422396-1425878), DCTD (ENSG00000129187.15:s:182917288-182917477;182915575-182917311), DCTD (ENSG00000129187.15:t:182915461-182915575;182915575-182917288), DECR1 (ENSG00000104325.7:s:90001405-90001561;90001561-90017124), DECR1 (ENSG00000104325.7:s:90001405-90001561;90001561-90018909), DECR1 (ENSG00000104325.7:t:90018564-90018966;90001561-90018909), DEF8 (ENSG00000140995.17:t:89954172-89954376;89949513-89954243), DGKD (ENSG00000077044.11:s:233390403-233390483;233390483-233392070), DGKZ (ENSG00000149091.15:t:46367291-46367399;46345585-46367291), DNM2 (ENSG00000079805.19:s:10795372-10795439;10795439-10797380), DOCK8 (ENSG00000107099.18:t:271627-271729;215029-271627), DUT (ENSG00000128951.14:t:48332103-48332737;48331479-48332268), DYRK1A (ENSG00000157540.22:s:37418905-37420384;37420384-37472684), DYSF (ENSG00000135636.16:t:71480883-71480938;71454086-71480883), EIF4G1 (ENSG00000114867.22:t:184317321-184317497;184314674-184317321), EIF4G3 (ENSG00000075151.24:t:20981048-20981227;20981227-20997601), EMC8 (ENSG00000131148.9:s:85781211-85781760;85779867-85781211), EPSTI1 (ENSG00000133106.15:s:42991978-42992271;42969177-42991978), EXOSC1 (ENSG00000171311.13:s:97438663-97438703;97437750-97438663), EXOSC1 (ENSG00000171311.13:t:97437700-97437750;97437750-97438663), F2RL3 (ENSG00000127533.4:s:16888999-16889298;16889298-16889577), FAM219A (ENSG00000164970.15:s:34405865-34405964;34402807-34405916), FAM3A (ENSG00000071889.17:s:154512823-154512936;154511871-154512823), FBXO44 (ENSG00000132879.14:t:11658736-11658871;11658628-11658736), FKBP1B (ENSG00000119782.14:s:24060814-24060926;24060926-24063019), FMNL3 (ENSG00000161791.14:t:49657082-49657190;49657190-49658442), FOXP1 (ENSG00000114861.24:t:71112536-71112637;71112637-71130498), G3BP1 (ENSG00000145907.16:t:151786572-151786715;151772036-151786603), GET1 (ENSG00000182093.16:t:39390698-39390803;39380556-39390698), GLUL (ENSG00000135821.19:t:182388572-182390304;182388750-182391175), GORASP1 (ENSG00000114745.15:s:39107479-39108063;39103553-39107479), GORASP1 (ENSG00000114745.15:t:39101016-39101102;39103553-39107479), GSE1 (ENSG00000131149.19:t:85648552-85648751;85556363-85648552), GSTT1 (ENSG00000277656.3:t:270997-271173;271173-278295), GUCD1 (ENSG00000138867.17:t:24548917-24549001;24549001-24555615), HDAC7 (ENSG00000061273.18:s:47796207-47796298;47796016-47796207), HES6 (ENSG00000144485.11:t:238239487-238239568;238239568-238239825), HUS1 (ENSG00000136273.13:s:47979410-47979615;47978816-47979410), IFI27 (ENSG00000275214.4:t:1230073-1230504;1229442-1230343), IGF2BP3 (ENSG00000136231.14:t:23342064-23342189;23342189-23343718), IKBKB (ENSG00000104365.16:t:42290156-42290273;42288728-42290156), IKBKG (ENSG00000269335.7:t:154551988-154552189;154542452-154551988), IKZF3 (ENSG00000161405.17:t:39777651-39777767;39777767-39788258), IL10RA (ENSG00000110324.12:t:117988382-117988502;117986534-117988382), IL15RA (ENSG00000134470.21:t:5960367-5960567;5960567-5963743), IMPDH1 (ENSG00000106348.18:t:128405767-128405948;128405865-128409289), INO80E (ENSG00000169592.15:s:30001414-30001575;30001530-30005221), INPP5D (ENSG00000281614.3:s:67445-67595;67595-71086), IP6K2 (ENSG00000068745.15:t:48694872-48695421;48695421-48717157), IRF7 (ENSG00000276561.4:s:144369-144427;144292-144369), IRF7 (ENSG00000276561.4:s:144676-144900;144424-144690), ITSN2 (ENSG00000198399.16:s:24246129-24246320;24242206-24246129), JAML (ENSG00000160593.19:t:118212407-118212561;118212561-118214824), JOSD2 (ENSG00000161677.12:t:50506378-50506572;50506572-50507574), KANK2 (ENSG00000197256.11:s:11195556-11195881;11194590-11195556), KANK2 (ENSG00000197256.11:t:11194553-11194590;11194590-11195556), KANSL3 (ENSG00000114982.19:s:96636739-96637228;96631543-96636921), KANSL3 (ENSG00000114982.19:t:96631312-96631543;96631543-96636921), KAT5 (ENSG00000172977.13:s:65712922-65713319;65713058-65713348), KATNB1 (ENSG00000140854.13:s:57755345-57755494;57755494-57755841), KDM5C (ENSG00000126012.13:s:53217796-53217966;53217274-53217796), KIAA1671 (ENSG00000197077.14:t:25170820-25170938;25049364-25170820), KIFC3 (ENSG00000140859.16:t:57772223-57772288;57772288-57785464), KIFC3 (ENSG00000140859.16:t:57798072-57798282;57798282-57802370), KMT2B (ENSG00000272333.8:t:35719784-35720940;35719541-35719784), LDB1 (ENSG00000198728.11:s:102109029-102109177;102108323-102109029), LDB2 (ENSG00000169744.13:s:16511981-16512104;16508686-16511981), LETMD1 (ENSG00000050426.16:s:51048301-51048518;51048478-51056350), LST1 (ENSG00000204482.11:s:31587117-31587318;31587318-31587641), LST1 (ENSG00000204482.11:t:31587944-31587966;31587318-31587944), LST1 (ENSG00000206433.10:s:2842938-2843143;2843139-2844386), LST1 (ENSG00000223465.8:s:2834852-2835057;2835053-2835376), LST1 (ENSG00000223465.8:t:2835679-2835701;2835053-2835679), LST1 (ENSG00000226182.8:t:3065231-3065253;3064605-3065231), LST1 (ENSG00000230791.8:s:2886832-2887014;2887014-2887225), LST1 (ENSG00000230791.8:t:2887225-2887247;2886599-2887225), LST1 (ENSG00000231048.8:s:2892158-2892363;2892359-2892682), LST1 (ENSG00000231048.8:t:2892985-2893007;2892359-2892985), LTB (ENSG00000204487.8:s:3059543-3059714;3058917-3059543), LTB (ENSG00000223448.7:s:2887297-2887468;2886671-2887297), LTB (ENSG00000238114.7:s:2881536-2881707;2880910-2881536), LTBP4 (ENSG00000090006.18:s:40619347-40619493;40619493-40622401), LTBP4 (ENSG00000090006.18:t:40619347-40619493;40614446-40619347), LTBP4 (ENSG00000090006.18:t:40623604-40623732;40623021-40623604), MAPK9 (ENSG00000050748.18:t:180269280-180269409;180269409-180279796), MARK2 (ENSG00000072518.22:s:63904786-63905043;63905043-63908260), MAST3 (ENSG00000099308.13:s:18146881-18147044;18147017-18147443), MAX (ENSG00000125952.20:t:65077193-65077428;65077428-65077913), MBNL1 (ENSG00000152601.18:t:152414941-152415111;152269092-152414941,) METTL9 (ENSG00000197006.15:s:21624931-21625233;21625115-21655227), MGLL (ENSG00000074416.16:t:127821694-127821838;127821838-127822309), MGRN1 (ENSG00000102858.13:s:4677463-4677572;4677572-4681550), MIEF1 (ENSG00000100335.15:s:39511849-39512026;39512026-39512232), MLX (ENSG00000108788.12:s:42567619-42567655;42567655-42568837), MMAB (ENSG00000139428.12:s:109561420-109561517;109561329-109561420), MPI (ENSG00000178802.18:s:74890005-74890201;74890089-74890527), MPRIP (ENSG00000133030.22:t:17142627-17142765;17138429-17142627), MRPL33 (ENSG00000243147.8:s:27772674-27772855;27772692-27779433), MSRB3 (ENSG00000174099.12:s:65278643-65278865;65278865-65326826), MTA1 (ENSG00000182979.18:s:105445418-105445511;105445511-105450058), MTA1 (ENSG00000182979.18:t:105450058-105450184;105445511-105450058), MTHFD2 (ENSG00000065911.13:t:74207704-74207826;74205889-74207704), MTMR12 (ENSG00000150712.11:s:32312758-32312987;32274122-32312758), MTMR12 (ENSG00000150712.11:t:32273980-32274122;32274122-32312758), NAP1L4 (ENSG00000273562.4:t:175479-176694;176694-182308), NCOA2 (ENSG00000140396.13:t:70141179-70141399;70141399-70148273), NCOR2 (ENSG00000196498.14:s:124330845-124330898;124326370-124330845), NCOR2 (ENSG00000196498.14:s:124378237-124378384;124372610-124378237), NCOR2 (ENSG00000196498.14:t:124372022-124372610;124372610-124378237), NDRG2 (ENSG00000165795.25:t:21022392-21022820;21022497-21022864), NDUFV3 (ENSG00000160194.18:t:42908864-42913304;42897047-42908864), NFE2L1 (ENSG00000082641.16:s:48056386-48056598;48056598-48057032), NFIA (ENSG00000162599.18:t:61455303-61462788;61406727-61455303), NKIRAS2 (ENSG00000168256.18:t:42023654-42025644;42022640-42023654), NOC2L (ENSG00000188976.11:s:945042-945146;944800-945057), NTAN1 (ENSG00000275779.4:s:613600-613788;613788-621580), NTPCR (ENSG00000135778.12:t:232956347-232956443;232950744-232956347), NUDT22 (ENSG00000149761.9:t:64229131-64229344;64227132-64229247), NUP214 (ENSG00000126883.19:s:131146129-131146304;131146304-131147490), NXT2 (ENSG00000101888.12:t:109538045-109538131;109537270-109538045), OCEL1 (ENSG00000099330.9:s:17226213-17226353;17226353-17226693), P2RX4 (ENSG00000135124.16:s:121210065-121210298;121210298-121217134), PABIR2 (ENSG00000156504.17:s:134781821-134781917;134772283-134781821), PALM2AKAP2 (ENSG00000157654.19:s:110138171-110138539;110138539-110168399), PANK4 (ENSG00000157881.16:t:2521101-2521315;2521315-2526464), PAPOLA (ENSG00000090060.19:s:96556175-96556413;96556413-96560649), PARL (ENSG00000175193.14:s:183862607-183862801;183844326-183862697), PARL (ENSG00000175193.14:t:183844231-183844774;183844326-183862753), PARVB (ENSG00000188677.15:t:44093928-44094017;44069162-44093928), PCYT1B (ENSG00000102230.14:t:24618985-24619084;24619084-24646989), PDE7A (ENSG00000205268.11:t:65782783-65782843;65782843-65841371), PEX26 (ENSG00000215193.14:s:18083437-18083732;18083732-18087972), PFKFB3 (ENSG00000170525.21:t:6213623-6213748;6203336-6213623), PKN1 (ENSG00000123143.13:t:14441143-14441443;14433542-14441143), PLEKHM1 (ENSG00000225190.12:s:45481603-45482612;45478147-45482433), PLS3 (ENSG00000102024.19:t:115610243-115610323;115561260-115610243), PML (ENSG00000140464.20:s:74034478-74034558;74034530-74042989), PML (ENSG00000140464.20:t:74033156-74033422;74032715-74033156), POLR2J3 (ENSG00000168255.20:t:102566997-102567083;102567083-102568010), POLR2J3 (ENSG00000285437.2:t:102566966-102567083;102567083-102568010), PPIE (ENSG000000084072.17:s:39752910-39753266;39753052-39753287), PPM1N (ENSG00000213889.11:t:45499949-45500066;45497343-45499949), PPP6R2 (ENSG00000100239.16:s:50436367-50436452;50436452-50436988), PPP6R2 (ENSG00000100239.16:t:50437506-50437603;50437068-50437506), PRKAR1B (ENSG00000188191.16:t:596146-596304;596304-602553), PRKCD (ENSG00000163932.16:t:53178404-53178537;53165215-53178404), PSEN1 (ENSG00000080815.20:t:73170797-73171923;73148094-73170797), PSMB8 (ENSG00000230034.8:t:4086512-4086659;4086659-4087849), PSMG4 (ENSG00000180822.12:s:3263684-3263759;3263759-3264209), PTK2B (ENSG00000120899.18:t:27450749-27450895;27445919-27450749), PTPN12 (ENSG00000127947.16:s:77600664-77600965;77600806-77607235), PTPN18 (ENSG00000072135.13:t:130368897-130369201;130356200-130369133), PUF60 (ENSG00000179950.15:s:143821118-143821743;143818534-143821597), PUF60 (ENSG00000179950.15:s:143821118-143821743;143820716-143821597), PUM1 (ENSG00000134644.16:s:30967099-30967310;30966278-30967167), PYM1 (ENSG00000170473.17:t:55903387-55903480;55903480-55927076), RAB11FIP1 (ENSG00000156675.16:t:37870387-37870528;37870528-37871278), RAB1FIP5 (ENSG00000135631.17:t:73075993-73076182;73076182-73088050), RAB4A (ENSG00000168118.12:t:229295848-229295910;229271370-229295848), RABGAP1L (ENSG00000152061.24:t:174969277-174969387;174957549-174969277), RAP1B (ENSG00000127314.18:t:68650244-68650882;68648781-68650400), RARA (ENSG00000131759.18:t:40348316-40348464;40331396-40348316), RBCK1 (ENSG00000125826.22:s:409830-410025;410025-417526), RBM10 (ENSG00000182872.16:t:47173128-47173197;47169498-47173128), RBM39 (ENSG00000131051.24:s:35713019-35713203;35709256-35713019), RCOR3 (ENSG00000117625.14:t:211313424-211316385;211312961-211313424), REPS2 (ENSG00000169891.18:s:17022123-17022371;17022271-17025059), RFFL (ENSG00000092871.17:s:35026374-35026568;35021781-35026374), RHD (ENSG00000187010.21:s:25303322-25303459;25303459-25328898), RMND5B (ENSG00000145916.19:t:178138108-178138396;178131054-178138108), RPUSD1 (ENSG00000007376.8:t:786826-786928;786928-787077), SEC16A (ENSG00000148396.19:s:136447227-136447364;136446949-136447227), SIGLEC10 (ENSG00000142512.15:t:51414422-51414515;51414515-51415181), SIRT3 (ENSG00000142082.15:s:218778-219041;216718-218778), SLA2 (ENSG00000101082.14:s:36615225-36615374;36614387-36615225), SLC66A2 (ENSG00000122490.19:t:79903446-79904183;79904183-79919184), SMARCA4 (ENSG00000127616.21:s:11034914-11035132;11035132-11041298), SMARCA4 (ENSG00000127616.21:t:11040634-11041560;11035132-11041298), SMARCC2 (ENSG00000139613.12:s:56173696-56173849;56173029-56173696), SNX20 (ENSG00000167208.16:s:50675770-50675921;50669148-50675770), SPIB (ENSG00000269404.7:s:50423605-50423755;50423751-50428038), SRSF6 (ENSG00000124193.16:s:43458361-43458509;43458509-43459153), SSX2IP (ENSG00000117155.17:t:84670646-84670815;84670815-84690371), STAMBP (ENSG00000124356.17:t:73830845-73831059;73829070-73830845), STAT3 (ENSG00000168610.16:t:42317182-42317224;42317224-42319856), SWI5 (ENSG00000175854.15:t:128276587-128276755;128276402-128276707), SYNE1 (ENSG00000131018.25:s:152301869-152302269;152300781-152301869), TANGO2 (ENSG00000183597.16:t:20061530-20061802;20056013-20061530), TCF3 (ENSG00000071564.19:s:1615686-1615804;1612433-1615686), TCF7L2 (ENSG00000148737.18:s:113146011-113146097;113146097-113150998), TECR (ENSG00000099797.15:s:14562356-14562575;14562575-14563174), TJP2 (ENSG00000119139.21:t:69212548-69212601;69151771-69212548), TLE4 (ENSG00000106829.21:s:79627374-79627752;79627448-79652593), TLE4 (ENSG00000106829.21:t:79652590-79652629;79627448-79652593), TMBIM1 (ENSG00000135926.15:s:218292466-218292586;218282181-218292466), TMBIM4 (ENSG00000155957.19:s:66169855-66170027;66161172-66169855), TMEM11 (ENSG00000178307.10:s:21214091-21214176;21211227-21214091), TMEM126B (ENSG00000171204.13:t:85631687-85631808;85628688-85631687), TMEM219 (ENSG00000149932.17:t:29962677-29963308;29962132-29963107), TNFSF12 (ENSG00000239697.12:t:7549466-7549618;7549312-7549474), TNK2 (ENSG00000061938.21:t:195888426-195888606;195888606-195908485), TNRC18 (ENSG00000182095.15:t:5325096-5325574;5325248-5329937), TRAPPC2 (ENSG00000196459.15:s:13734525-13734635;13719982-13734525), TRAPPC6A (ENSG00000007255.10:t:45164725-45164970;45164970-45165127), TSC22D3 (ENSG00000157514.18:t:107715773-107715950;107715950-107775100), TSPAN32 (ENSG00000064201.16:s:2314485-2314666;2314571-2316229), UBAC2 (ENSG00000134882.16:t:99238427-99238554;99200939-99238427), UFD1 (ENSG00000070010.19:t:19471687-19471808;19471808-19479083), URI1 (ENSG00000105176.18:s:29942239-29942664;29942664-29985223), URI1 (ENSG00000105176.18:t:29985223-29985301;29942664-29985223), USP15 (ENSG00000135655.16:s:62325872-62325933;62325933-62349221), USP22 (ENSG00000124422.12:t:21028542-21028674;21028674-21043321), VGLL4 (ENSG00000144560.16:t:11601833-11602022;11602022-11702971), WWP2 (ENSG00000198373.13:t:69786996-69787080;69762391-69786996), YAF2 (ENSG00000015153.15:s:42237599-42237800;42199235-42237599), YIPF6 (ENSG00000181704.12:s:68498562-68499157;68499123-68511849), YIPF6 (ENSG00000181704.12:t:68513327-68515340;68511977-68513327), ZBTB7B (ENSG00000160685.14:t:155014448-155015814;155002943-155014655), ZEB2 (ENSG00000169554.23:t:144517278-144517557;144517419-144517612), ZFAND1 (ENSG00000104231.11:s:81718182-81718224;81715114-81718182), ZFAND1 (ENSG00000104231.11:t:81714987-81715114;81715114-81718182), ZFAND2B (ENSG00000158552.13:s:219207648-219207779;219207774-219207887), ZMIZ2 (ENSG00000122515.16:s:44759281-44759460;44759460-44760151), ZMYND8 (ENSG00000101040.20:t:47236326-47236516;47236516-47238758), ZNF185 (ENSG00000147394.20:s:152922720-152922809;152922809-152928575), and ZNF451 (ENSG00000112200.17:t:57099061-57099141;57090274-57099061). In some cases, the expressed splice junctions may comprise expressed genes.

The identified gene may include ABLIM1 (ENSG00000099204.21:t:114491791-114491878;114491878-114545005). The identified gene may include ACAA1 (ENSG00000060971.19:s:38126493-38126700;38126341-38126510). The identified gene may include ACAP1 (ENSG00000072818.12:t:7341948-7342067;7336787-7341948). The identified gene may include ACOT8 (ENSG00000101473.17:t:45848450-45849110;45848675-45857188). The identified gene may include ACP1 (ENSG00000143727.16:t:272192-273155;272065-272192). The identified gene may include ADD3 (ENSG00000148700.15:t:110100625-110100848;110008299-110100625). The identified gene may include ADGRE5 (ENSG00000123146.20:t:14397414-14397804;14391079-14397658). The identified gene may include ADK (ENSG00000156110.15:t:74200764-74200838;74151343-74200764). The identified gene may include AGAP3 (ENSG00000133612.19:s:151119987-151120145;151120145-151122725). The identified gene may include AKAP13 (ENSG0000170776.22:t:85575131-85575329;85543955-85575131). The identified gene may include ALAD (ENSG00000148218.16:s:113393161-113393634;113392169-113393447). The identified gene may include AMPD2 (ENSG00000116337.20:t:109625303-109625433;109621266-109625303). The identified gene may include ANAPC11 (ENSG00000141552.18:t:81894298-81894624;81891833-81894467). The identified gene may include AP1B1 (ENSG00000100280.17:s:29330378-29330532;29329720-29330378). The identified gene may include AP1B1 (ENSG00000100280.17:t:29327680-29328895;29328895-29329712). The identified gene may include APP (ENSG00000142192.21:s:26000015-26000182;25982477-26000015). The identified gene may include APP (ENSG00000142192.21:t:25897573-25897983;25897673-25911741). The identified gene may include ARAP1 (ENSG00000186635.15:s:72693325-72693470;72688537-72693325). The identified gene may include ARFRP1 (ENSG00000101246.20:t:63706999-63707658;63707097-63707867). The identified gene may include ARHGAP17 (ENSG00000140750.17:t:24935470-24936827;24935639-24941987). The identified gene may include ARHGAP17 (ENSG00000288353.1:t:188953-190310;189122-195470). The identified gene may include ARHGEF10 (ENSG00000274726.4:s:43270-43560;43560-47749). The identified gene may include ARHGEF1 (ENSG00000076928.19:s:41896377-41896878;41896482-41897315). The identified gene may include ARHGEF7 (ENSG00000102606.20:t:111217679-111217885;111210002-111217679). The identified gene may include ARMC10 (ENSG0000070632.14:s:103074881-103075411;103075411-103075777). The identified gene may include ARRDC2 (ENSG00000105643.11:t:18008711-18008861;18001573-18008711). The identified gene may include ATE1 (ENSG00000107669.19:t:121924266-121924329;121924329-121928347). The identified gene may include ATP6V1D (ENSG00000100554.12:t:67350611-67350690;67350690-67359658). The identified gene may include ATXN2L (ENSG00000168488.19:s:28835933-28836176;28836122-28836748). The identified gene may include ATXN2L (ENSG00000168488.19:t:28836728-28837237;28836122-28836748). The identified gene may include ATXN2L (ENSG00000168488.19:t:28836728-28837237;28836485-28836728). The identified gene may include AURKB (ENSG00000178999.13:t:8207738-8208225;8207840-8210530). The identified gene may include BLOC1S6 (ENSG00000104164.12:s:45592135-45592276;45592276-45605428). The identified gene may include C12orf76 (ENSG00000174456.16:s:110048363-110048870;110042459-110048363). The identified gene may include CAMTA1 (ENSG00000171735.20:t:6825092-6825210;6820250-6825092). The identified gene may include CBFA2T3 (ENSG00000129993.15:t:88892244-88892485;88892485-88898078). The identified gene may include CBFB (ENSG00000067955.15:t:67066682-67066962;67036755-67066682). The identified gene may include CCDC28B (ENSG00000160050.16:t:32204598-32204620;32204379-32204598). The identified gene may include CCDC92 (ENSG00000119242.9:s:123972529-123972831;123943493-123972529). The identified gene may include CCDC92 (ENSG00000119242.9:t:123943347-123943495;123943493-123972529). The identified gene may include CD300H (ENSG00000284690.3:s:74563919-74564275;74560824-74563919). The identified gene may include CD34 (ENSG00000174059.17:s:207888682-207888846;207887923-207888682). The identified gene may include CDAN1 (ENSG00000140326.13:s:42736989-42737128;42736780-42737013). The identified gene may include CDC14B (ENSG00000081377.17:t:96565393-96565512;96565483-96619219). The identified gene may include CDK5RAP2 (ENSG00000136861.19:t:120402806-120403071;120403071-120403316). The identified gene may include CELF2 (ENSG00000048740.19:t:11249153-11249201;11165682-11249153). The identified gene may include CEP164 (ENSG00000110274.16:t:117409618-117410701;117409028-117409618). The identified gene may include CLEC1B (ENSG00000165682.15:s:9995140-9995246;9986158-9995140). The identified gene may include COX20 (ENSG00000203667.10:s:244835616-244835756;244835756-244842195). The identified gene may include CPNE1 (ENSG00000214078.13:s:35627280-35627531;35626800-35627280). The identified gene may include CYTH2 (ENSG00000105443.17:s:48474838-48476276;48474949-48477717). The identified gene may include CYTH2 (ENSG00000105443.17:t:48478066-48478145;48477719-48478069). The identified gene may include DAZAP1 (ENSG00000071626.17:s:1422348-1422396;1422396-1425878). The identified gene may include DCTD (ENSG0000029187.15:s:182917288-182917477;182915575-182917311). The identified gene may include DCTD (ENSG00000129187.15:t:182915461-182915575;182915575-182917288). The identified gene may include DECR1 (ENSG00000104325.7:s:90001405-90001561;90001561-90017124). The identified gene may include DECR1 (ENSG00000104325.7:s:90001405-90001561;90001561-90018909). The identified gene may include DECR1 (ENSG00000104325.7:t:90018564-90018966;90001561-90018909). The identified gene may include DEF8 (ENSG00000140995.17:t:89954172-89954376;89949513-89954243). The identified gene may include DGKD (ENSG00000077044.11:s:233390403-233390483;233390483-233392070). The identified gene may include DGKZ (ENSG00000149091.15:t:46367291-46367399;46345585-46367291). The identified gene may include DNM2 (ENSG00000079805.19:s:10795372-10795439;10795439-10797380). The identified gene may include DOCK8 (ENSG00000107099.18:t:271627-271729;215029-271627). The identified gene may include DUT (ENSG00000128951.14:t:48332103-48332737;48331479-48332268). The identified gene may include DYRK1A (ENSG00000157540.22:s:37418905-37420384;37420384-37472684). The identified gene may include DYSF (ENSG00000135636.16:t:71480883-71480938;71454086-71480883). The identified gene may include EIF4G1 (ENSG00000114867.22:t:184317321-184317497;184314674-184317321). The identified gene may include EIF4G3 (ENSG00000075151.24:t:20981048-20981227;20981227-20997601). The identified gene may include EMC8 (ENSG00000131148.9:s:85781211-85781760;85779867-85781211). The identified gene may include EPSTI1 (ENSG00000133106.15:s:42991978-42992271;42969177-42991978). The identified gene may include EXOSC1 (ENSG00000171311.13:s:97438663-97438703;97437750-97438663). The identified gene may include EXOSC1 (ENSG00000171311.13:t:97437700-97437750;97437750-97438663). The identified gene may include F2RL3 (ENSG00000127533.4:s:16888999-16889298;16889298-16889577). The identified gene may include FAM219A (ENSG00000164970.15:s:34405865-34405964;34402807-34405916). The identified gene may include FAM3A (ENSG00000071889.17:s:154512823-154512936;154511871-154512823). The identified gene may include FBXO44 (ENSG00000132879.14:t:11658736-11658871;11658628-11658736). The identified gene may include FKBP1B (ENSG00000119782.14:s:24060814-24060926;24060926-24063019). The identified gene may include FMNL3 (ENSG00000161791.14:t:49657082-49657190;49657190-49658442). The identified gene may include FOXP1 (ENSG00000114861.24:t:71112536-71112637;71112637-71130498). The identified gene may include G3BP1 (ENSG00000145907.16:t:151786572-151786715;151772036-151786603). The identified gene may include GET1 (ENSG00000182093.16:t:39390698-39390803;39380556-39390698). The identified gene may include GLUL (ENSG00000135821.19:t:182388572-182390304;182388750-182391175). The identified gene may include GORASP1 (ENSG00000114745.15:s:39107479-39108063;39103553-39107479). The identified gene may include GORASP1 (ENSG00000114745.15:t:39101016-39101102;39103553-39107479). The identified gene may include GSE1 (ENSG00000131149.19:t:85648552-85648751;85556363-85648552). The identified gene may include GSTT1 (ENSG00000277656.3:t:270997-271173;271173-278295). The identified gene may include GUCD1 (ENSG00000138867.17:t:24548917-24549001;24549001-24555615). The identified gene may include HDAC7 (ENSG00000061273.18:s:47796207-47796298;47796016-47796207). The identified gene may include HES6 (ENSG00000144485.11:t:238239487-238239568;238239568-238239825) The identified gene may include HUS1 (ENSG00000136273.13:s:47979410-47979615;47978816-47979410). The identified gene may include IFI27 (ENSG00000275214.4:t:1230073-1230504;1229442-1230343). The identified gene may include IGF2BP3 (ENSG00000136231.14:t:23342064-23342189;23342189-23343718). The identified gene may include IKBKB (ENSG00000104365.16:t:42290156-42290273;42288728-42290156). The identified gene may include IKBKG (ENSG00000269335.7:t:154551988-154552189;154542452-154551988). The identified gene may include IKZF3 (ENSG00000161405.17:t:39777651-39777767;39777767-39788258). The identified gene may include IL10RA (ENSG00000110324.12:t:117988382-117988502;117986534-117988382). The identified gene may include IL15RA (ENSG00000134470.21:t:5960367-5960567;5960567-5963743). The identified gene may include IMPDH1 (ENSG00000106348.18:t:128405767-128405948;128405865-128409289). The identified gene may include INO80E (ENSG00000169592.15:s:30001414-30001575;30001530-30005221). The identified gene may include INPP5D (ENSG00000281614.3:s:67445-67595;67595-71086). The identified gene may include IP6K2 (ENSG00000068745.15:t:48694872-48695421;48695421-48717157). The identified gene may include IRF7 (ENSG00000276561.4:s:144369-144427;144292-144369). The identified gene may include IRF7 (ENSG00000276561.4:s:144676-144900;144424-144690). The identified gene may include ITSN2 (ENSG00000198399.16:s:24246129-24246320;24242206-24246129). The identified gene may include JAML (ENSG00000160593.19:t:118212407-118212561;118212561-118214824). The identified gene may include JOSD2 (ENSG00000161677.12:t:50506378-50506572;50506572-50507574). The identified gene may include KANK2 (ENSG00000197256.11:s:11195556-11195881;11194590-11195556). The identified gene may include KANK2 (ENSG0000197256.11:t:11194553-11194590;11194590-11195556). The identified gene may include KANSL3 (ENSG00000114982.19:s:96636739-96637228;96631543-96636921). The identified gene may include KANSL3 (ENSG00000114982.19:t:96631312-96631543;96631543-96636921). The identified gene may include KAT5 (ENSG00000172977.13:s:65712922-65713319;65713058-65713348). The identified gene may include KATNB1 (ENSG00000140854.13:s:57755345-57755494;57755494-57755841). The identified gene may include KDM5C (ENSG00000126012.13:s:53217796-53217966;53217274-53217796). The identified gene may include KIAA1671 (ENSG00000197077.14:t:25170820-25170938;25049364-25170820). The identified gene may include KIFC3 (ENSG000000140859.16:t:57772223-57772288;57772288-57785464). The identified gene may include KIFC3 (ENSG00000140859.16:t:57798072-57798282;57798282-57802370). The identified gene may include KMT2B (ENSG00000272333.8:t:35719784-35720940;35719541-35719784). The identified gene may include LDB1 (ENSG00000198728.11:s:102109029-102109177;102108323-102109029). The identified gene may include LDB2 (ENSG00000169744.13:s:16511981-16512104;16508686-16511981). The identified gene may include LETMD1 (ENSG00000050426.16:s:51048301-51048518;51048478-51056350). The identified gene may include LST1 (ENSG00000204482.11:s:31587117-31587318;31587318-31587641). The identified gene may include LST1 (ENSG00000204482.11:t:31587944-31587966;31587318-31587944). The identified gene may include LST1 (ENSG00000206433.10:s:2842938-2843143;2843139-2844386). The identified gene may include LST1 (ENSG00000223465.8:s:2834852-2835057;2835053-2835376). The identified gene may include LST1 (ENSG00000223465.8:t:2835679-2835701;2835053-2835679). The identified gene may include LST1 (ENSG00000226182.8:t:3065231-3065253;3064605-3065231). The identified gene may include LST1 (ENSG00000230791.8:s:2886832-2887014;2887014-2887225). The identified gene may include LST1 (ENSG00000230791.8:t:2887225-2887247;2886599-2887225). The identified gene may include LST1 (ENSG00000231048.8:s:2892158-2892363;2892359-2892682). The identified gene may include LST1 (ENSG00000231048.8:t:2892985-2893007;2892359-2892985). The identified gene may include LTB (ENSG00000204487.8:s:3059543-3059714;3058917-3059543). The identified gene may include LTB (ENSG00000223448.7:s:2887297-2887468;2886671-2887297). The identified gene may include LTB (ENSG00000238114.7:s:2881536-2881707;2880910-2881536). The identified gene may include LTBP4 (ENSG00000090006.18:s:40619347-40619493;40619493-40622401). The identified gene may include LTBP4 (ENSG00000090006.18:t:40619347-40619493;40614446-40619347). The identified gene may include LTBP4 (ENSG00000090006.18:t:40623604-40623732;40623021-40623604). The identified gene may include MAPK9 (ENSG00000050748.18:t:180269280-180269409;180269409-180279796). The identified gene may include MARK2 (ENSG00000072518.22:s:63904786-63905043;63905043-63908260). The identified gene may include MAST3 (ENSG00000099308.13:s:18146881-18147044;18147017-18147443). The identified gene may include MAX (ENSG000000125952.20:t:65077193-65077428;65077428-65077913). The identified gene may include MBNL1 (ENSG00000152601.18:t:152414941-152415111;152269092-152414941. The identified gene may include METTL9 (ENSG00000197006.15:s:21624931-21625233;21625115-21655227). The identified gene may include MGLL (ENSG00000074416.16:t:127821694-127821838;127821838-127822309). The identified gene may include MGRN1 (ENSG00000102858.13:s:4677463-4677572;4677572-4681550). The identified gene may include MIEF1 (ENSG00000100335.15:s:39511849-39512026;39512026-39512232). The identified gene may include MLX (ENSG00000108788.12:s:42567619-42567655;42567655-42568837). The identified gene may include MMAB (ENSG00000139428.12:s:109561420-109561517;109561329-109561420). The identified gene may include MPI (ENSG00000178802.18:s:74890005-74890201;74890089-74890527). The identified gene may include MPRIP (ENSG00000133030.22:t:17142627-17142765;17138429-17142627). The identified gene may include MRPL33 (ENSG00000243147.8:s:27772674-27772855;27772692-27779433). The identified gene may include MSRB3 (ENSG00000174099.12:s:65278643-65278865;65278865-65326826). The identified gene may include MTA1 (ENSG00000182979.18:s:105445418-105445511;105445511-105450058). The identified gene may include MTA1 (ENSG00000182979.18:t:105450058-105450184;105445511-105450058). The identified gene may include MTHFD2 (ENSG00000065911.13:t:74207704-74207826;74205889-74207704). The identified gene may include MTMR12 (ENSG00000150712.11:s:32312758-32312987;32274122-32312758). The identified gene may include MTMR12 (ENSG00000150712.11:t:32273980-32274122;32274122-32312758). The identified gene may include NAP1L4 (ENSG00000273562.4:t:175479-176694;176694-182308). The identified gene may include NCOA2 (ENSG00000140396.13:t:70141179-70141399;70141399-70148273). The identified gene may include NCOR2 (ENSG00000196498.14:s:124330845-124330898;124326370-124330845). The identified gene may include NCOR2 (ENSG00000196498.14:s:124378237-124378384;124372610-124378237). The identified gene may include NCOR2 (ENSG00000196498.14:t:124372022-124372610;124372610-124378237). The identified gene may include NDRG2 (ENSG00000165795.25:t:21022392-21022820;21022497-21022864). The identified gene may include NDUFV3 (ENSG00000160194.18:t:42908864-42913304;42897047-42908864). The identified gene may include NFE2L1 (ENSG00000082641.16:s:48056386-48056598;48056598-48057032). The identified gene may include NFIA (ENSG00000162599.18:t:61455303-61462788;61406727-61455303). The identified gene may include NKIRAS2 (ENSG00000168256.18:t:42023654-42025644;42022640-42023654). The identified gene may include NOC2L (ENSG00000188976.11:s:945042-945146;944800-945057). The identified gene may include NTAN1 (ENSG00000275779.4:s:613600-613788;613788-621580). The identified gene may include NTPCR (ENSG00000135778.12:t:232956347-232956443;232950744-232956347). The identified gene may include NUDT22 (ENSG00000149761.9:t:64229131-64229344;64227132-64229247). The identified gene may include NUP214 (ENSG00000126883.19:s:131146129-131146304;131146304-131147490). The identified gene may include NXT2 (ENSG00000101888.12:t:109538045-109538131;109537270-109538045). The identified gene may include OCEL1 (ENSG00000099330.9:s:17226213-17226353;17226353-17226693). The identified gene may include P2RX4 (ENSG00000135124.16:s:121210065-121210298;121210298-121217134). The identified gene may include PABIR2 (ENSG00000156504.17:s:134781821-134781917;134772283-134781821). The identified gene may include PALM2AKAP2 (ENSG00000157654.19:s:110138171-110138539;110138539-110168399). The identified gene may include PANK4 (ENSG00000157881.16:t:2521101-2521315;2521315-2526464). The identified gene may include PAPOLA (ENSG00000090060.19:s:96556175-96556413;96556413-96560649). The identified gene may include PARL (ENSG00000175193.14:s:183862607-183862801;183844326-183862697). The identified gene may include PARL (ENSG00000175193.14:t:183844231-183844774;183844326-183862753). The identified gene may include PARVB (ENSG00000188677.15:t:44093928-44094017;44069162-44093928). The identified gene may include PCYT1B (ENSG00000102230.14:t:24618985-24619084;24619084-24646989). The identified gene may include PDE7A (ENSG00000205268.11:t:65782783-65782843;65782843-65841371). The identified gene may include PEX26 (ENSG00000215193.14:s:18083437-18083732;18083732-18087972). The identified gene may include PFKFB3 (ENSG00000170525.21:t:6213623-6213748;6203336-6213623). The identified gene may include PKN1 (ENSG00000123143.13:t:14441143-14441443;14433542-14441143). The identified gene may include PLEKHM1 (ENSG00000225190.12:s:45481603-45482612;45478147-45482433). The identified gene may include PLS3 (ENSG00000102024.19:t:115610243-115610323;115561260-115610243). The identified gene may include PML (ENSG00000140464.20:s:74034478-74034558;74034530-74042989). The identified gene may include PML (ENSG00000140464.20:t:74033156-74033422;74032715-74033156). The identified gene may include POLR2J3 (ENSG00000168255.20:t:102566997-102567083;102567083-102568010). The identified gene may include POLR2J3 (ENSG00000285437.2:t:102566966-102567083;102567083-102568010). The identified gene may include PPIE (ENSG00000084072.17:s:39752910-39753266;39753052-39753287). The identified gene may include PPM1N (ENSG00000213889.11:t:45499949-45500066;45497343-45499949). The identified gene may include PPP6R2 (ENSG00000100239.16:s:50436367-50436452;50436452-50436988) The identified gene may include PPP6R2 (ENSG00000100239.16:t:50437506-50437603;50437068-50437506). The identified gene may include PRKAR1B (ENSG00000188191.16:t:596146-596304;596304-602553). The identified gene may include PRKCD (ENSG00000163932.16:t:53178404-53178537;53165215-53178404). The identified gene may include PSEN1 (ENSG00000080815.20:t:73170797-73171923;73148094-73170797). The identified gene may include PSMB8 (ENSG00000230034.8:t:4086512-4086659;4086659-4087849). The identified gene may include PSMG4 (ENSG00000180822.12:s:3263684-3263759;3263759-3264209). The identified gene may include PTK2B (ENSG00000120899.18:t:27450749-27450895;27445919-27450749). The identified gene may include PTPN12 (ENSG00000127947.16:s:77600664-77600965;77600806-77607235). The identified gene may include PTPN18 (ENSG00000072135.13:t:130368897-130369201;130356200-130369133). The identified gene may include PUF60 (ENSG00000179950.15:s:143821118-143821743;143818534-143821597). The identified gene may include PUF60 (ENSG00000179950.15:s:143821118-143821743;143820716-143821597). The identified gene may include PUM1 (ENSG00000134644.16:s:30967099-30967310;30966278-30967167). The identified gene may include PYM1 (ENSG00000170473.17:t:55903387-55903480;55903480-55927076). The identified gene may include RAB11FIP1 (ENSG00000156675.16:t:37870387-37870528;37870528-37871278). The identified gene may include RAB11FIP5 (ENSG00000135631.17:t:73075993-73076182;73076182-73088050). The identified gene may include RAB4A (ENSG00000168118.12:t:229295848-229295910;229271370-229295848). The identified gene may include RABGAP1L (ENSG00000152061.24:t:174969277-174969387;174957549-174969277). The identified gene may include RAP1B (ENSG00000127314.18:t:68650244-68650882;68648781-68650400). The identified gene may include RARA (ENSG00000131759.18:t:40348316-40348464;40331396-40348316). The identified gene may include RBCK1 (ENSG00000125826.22:s:409830-410025;410025-417526). The identified gene may include RBM10 (ENSG00000182872.16:t:47173128-47173197;47169498-47173128). The identified gene may include RBM39 (ENSG00000131051.24:s:35713019-35713203;35709256-35713019). The identified gene may include RCOR3 (ENSG00000117625.14:t:211313424-211316385;211312961-211313424). The identified gene may include REPS2 (ENSG00000169891.18:s:17022123-17022371;17022271-17025059). The identified gene may include RFFL (ENSG00000092871.17:s:35026374-35026568;35021781-35026374). The identified gene may include RHD (ENSG00000187010.21:s:25303322-25303459;25303459-25328898). The identified gene may include RMND5B (ENSG00000145916.19:t:178138108-178138396;178131054-178138108). The identified gene may include RPUSD1 (ENSG00000007376.8:t:786826-786928;786928-787077). The identified gene may include SEC16A (ENSG00000148396.19:s:136447227-136447364;136446949-136447227). The identified gene may include SIGLEC10 (ENSG00000142512.15:t:51414422-51414515;51414515-51415181). The identified gene may include SIRT3 (ENSG00000142082.15:s:218778-219041;216718-218778). The identified gene may include SLA2 (ENSG00000101082.14:s:36615225-36615374;36614387-36615225). The identified gene may include SLC66A2 (ENSG00000122490.19:t:79903446-79904183;79904183-79919184). The identified gene may include SMARCA4 (ENSG00000127616.21:s:11034914-11035132;11035132-11041298). The identified gene may include SMARCA4 (ENSG00000127616.21:t:11040634-11041560;11035132-11041298). The identified gene may include SMARCC2 (ENSG00000139613.12:s:56173696-56173849;56173029-56173696). The identified gene may include SNX20 (ENSG00000167208.16:s:50675770-50675921;50669148-50675770). The identified gene may include SPIB (ENSG00000269404.7:s:50423605-50423755;50423751-50428038). The identified gene may include SRSF6 (ENSG00000124193.16:s:43458361-43458509;43458509-43459153). The identified gene may include SSX2IP (ENSG00000117155.17:t:84670646-84670815;84670815-84690371). The identified gene may include STAMBP (ENSG00000124356.17:t:73830845-73831059;73829070-73830845). The identified gene may include STAT3 (ENSG00000168610.16:t:42317182-42317224;42317224-42319856). The identified gene may include SWI5 (ENSG00000175854.15:t:128276587-128276755;128276402-128276707). The identified gene may include SYNE1 (ENSG00000131018.25:s:152301869-152302269;152300781-152301869). The identified gene may include TANGO2 (ENSG00000183597.16:t:20061530-20061802;20056013-20061530). The identified gene may include TCF3 (ENSG00000071564.19:s:1615686-1615804;1612433-1615686). The identified gene may include TCF7L2 (ENSG00000148737.18:s:113146011-113146097;113146097-113150998). The identified gene may include TECR (ENSG00000099797.15:s:14562356-14562575;14562575-14563174). The identified gene may include TJP2 (ENSG00000119139.21:t:69212548-69212601;69151771-69212548). The identified gene may include TLE4 (ENSG00000106829.21:s:79627374-79627752;79627448-79652593). The identified gene may include TLE4 (ENSG00000106829.21:t:79652590-79652629;79627448-79652593). The identified gene may include TMBIM1 (ENSG00000135926.15:s:218292466-218292586;218282181-218292466). The identified gene may include TMBIM4 (ENSG00000155957.19:s:66169855-66170027;66161172-66169855). The identified gene may include TMEM11 (ENSG00000178307.10:s:21214091-21214176;21211227-21214091). The identified gene may include TMEM126B (ENSG00000171204.13:t:85631687-85631808;85628688-85631687). The identified gene may include TMEM219 (ENSG000000149932.17:t:29962677-29963308;29962132-29963107). The identified gene may include TNFSF12 (ENSG00000239697.12:t:7549466-7549618;7549312-7549474). The identified gene may include TNK2 (ENSG00000061938.21:t:195888426-195888606;195888606-195908485). The identified gene may include TNRC18 (ENSG00000182095.15:t:5325096-5325574;5325248-5329937). The identified gene may include TRAPPC2 (ENSG00000196459.15:s:13734525-13734635;13719982-13734525). The identified gene may include TRAPPC6A (ENSG00000007255.10:t:45164725-45164970;45164970-45165127). The identified gene may include TSC22D3 (ENSG00000157514.18:t:107715773-107715950;107715950-107775100). The identified gene may include TSPAN32 (ENSG00000064201.16:s:2314485-2314666;2314571-2316229). The identified gene may include UBAC2 (ENSG00000134882.16:t:99238427-99238554;99200939-99238427). The identified gene may include UFD1 (ENSG00000070010.19:t:19471687-19471808;19471808-19479083). The identified gene may include URI1 (ENSG00000105176.18:s:29942239-29942664;29942664-29985223). The identified gene may include URI1 (ENSG00000105176.18:t:29985223-29985301;29942664-29985223). The identified gene may include USP15 (ENSG000000135655.16:s:62325872-62325933;62325933-62349221). The identified gene may include USP22 (ENSG00000124422.12:t:21028542-21028674;21028674-21043321). The identified gene may include VGLL4 (ENSG00000144560.16:t:11601833-11602022;11602022-11702971). The identified gene may include WWP2 (ENSG00000198373.13:t:69786996-69787080;69762391-69786996). The identified gene may include YAF2 (ENSG00000015153.15:s:42237599-42237800;42199235-42237599). The identified gene may include YIPF6 (ENSG00000181704.12:s:68498562-68499157;68499123-68511849). The identified gene may include YIPF6 (ENSG00000181704.12:t:68513327-68515340;68511977-68513327). The identified gene may include ZBTB7B (ENSG00000160685.14:t:155014448-155015814;155002943-155014655). The identified gene may include ZEB2 (ENSG00000169554.23:t:144517278-144517557;144517419-144517612). The identified gene may include ZFAND1 (ENSG00000104231.11:s:81718182-81718224;81715114-81718182). The identified gene may include ZFAND1 (ENSG00000104231.11:t:81714987-81715114;81715114-81718182). The identified gene may include ZFAND2B (ENSG00000158552.13:s:219207648-219207779;219207774-219207887). The identified gene may include ZMIZ2 (ENSG00000122515.16:s:44759281-44759460;44759460-44760151). The identified gene may include ZMYND8 (ENSG00000101040.20:t:47236326-47236516;47236516-47238758). The identified gene may include ZNF185 (ENSG00000147394.20:s:152922720-152922809;152922809-152928575). The identified gene may include ZNF451 (ENSG00000112200.17:t:57099061-57099141;57090274-57099061. The identified gene may include any of the genes listed in Tables 1-3.

The methods may utilize computer processing. The computer processing may make use of machine learning as disclosed herein. The computer processing may use of a classifier as disclosed herein. The computer processing may make use of prediction or classification. The classifier may be a trained classifier. The classifier may be trained by the methods disclosed herein.

The methods may detect a disease state based at least in part on the computer processing. The methods may comprise detecting the disease state with an accuracy. In some cases, the disease state is detected with an accuracy of more than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The methods may comprise detecting the disease state with a particular sensitivity. In some cases, the disease state is detected with a sensitivity of more than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The methods may comprise detecting the disease state using a receiver operating characteristic (ROC) curve with an area under the curve (AUC) value. In some cases, the disease state is detected with an AUC value of an ROC curve of more than or equal to 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99.

The methods may include administering a treatment to a subject. The treatment may treat a disease state of the subject. The treatment may treat one or more disease states of the subject. The treatment may be administered orally, intravenously, intramuscularly, subcutaneously, intrathecally, rectally, vaginally, topically, intranasally, or any combination thereof. The treatment may be in the form of a liquid, a tablet, or a capsule. The treatment may be in the form of a cream, a lotion, or an ointment. The treatment may be in the form of a droplet, an inhaler, an injection, a patch, an implant, or a suppository.

The treatment may treat memory loss, behavioral changes, sleep problems, and other symptoms associated with Alzheimer's disease. For example, citalopram, fluoxetine, paroxetine, and sertraline can be used to treat issues relating to mood, depression, and irritability experienced by Alzheimer's disease. As another example, alprazolam, buspirone, lorazepam, and oxazepam can be used to treat anxiety or restlessness associated with Alzheimer's disease. Further, unconventional therapies, such as hormone replacement therapy, art and music therapies, and supplements (e.g., vitamins such as vitamin E) can be used alternatively or additionally to treat the Alzheimer's disease.

The treatment may be a medicinal therapy. The medicinal therapy may be a cholinesterase inhibitor. The medicinal therapy may be a N-methyl-D-aspartate (NMDA) antagonist. The medicinal therapy may be an atypical antipsychotic. The medicinal therapy may be a disease-modifying immunotherapy. The medicinal therapy may be a monoclonal antibody (mab) therapy. The medicinal therapy may be an amyloid monoclonal antibody (mab) therapy.

The treatment may be a behavioral therapy. The behavioral therapy may include cognitive behavioral therapy, cognitive behavioral play therapy, dialectical behavioral therapy, exposure therapy, rational emotive behavior therapy, cognitive restructuring, aversion therapy, interpersonal psychotherapy, multisensory stimulation, active music therapy, cognitive stimulation, or any combination thereof. The treatment may be a sleep therapy.

### Determining a Risk of a Disease State

Provided herein are methods of determining a risk of a disease state in a subject. The methods may comprise obtaining a biological sample from the subject. The methods may comprise assaying cell-free messenger RNA (cf-mRNA) in the biological sample. The assaying may be to detect one or more splice junctions in the cf-mRNA. The one or more splice junctions may correspond to one or more genes.

The methods may comprise determining a risk of a disease state. The disease state may comprise a presence or an absence of a disease state. The disease state may be a stage of a disease, for example an incubation stage, a prodromal stage, an illness stage, a decline stage, or a convalescence stage. The disease state may be a likelihood of having a disease. The disease state may be one or more diseases, for example, two or more, three or more, four or more, or five or more diseases. The disease state may be a combination of disease states. The disease state may be an infectious disease, a deficiency disease, a hereditary disease (e.g., genetic or non-genetic), or a physiological disease. The disease state may be a disease of a bodily region or system, for example, a vascular disease, a gastrointestinal disease, a chest disease, or the like. The disease state may include a resilience state. The disease state may be a disease of an organ or a tissue, for example, a disease state of the heart, a disease state of the liver, a disease of the lung, a disease state of the skin, a disease state of the kidney, a disease state of the brain, or the like. The disease state may originate from an organ or a tissue, for example, the heart, the liver, the lung, the skin, the brain, the kidney, or the like. The disease state may impact one or more organs or tissues, for example, one or more of the heart, the liver, the lung, the skin, the brain, the kidney, or the like. The disease state may be a disease of a bodily function, for example, a metabolic disease, or the like.

The disease state may relate to a dementia. The disease state may be Alzheimer's disease. The Alzheimer's disease may be a stage of Alzheimer's disease, such as preclinical Alzheimer's disease, mild cognitive impairment due to Alzheimer's disease, mild dementia due to Alzheimer's disease, moderate dementia due to Alzheimer's disease, or severe dementia due to Alzheimer's disease.

The disease state may relate to memory. The disease state may relate to changes in mood, personality, disorientation, or the like. The disease state may relate to problems with speech, movement, problem solving, communication, or the like. The disease state may relate to confusion. The disease state may relate to spatial awareness. The disease state may relate to judgement and decision making. The disease state may be Huntington disease, frontotemporal dementia, Lewy Body Dementia (LBD), normal pressure hydrocephalus, vascular dementia, mixed dementia, corticobasal degeneration, progressive supranuclear palsy, chronic traumatic encephalopathy, multiple sclerosis, depression, general dementia, or the like. The disease state may be major depression, dysthymia, bipolar disorder, substance-induced mood disorders, or any other mood disorders. The disease state may relate to articulation disorders, phonological disorders, disfluency, voice disorders, or the like.

The methods may comprise determining a risk of a disease state in a subject. The subject may be an animal. The subject may be a mammal, such as a human, a non-human primate, a rodent (e.g., a rat, a mouse, a guinea pig, a hamster, or the like), a dog, a cat, a pig, a sheep, a cow, a goat, or a rabbit. The subject may be a fish, a reptile, or a bird. The subject may be a human. The subject may be an adult (e.g., 18 years of age or older). The subject may be a child (e.g., less than 18 years of age). The subject may comprise an age of greater than or equal to 1, 2, 5, 10, 15, 20, 25, 30, 35, 40, 45, 50, 55, 60, 65, 70, 75, 80, 85, 90, or 95 years of age. The subject may be from about 50 to about 85 years of age. The subject may be from about 60 to about 80 years of age. The subject may be about 70 years of age. The subject may have or be suspected of having a disease state disclosed herein. For example, the subject may have or be suspected of having a dementia, for example, Alzheimer's disease. The subject may be asymptomatic. The subject may be healthy. The subject may be suspected of having a risk of a disease state disclosed herein. The subject may have one or more risk factors associated with a disease state. For example, the subject may have risk factors such as diabetes, hypertension, or the like. The subject may be predisposed to having a disease state disclosed herein. For example, the subject may be predisposed to having Alzheimer's disease. The subject may be in remission from a treatment to the disease state. The subject may have one or more symptoms of a disease state disclosed herein. For example, the subject may have symptoms such as memory loss, misplacement of items, difficulty in decision making and judging, confusion, mood swings, social withdrawal, inability to problem solve or complete tasks, or the like.

The methods may comprise obtaining a biological sample from a subject. The biological sample may be a blood sample. The biological sample may be a plasma sample. The biological sample may be a serum sample. The biological sample may be a buffy coat sample. The biological sample may be a urine sample. The biological sample may be a saliva sample. The biological sample may be a sweat sample. The biological sample may be a semen sample. The biological sample may be a vaginal discharge sample. The biological sample may be a cell-free sample. The cell-free sample may comprise cell-free RNA, such as cell-free mRNA (cf-mRNA). The biological sample may be a tissue sample. The biological sample may be a tumor biopsy sample. The biological sample may be a bone marrow sample.

The biological sample may comprise nucleic acids. The biological sample may comprise ribonucleic acids (RNAs), such as messenger RNAs (mRNAs). The RNA may be cell-free. The cell-free RNAs may be cell-free mRNAs. The RNA may be pre-mRNA. The RNA may comprise a coding region. The RNA may comprise a non-coding region. The RNA may comprise small nuclear RNAs (snRNAs), micro RNAs (miRNAs), or small interfering RNAs (siRNAs). The biological sample may comprise deoxyribonucleic acids (DNAs). The biological sample may comprise proteins.

The methods may comprise assaying the biological sample. In some cases, the methods may comprise assaying cf-mRNA in the biological sample to detect one or more splice junctions in the cf-mRNA. The one or more splice junctions may correspond to one or more genes.

The methods may include sequencing. Non-limiting examples of sequencing include sequencing by synthesis (SBS), pyrosequencing, sequencing by reversible terminator chemistry, sequencing by ligation, phospholinked fluorescent nucleotide sequencing, real-time sequencing, and the like. The method may include next generation sequencing (NGS). NGS utilizes the concept of massively parallel processing to obtain high-throughput, speed, and scalability. NGS may be referred to as massive parallel sequencing, massively parallel sequencing, or second-generation sequencing. The methods may include RNA sequencing. Non-limiting examples of RNA sequencing include mRNA sequencing, total RNA sequencing, low-input RNA sequencing, ultra-low-input RNA sequencing, small RNA sequencing, single cell RNA sequencing, and the like. The methods may include DNA sequencing. Non-limiting examples of DNA sequencing include sanger sequencing, capillary electrophoresis, sequencing by synthesis, shotgun sequencing, pyrosequencing, combinatorial probe anchor synthesis, sequencing by ligation, nanopore sequencing, single molecular real time sequencing, ion torrent sequencing, nanoball sequencing, next generation sequencing, and the like.

The methods may include array hybridization. Array hybridization may include us of a microarray. A microarray is a laboratory tool that may be used to detect the expression of multiple genes at the same time. The microarray may be an analytical microarray, an antibody microarray, a functional microarray, a spotted array, a cellular microarray, an oligonucleotide DNA microarray, or the like. The microarray may use fluorescent dyes. The microarray may use probes, such as nucleotide probes. The microarray may comprise one or more wells, such as a 16-well plate, a 24-well plate, a 96 well plate, a 384-well plate, or the like. The one or more wells may be organized in rows and columns on the microarray.

The methods may include nucleic acid amplification. Nucleic acid amplification may include polymerase chain reaction (PCR), for example, multiplex PCR, long-range PCR, single-cell PCR, fast cycling PCR, methylation specific PCR, digital PCR, hot start PCR, real-time PCR (RT-PCR), quantitative PCR (qPCR), or the like. The nucleic acid amplification may include loop mediated isothermal amplification (LAMP). The nucleic acid amplification may include nucleic acid sequence-based amplification (NASBA). The nucleic acid amplification may include a strand displacement amplification (SDA). The nucleic acid amplification may include a multiple displacement amplification (MDA). The nucleic acid amplification may include rolling circle amplification (RCA). The nucleic acid amplification may include ligase chain reaction (LCR). The nucleic acid amplification may include helicase dependent amplification (HDA). The nucleic acid amplification may include a ramification amplification method (RAM). The nucleic acid amplification may include a transcription-mediated assay (TMA).

The methods may further include identifying a tissue of a disease state. The methods may comprise analyzing the cf-mRNA in the biological sample and determining a tissue that the cf-mRNA originated from. The tissue may be identified to be under duress. The tissue may be identified to be impacted by the disease state. The tissue may be identified to be the origin of the disease state. The tissue may be nervous tissue, such as tissue of the brain, spinal cord, or nerves. The tissue may comprise circulating immune cells. The tissue may be muscle tissue, such as cardiac muscle tissue, smooth muscle tissue, or skeletal muscle tissue. The muscle tissue may originate from muscles in the body. The tissue may be epithelial tissue, such as lining of the gastrointestinal tract of organs or the skin surface (epidermis). The tissue may be connective tissue, such as tissue from fat (or other soft padding tissue), bone, or tendons. The tissue may be any tissue in the body.

The methods may further comprise identifying an organ of a disease state. One or more organs may be identified of the disease state. The methods may comprise analyzing the cf-mRNA in the biological sample and determining an organ that the cf-mRNA originated from. The organ may be identified to be under duress. The organ may be identified to be impacted by the disease state. The organ may be identified to be the origin of the disease state. The organ may be the lungs. The organ may be the liver. The organ may be the bladder. The organ may be the kidneys. The organ may be the heart. The organ may be the stomach. The organ may be the intestines, such as the small intestine or the large intestine. The organ may be the brain. The organ may be the pancreas. The organ may be the gallbladder. The organ may be any organ in the body.

The methods may further comprise identifying one or more biological pathways of the disease state. The biological pathways may be identified to be under duress. The biological pathways may be identified to be impacted by the disease state. The biological pathways may be identified to be the origin of the disease state. The biological pathways may include neurological pathways, digestive pathways, muscular pathways, respiratory pathways, endocrine pathways, reproductive pathways, skeletal pathways, lymphatic pathways, immune pathways, immunological pathways, gastrointestinal pathways, nervous system pathways, or any combination thereof. The biological pathways may relate to the disease state. For example, the biological pathways may relate to a neurodegenerative disease. In some cases, the neurodegenerative disease is Alzheimer's disease.

The methods may include producing complementary deoxyribonucleic acid (cDNA) from RNA. In some cases, the methods may include converting RNA, for example cf-mRNA, to cDNA using a reverse transcription protocol. Reverse transcription is a process that converts RNA to cDNA using, among other things, a reverse transcriptase enzyme and deoxyribonucleotide triphosphates (dNTPs). Reverse transcriptase is an enzyme that is an RNA-dependent DNA polymerase. Reverse transcription may utilize several reaction components, such as an RNA template, one or more primers, one or more reaction buffers, dNTPs, DTT, RNase inhibitor, DNA polymerase, DNA ligase, water, or a combination thereof. The reverse transcription reaction may generally follow the steps of annealing, polymerization, and deactivation.

In some cases, a sample cDNA is produced from cf-mRNA by reverse transcription. In some cases, a cDNA library may be produced from the produced cDNA sample. The cDNA library may contain DNA copies of the cf-mRNA obtained from the biological sample. The cDNA library may be compared to a reference library. The reference library may be generated from a biological sample of a subject known not to have the disease state, for example, a subject known to be non-cognitively impaired, or a subject known not to have Alzheimer's disease.

The methods may include comparing a sample cDNA to a reference sample. The reference sample may be obtained from a healthy subject known not to have the disease state. For example, the reference sample may be obtained from a non-cognitively impaired subject. The methods may comprise identifying differences between the sample cDNA and the reference sample. For example, non-contiguous junctions may be present in the sample cDNA and not present in the reference sample. For example, one or more splice junctions may be present in the sample cDNA and not present in the reference sample. Additional differences, such as differences in nucleotide sequences, may be identified between the sample cDNA and the reference sample. In some cases, the reference sample may comprise aggregated least variant gene cf-mRNAs. In some cases, the reference sample may comprise prior sampling. In some cases, the reference sample may comprise a reference interval.

In some cases, the methods may comprise detecting one or more splice junctions. Splice junctions may be referred to as the boundaries between introns and/or exons during RNA splicing in transcription. In some cases, splice junctions may comprise non-coding splice junctions, such as splice junctions in 5′ or 3′ untranslated regions. Transcription is the process by which a cell makes an RNA copy of a piece of DNA. Splicing is the process in which introns, which are the noncoding regions of genes, are excised out of the primary messenger RNA transcript, and the exons, which are the coding regions, are joined together to generate a mature messenger RNA. Non-limiting examples of splice junctions include exon-exon splice junctions (e.g., the boundary between two exons), exon-intron splice junctions (e.g., the boundary between an exon and an intron), and intron-intron splice junctions (e.g., the boundary between two introns). The identification of splice junctions involves the recognition of exon-exon, exon-intron, and intron-intron boundaries during transcription. A splice junction may comprise a boundary between two nucleotides. A splice junction may comprise more than or equal to one nucleotide, for example, more than or equal to two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

The one or more splice junctions may comprise one or more isoforms. An isoform is a specific combination of splice junctions that can result from alternative splicing. Alternative splicing, also called alternative RNA splicing or differential splicing, is a process that allows a single gene to code for multiple proteins. Alternative splicing may generate different mRNAs that are translated differentially into proteins. In alternative splicing, exons/introns from the same gene are joined together in different combinations, leading to different but related resulting mRNA transcripts during transcription. There are several modes of alternative splicing, including exon skipping alternative splicing, mutually exclusive exon alternative slicing, alternative 3′ alternative splicing, alternative 5′ alternative splicing, and intron retention alternative splicing. In exon skipping alternative splicing, an exon may be retained or spliced out of the transcript. Exon skipping alternative splicing is the most common form of alternative splicing and results in the loss of an exon in the alternatively spliced transcript. In mutually exclusive exon alternative splicing, alternative isoforms are generated by retaining only one exon of a cluster of neighboring internal exons in the mature transcript. Mutually exclusive exon alternative splicing indicates that one out of two exons (or one group out of two exon groups) is retained, while the other exon/group is spliced out. In alternative 5′ alternative splicing, an alternative 5′ splice junction is used, which changes the 3′ boundary of the upstream exon. In alternative 3′ alternative splicing, an alternative 3′ splice junction is used, which changes the 5′ boundary of the downstream exon. In intron retention alternative splicing, an intron is retained in the mature mRNA transcript. In some cases, splicing occurs in a 3′ or 5′ untranslated region.

The one or more splice junctions may correspond to one or more genes. The methods may comprise determining that more than or equal to one splice junction corresponds to more than or equal to one gene. In some cases, one splice junction corresponds to more than or equal to one, two, three, four, five six, seven, eight, nine, 10, 11, 12, 13, 14, 15, 16, 17, 18, 19, 20, 21, 22, 23, 24, or 25 genes. In some cases, two splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, three splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, four splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, five splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 10 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 15 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 20 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 25 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 50 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 100 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 250 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 500 splice junctions correspond to more than or equal to one, two, three, four, five six, seven, eight, nine, 10, 11, 12, 13, 14, 15, 16, 17, 18, 19, 20, 21, 22, 23, 24, or 25 genes.

In some cases, the methods disclosed herein may identify one or more genes that are expressed. For example, the genes may be expressed in a first population of subjects with a disease state, such as Alzheimer's disease, as compared to a second population of subjects known not to have the disease state. The second population of subjects may be non-cognitively impaired. The second population of subjects may be healthy. The second population of subjects may be known not have Alzheimer's disease.

The methods may comprise identifying one or more expressed genes. In some cases, the methods comprise identifying one or more, five or more, 10 or more, 20 or more, 30 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, 200 or more, 210 or more, 220 or more, 230 or more, 240 or more, 250 or more, 260 or more, 270 or more, 280 or more, 290 or more, 300 or more, 310 or more, 320 or more, 330 or more, 340 or more, 350 or more, 360 or more, 370 or more, 380 or more, 390 or more, 400 or more, 410 or more, 420 or more, 430 or more, 440 or more, 450 or more, 460 or more, 470 or more, 480 or more, 490 or more, or 500 or more expressed genes. In some cases, the methods comprise identifying 500 or less, 490 or less, 480 or less, 470 or less, 460 or less, 450 or less, 440 or less, 430 or less, 420 or less, 410 or less, 400 or less, 390 or less, 380 or less, 370 or less, 360 or less, 350 or less, 340 or less, 330 or less, 320 or less, 310 or less, 300 or less, 290 or less, 280 or less, 270 or less, 260 or less, 250 or less, 240 or less, 230 or less, 220 or less, 210 or less, 200 or less, 190 or less, 180 or less, 170 or less, 160 or less, 150 or less, 140 or less, 130 or less, 120 or less, 110 or less, 100 or less, 90 or less, 80 or less, 70 or less, 60 or less, 50 or less, 40 or less, 30 or less, 20 or less, 10 or less expressed genes.

The methods may comprise identifying the genes present in Tables 1-3 to be expressed between a first population of disease state subjects as compared to a second population of non-disease state subjects.

Modeling Alternative Junction Inclusion Quantification (“MAJIQ”) is a software package that can detect, quantify, and visualize local splicing variations (“LSV”) from RNA sequencing data. LSVs can include two or more splice junctions that can emanate out from a reference exon (e.g., a source LSV) or converge into a reference exon (e.g., a target LSV). LSV's can capture the classical, binary, alternative splicing events involving two alternative splice junctions. LSV's can also capture more complex (e.g., non-binary) splicing variations. A LSV ID (local splicing variations identifier), as used herein, is a unique identifier for the LSV that can be generated by the MAJIQ package. The LSV ID may be comprised of an ENSG Ensembl ID, whether it is a source (s) or target (t) LSV, exon coordinates, intron coordinates, and combinations thereof. For each gene listed herein, the LSV ID is provided in parentheses, for example Gene (LSV ID (e.g., ENSG number, source (s) or target (t), and exon/intron coordinates).

In some cases, the expressed splice junctions may comprise a member of one or more of the group consisting of: ABLIM1 (ENSG00000099204.21:t:114491791-114491878;114491878-114545005), ACAA1 (ENSG00000060971.19:s:38126493-38126700;38126341-38126510), ACAP1 (ENSG00000072818.12:t:7341948-7342067;7336787-7341948), ACOT8 (ENSG00000101473.17:t:45848450-45849110;45848675-45857188), ACP1 (ENSG00000143727.16:t:272192-273155;272065-272192), ADD3 (ENSG00000148700.15:t:110100625-110100848;110008299-110100625), ADGRE5 (ENSG00000123146.20:t:14397414-14397804;14391079-14397658), ADK (ENSG00000156110.15:t:74200764-74200838;74151343-74200764), AGAP3 (ENSG00000133612.19:s:151119987-151120145;151120145-151122725), AKAP13 (ENSG00000170776.22:t:85575131-85575329;85543955-85575131), ALAD (ENSG00000148218.16:s:113393161-113393634;113392169-113393447), AMPD2 (ENSG00000116337.20:t:109625303-109625433;109621266-109625303), ANAPC11 (ENSG00000141552.18:t:81894298-81894624;81891833-81894467), AP1B1 (ENSG00000100280.17:s:29330378-29330532;29329720-29330378), AP1B1 (ENSG00000100280.17:t:29327680-29328895;29328895-29329712), APP (ENSG00000142192.21:s:26000015-26000182;25982477-26000015), APP (ENSG00000142192.21:t:25897573-25897983;25897673-25911741), ARAP1 (ENSG00000186635.15:s:72693325-72693470;72688537-72693325), ARFRP1 (ENSG00000101246.20:t:63706999-63707658;63707097-63707867), ARHGAP17 (ENSG00000140750.17:t:24935470-24936827;24935639-24941987), ARHGAP17 (ENSG00000288353.1:t:188953-190310;189122-195470), ARHGEF10 (ENSG00000274726.4:s:43270-43560;43560-47749), ARHGEF1 (ENSG00000076928.19:s:41896377-41896878;41896482-41897315), ARHGEF7 (ENSG0000002606.20:t:111217679-111217885;111210002-111217679), ARMC10 (ENSG00000170632.14:s:103074881-103075411;103075411-103075777), ARRDC2 (ENSG00000105643.11:t:18008711-18008861;18001573-18008711), ATE1 (ENSG00000107669.19:t:121924266-121924329;121924329-121928347), ATP6V1D (ENSG00000100554.12:t:67350611-67350690;67350690-67359658), ATXN2L (ENSG00000168488.19:s:28835933-28836176;28836122-28836748), ATXN2L (ENSG00000168488.19:t:28836728-28837237;28836122-28836748), ATXN2L (ENSG00000168488.19:t:28836728-28837237;28836485-28836728), AURKB (ENSG00000178999.13:t:8207738-8208225;8207840-8210530), BLOC1S6 (ENSG00000104164.12:s:45592135-45592276;45592276-45605428), C12orf76 (ENSG00000174456.16:s:110048363-110048870;110042459-110048363), CAMTA1 (ENSG00000171735.20:t:6825092-6825210;6820250-6825092), CBFA2T3 (ENSG00000129993.15:t:88892244-88892485;88892485-88898078), CBFB (ENSG00000067955.15:t:67066682-67066962;67036755-67066682), CCDC28B (ENSG00000160050.16:t:32204598-32204620;32204379-32204598), CCDC92 (ENSG00000119242.9:s:123972529-123972831;123943493-123972529), CCDC92 (ENSG00000119242.9:t:123943347-123943495;123943493-123972529), CD300H (ENSG00000284690.3:s:74563919-74564275;74560824-74563919), CD34 (ENSG00000174059.17:s:207888682-207888846;207887923-207888682), CDAN1 (ENSG00000140326.13:s:42736989-42737128;42736780-42737013), CDC14B (ENSG00000081377.17:t:96565393-96565512;96565483-96619219), CDK5RAP2 (ENSG00000136861.19:t:120402806-120403071;120403071-120403316), CELF2 (ENSG00000048740.19:t:11249153-11249201;11165682-11249153), CEP164 (ENSG00000110274.16:t:117409618-117410701;117409028-117409618), CLEC1B (ENSG00000165682.15:s:9995140-9995246;9986158-9995140), COX20 (ENSG00000203667.10:s:244835616-244835756;244835756-244842195), CPNE1 (ENSG00000214078.13:s:35627280-35627531;35626800-35627280), CYTH2 (ENSG00000105443.17:s:48474838-48476276;48474949-48477717), CYTH2 (ENSG00000105443.17:t:48478066-48478145;48477719-48478069), DAZAP1 (ENSG00000071626.17:s:1422348-1422396;1422396-1425878), DCTD (ENSG00000129187.15:s:182917288-182917477;182915575-182917311), DCTD (ENSG00000129187.15:t:182915461-182915575;182915575-182917288), DECR1 (ENSG00000104325.7:s:90001405-90001561;90001561-90017124), DECR1 (ENSG00000104325.7:s:90001405-90001561;90001561-90018909), DECR1 (ENSG00000104325.7:t:90018564-90018966;90001561-90018909), DEF8 (ENSG00000140995.17:t:89954172-89954376;89949513-89954243), DGKD (ENSG00000077044.11:s:233390403-233390483;233390483-233392070), DGKZ (ENSG00000149091.15:t:46367291-46367399;46345585-46367291), DNM2 (ENSG00000079805.19:s:10795372-10795439;10795439-10797380), DOCK8 (ENSG00000107099.18:t:271627-271729;215029-271627), DUT (ENSG00000128951.14:t:48332103-48332737;48331479-48332268), DYRK1A (ENSG00000157540.22:s:37418905-37420384;37420384-37472684), DYSF (ENSG00000135636.16:t:71480883-71480938;71454086-71480883), EIF4G1 (ENSG00000114867.22:t:184317321-184317497;184314674-184317321), EIF4G3 (ENSG00000075151.24:t:20981048-20981227;20981227-20997601), EMC8 (ENSG00000131148.9:s:85781211-85781760;85779867-85781211), EPSTI1 (ENSG00000133106.15:s:42991978-42992271;42969177-42991978), EXOSC1 (ENSG00000171311.13:s:97438663-97438703;97437750-97438663), EXOSC1 (ENSG00000171311.13:t:97437700-97437750;97437750-97438663), F2RL3 (ENSG00000127533.4:s:16888999-16889298;16889298-16889577), FAM219A (ENSG00000164970.15:s:34405865-34405964;34402807-34405916), FAM3A (ENSG00000071889.17:s:154512823-154512936;154511871-154512823), FBXO44 (ENSG00000132879.14:t:11658736-11658871;11658628-11658736), FKBP1B (ENSG00000119782.14:s:24060814-24060926;24060926-24063019), FMNL3 (ENSG00000161791.14:t:49657082-49657190;49657190-49658442), FOXP1 (ENSG00000114861.24:t:71112536-71112637;71112637-71130498), G3BP1 (ENSG00000145907.16:t:151786572-151786715;151772036-151786603), GET1 (ENSG00000182093.16:t:39390698-39390803;39380556-39390698), GLUL (ENSG00000135821.19:t:182388572-182390304;182388750-182391175), GORASP1 (ENSG00000114745.15:s:39107479-39108063;39103553-39107479), GORASP1 (ENSG00000114745.15:t:39101016-39101102;39103553-39107479), GSE1 (ENSG00000131149.19:t:85648552-85648751;85556363-85648552), GSTT1 (ENSG00000277656.3:t:270997-271173;271173-278295), GUCD1 (ENSG00000138867.17:t:24548917-24549001;24549001-24555615), HDAC7 (ENSG00000061273.18:s:47796207-47796298;47796016-47796207), HES6 (ENSG00000144485.11:t:238239487-238239568;238239568-238239825), HUS1 (ENSG00000136273.13:s:47979410-47979615;47978816-47979410), IFI27 (ENSG00000275214.4:t:1230073-1230504;1229442-1230343), IGF2BP3 (ENSG00000136231.14:t:23342064-23342189;23342189-23343718), IKBKB (ENSG00000104365.16:t:42290156-42290273;42288728-42290156), IKBKG (ENSG00000269335.7:t:154551988-154552189;154542452-154551988), IKZF3 (ENSG00000161405.17:t:39777651-39777767;39777767-39788258), IL10RA (ENSG00000110324.12:t:117988382-117988502;117986534-117988382), IL15RA (ENSG00000134470.21:t:5960367-5960567;5960567-5963743), IMPDH1 (ENSG00000106348.18:t:128405767-128405948;128405865-128409289), INO80E (ENSG00000169592.15:s:30001414-30001575;30001530-30005221), INPP5D (ENSG00000281614.3:s:67445-67595;67595-71086), IP6K2 (ENSG00000068745.15:t:48694872-48695421;48695421-48717157), IRF7 (ENSG00000276561.4:s:144369-144427;144292-144369), IRF7 (ENSG00000276561.4:s:144676-144900;144424-144690), ITSN2 (ENSG00000198399.16:s:24246129-24246320;24242206-24246129), JAML (ENSG00000160593.19:t:118212407-118212561;118212561-118214824), JOSD2 (ENSG00000161677.12:t:50506378-50506572;50506572-50507574), KANK2 (ENSG00000197256.11:s:11195556-11195881;11194590-11195556), KANK2 (ENSG00000197256.11:t:11194553-11194590;11194590-11195556), KANSL3 (ENSG00000114982.19:s:96636739-96637228;96631543-96636921), KANSL3 (ENSG00000114982.19:t:96631312-96631543;96631543-96636921), KAT5 (ENSG00000172977.13:s:65712922-65713319;65713058-65713348), KATNB1 (ENSG00000140854.13:s:57755345-57755494;57755494-57755841), KDM5C (ENSG00000126012.13:s:53217796-53217966;53217274-53217796), KIAA1671 (ENSG00000197077.14:t:25170820-25170938;25049364-25170820), KIFC3 (ENSG00000140859.16:t:57772223-57772288;57772288-57785464), KIFC3 (ENSG00000140859.16:t:57798072-57798282;57798282-57802370), KMT2B (ENSG00000272333.8:t:35719784-35720940;35719541-35719784), LDB1 (ENSG00000198728.11:s:102109029-102109177;102108323-102109029), LDB2 (ENSG00000169744.13:s:16511981-16512104;16508686-16511981), LETMD1 (ENSG00000050426.16:s:51048301-51048518;51048478-51056350), LST1 (ENSG00000204482.11:s:31587117-31587318;31587318-31587641), LST1 (ENSG00000204482.11:t:31587944-31587966;31587318-31587944), LST1 (ENSG00000206433.10:s:2842938-2843143;2843139-2844386), LST1 (ENSG00000223465.8:s:2834852-2835057;2835053-2835376), LST1 (ENSG00000223465.8:t:2835679-2835701;2835053-2835679), LST1 (ENSG00000226182.8:t:3065231-3065253;3064605-3065231), LST1 (ENSG00000230791.8:s:2886832-2887014;2887014-2887225), LST1 (ENSG00000230791.8:t:2887225-2887247;2886599-2887225), LST1 (ENSG00000231048.8:s:2892158-2892363;2892359-2892682), LST1 (ENSG00000231048.8:t:2892985-2893007;2892359-2892985), LTB (ENSG00000204487.8:s:3059543-3059714;3058917-3059543), LTB (ENSG00000223448.7:s:2887297-2887468;2886671-2887297), LTB (ENSG00000238114.7:s:2881536-2881707;2880910-2881536), LTBP4 (ENSG00000090006.18:s:40619347-40619493;40619493-40622401), LTBP4 (ENSG00000090006.18:t:40619347-40619493;40614446-40619347), LTBP4 (ENSG00000090006.18:t:40623604-40623732;40623021-40623604), MAPK9 (ENSG00000050748.18:t:180269280-180269409;180269409-180279796), MARK2 (ENSG00000072518.22:s:63904786-63905043;63905043-63908260), MAST3 (ENSG00000099308.13:s:18146881-18147044;18147017-18147443), MAX (ENSG00000125952.20:t:65077193-65077428;65077428-65077913), MBNL1 (ENSG00000152601.18:t:152414941-152415111;152269092-152414941,) METTL9 (ENSG00000197006.15:s:21624931-21625233;21625115-21655227), MGLL (ENSG00000074416.16:t:127821694-127821838;127821838-127822309), MGRN1 (ENSG00000102858.13:s:4677463-4677572;4677572-4681550), MIEF1 (ENSG00000100335.15:s:39511849-39512026;39512026-39512232), MLX (ENSG00000108788.12:s:42567619-42567655;42567655-42568837), MMAB (ENSG00000139428.12:s:109561420-109561517;109561329-109561420), MPI (ENSG00000178802.18:s:74890005-74890201;74890089-74890527), MPRIP (ENSG00000133030.22:t:17142627-17142765;17138429-17142627), MRPL33 (ENSG00000243147.8:s:27772674-27772855;27772692-27779433), MSRB3 (ENSG00000174099.12:s:65278643-65278865;65278865-65326826), MTA1 (ENSG00000182979.18:s:105445418-105445511;105445511-105450058), MTA1 (ENSG00000182979.18:t:105450058-105450184;105445511-105450058), MTHFD2 (ENSG00000065911.13:t:74207704-74207826;74205889-74207704), MTMR12 (ENSG00000150712.11:s:32312758-32312987;32274122-32312758), MTMR12 (ENSG00000150712.11:t:32273980-32274122;32274122-32312758), NAP1L4 (ENSG00000273562.4:t:175479-176694;176694-182308), NCOA2 (ENSG00000140396.13:t:70141179-70141399;70141399-70148273), NCOR2 (ENSG00000196498.14:s:124330845-124330898;124326370-124330845), NCOR2 (ENSG00000196498.14:s:124378237-124378384;124372610-124378237), NCOR2 (ENSG00000196498.14:t:124372022-124372610;124372610-124378237), NDRG2 (ENSG00000165795.25:t:21022392-21022820;21022497-21022864), NDUFV3 (ENSG00000160194.18:t:42908864-42913304;42897047-42908864), NFE2L1 (ENSG00000082641.16:s:48056386-48056598;48056598-48057032), NFIA (ENSG00000162599.18:t:61455303-61462788;61406727-61455303), NKIRAS2 (ENSG00000168256.18:t:42023654-42025644;42022640-42023654), NOC2L (ENSG00000188976.11:s:945042-945146;944800-945057), NTAN1 (ENSG00000275779.4:s:613600-613788;613788-621580), NTPCR (ENSG00000135778.12:t:232956347-232956443;232950744-232956347), NUDT22 (ENSG00000149761.9:t:64229131-64229344;64227132-64229247), NUP214 (ENSG00000126883.19:s:131146129-131146304;131146304-131147490), NXT2 (ENSG00000101888.12:t:109538045-109538131;109537270-109538045), OCEL1 (ENSG00000099330.9:s:17226213-17226353;17226353-17226693), P2RX4 (ENSG00000135124.16:s:121210065-121210298;121210298-121217134), PABIR2 (ENSG00000156504.17:s:134781821-134781917;134772283-134781821), PALM2AKAP2 (ENSG00000157654.19:s:110138171-110138539;110138539-110168399), PANK4 (ENSG00000157881.16:t:2521101-2521315;2521315-2526464), PAPOLA (ENSG00000090060.19:s:96556175-96556413;96556413-96560649), PARL (ENSG00000175193.14:s:183862607-183862801;183844326-183862697), PARL (ENSG00000175193.14:t:183844231-183844774;183844326-183862753), PARVB (ENSG00000188677.15:t:44093928-44094017;44069162-44093928), PCYT1B (ENSG00000102230.14:t:24618985-24619084;24619084-24646989), PDE7A (ENSG00000205268.11:t:65782783-65782843;65782843-65841371), PEX26 (ENSG00000215193.14:s:18083437-18083732;18083732-18087972), PFKFB3 (ENSG00000170525.21:t:6213623-6213748;6203336-6213623), PKN1 (ENSG00000123143.13:t:14441143-14441443;14433542-14441143), PLEKHM1 (ENSG00000225190.12:s:45481603-45482612;45478147-45482433), PLS3 (ENSG00000102024.19:t:115610243-115610323;115561260-115610243), PML (ENSG00000140464.20:s:74034478-74034558;74034530-74042989), PML (ENSG00000140464.20:t:74033156-74033422;74032715-74033156), POLR2J3 (ENSG00000168255.20:t:102566997-102567083;102567083-102568010), POLR2J3 (ENSG00000285437.2:t:102566966-102567083;102567083-102568010), PPIE (ENSG000000084072.17:s:39752910-39753266;39753052-39753287), PPM1N (ENSG00000213889.11:t:45499949-45500066;45497343-45499949), PPP6R2 (ENSG00000100239.16:s:50436367-50436452;50436452-50436988), PPP6R2 (ENSG00000100239.16:t:50437506-50437603;50437068-50437506), PRKAR1B (ENSG00000188191.16:t:596146-596304;596304-602553), PRKCD (ENSG00000163932.16:t:53178404-53178537;53165215-53178404), PSEN1 (ENSG00000080815.20:t:73170797-73171923;73148094-73170797), PSMB8 (ENSG00000230034.8:t:4086512-4086659;4086659-4087849), PSMG4 (ENSG00000180822.12:s:3263684-3263759;3263759-3264209), PTK2B (ENSG00000120899.18:t:27450749-27450895;27445919-27450749), PTPN12 (ENSG00000127947.16:s:77600664-77600965;77600806-77607235), PTPN18 (ENSG00000072135.13:t:130368897-130369201;130356200-130369133), PUF60 (ENSG00000179950.15:s:143821118-143821743;143818534-143821597), PUF60 (ENSG00000179950.15:s:143821118-143821743;143820716-143821597), PUM1 (ENSG00000134644.16:s:30967099-30967310;30966278-30967167), PYM1 (ENSG00000170473.17:t:55903387-55903480;55903480-55927076), RAB11FIP1 (ENSG00000156675.16:t:37870387-37870528;37870528-37871278), RAB1FIP5 (ENSG00000135631.17:t:73075993-73076182;73076182-73088050), RAB4A (ENSG00000168118.12:t:229295848-229295910;229271370-229295848), RABGAP1L (ENSG00000152061.24:t:174969277-174969387;174957549-174969277), RAP1B (ENSG00000127314.18:t:68650244-68650882;68648781-68650400), RARA (ENSG00000131759.18:t:40348316-40348464;40331396-40348316), RBCK1 (ENSG00000125826.22:s:409830-410025;410025-417526), RBM10 (ENSG00000182872.16:t:47173128-47173197;47169498-47173128), RBM39 (ENSG00000131051.24:s:35713019-35713203;35709256-35713019), RCOR3 (ENSG00000117625.14:t:211313424-211316385;211312961-211313424), REPS2 (ENSG00000169891.18:s:17022123-17022371;17022271-17025059), RFFL (ENSG00000092871.17:s:35026374-35026568;35021781-35026374), RHD (ENSG00000187010.21:s:25303322-25303459;25303459-25328898), RMND5B (ENSG00000145916.19:t:178138108-178138396;178131054-178138108), RPUSD1 (ENSG00000007376.8:t:786826-786928;786928-787077), SEC16A (ENSG00000148396.19:s:136447227-136447364;136446949-136447227), SIGLEC10 (ENSG00000142512.15:t:51414422-51414515;51414515-51415181), SIRT3 (ENSG00000142082.15:s:218778-219041;216718-218778), SLA2 (ENSG00000101082.14:s:36615225-36615374;36614387-36615225), SLC66A2 (ENSG00000122490.19:t:79903446-79904183;79904183-79919184), SMARCA4 (ENSG00000127616.21:s:11034914-11035132;11035132-11041298), SMARCA4 (ENSG00000127616.21:t:11040634-11041560;11035132-11041298), SMARCC2 (ENSG00000139613.12:s:56173696-56173849;56173029-56173696), SNX20 (ENSG00000167208.16:s:50675770-50675921;50669148-50675770), SPIB (ENSG00000269404.7:s:50423605-50423755;50423751-50428038), SRSF6 (ENSG00000124193.16:s:43458361-43458509;43458509-43459153), SSX2IP (ENSG00000117155.17:t:84670646-84670815;84670815-84690371), STAMBP (ENSG00000124356.17:t:73830845-73831059;73829070-73830845), STAT3 (ENSG00000168610.16:t:42317182-42317224;42317224-42319856), SWI5 (ENSG00000175854.15:t:128276587-128276755;128276402-128276707), SYNE1 (ENSG00000131018.25:s:152301869-152302269;152300781-152301869), TANGO2 (ENSG00000183597.16:t:20061530-20061802;20056013-20061530), TCF3 (ENSG00000071564.19:s:1615686-1615804;1612433-1615686), TCF7L2 (ENSG00000148737.18:s:113146011-113146097;113146097-113150998), TECR (ENSG00000099797.15:s:14562356-14562575;14562575-14563174), TJP2 (ENSG00000119139.21:t:69212548-69212601;69151771-69212548), TLE4 (ENSG00000106829.21:s:79627374-79627752;79627448-79652593), TLE4 (ENSG00000106829.21:t:79652590-79652629;79627448-79652593), TMBIM1 (ENSG00000135926.15:s:218292466-218292586;218282181-218292466), TMBIM4 (ENSG00000155957.19:s:66169855-66170027;66161172-66169855), TMEM11 (ENSG00000178307.10:s:21214091-21214176;21211227-21214091), TMEM126B (ENSG00000171204.13:t:85631687-85631808;85628688-85631687), TMEM219 (ENSG00000149932.17:t:29962677-29963308;29962132-29963107), TNFSF12 (ENSG00000239697.12:t:7549466-7549618;7549312-7549474), TNK2 (ENSG00000061938.21:t:195888426-195888606;195888606-195908485), TNRC18 (ENSG00000182095.15:t:5325096-5325574;5325248-5329937), TRAPPC2 (ENSG00000196459.15:s:13734525-13734635;13719982-13734525), TRAPPC6A (ENSG00000007255.10:t:45164725-45164970;45164970-45165127), TSC22D3 (ENSG00000157514.18:t:107715773-107715950;107715950-107775100), TSPAN32 (ENSG00000064201.16:s:2314485-2314666;2314571-2316229), UBAC2 (ENSG00000134882.16:t:99238427-99238554;99200939-99238427), UFD1 (ENSG00000070010.19:t:19471687-19471808;19471808-19479083), URI1 (ENSG00000105176.18:s:29942239-29942664;29942664-29985223), URI1 (ENSG00000105176.18:t:29985223-29985301;29942664-29985223), USP15 (ENSG00000135655.16:s:62325872-62325933;62325933-62349221), USP22 (ENSG00000124422.12:t:21028542-21028674;21028674-21043321), VGLL4 (ENSG00000144560.16:t:11601833-11602022;11602022-11702971), WWP2 (ENSG00000198373.13:t:69786996-69787080;69762391-69786996), YAF2 (ENSG00000015153.15:s:42237599-42237800;42199235-42237599), YIPF6 (ENSG00000181704.12:s:68498562-68499157;68499123-68511849), YIPF6 (ENSG00000181704.12:t:68513327-68515340;68511977-68513327), ZBTB7B (ENSG00000160685.14:t:155014448-155015814;155002943-155014655), ZEB2 (ENSG00000169554.23:t:144517278-144517557;144517419-144517612), ZFAND1 (ENSG00000104231.11:s:81718182-81718224;81715114-81718182), ZFAND1 (ENSG00000104231.11:t:81714987-81715114;81715114-81718182), ZFAND2B (ENSG00000158552.13:s:219207648-219207779;219207774-219207887), ZMIZ2 (ENSG00000122515.16:s:44759281-44759460;44759460-44760151), ZMYND8 (ENSG00000101040.20:t:47236326-47236516;47236516-47238758), ZNF185 (ENSG00000147394.20:s:152922720-152922809;152922809-152928575), and ZNF451 (ENSG00000112200.17:t:57099061-57099141;57090274-57099061). In some cases, the expressed spliced junctions may comprise expressed genes.

The identified gene may include ABLIM1 (ENSG00000099204.21:t:114491791-114491878;114491878-114545005). The identified gene may include ACAA1 (ENSG00000060971.19:s:38126493-38126700;38126341-38126510). The identified gene may include ACAP1 (ENSG00000072818.12:t:7341948-7342067;7336787-7341948). The identified gene may include ACOT8 (ENSG00000101473.17:t:45848450-45849110;45848675-45857188). The identified gene may include ACP1 (ENSG00000143727.16:t:272192-273155;272065-272192). The identified gene may include ADD3 (ENSG00000148700.15:t:110100625-110100848;110008299-110100625). The identified gene may include ADGRE5 (ENSG00000123146.20:t:14397414-14397804;14391079-14397658). The identified gene may include ADK (ENSG00000156110.15:t:74200764-74200838;74151343-74200764). The identified gene may include AGAP3 (ENSG00000133612.19:s:151119987-151120145;151120145-151122725). The identified gene may include AKAP13 (ENSG00000170776.22:t:85575131-85575329;85543955-85575131). The identified gene may include ALAD (ENSG00000148218.16:s:113393161-113393634;113392169-113393447). The identified gene may include AMPD2 (ENSG00000116337.20:t:109625303-109625433;109621266-109625303). The identified gene may include ANAPC11 (ENSG00000141552.18:t:81894298-81894624;81891833-81894467). The identified gene may include AP1B1 (ENSG00000100280.17:s:29330378-29330532;29329720-29330378). The identified gene may include AP1B1 (ENSG00000100280.17:t:29327680-29328895;29328895-29329712). The identified gene may include APP (ENSG00000142192.21:s:26000015-26000182;25982477-26000015). The identified gene may include APP (ENSG00000142192.21:t:25897573-25897983;25897673-25911741). The identified gene may include ARAP1 (ENSG00000186635.15:s:72693325-72693470;72688537-72693325). The identified gene may include ARFRP1 (ENSG00000101246.20:t:63706999-63707658;63707097-63707867). The identified gene may include ARHGAP17 (ENSG00000140750.17:t:24935470-24936827;24935639-24941987). The identified gene may include ARHGAP17 (ENSG00000288353.1:t:188953-190310;189122-195470). The identified gene may include ARHGEF10 (ENSG00000274726.4:s:43270-43560;43560-47749). The identified gene may include ARHGEF1 (ENSG00000076928.19:s:41896377-41896878;41896482-41897315). The identified gene may include ARHGEF7 (ENSG00000102606.20:t:111217679-111217885;111210002-111217679). The identified gene may include ARMC10 (ENSG00000170632.14:s:103074881-103075411;103075411-103075777). The identified gene may include ARRDC2 (ENSG00000105643.11:t:18008711-18008861;18001573-18008711). The identified gene may include ATE1 (ENSG00000107669.19:t:121924266-121924329;121924329-121928347). The identified gene may include ATP6V1D (ENSG00000100554.12:t:67350611-67350690;67350690-67359658). The identified gene may include ATXN2L (ENSG00000168488.19:s:28835933-28836176;28836122-28836748). The identified gene may include ATXN2L (ENSG00000168488.19:t:28836728-28837237;28836122-28836748). The identified gene may include ATXN2L (ENSG00000168488.19:t:28836728-28837237;28836485-28836728). The identified gene may include AURKB (ENSG000000178999.13:t:8207738-8208225;8207840-8210530). The identified gene may include BLOC1S6 (ENSG00000104164.12:s:45592135-45592276;45592276-45605428). The identified gene may include C12orf76 (ENSG00000174456.16:s:110048363-110048870;110042459-110048363). The identified gene may include CAMTA1 (ENSG00000171735.20:t:6825092-6825210;6820250-6825092). The identified gene may include CBFA2T3 (ENSG00000129993.15:t:88892244-88892485;88892485-88898078). The identified gene may include CBFB (ENSG00000067955.15:t:67066682-67066962;67036755-67066682). The identified gene may include CCDC28B (ENSG000000160050.16:t:32204598-32204620;32204379-32204598). The identified gene may include CCDC92 (ENSG00000119242.9:s:123972529-123972831;123943493-123972529). The identified gene may include CCDC92 (ENSG00000119242.9:t:123943347-123943495;123943493-123972529). The identified gene may include CD300H (ENSG00000284690.3:s:74563919-74564275;74560824-74563919). The identified gene may include CD34 (ENSG00000174059.17:s:207888682-207888846;207887923-207888682). The identified gene may include CDAN1 (ENSG00000140326.13:s:42736989-42737128;42736780-42737013). The identified gene may include CDC14B (ENSG00000081377.17:t:96565393-96565512;96565483-96619219). The identified gene may include CDK5RAP2 (ENSG00000136861.19:t:120402806-120403071;120403071-120403316). The identified gene may include CELF2 (ENSG00000048740.19:t:11249153-11249201;11165682-11249153). The identified gene may include CEP164 (ENSG00000110274.16:t:117409618-117410701;117409028-117409618). The identified gene may include CLEC1B (ENSG00000165682.15:s:9995140-9995246;9986158-9995140). The identified gene may include COX20 (ENSG00000203667.10:s:244835616-244835756;244835756-244842195). The identified gene may include CPNE1 (ENSG00000214078.13:s:35627280-35627531;35626800-35627280). The identified gene may include CYTH2 (ENSG00000105443.17:s:48474838-48476276;48474949-48477717). The identified gene may include CYTH2 (ENSG00000105443.17:t:48478066-48478145;48477719-48478069). The identified gene may include DAZAP1 (ENSG00000071626.17:s:1422348-1422396;1422396-1425878). The identified gene may include DCTD (ENSG00000129187.15:s:182917288-182917477;182915575-182917311). The identified gene may include DCTD (ENSG00000129187.15:t:182915461-182915575;182915575-182917288). The identified gene may include DECR1 (ENSG00000104325.7:s:90001405-90001561;90001561-90017124). The identified gene may include DECR1 (ENSG00000104325.7:s:90001405-90001561;90001561-90018909). The identified gene may include DECR1 (ENSG00000104325.7:t:90018564-90018966;90001561-90018909). The identified gene may include DEF8 (ENSG00000140995.17:t:89954172-89954376;89949513-89954243). The identified gene may include DGKD (ENSG00000077044.11:s:233390403-233390483;233390483-233392070). The identified gene may include DGKZ (ENSG00000149091.15:t:46367291-46367399;46345585-46367291). The identified gene may include DNM2 (ENSG00000079805.19:s:10795372-10795439;10795439-10797380). The identified gene may include DOCK8 (ENSG00000107099.18:t:271627-271729;215029-271627). The identified gene may include DUT (ENSG00000128951.14:t:48332103-48332737;48331479-48332268). The identified gene may include DYRK1A (ENSG00000157540.22:s:37418905-37420384;37420384-37472684). The identified gene may include DYSF (ENSG00000135636.16:t:71480883-71480938;71454086-71480883). The identified gene may include EIF4G1 (ENSG00000114867.22:t:184317321-184317497;184314674-184317321). The identified gene may include EIF4G3 (ENSG00000075151.24:t:20981048-20981227;20981227-20997601). The identified gene may include EMC8 (ENSG000000131148.9:s:85781211-85781760;85779867-85781211). The identified gene may include EPSTI1 (ENSG00000133106.15:s:42991978-42992271;42969177-42991978). The identified gene may include EXOSC1 (ENSG00000171311.13:s:97438663-97438703;97437750-97438663). The identified gene may include EXOSC1 (ENSG00000171311.13:t:97437700-97437750;97437750-97438663). The identified gene may include F2RL3 (ENSG00000127533.4:s:16888999-16889298;16889298-16889577). The identified gene may include FAM219A (ENSG00000164970.15:s:34405865-34405964;34402807-34405916). The identified gene may include FAM3A (ENSG00000071889.17:s:154512823-154512936;154511871-154512823). The identified gene may include FBXO44 (ENSG00000132879.14:t:11658736-11658871;11658628-11658736). The identified gene may include FKBP1B (ENSG00000119782.14:s:24060814-24060926;24060926-24063019). The identified gene may include FMNL3 (ENSG00000161791.14:t:49657082-49657190;49657190-49658442). The identified gene may include FOXP1 (ENSG00000114861.24:t:71112536-71112637;71112637-71130498). The identified gene may include G3BP1 (ENSG00000145907.16:t:151786572-151786715;151772036-151786603). The identified gene may include GET1 (ENSG00000182093.16:t:39390698-39390803;39380556-39390698). The identified gene may include GLUL (ENSG00000135821.19:t:182388572-182390304;182388750-182391175). The identified gene may include GORASP1 (ENSG00000114745.15:s:39107479-39108063;39103553-39107479). The identified gene may include GORASP1 (ENSG00000114745.15:t:39101016-39101102;39103553-39107479). The identified gene may include GSE1 (ENSG00000131149.19:t:85648552-85648751;85556363-85648552). The identified gene may include GSTT1 (ENSG00000277656.3:t:270997-271173;271173-278295). The identified gene may include GUCD1 (ENSG00000138867.17:t:24548917-24549001;24549001-24555615). The identified gene may include HDAC7 (ENSG00000061273.18:s:47796207-47796298;47796016-47796207). The identified gene may include HES6 (ENSG00000144485.11:t:238239487-238239568;238239568-238239825) The identified gene may include HUS1 (ENSG00000136273.13:s:47979410-47979615;47978816-47979410). The identified gene may include IFI27 (ENSG00000275214.4:t:1230073-1230504;1229442-1230343). The identified gene may include IGF2BP3 (ENSG00000136231.14:t:23342064-23342189;23342189-23343718). The identified gene may include IKBKB (ENSG00000104365.16:t:42290156-42290273;42288728-42290156). The identified gene may include IKBKG (ENSG00000269335.7:t:154551988-154552189;154542452-154551988). The identified gene may include IKZF3 (ENSG00000161405.17:t:39777651-39777767;39777767-39788258). The identified gene may include IL10RA (ENSG00000110324.12:t:117988382-117988502;117986534-117988382). The identified gene may include IL15RA (ENSG00000134470.21:t:5960367-5960567;5960567-5963743). The identified gene may include IMPDH1 (ENSG00000106348.18:t:128405767-128405948;128405865-128409289). The identified gene may include INO80E (ENSG00000169592.15:s:30001414-30001575;30001530-30005221). The identified gene may include INPP5D (ENSG00000281614.3:s:67445-67595;67595-71086). The identified gene may include IP6K2 (ENSG00000068745.15:t:48694872-48695421;48695421-48717157). The identified gene may include IRF7 (ENSG00000276561.4:s:144369-144427;144292-144369). The identified gene may include IRF7 (ENSG00000276561.4:s:144676-144900;144424-144690). The identified gene may include ITSN2 (ENSG00000198399.16:s:24246129-24246320;24242206-24246129). The identified gene may include JAML (ENSG00000160593.19:t:118212407-118212561;118212561-118214824). The identified gene may include JOSD2 (ENSG00000161677.12:t:50506378-50506572;50506572-50507574). The identified gene may include KANK2 (ENSG00000197256.11:s:11195556-11195881;11194590-11195556). The identified gene may include KANK2 (ENSG00000197256.11:t:11194553-11194590;11194590-11195556). The identified gene may include KANSL3 (ENSG00000114982.19:s:96636739-96637228;96631543-96636921). The identified gene may include KANSL3 (ENSG00000114982.19:t:96631312-96631543;96631543-96636921). The identified gene may include KAT5 (ENSG00000172977.13:s:65712922-65713319;65713058-65713348). The identified gene may include KATNB1 (ENSG00000140854.13:s:57755345-57755494;57755494-57755841). The identified gene may include KDM5C (ENSG00000126012.13:s:53217796-53217966;53217274-53217796). The identified gene may include KIAA1671 (ENSG00000197077.14:t:25170820-25170938;25049364-25170820). The identified gene may include KIFC3 (ENSG00000140859.16:t:57772223-57772288;57772288-57785464). The identified gene may include KIFC3 (ENSG00000140859.16:t:57798072-57798282;57798282-57802370). The identified gene may include KMT2B (ENSG00000272333.8:t:35719784-35720940;35719541-35719784). The identified gene may include LDB1 (ENSG00000198728.11:s:102109029-102109177;102108323-102109029). The identified gene may include LDB2 (ENSG00000169744.13:s:16511981-16512104;16508686-16511981). The identified gene may include LETMD1 (ENSG00000050426.16:s:51048301-51048518;51048478-51056350). The identified gene may include LST1 (ENSG00000204482.11:s:31587117-31587318;31587318-31587641). The identified gene may include LST1 (ENSG00000204482.11:t:31587944-31587966;31587318-31587944). The identified gene may include LST1 (ENSG00000206433.10:s:2842938-2843143;2843139-2844386). The identified gene may include LST1 (ENSG00000223465.8:s:2834852-2835057;2835053-2835376). The identified gene may include LST1 (ENSG00000223465.8:t:2835679-2835701;2835053-2835679). The identified gene may include LST1 (ENSG00000226182.8:t:3065231-3065253;3064605-3065231). The identified gene may include LST1 (ENSG00000230791.8:s:2886832-2887014;2887014-2887225). The identified gene may include LST1 (ENSG00000230791.8:t:2887225-2887247;2886599-2887225). The identified gene may include LST1 (ENSG00000231048.8:s:2892158-2892363;2892359-2892682). The identified gene may include LST1 (ENSG00000231048.8:t:2892985-2893007;2892359-2892985). The identified gene may include LTB (ENSG00000204487.8:s:3059543-3059714;3058917-3059543). The identified gene may include LTB (ENSG00000223448.7:s:2887297-2887468;2886671-2887297). The identified gene may include LTB (ENSG00000238114.7:s:2881536-2881707;2880910-2881536). The identified gene may include LTBP4 (ENSG00000090006.18:s:40619347-40619493;40619493-40622401). The identified gene may include LTBP4 (ENSG00000090006.18:t:40619347-40619493;40614446-40619347). The identified gene may include LTBP4 (ENSG00000090006.18:t:40623604-40623732;40623021-40623604). The identified gene may include MAPK9 (ENSG00000050748.18:t:180269280-180269409;180269409-180279796). The identified gene may include MARK2 (ENSG00000072518.22:s:63904786-63905043;63905043-63908260). The identified gene may include MAST3 (ENSG00000099308.13:s:18146881-18147044;18147017-18147443). The identified gene may include MAX (ENSG00000125952.20:t:65077193-65077428;65077428-65077913). The identified gene may include MBNL1 (ENSG00000152601.18:t:152414941-152415111;152269092-152414941. The identified gene may include METTL9 (ENSG00000197006.15:s:21624931-21625233;21625115-21655227). The identified gene may include MGLL (ENSG00000074416.16:t:127821694-127821838;127821838-127822309). The identified gene may include MGRN1 (ENSG00000102858.13:s:4677463-4677572;4677572-4681550). The identified gene may include MIEF1 (ENSG00000100335.15:s:39511849-39512026;39512026-39512232). The identified gene may include MLX (ENSG00000108788.12:s:42567619-42567655;42567655-42568837). The identified gene may include MMAB (ENSG00000139428.12:s:109561420-109561517;109561329-109561420). The identified gene may include MPI (ENSG00000178802.18:s:74890005-74890201;74890089-74890527). The identified gene may include MPRIP (ENSG00000133030.22:t:17142627-17142765;17138429-17142627). The identified gene may include MRPL33 (ENSG00000243147.8:s:27772674-27772855;27772692-27779433). The identified gene may include MSRB3 (ENSG00000174099.12:s:65278643-65278865;65278865-65326826). The identified gene may include MTA1 (ENSG00000182979.18:s:105445418-105445511;105445511-105450058). The identified gene may include MTA1 (ENSG00000182979.18:t:105450058-105450184;105445511-105450058). The identified gene may include MTHFD2 (ENSG00000065911.13:t:74207704-74207826;74205889-74207704). The identified gene may include MTMR12 (ENSG00000150712.11:s:32312758-32312987;32274122-32312758). The identified gene may include MTMR12 (ENSG00000150712.11:t:32273980-32274122;32274122-32312758). The identified gene may include NAP1L4 (ENSG00000273562.4:t:175479-176694;176694-182308). The identified gene may include NCOA2 (ENSG00000140396.13:t:70141179-70141399;70141399-70148273). The identified gene may include NCOR2 (ENSG00000196498.14:s:124330845-124330898;124326370-124330845). The identified gene may include NCOR2 (ENSG00000196498.14:s:124378237-124378384;124372610-124378237). The identified gene may include NCOR2 (ENSG00000196498.14:t:124372022-124372610;124372610-124378237). The identified gene may include NDRG2 (ENSG00000165795.25:t:21022392-21022820;21022497-21022864). The identified gene may include NDUFV3 (ENSG00000160194.18:t:42908864-42913304;42897047-42908864). The identified gene may include NFE2L1 (ENSG00000082641.16:s:48056386-48056598;48056598-48057032). The identified gene may include NFIA (ENSG00000162599.18:t:61455303-61462788;61406727-61455303). The identified gene may include NKIRAS2 (ENSG00000168256.18:t:42023654-42025644;42022640-42023654). The identified gene may include NOC2L (ENSG00000188976.11:s:945042-945146;944800-945057). The identified gene may include NTAN1 (ENSG00000275779.4:s:613600-613788;613788-621580). The identified gene may include NTPCR (ENSG00000135778.12:t:232956347-232956443;232950744-232956347). The identified gene may include NUDT22 (ENSG00000149761.9:t:64229131-64229344;64227132-64229247). The identified gene may include NUP214 (ENSG00000126883.19:s:131146129-131146304;131146304-131147490). The identified gene may include NXT2 (ENSG00000101888.12:t:109538045-109538131;109537270-109538045). The identified gene may include OCEL1 (ENSG00000099330.9:s:17226213-17226353;17226353-17226693). The identified gene may include P2RX4 (ENSG00000135124.16:s:121210065-121210298;121210298-121217134). The identified gene may include PABIR2 (ENSG00000156504.17:s:134781821-134781917;134772283-134781821). The identified gene may include PALM2AKAP2 (ENSG00000157654.19:s:110138171-110138539;110138539-110168399). The identified gene may include PANK4 (ENSG00000157881.16:t:2521101-2521315;2521315-2526464). The identified gene may include PAPOLA (ENSG00000090060.19:s:96556175-96556413;96556413-96560649). The identified gene may include PARL (ENSG00000175193.14:s:183862607-183862801;183844326-183862697). The identified gene may include PARL (ENSG00000175193.14:t:183844231-183844774;183844326-183862753). The identified gene may include PARVB (ENSG00000188677.15:t:44093928-44094017;44069162-44093928). The identified gene may include PCYT1B (ENSG00000102230.14:t:24618985-24619084;24619084-24646989). The identified gene may include PDE7A (ENSG00000205268.11:t:65782783-65782843;65782843-65841371). The identified gene may include PEX26 (ENSG00000215193.14:s:18083437-18083732;18083732-18087972). The identified gene may include PFKFB3 (ENSG00000170525.21:t:6213623-6213748;6203336-6213623). The identified gene may include PKN1 (ENSG00000123143.13:t:14441143-14441443;14433542-14441143). The identified gene may include PLEKHM1 (ENSG00000225190.12:s:45481603-45482612;45478147-45482433). The identified gene may include PLS3 (ENSG00000102024.19:t:115610243-115610323;115561260-115610243). The identified gene may include PML (ENSG00000140464.20:s:74034478-74034558;74034530-74042989). The identified gene may include PML (ENSG00000140464.20:t:74033156-74033422;74032715-74033156). The identified gene may include POLR2J3 (ENSG00000168255.20:t:102566997-102567083;102567083-102568010). The identified gene may include POLR2J3 (ENSG00000285437.2:t:102566966-102567083;102567083-102568010). The identified gene may include PPIE (ENSG00000084072.17:s:39752910-39753266;39753052-39753287). The identified gene may include PPM1N (ENSG00000213889.11:t:45499949-45500066;45497343-45499949). The identified gene may include PPP6R2 (ENSG00000100239.16:s:50436367-50436452;50436452-50436988) The identified gene may include PPP6R2 (ENSG00000100239.16:t:50437506-50437603;50437068-50437506). The identified gene may include PRKAR1B (ENSG00000188191.16:t:596146-596304;596304-602553). The identified gene may include PRKCD (ENSG00000163932.16:t:53178404-53178537;53165215-53178404). The identified gene may include PSEN1 (ENSG00000080815.20:t:73170797-73171923;73148094-73170797). The identified gene may include PSMB8 (ENSG00000230034.8:t:4086512-4086659;4086659-4087849). The identified gene may include PSMG4 (ENSG00000180822.12:s:3263684-3263759;3263759-3264209). The identified gene may include PTK2B (ENSG00000120899.18:t:27450749-27450895;27445919-27450749). The identified gene may include PTPN12 (ENSG00000127947.16:s:77600664-77600965;77600806-77607235). The identified gene may include PTPN18 (ENSG00000072135.13:t:130368897-130369201;130356200-130369133). The identified gene may include PUF60 (ENSG00000179950.15:s:143821118-143821743;143818534-143821597). The identified gene may include PUF60 (ENSG00000179950.15:s:143821118-143821743;143820716-143821597). The identified gene may include PUM1 (ENSG00000134644.16:s:30967099-30967310;30966278-30967167). The identified gene may include PYM1 (ENSG00000170473.17:t:55903387-55903480;55903480-55927076). The identified gene may include RAB11FIP1 (ENSG00000156675.16:t:37870387-37870528;37870528-37871278). The identified gene may include RAB11FIP5 (ENSG00000135631.17:t:73075993-73076182;73076182-73088050). The identified gene may include RAB4A (ENSG00000168118.12:t:229295848-229295910;229271370-229295848). The identified gene may include RABGAP1L (ENSG00000152061.24:t:174969277-174969387;174957549-174969277). The identified gene may include RAP1B (ENSG00000127314.18:t:68650244-68650882;68648781-68650400). The identified gene may include RARA (ENSG00000131759.18:t:40348316-40348464;40331396-40348316). The identified gene may include RBCK1 (ENSG00000125826.22:s:409830-410025;410025-417526). The identified gene may include RBM10 (ENSG00000182872.16:t:47173128-47173197;47169498-47173128). The identified gene may include RBM39 (ENSG00000131051.24:s:35713019-35713203;35709256-35713019). The identified gene may include RCOR3 (ENSG00000117625.14:t:211313424-211316385;211312961-211313424). The identified gene may include REPS2 (ENSG00000169891.18:s:17022123-17022371;17022271-17025059). The identified gene may include RFFL (ENSG00000092871.17:s:35026374-35026568;35021781-35026374). The identified gene may include RHD (ENSG00000187010.21:s:25303322-25303459;25303459-25328898). The identified gene may include RMND5B (ENSG00000145916.19:t:178138108-178138396;178131054-178138108). The identified gene may include RPUSD1 (ENSG00000007376.8:t:786826-786928;786928-787077). The identified gene may include SEC16A (ENSG00000148396.19:s:136447227-136447364;136446949-136447227). The identified gene may include SIGLEC10 (ENSG00000142512.15:t:51414422-51414515;51414515-51415181). The identified gene may include SIRT3 (ENSG00000142082.15:s:218778-219041;216718-218778). The identified gene may include SLA2 (ENSG00000101082.14:s:36615225-36615374;36614387-36615225). The identified gene may include SLC66A2 (ENSG00000122490.19:t:79903446-79904183;79904183-79919184). The identified gene may include SMARCA4 (ENSG00000127616.21:s:11034914-11035132;11035132-11041298). The identified gene may include SMARCA4 (ENSG00000127616.21:t:11040634-11041560;11035132-11041298). The identified gene may include SMARCC2 (ENSG00000139613.12:s:56173696-56173849;56173029-56173696). The identified gene may include SNX20 (ENSG00000167208.16:s:50675770-50675921;50669148-50675770). The identified gene may include SPIB (ENSG00000269404.7:s:50423605-50423755;50423751-50428038). The identified gene may include SRSF6 (ENSG00000124193.16:s:43458361-43458509;43458509-43459153). The identified gene may include SSX2IP (ENSG00000117155.17:t:84670646-84670815;84670815-84690371). The identified gene may include STAMBP (ENSG00000124356.17:t:73830845-73831059;73829070-73830845). The identified gene may include STAT3 (ENSG00000168610.16:t:42317182-42317224;42317224-42319856). The identified gene may include SWI5 (ENSG00000175854.15:t:128276587-128276755;128276402-128276707). The identified gene may include SYNE1 (ENSG00000131018.25:s:152301869-152302269;152300781-152301869). The identified gene may include TANGO2 (ENSG00000183597.16:t:20061530-20061802;20056013-20061530). The identified gene may include TCF3 (ENSG00000071564.19:s:1615686-1615804;1612433-1615686). The identified gene may include TCF7L2 (ENSG00000148737.18:s:113146011-113146097;113146097-113150998). The identified gene may include TECR (ENSG00000099797.15:s:14562356-14562575;14562575-14563174). The identified gene may include TJP2 (ENSG00000119139.21:t:69212548-69212601;69151771-69212548). The identified gene may include TLE4 (ENSG00000106829.21:s:79627374-79627752;79627448-79652593). The identified gene may include TLE4 (ENSG00000106829.21:t:79652590-79652629;79627448-79652593). The identified gene may include TMBIM1 (ENSG00000135926.15:s:218292466-218292586;218282181-218292466). The identified gene may include TMBIM4 (ENSG00000155957.19:s:66169855-66170027;66161172-66169855). The identified gene may include TMEM11 (ENSG00000178307.10:s:21214091-21214176;21211227-21214091). The identified gene may include TMEM126B (ENSG00000171204.13:t:85631687-85631808;85628688-85631687). The identified gene may include TMEM219 (ENSG000000149932.17:t:29962677-29963308;29962132-29963107). The identified gene may include TNFSF12 (ENSG00000239697.12:t:7549466-7549618;7549312-7549474). The identified gene may include TNK2 (ENSG00000061938.21:t:195888426-195888606;195888606-195908485). The identified gene may include TNRC18 (ENSG00000182095.15:t:5325096-5325574;5325248-5329937). The identified gene may include TRAPPC2 (ENSG00000196459.15:s:13734525-13734635;13719982-13734525). The identified gene may include TRAPPC6A (ENSG00000007255.10:t:45164725-45164970;45164970-45165127). The identified gene may include TSC22D3 (ENSG00000157514.18:t:107715773-107715950;107715950-107775100). The identified gene may include TSPAN32 (ENSG00000064201.16:s:2314485-2314666;2314571-2316229). The identified gene may include UBAC2 (ENSG00000134882.16:t:99238427-99238554;99200939-99238427). The identified gene may include UFD1 (ENSG00000070010.19:t:19471687-19471808;19471808-19479083). The identified gene may include URI1 (ENSG00000105176.18:s:29942239-29942664;29942664-29985223). The identified gene may include URI1 (ENSG00000105176.18:t:29985223-29985301;29942664-29985223). The identified gene may include USP15 (ENSG000000135655.16:s:62325872-62325933;62325933-62349221). The identified gene may include USP22 (ENSG00000124422.12:t:21028542-21028674;21028674-21043321). The identified gene may include VGLL4 (ENSG00000144560.16:t:11601833-11602022;11602022-11702971). The identified gene may include WWP2 (ENSG00000198373.13:t:69786996-69787080;69762391-69786996). The identified gene may include YAF2 (ENSG00000015153.15:s:42237599-42237800;42199235-42237599). The identified gene may include YIPF6 (ENSG00000181704.12:s:68498562-68499157;68499123-68511849). The identified gene may include YIPF6 (ENSG00000181704.12:t:68513327-68515340;68511977-68513327). The identified gene may include ZBTB7B (ENSG00000160685.14:t:155014448-155015814;155002943-155014655). The identified gene may include ZEB2 (ENSG00000169554.23:t:144517278-144517557;144517419-144517612). The identified gene may include ZFAND1 (ENSG00000104231.11:s:81718182-81718224;81715114-81718182). The identified gene may include ZFAND1 (ENSG00000104231.11:t:81714987-81715114;81715114-81718182). The identified gene may include ZFAND2B (ENSG00000158552.13:s:219207648-219207779;219207774-219207887). The identified gene may include ZMIZ2 (ENSG00000122515.16:s:44759281-44759460;44759460-44760151). The identified gene may include ZMYND8 (ENSG00000101040.20:t:47236326-47236516;47236516-47238758). The identified gene may include ZNF185 (ENSG00000147394.20:s:152922720-152922809;152922809-152928575). The identified gene may include ZNF451 (ENSG00000112200.17:t:57099061-57099141;57090274-57099061). The identified gene may include any of the genes listed in Tables 1-3.

The methods may utilize computer processing. The computer processing may make use of machine learning as disclosed herein. The computer processing may use of a classifier as disclosed herein. The computer processing may make use of prediction or classification. The classification or classifier may be a trained classifier. The classifier may be trained by the methods disclosed herein. In some cases, one or more splice junctions are computer processed as disclosed herein.

The methods may determine a risk of a disease state based at least in part on the computer processing. The methods may comprise determining a risk of a disease state with an accuracy. In some cases, the risk of a disease state is determined with an accuracy of more than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The methods may comprise determining a risk of a disease state with a particular sensitivity. In some cases, the risk of a disease state is determined with a sensitivity of more than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The methods may comprise determining a risk of a disease state using a receiver operating characteristic (ROC) curve with an area under the curve (AUC) value. In some cases, the risk of the disease state is determined with an AUC value of an ROC curve of more than or equal to 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99.

The methods may include administering a treatment to a subject. The treatment may treat a disease state of the subject. The treatment may treat one or more disease states of the subject. The treatment may be administered orally, intravenously, intramuscularly, subcutaneously, intrathecally, rectally, vaginally, topically, intranasally, or any combination thereof. The treatment may be in the form of a liquid, a tablet, or a capsule. The treatment may be in the form of a cream, a lotion, or an ointment. The treatment may be in the form of a droplet, an inhaler, an injection, a patch, an implant, or a suppository.

The treatment may treat memory loss, behavioral changes, sleep problems, and other symptoms associated with Alzheimer's disease. For example, citalopram, fluoxetine, paroxetine, and sertraline can be used to treat issues relating to mood, depression, and irritability experienced by Alzheimer's disease. As another example, alprazolam, buspirone, lorazepam, and oxazepam can be used to treat anxiety or restlessness associated with Alzheimer's disease. Further, unconventional therapies, such as hormone replacement therapy, art and music therapies, and supplements (e.g., vitamins such as vitamin E) can be used alternatively or additionally to treat the Alzheimer's disease.

The treatment may be a medicinal therapy. The medicinal therapy may be a cholinesterase inhibitor. The medicinal therapy may be a N-methyl-D-aspartate (NMDA) antagonist. The medicinal therapy may be an atypical antipsychotic. The medicinal therapy may be a disease-modifying immunotherapy. The medicinal therapy may be a monoclonal antibody (mab) therapy. The medicinal therapy may be an amyloid monoclonal antibody (mab) therapy.

The treatment may be a behavioral therapy. The behavioral therapy may include cognitive behavioral therapy, cognitive behavioral play therapy, dialectical behavioral therapy, exposure therapy, rational emotive behavior therapy, cognitive restructuring, aversion therapy, interpersonal psychotherapy, multisensory stimulation, active music therapy, cognitive stimulation, or any combination thereof. The treatment may be a sleep therapy.

Methods disclosed herein for determining a risk of a disease can determine the risk with an accuracy that is greater than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. Such methods can determine a risk of Alzheimer's disease with an accuracy that is greater than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Methods disclosed herein for determining a risk of a disease can determine the risk with a sensitivity that is greater than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. Such methods can determine a risk of Alzheimer's disease with a sensitivity that is greater than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The methods disclosed herein can determine a risk of a disease using a receiver operating characteristic (ROC) curve with an area under the curve (AUC) value of greater than or equal to 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99. The methods disclosed herein can determine a risk of Alzheimer's disease using a receiver operating characteristic (ROC) curve with an area under the curve (AUC) value of greater than or equal to 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99.

### Assessing an Effect of a Compound

Provided herein are methods of assessing an effect of a compound. The methods may comprise assaying a first expression profile of a first cell-free biological sample. The biological sample may be obtained from a subject. The biological sample may be derived from a subject. The biological sample may be obtained or derived from a subject at a first time point. The methods may comprise detecting a first set of splice junctions. The methods may comprise administering a compound to the subject. The methods may comprise assaying a second expression profile of a second cell-free biological sample. The biological sample may be obtained from a subject. The biological sample may be derived from the subject. The biological sample may be obtained or derived from a subject at a second time point. The second time point may be subsequent to the administering. The methods may comprise detecting a second set of splice junctions. The methods may comprise computer processing the detected first and second sets of splice junctions. The methods may comprise assessing the effect of the compound based at least in part on the computer processing.

The methods may comprise assaying a first expression profile. The first expression profile may be of a first cell-free biological sample. The biological sample may be a blood sample. The biological sample may be a plasma sample. The biological sample may be a serum sample. The biological sample may be a buffy coat sample. The biological sample may be a urine sample. The biological sample may be a saliva sample. The biological sample may be a sweat sample. The biological sample may be a semen sample. The biological sample may be a vaginal discharge sample. The biological sample may be a cell-free sample. The cell-free sample may comprise cell-free RNA, such as cell-free mRNA (cf-mRNA). The biological sample may be a tissue sample. The biological sample may be a tumor biopsy sample. The biological sample may be a bone marrow sample.

The biological sample may comprise nucleic acids. The biological sample may comprise ribonucleic acids (RNAs), such as messenger RNAs (mRNAs). The RNA may be cell-free. The cell-free RNAs may be cell-free mRNAs. The RNA may be pre-mRNA. The RNA may comprise a coding region. The RNA may comprise a non-coding region. The RNA may comprise small nuclear RNAs (snRNAs), micro RNAs (miRNAs), or small interfering RNAs (siRNAs). The biological sample may comprise deoxyribonucleic acids (DNAs). The biological sample may comprise proteins.

The cell-free biological sample may be obtained or derived from a subject. The subject may be an animal. The subject may be a mammal, such as a human, a non-human primate, a rodent (e.g., a rat, a mouse, a guinea pig, a hamster, or the like), a dog, a cat, a pig, a sheep, a cow, a goat, or a rabbit. The subject may be a fish, a reptile, or a bird. The subject may be a human. The subject may be an adult (e.g., 18 years of age or older). The subject may be a child (e.g., less than 18 years of age). The subject may comprise an age of greater than or equal to 1, 2, 5, 10, 15, 20, 25, 30, 35, 40, 45, 50, 55, 60, 65, 70, 75, 80, 85, 90, or 95 years of age. The subject may be from about 50 to about 85 years of age. The subject may be from about 60 to about 80 years of age. The subject may be about 70 years of age. The subject may have or be suspected of having a disease state disclosed herein. For example, the subject may have or be suspected of having a dementia, for example, Alzheimer's disease. The subject may be asymptomatic. The subject may be healthy. The subject may be suspected of having a risk of a disease state disclosed herein. The subject may have one or more risk factors associated with a disease state. For example, the subject may have risk factors such as diabetes, hypertension, or the like. The subject may be predisposed to having a disease state disclosed herein. For example, the subject may be predisposed to having Alzheimer's disease. The subject may be in remission from a treatment to the disease state. The subject may have one or more symptoms of a disease state disclosed herein. For example, the subject may have symptoms such as memory loss, misplacement of items, difficulty in decision making and judging, confusion, mood swings, social withdrawal, inability to problem solve or complete tasks, or the like.

The methods may comprise assaying the biological sample. In some cases, the methods may comprise assaying cf-mRNA in the biological sample to detect one or more splice junctions in the cf-mRNA. The one or more splice junctions may correspond to one or more genes.

The methods may include sequencing. Non-limiting examples of sequencing include sequencing by synthesis (SBS), pyrosequencing, sequencing by reversible terminator chemistry, sequencing by ligation, phospholinked fluorescent nucleotide sequencing, real-time sequencing, and the like. The method may include next generation sequencing (NGS). NGS utilizes the concept of massively parallel processing to obtain high-throughput, speed, and scalability. NGS may be referred to as massive parallel sequencing, massively parallel sequencing, or second-generation sequencing. The methods may include RNA sequencing. Non-limiting examples of RNA sequencing include mRNA sequencing, total RNA sequencing, low-input RNA sequencing, ultra-low-input RNA sequencing, small RNA sequencing, single cell RNA sequencing, and the like. The methods may include DNA sequencing. Non-limiting examples of DNA sequencing include sanger sequencing, capillary electrophoresis, sequencing by synthesis, shotgun sequencing, pyrosequencing, combinatorial probe anchor synthesis, sequencing by ligation, nanopore sequencing, single molecular real time sequencing, ion torrent sequencing, nanoball sequencing, next generation sequencing, and the like.

The methods may include array hybridization. Array hybridization may include us of a microarray. A microarray is a laboratory tool that may be used to detect the expression of multiple genes at the same time. The microarray may be an analytical microarray, an antibody microarray, a functional microarray, a spotted array, a cellular microarray, an oligonucleotide DNA microarray, or the like. The microarray may use fluorescent dyes. The microarray may use probes, such as nucleotide probes. The microarray may comprise one or more wells, such as a 16-well plate, a 24-well plate, a 96 well plate, a 384-well plate, or the like. The one or more wells may be organized in rows and columns on the microarray.

The methods may include nucleic acid amplification. Nucleic acid amplification may include polymerase chain reaction (PCR), for example, multiplex PCR, long-range PCR, single-cell PCR, fast cycling PCR, methylation specific PCR, digital PCR, hot start PCR, real-time PCR (RT-PCR), quantitative PCR (qPCR), or the like. The nucleic acid amplification may include loop mediated isothermal amplification (LAMP). The nucleic acid amplification may include nucleic acid sequence-based amplification (NASBA). The nucleic acid amplification may include a strand displacement amplification (SDA). The nucleic acid amplification may include a multiple displacement amplification (MDA). The nucleic acid amplification may include rolling circle amplification (RCA). The nucleic acid amplification may include ligase chain reaction (LCR). The nucleic acid amplification may include helicase dependent amplification (HDA). The nucleic acid amplification may include a ramification amplification method (RAM). The nucleic acid amplification may include a transcription-mediated assay (TMA).

The methods may comprise administering a compound. The compound may be administered to a subject.

The compound may comprise a treatment. The treatment may treat a disease state of the subject. The treatment may treat one or more disease states of the subject. The treatment may be administered orally, intravenously, intramuscularly, subcutaneously, intrathecally, rectally, vaginally, topically, intranasally, or any combination thereof. The treatment may be in the form of a liquid, a tablet, or a capsule. The treatment may be in the form of a cream, a lotion, or an ointment. The treatment may be in the form of a droplet, an inhaler, an injection, a patch, an implant, or a suppository.

The treatment may treat memory loss, behavioral changes, sleep problems, and other symptoms associated with Alzheimer's disease. For example, citalopram, fluoxetine, paroxetine, and sertraline can be used to treat issues relating to mood, depression, and irritability experienced by Alzheimer's disease. As another example, alprazolam, buspirone, lorazepam, and oxazepam can be used to treat anxiety or restlessness associated with Alzheimer's disease. Further, unconventional therapies, such as hormone replacement therapy, art and music therapies, and supplements (e.g., vitamins such as vitamin E) can be used alternatively or additionally to treat the Alzheimer's disease.

The treatment may be a medicinal therapy. The medicinal therapy may be a cholinesterase inhibitor. The medicinal therapy may be a N-methyl-D-aspartate (NMDA) antagonist. The medicinal therapy may be an atypical antipsychotic. The medicinal therapy may be a disease-modifying immunotherapy. The medicinal therapy may be a monoclonal antibody (mab) therapy. The medicinal therapy may be an amyloid monoclonal antibody (mab) therapy.

The treatment may be a behavioral therapy. The behavioral therapy may include cognitive behavioral therapy, cognitive behavioral play therapy, dialectical behavioral therapy, exposure therapy, rational emotive behavior therapy, cognitive restructuring, aversion therapy, interpersonal psychotherapy, multisensory stimulation, active music therapy, cognitive stimulation, or any combination thereof. The treatment may be a sleep therapy.

The methods may include producing complementary deoxyribonucleic acid (cDNA) from RNA. In some cases, the methods may include converting RNA, for example cf-mRNA, to cDNA using a reverse transcription protocol. Reverse transcription is a process that converts RNA to cDNA using, among other things, a reverse transcriptase enzyme and deoxyribonucleotide triphosphates (dNTPs). Reverse transcriptase is an enzyme that is an RNA-dependent DNA polymerase. Reverse transcription may utilize several reaction components, such as an RNA template, one or more primers, one or more reaction buffers, dNTPs, DTT, RNase inhibitor, DNA polymerase, DNA ligase, water, or a combination thereof. The reverse transcription reaction may generally follow the steps of annealing, polymerization, and deactivation.

In some cases, a sample cDNA is produced from cf-mRNA by reverse transcription. In some cases, a cDNA library may be produced from the produced cDNA sample. The cDNA library may contain DNA copies of the cf-mRNA obtained from the biological sample. The cDNA library may be compared to a reference library. The reference library may be generated from a biological sample of a subject known not to have the disease state, for example, a subject known to be non-cognitively impaired, or a subject known not to have Alzheimer's disease.

The methods may include comparing a sample cDNA to a reference sample. The reference sample may be obtained from a healthy subject known not to have the disease state. For example, the reference sample may be obtained from a non-cognitively impaired subject. The methods may comprise identifying differences between the sample cDNA and the reference sample. For example, non-contiguous junctions may be present in the sample cDNA and not present in the reference sample. For example, one or more splice junctions may be present in the sample cDNA and not present in the reference sample. Additional differences, such as differences in nucleotide sequences, may be identified between the sample cDNA and the reference sample. In some cases, the reference sample may comprise aggregated least variant gene cf-mRNAs. In some cases, the reference sample may comprise prior sampling. In some cases, the reference sample may comprise a reference interval.

In some cases, the methods may comprise detecting one or more splice junctions. Splice junctions may be referred to as the boundaries between introns and/or exons during RNA splicing in transcription. In some cases, splice junctions may comprise non-coding splice junctions, such as splice junctions in 5′ or 3′ untranslated regions. Transcription is the process by which a cell makes an RNA copy of a piece of DNA. Splicing is the process in which introns, which are the noncoding regions of genes, are excised out of the primary messenger RNA transcript, and the exons, which are the coding regions, are joined together to generate a mature messenger RNA. Non-limiting examples of splice junctions include exon-exon splice junctions (e.g., the boundary between two exons), exon-intron splice junctions (e.g., the boundary between an exon and an intron), and intron-intron splice junctions (e.g., the boundary between two introns). The identification of splice junctions involves the recognition of exon-exon, exon-intron, and intron-intron boundaries during transcription. A splice junction may comprise a boundary between two nucleotides. A splice junction may comprise more than or equal to one nucleotide, for example, more than or equal to two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

The one or more splice junctions may comprise one or more isoforms. An isoform is a specific combination of splice junctions that can result from alternative splicing. Alternative splicing, also called alternative RNA splicing or differential splicing, is a process that allows a single gene to code for multiple proteins. Alternative splicing may generate different RNAs that are translated into proteins. In alternative splicing, exons/introns from the same gene are joined together in different combinations, leading to different but related resulting mRNA transcripts during transcription. There are several modes of alternative splicing, including exon skipping alternative splicing, mutually exclusive exon alternative slicing, alternative 3′ alternative splicing, alternative 5′ alternative splicing, and intron retention alternative splicing. In exon skipping alternative splicing, an exon may be retained or spliced out of the transcript. Exon skipping alternative splicing is the most common form of alternative splicing and results in the loss of an exon in the alternatively spliced transcript. In mutually exclusive exon alternative splicing, alternative isoforms are generated by retaining only one exon of a cluster of neighboring internal exons in the mature transcript. Mutually exclusive exon alternative splicing indicates that one out of two exons (or one group out of two exon groups) is retained, while the other exon/group is spliced out. In alternative 5′ alternative splicing, an alternative 5′ splice junction is used, which changes the 3′ boundary of the upstream exon. In alternative 3′ alternative splicing, an alternative 3′ splice junction is used, which changes the 5′ boundary of the downstream exon. In intron retention alternative splicing, an intron is retained in the mature mRNA transcript. In some cases, splicing occurs in a 3′ or 5′ untranslated region.

The one or more splice junctions may correspond to one or more genes. The methods may comprise determining that more than or equal to one splice junction corresponds to more than or equal to one gene. In some cases, one splice junction corresponds to more than or equal to one, two, three, four, five six, seven, eight, nine, 10, 11, 12, 13, 14, 15, 16, 17, 18, 19, 20, 21, 22, 23, 24, or 25 genes. In some cases, two splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, three splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, four splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, five splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 10 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 15 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 20 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 25 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 50 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 100 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 250 splice junctions correspond to more than or equal to one, two, three, four, five six, seven, eight, nine, 10, 11, 12, 13, 14, 15, 16, 17, 18, 19, 20, 21, 22, 23, 24, or 25 genes. In some cases, 500 splice junctions correspond to more than or equal to one, two, three, four, five six, seven, eight, nine, 10, 11, 12, 13, 14, 15, 16, 17, 18, 19, 20, 21, 22, 23, 24, or 25 genes.

In some cases, the methods disclosed herein may identify one or more genes that are expressed. For example, the genes may be expressed in a first population of subjects with a disease state, such as Alzheimer's disease, as compared to a second population of subjects known not to have the disease state. The second population of subjects may be non-cognitively impaired. The second population of subjects may be healthy. The second population of subjects may be known not have Alzheimer's disease.

The methods may comprise assaying a second expression profile. The second expression profile may be of a second cell-free biological sample. The second cell-free biological sample may be a biological sample disclosed herein. The cell-free biological sample may be obtained or derived from a subject as disclosed herein. The methods may comprise assaying the biological sample. In some cases, the methods may comprise assaying cf-mRNA in the biological sample to detect one or more splice junctions in the cf-mRNA. The one or more splice junctions may correspond to one or more genes. The assaying may comprise one or more of sequencing, array hybridization, or nucleic acid amplification as disclosed herein.

The methods may utilize computer processing. The computer processing may make use of machine learning as disclosed herein. The computer processing may use of a classifier as disclosed herein. The computer processing may make use of classification or prediction. The classifier may be a trained classifier. The classifier may be trained by the methods disclosed herein. In some cases, one or more sets of splice junctions are computer processed. In some cases, a first and second set of splice junctions are computer processed. In some cases, a first, a second, and third set of splice junctions are computer processed. In some cases, a first, a second, a third, and a fourth set of splice junctions are computer processed. In some cases, a first, a second, a third, a fourth, and a fifth set of splice junctions are computer processed. In some cases, a first, a second, a third, a fourth, a fifth, and a sixth set of splice junctions are computer processed. The sets of splice junctions may be detected by the methods disclosed herein.

The computer processing may comprise comparing detected sets of splice junctions. As disclosed herein, the methods may comprise detecting one or more sets of splice junctions. In some cases, a first and a second set of splice junctions are detected. In some cases, the methods comprise detecting more than or equal to two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 46, 47, 48, 49, or 50 sets of splice junctions. The computer processing disclosed herein may comprise comparing the one or more sets of splice junctions. For example, the computer processing may comprise comparing a first set and a second set of splice junctions. As another example, the computer processing may comprise comparing a first set, a second set, and a third set of splice junctions. As yet another example, the computer processing may comprise comparing a first set, a second set, a third set, and a fourth set of splice junctions.

The computer processing may comprise determining a difference between the detected sets of splice junctions. In some cases, the computer processing may determine one or more differences between the detected sets of splice junctions. In some cases, the computer processing may determine more than or equal to two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 46, 47, 48, 49, or 50 differences between the detected sets of splice junctions. In some cases, the identified difference may indicate the effect of the compound.

In some cases, the identified difference may comprise one or more expressed genes. In some cases, the identified difference may comprise one or more, five or more, 10 or more, 20 or more, 30 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, 200 or more, 210 or more, 220 or more, 230 or more, 240 or more, 250 or more, 260 or more, 270 or more, 280 or more, 290 or more, 300 or more, 310 or more, 320 or more, 330 or more, 340 or more, 350 or more, 360 or more, 370 or more, 380 or more, 390 or more, 400 or more, 410 or more, 420 or more, 430 or more, 440 or more, 450 or more, 460 or more, 470 or more, 480 or more, 490 or more, or 500 or more expressed genes. In some cases, the identified difference may comprise 500 or less, 490 or less, 480 or less, 470 or less, 460 or less, 450 or less, 440 or less, 430 or less, 420 or less, 410 or less, 400 or less, 390 or less, 380 or less, 370 or less, 360 or less, 350 or less, 340 or less, 330 or less, 320 or less, 310 or less, 300 or less, 290 or less, 280 or less, 270 or less, 260 or less, 250 or less, 240 or less, 230 or less, 220 or less, 210 or less, 200 or less, 190 or less, 180 or less, 170 or less, 160 or less, 150 or less, 140 or less, 130 or less, 120 or less, 110 or less, 100 or less, 90 or less, 80 or less, 70 or less, 60 or less, 50 or less, 40 or less, 30 or less, 20 or less, 10 or less expressed genes.

The identified difference may comprise any of the expressed genes present in Tables 1-3.

Modeling Alternative Junction Inclusion Quantification (“MAJIQ”) is a software package that can detect, quantify, and visualize local splicing variations (“LSV”) from RNA sequencing data. LSVs can include two or more splice junctions that can emanate out from a reference exon (e.g., a source LSV) or converge into a reference exon (e.g., a target LSV). LSV's can capture the classical, binary, alternative splicing events involving two alternative splice junctions. LSV's can also capture more complex (e.g., non-binary) splicing variations. A LSV ID (local splicing variations identifier), as used herein, is a unique identifier for the LSV that can be generated by the MAJIQ package. The LSV ID may be comprised of an ENSG Ensembl ID, whether it is a source (s) or target (t) LSV, exon coordinates, intron coordinates, and combinations thereof. For each gene listed herein, the LSV ID is provided in parentheses, for example Gene (LSV ID (e.g., ENSG number, source (s) or target (t), and exon/intron coordinates).

In some cases, the expressed splice junctions may comprise a member of one or more of the group consisting of: ABLIM1 (ENSG00000099204.21:t:114491791-114491878;114491878-114545005), ACAA1 (ENSG00000060971.19:s:38126493-38126700;38126341-38126510), ACAP1 (ENSG00000072818.12:t:7341948-7342067;7336787-7341948), ACOT8 (ENSG00000101473.17:t:45848450-45849110;45848675-45857188), ACP1 (ENSG00000143727.16:t:272192-273155;272065-272192), ADD3 (ENSG00000148700.15:t:110100625-110100848;110008299-110100625), ADGRE5 (ENSG00000123146.20:t:14397414-14397804;14391079-14397658), ADK (ENSG00000156110.15:t:74200764-74200838;74151343-74200764), AGAP3 (ENSG00000133612.19:s:151119987-151120145;151120145-151122725), AKAP13 (ENSG00000170776.22:t:85575131-85575329;85543955-85575131), ALAD (ENSG00000148218.16:s:113393161-113393634;113392169-113393447), AMPD2 (ENSG00000116337.20:t:109625303-109625433;109621266-109625303), ANAPC11 (ENSG00000141552.18:t:81894298-81894624;81891833-81894467), AP1B1 (ENSG00000100280.17:s:29330378-29330532;29329720-29330378), AP1B1 (ENSG00000100280.17:t:29327680-29328895;29328895-29329712), APP (ENSG00000142192.21:s:26000015-26000182;25982477-26000015), APP (ENSG00000142192.21:t:25897573-25897983;25897673-25911741), ARAP1 (ENSG00000186635.15:s:72693325-72693470;72688537-72693325), ARFRP1 (ENSG00000101246.20:t:63706999-63707658;63707097-63707867), ARHGAP17 (ENSG00000140750.17:t:24935470-24936827;24935639-24941987), ARHGAP17 (ENSG00000288353.1:t:188953-190310;189122-195470), ARHGEF10 (ENSG00000274726.4:s:43270-43560;43560-47749), ARHGEF1 (ENSG00000076928.19:s:41896377-41896878;41896482-41897315), ARHGEF7 (ENSG00000102606.20:t:111217679-111217885;111210002-111217679), ARMC10 (ENSG00000170632.14:s:103074881-103075411;103075411-103075777), ARRDC2 (ENSG00000105643.11:t:18008711-18008861;18001573-18008711), ATE1 (ENSG00000107669.19:t:121924266-121924329;121924329-121928347), ATP6V1D (ENSG00000100554.12:t:67350611-67350690;67350690-67359658), ATXN2L (ENSG00000168488.19:s:28835933-28836176;28836122-28836748), ATXN2L (ENSG00000168488.19:t:28836728-28837237;28836122-28836748), ATXN2L (ENSG00000168488.19:t:28836728-28837237;28836485-28836728), AURKB (ENSG00000178999.13:t:8207738-8208225;8207840-8210530), BLOC1S6 (ENSG00000104164.12:s:45592135-45592276;45592276-45605428), C12orf76 (ENSG00000174456.16:s:110048363-110048870;110042459-110048363), CAMTA1 (ENSG00000171735.20:t:6825092-6825210;6820250-6825092), CBFA2T3 (ENSG00000129993.15:t:88892244-88892485;88892485-88898078), CBFB (ENSG00000067955.15:t:67066682-67066962;67036755-67066682), CCDC28B (ENSG00000160050.16:t:32204598-32204620;32204379-32204598), CCDC92 (ENSG00000119242.9:s:123972529-123972831;123943493-123972529), CCDC92 (ENSG00000119242.9:t:123943347-123943495;123943493-123972529), CD300H (ENSG00000284690.3:s:74563919-74564275;74560824-74563919), CD34 (ENSG00000174059.17:s:207888682-207888846;207887923-207888682), CDAN1 (ENSG00000140326.13:s:42736989-42737128;42736780-42737013), CDC14B (ENSG00000081377.17:t:96565393-96565512;96565483-96619219), CDK5RAP2 (ENSG00000136861.19:t:120402806-120403071;120403071-120403316), CELF2 (ENSG00000048740.19:t:11249153-11249201;11165682-11249153), CEP164 (ENSG00000110274.16:t:117409618-117410701;117409028-117409618), CLEC1B (ENSG00000165682.15:s:9995140-9995246;9986158-9995140), COX20 (ENSG00000203667.10:s:244835616-244835756;244835756-244842195), CPNE1 (ENSG00000214078.13:s:35627280-35627531;35626800-35627280), CYTH2 (ENSG00000105443.17:s:48474838-48476276;48474949-48477717), CYTH2 (ENSG00000105443.17:t:48478066-48478145;48477719-48478069), DAZAP1 (ENSG00000071626.17:s:1422348-1422396;1422396-1425878), DCTD (ENSG00000129187.15:s:182917288-182917477;182915575-182917311), DCTD (ENSG00000129187.15:t:182915461-182915575;182915575-182917288), DECR1 (ENSG00000104325.7:s:90001405-90001561;90001561-90017124), DECR1 (ENSG00000104325.7:s:90001405-90001561;90001561-90018909), DECR1 (ENSG00000104325.7:t:90018564-90018966;90001561-90018909), DEF8 (ENSG00000140995.17:t:89954172-89954376;89949513-89954243), DGKD (ENSG00000077044.11:s:233390403-233390483;233390483-233392070), DGKZ (ENSG00000149091.15:t:46367291-46367399;46345585-46367291), DNM2 (ENSG00000079805.19:s:10795372-10795439;10795439-10797380), DOCK8 (ENSG00000107099.18:t:271627-271729;215029-271627), DUT (ENSG00000128951.14:t:48332103-48332737;48331479-48332268), DYRK1A (ENSG00000157540.22:s:37418905-37420384;37420384-37472684), DYSF (ENSG00000135636.16:t:71480883-71480938;71454086-71480883), EIF4G1 (ENSG00000114867.22:t:184317321-184317497;184314674-184317321), EIF4G3 (ENSG00000075151.24:t:20981048-20981227;20981227-20997601), EMC8 (ENSG00000131148.9:s:85781211-85781760;85779867-85781211), EPSTI1 (ENSG00000133106.15:s:42991978-42992271;42969177-42991978), EXOSC1 (ENSG00000171311.13:s:97438663-97438703;97437750-97438663), EXOSC1 (ENSG00000171311.13:t:97437700-97437750;97437750-97438663), F2RL3 (ENSG00000127533.4:s:16888999-16889298;16889298-16889577), FAM219A (ENSG00000164970.15:s:34405865-34405964;34402807-34405916), FAM3A (ENSG00000071889.17:s:154512823-154512936;154511871-154512823), FBXO44 (ENSG00000132879.14:t:11658736-11658871;11658628-11658736), FKBP1B (ENSG00000119782.14:s:24060814-24060926;24060926-24063019), FMNL3 (ENSG00000161791.14:t:49657082-49657190;49657190-49658442), FOXP1 (ENSG00000114861.24:t:71112536-71112637;71112637-71130498), G3BP1 (ENSG00000145907.16:t:151786572-151786715;151772036-151786603), GET1 (ENSG00000182093.16:t:39390698-39390803;39380556-39390698), GLUL (ENSG00000135821.19:t:182388572-182390304;182388750-182391175), GORASP1 (ENSG00000114745.15:s:39107479-39108063;39103553-39107479), GORASP1 (ENSG00000114745.15:t:39101016-39101102;39103553-39107479), GSE1 (ENSG00000131149.19:t:85648552-85648751;85556363-85648552), GSTT1 (ENSG00000277656.3:t:270997-271173;271173-278295), GUCD1 (ENSG00000138867.17:t:24548917-24549001;24549001-24555615), HDAC7 (ENSG00000061273.18:s:47796207-47796298;47796016-47796207), HES6 (ENSG00000144485.11:t:238239487-238239568;238239568-238239825), HUS1 (ENSG00000136273.13:s:47979410-47979615;47978816-47979410), IFI27 (ENSG00000275214.4:t:1230073-1230504;1229442-1230343), IGF2BP3 (ENSG00000136231.14:t:23342064-23342189;23342189-23343718), IKBKB (ENSG00000104365.16:t:42290156-42290273;42288728-42290156), IKBKG (ENSG00000269335.7:t:154551988-154552189;154542452-154551988), IKZF3 (ENSG00000161405.17:t:39777651-39777767;39777767-39788258), IL10RA (ENSG00000110324.12:t:117988382-117988502;117986534-117988382), IL15RA (ENSG00000134470.21:t:5960367-5960567;5960567-5963743), IMPDH1 (ENSG00000106348.18:t:128405767-128405948;128405865-128409289), INO80E (ENSG00000169592.15:s:30001414-30001575;30001530-30005221), INPP5D (ENSG00000281614.3:s:67445-67595;67595-71086), IP6K2 (ENSG00000068745.15:t:48694872-48695421;48695421-48717157), IRF7 (ENSG00000276561.4:s:144369-144427;144292-144369), IRF7 (ENSG00000276561.4:s:144676-144900;144424-144690), ITSN2 (ENSG000000198399.16:s:24246129-24246320;24242206-24246129), JAML (ENSG00000160593.19:t:118212407-118212561;118212561-118214824), JOSD2 (ENSG00000161677.12:t:50506378-50506572;50506572-50507574), KANK2 (ENSG00000197256.11:s:11195556-11195881;11194590-11195556), KANK2 (ENSG00000197256.11:t:11194553-11194590;11194590-11195556), KANSL3 (ENSG00000114982.19:s:96636739-96637228;96631543-96636921), KANSL3 (ENSG00000114982.19:t:96631312-96631543;96631543-96636921), KAT5 (ENSG00000172977.13:s:65712922-65713319;65713058-65713348), KATNB1 (ENSG00000140854.13:s:57755345-57755494;57755494-57755841), KDM5C (ENSG00000126012.13:s:53217796-53217966;53217274-53217796), KIAA1671 (ENSG00000197077.14:t:25170820-25170938;25049364-25170820), KIFC3 (ENSG00000140859.16:t:57772223-57772288;57772288-57785464), KIFC3 (ENSG00000140859.16:t:57798072-57798282;57798282-57802370), KMT2B (ENSG00000272333.8:t:35719784-35720940;35719541-35719784), LDB1 (ENSG00000198728.11:s:102109029-102109177;102108323-102109029), LDB2 (ENSG00000169744.13:s:16511981-16512104;16508686-16511981), LETMD1 (ENSG00000050426.16:s:51048301-51048518;51048478-51056350), LST1 (ENSG00000204482.11:s:31587117-31587318;31587318-31587641), LST1 (ENSG00000204482.11:t:31587944-31587966;31587318-31587944), LST1 (ENSG00000206433.10:s:2842938-2843143;2843139-2844386), LST1 (ENSG00000223465.8:s:2834852-2835057;2835053-2835376), LST1 (ENSG00000223465.8:t:2835679-2835701;2835053-2835679), LST1 (ENSG00000226182.8:t:3065231-3065253;3064605-3065231), LST1 (ENSG00000230791.8:s:2886832-2887014;2887014-2887225), LST1 (ENSG00000230791.8:t:2887225-2887247;2886599-2887225), LST1 (ENSG00000231048.8:s:2892158-2892363;2892359-2892682), LST1 (ENSG00000231048.8:t:2892985-2893007;2892359-2892985), LTB (ENSG00000204487.8:s:3059543-3059714;3058917-3059543), LTB (ENSG00000223448.7:s:2887297-2887468;2886671-2887297), LTB (ENSG00000238114.7:s:2881536-2881707;2880910-2881536), LTBP4 (ENSG00000090006.18:s:40619347-40619493;40619493-40622401), LTBP4 (ENSG00000090006.18:t:40619347-40619493;40614446-40619347), LTBP4 (ENSG00000090006.18:t:40623604-40623732;40623021-40623604), MAPK9 (ENSG00000050748.18:t:180269280-180269409;180269409-180279796), MARK2 (ENSG00000072518.22:s:63904786-63905043;63905043-63908260), MAST3 (ENSG00000099308.13:s:18146881-18147044;18147017-18147443), MAX (ENSG00000125952.20:t:65077193-65077428;65077428-65077913), MBNL1 (ENSG00000152601.18:t:152414941-152415111;152269092-152414941,) METTL9 (ENSG00000197006.15:s:21624931-21625233;21625115-21655227), MGLL (ENSG00000074416.16:t:127821694-127821838;127821838-127822309), MGRN1 (ENSG00000102858.13:s:4677463-4677572;4677572-4681550), MIEF1 (ENSG00000100335.15:s:39511849-39512026;39512026-39512232), MLX (ENSG00000108788.12:s:42567619-42567655;42567655-42568837), MMAB (ENSG00000139428.12:s:109561420-109561517;109561329-109561420), MPI (ENSG00000178802.18:s:74890005-74890201;74890089-74890527), MPRIP (ENSG00000133030.22:t:17142627-17142765;17138429-17142627), MRPL33 (ENSG00000243147.8:s:27772674-27772855;27772692-27779433), MSRB3 (ENSG00000174099.12:s:65278643-65278865;65278865-65326826), MTA1 (ENSG00000182979.18:s:105445418-105445511;105445511-105450058), MTA1 (ENSG00000182979.18:t:105450058-105450184;105445511-105450058), MTHFD2 (ENSG00000065911.13:t:74207704-74207826;74205889-74207704), MTMR12 (ENSG00000150712.11:s:32312758-32312987;32274122-32312758), MTMR12 (ENSG00000150712.11:t:32273980-32274122;32274122-32312758), NAP1L4 (ENSG00000273562.4:t:175479-176694;176694-182308), NCOA2 (ENSG00000140396.13:t:70141179-70141399;70141399-70148273), NCOR2 (ENSG00000196498.14:s:124330845-124330898;124326370-124330845), NCOR2 (ENSG00000196498.14:s:124378237-124378384;124372610-124378237), NCOR2 (ENSG00000196498.14:t:124372022-124372610;124372610-124378237), NDRG2 (ENSG00000165795.25:t:21022392-21022820;21022497-21022864), NDUFV3 (ENSG00000160194.18:t:42908864-42913304;42897047-42908864), NFE2L1 (ENSG00000082641.16:s:48056386-48056598;48056598-48057032), NFIA (ENSG00000162599.18:t:61455303-61462788;61406727-61455303), NKIRAS2 (ENSG00000168256.18:t:42023654-42025644;42022640-42023654), NOC2L (ENSG00000188976.11:s:945042-945146;944800-945057), NTAN1 (ENSG00000275779.4:s:613600-613788;613788-621580), NTPCR (ENSG00000135778.12:t:232956347-232956443;232950744-232956347), NUDT22 (ENSG00000149761.9:t:64229131-64229344;64227132-64229247), NUP214 (ENSG00000126883.19:s:131146129-131146304;131146304-131147490), NXT2 (ENSG00000101888.12:t:109538045-109538131;109537270-109538045), OCEL1 (ENSG00000099330.9:s:17226213-17226353;17226353-17226693), P2RX4 (ENSG00000135124.16:s:121210065-121210298;121210298-121217134), PABIR2 (ENSG00000156504.17:s:134781821-134781917;134772283-134781821), PALM2AKAP2 (ENSG00000157654.19:s:110138171-110138539;110138539-110168399), PANK4 (ENSG00000157881.16:t:2521101-2521315;2521315-2526464), PAPOLA (ENSG00000090060.19:s:96556175-96556413;96556413-96560649), PARL (ENSG00000175193.14:s:183862607-183862801;183844326-183862697), PARL (ENSG00000175193.14:t:183844231-183844774;183844326-183862753), PARVB (ENSG00000188677.15:t:44093928-44094017;44069162-44093928), PCYT1B (ENSG00000102230.14:t:24618985-24619084;24619084-24646989), PDE7A (ENSG00000205268.11:t:65782783-65782843;65782843-65841371), PEX26 (ENSG00000215193.14:s:18083437-18083732;18083732-18087972), PFKFB3 (ENSG00000170525.21:t:6213623-6213748;6203336-6213623), PKN1 (ENSG00000123143.13:t:14441143-14441443;14433542-14441143), PLEKHM1 (ENSG00000225190.12:s:45481603-45482612;45478147-45482433), PLS3 (ENSG00000102024.19:t:115610243-115610323;115561260-115610243), PML (ENSG00000140464.20:s:74034478-74034558;74034530-74042989), PML (ENSG00000140464.20:t:74033156-74033422;74032715-74033156), POLR2J3 (ENSG00000168255.20:t:102566997-102567083;102567083-102568010), POLR2J3 (ENSG00000285437.2:t:102566966-102567083;102567083-102568010), PPIE (ENSG000000084072.17:s:39752910-39753266;39753052-39753287), PPM1N (ENSG00000213889.11:t:45499949-45500066;45497343-45499949), PPP6R2 (ENSG00000100239.16:s:50436367-50436452;50436452-50436988), PPP6R2 (ENSG00000100239.16:t:50437506-50437603;50437068-50437506), PRKAR1B (ENSG00000188191.16:t:596146-596304;596304-602553), PRKCD (ENSG000000163932.16:t:53178404-53178537;53165215-53178404), PSEN1 (ENSG00000080815.20:t:73170797-73171923;73148094-73170797), PSMB8 (ENSG00000230034.8:t:4086512-4086659;4086659-4087849), PSMG4 (ENSG000000180822.12:s:3263684-3263759;3263759-3264209), PTK2B (ENSG00000120899.18:t:27450749-27450895;27445919-27450749), PTPN12 (ENSG00000127947.16:s:77600664-77600965;77600806-77607235), PTPN18 (ENSG00000072135.13:t:130368897-130369201;130356200-130369133), PUF60 (ENSG00000179950.15:s:143821118-143821743;143818534-143821597), PUF60 (ENSG00000179950.15:s:143821118-143821743;143820716-143821597), PUM1 (ENSG00000134644.16:s:30967099-30967310;30966278-30967167), PYM1 (ENSG00000170473.17:t:55903387-55903480;55903480-55927076), RAB11FIP1 (ENSG00000156675.16:t:37870387-37870528;37870528-37871278), RAB1FIP5 (ENSG00000135631.17:t:73075993-73076182;73076182-73088050), RAB4A (ENSG00000168118.12:t:229295848-229295910;229271370-229295848), RABGAP1L (ENSG00000152061.24:t:174969277-174969387;174957549-174969277), RAP1B (ENSG00000127314.18:t:68650244-68650882;68648781-68650400), RARA (ENSG00000131759.18:t:40348316-40348464;40331396-40348316), RBCK1 (ENSG000000125826.22:s:409830-410025;410025-417526), RBM10 (ENSG00000182872.16:t:47173128-47173197;47169498-47173128), RBM39 (ENSG000000131051.24:s:35713019-35713203;35709256-35713019), RCOR3 (ENSG00000117625.14:t:211313424-211316385;211312961-211313424), REPS2 (ENSG00000169891.18:s:17022123-17022371;17022271-17025059), RFFL (ENSG00000092871.17:s:35026374-35026568;35021781-35026374), RHD (ENSG00000187010.21:s:25303322-25303459;25303459-25328898), RMND5B (ENSG00000145916.19:t:178138108-178138396;178131054-178138108), RPUSD1 (ENSG00000007376.8:t:786826-786928;786928-787077), SEC16A (ENSG00000148396.19:s:136447227-136447364;136446949-136447227), SIGLEC10 (ENSG00000142512.15:t:51414422-51414515;51414515-51415181), SIRT3 (ENSG00000142082.15:s:218778-219041;216718-218778), SLA2 (ENSG00000101082.14:s:36615225-36615374;36614387-36615225), SLC66A2 (ENSG00000122490.19:t:79903446-79904183;79904183-79919184), SMARCA4 (ENSG00000127616.21:s:11034914-11035132;11035132-11041298), SMARCA4 (ENSG00000127616.21:t:11040634-11041560;11035132-11041298), SMARCC2 (ENSG00000139613.12:s:56173696-56173849;56173029-56173696), SNX20 (ENSG00000167208.16:s:50675770-50675921;50669148-50675770), SPIB (ENSG00000269404.7:s:50423605-50423755;50423751-50428038), SRSF6 (ENSG00000124193.16:s:43458361-43458509;43458509-43459153), SSX2IP (ENSG00000117155.17:t:84670646-84670815;84670815-84690371), STAMBP (ENSG00000124356.17:t:73830845-73831059;73829070-73830845), STAT3 (ENSG00000168610.16:t:42317182-42317224;42317224-42319856), SWI5 (ENSG00000175854.15:t:128276587-128276755;128276402-128276707), SYNE1 (ENSG00000131018.25:s:152301869-152302269;152300781-152301869), TANGO2 (ENSG00000183597.16:t:20061530-20061802;20056013-20061530), TCF3 (ENSG00000071564.19:s:1615686-1615804;1612433-1615686), TCF7L2 (ENSG00000148737.18:s:113146011-113146097;113146097-113150998), TECR (ENSG00000099797.15:s:14562356-14562575;14562575-14563174), TJP2 (ENSG00000119139.21:t:69212548-69212601;69151771-69212548), TLE4 (ENSG00000106829.21:s:79627374-79627752;79627448-79652593), TLE4 (ENSG00000106829.21:t:79652590-79652629;79627448-79652593), TMBIM1 (ENSG00000135926.15:s:218292466-218292586;218282181-218292466), TMBIM4 (ENSG00000155957.19:s:66169855-66170027;66161172-66169855), TMEM11 (ENSG00000178307.10:s:21214091-21214176;21211227-21214091), TMEM126B (ENSG00000171204.13:t:85631687-85631808;85628688-85631687), TMEM219 (ENSG00000149932.17:t:29962677-29963308;29962132-29963107), TNFSF12 (ENSG00000239697.12:t:7549466-7549618;7549312-7549474), TNK2 (ENSG00000061938.21:t:195888426-195888606;195888606-195908485), TNRC18 (ENSG00000182095.15:t:5325096-5325574;5325248-5329937), TRAPPC2 (ENSG00000196459.15:s:13734525-13734635;13719982-13734525), TRAPPC6A (ENSG00000007255.10:t:45164725-45164970;45164970-45165127), TSC22D3 (ENSG00000157514.18:t:107715773-107715950;107715950-107775100), TSPAN32 (ENSG00000064201.16:s:2314485-2314666;2314571-2316229), UBAC2 (ENSG00000134882.16:t:99238427-99238554;99200939-99238427), UFD1 (ENSG00000070010.19:t:19471687-19471808;19471808-19479083), URI1 (ENSG00000105176.18:s:29942239-29942664;29942664-29985223), URI1 (ENSG00000105176.18:t:29985223-29985301;29942664-29985223), USP15 (ENSG00000135655.16:s:62325872-62325933;62325933-62349221), USP22 (ENSG00000124422.12:t:21028542-21028674;21028674-21043321), VGLL4 (ENSG00000144560.16:t:11601833-11602022;11602022-11702971), WWP2 (ENSG00000198373.13:t:69786996-69787080;69762391-69786996), YAF2 (ENSG00000015153.15:s:42237599-42237800;42199235-42237599), YIPF6 (ENSG00000181704.12:s:68498562-68499157;68499123-68511849), YIPF6 (ENSG00000181704.12:t:68513327-68515340;68511977-68513327), ZBTB7B (ENSG00000160685.14:t:155014448-155015814;155002943-155014655), ZEB2 (ENSG00000169554.23:t:144517278-144517557;144517419-144517612), ZFAND1 (ENSG00000104231.11:s:81718182-81718224;81715114-81718182), ZFAND1 (ENSG00000104231.11:t:81714987-81715114;81715114-81718182), ZFAND2B (ENSG00000158552.13:s:219207648-219207779;219207774-219207887), ZMIZ2 (ENSG00000122515.16:s:44759281-44759460;44759460-44760151), ZMYND8 (ENSG00000101040.20:t:47236326-47236516;47236516-47238758), ZNF185 (ENSG00000147394.20:s:152922720-152922809;152922809-152928575), and ZNF451 (ENSG00000112200.17:t:57099061-57099141;57090274-57099061). In some cases, the expressed splice junctions may comprise expressed genes.

The identified gene may include ABLIM1 (ENSG00000099204.21:t:114491791-114491878;114491878-114545005). The identified gene may include ACAA1 (ENSG00000060971.19:s:38126493-38126700;38126341-38126510). The identified gene may include ACAP1 (ENSG00000072818.12:t:7341948-7342067;7336787-7341948). The identified gene may include ACOT8 (ENSG00000101473.17:t:45848450-45849110;45848675-45857188). The identified gene may include ACP1 (ENSG00000143727.16:t:272192-273155;272065-272192). The identified gene may include ADD3 (ENSG00000148700.15:t:110100625-110100848;110008299-110100625). The identified gene may include ADGRE5 (ENSG00000123146.20:t:14397414-14397804;14391079-14397658). The identified gene may include ADK (ENSG00000156110.15:t:74200764-74200838;74151343-74200764). The identified gene may include AGAP3 (ENSG00000133612.19:s:151119987-151120145;151120145-151122725). The identified gene may include AKAP13 (ENSG00000170776.22:t:85575131-85575329;85543955-85575131). The identified gene may include ALAD (ENSG00000148218.16:s:113393161-113393634;113392169-113393447). The identified gene may include AMPD2 (ENSG00000116337.20:t:109625303-109625433;109621266-109625303). The identified gene may include ANAPC11 (ENSG00000141552.18:t:81894298-81894624;81891833-81894467). The identified gene may include AP1B1 (ENSG00000100280.17:s:29330378-29330532;29329720-29330378). The identified gene may include AP1B1 (ENSG00000100280.17:t:29327680-29328895;29328895-29329712). The identified gene may include APP (ENSG00000142192.21:s:26000015-26000182;25982477-26000015). The identified gene may include APP (ENSG00000142192.21:t:25897573-25897983;25897673-25911741). The identified gene may include ARAP1 (ENSG00000186635.15:s:72693325-72693470;72688537-72693325). The identified gene may include ARFRP1 (ENSG00000101246.20:t:63706999-63707658;63707097-63707867). The identified gene may include ARHGAP17 (ENSG00000140750.17:t:24935470-24936827;24935639-24941987). The identified gene may include ARHGAP17 (ENSG00000288353.1:t:188953-190310;189122-195470). The identified gene may include ARHGEF10 (ENSG00000274726.4:s:43270-43560;43560-47749). The identified gene may include ARHGEF1 (ENSG00000076928.19:s:41896377-41896878;41896482-41897315). The identified gene may include ARHGEF7 (ENSG00000102606.20:t:111217679-111217885;111210002-111217679). The identified gene may include ARMC10 (ENSG00000170632.14:s:103074881-103075411;103075411-103075777). The identified gene may include ARRDC2 (ENSG00000105643.11:t:18008711-18008861;18001573-18008711). The identified gene may include ATE1 (ENSG00000107669.19:t:121924266-121924329;121924329-121928347). The identified gene may include ATP6V1D (ENSG00000100554.12:t:67350611-67350690;67350690-67359658). The identified gene may include ATXN2L (ENSG00000168488.19:s:28835933-28836176;28836122-28836748). The identified gene may include ATXN2L (ENSG00000168488.19:t:28836728-28837237;28836122-28836748). The identified gene may include ATXN2L (ENSG00000168488.19:t:28836728-28837237;28836485-28836728). The identified gene may include AURKB (ENSG00000178999.13:t:8207738-8208225;8207840-8210530). The identified gene may include BLOC1S6 (ENSG00000104164.12:s:45592135-45592276;45592276-45605428). The identified gene may include C12orf76 (ENSG00000174456.16:s:110048363-110048870;110042459-110048363). The identified gene may include CAMTA1 (ENSG00000171735.20:t:6825092-6825210;6820250-6825092). The identified gene may include CBFA2T3 (ENSG00000129993.15:t:88892244-88892485;88892485-88898078). The identified gene may include CBFB (ENSG00000067955.15:t:67066682-67066962;67036755-67066682). The identified gene may include CCDC28B (ENSG00000160050.16:t:32204598-32204620;32204379-32204598). The identified gene may include CCDC92 (ENSG00000119242.9:s:123972529-123972831;123943493-123972529). The identified gene may include CCDC92 (ENSG00000119242.9:t:123943347-123943495;123943493-123972529). The identified gene may include CD300H (ENSG00000284690.3:s:74563919-74564275;74560824-74563919). The identified gene may include CD34 (ENSG00000174059.17:s:207888682-207888846;207887923-207888682). The identified gene may include CDAN1 (ENSG00000140326.13:s:42736989-42737128;42736780-42737013). The identified gene may include CDC14B (ENSG00000081377.17:t:96565393-96565512;96565483-96619219). The identified gene may include CDK5RAP2 (ENSG00000136861.19:t:120402806-120403071;120403071-120403316). The identified gene may include CELF2 (ENSG00000048740.19:t:11249153-11249201;11165682-11249153). The identified gene may include CEP164 (ENSG00000110274.16:t:117409618-117410701;117409028-117409618). The identified gene may include CLEC1B (ENSG00000165682.15:s:9995140-9995246;9986158-9995140). The identified gene may include COX20 (ENSG00000203667.10:s:244835616-244835756;244835756-244842195). The identified gene may include CPNE1 (ENSG00000214078.13:s:35627280-35627531;35626800-35627280). The identified gene may include CYTH2 (ENSG00000105443.17:s:48474838-48476276;48474949-48477717). The identified gene may include CYTH2 (ENSG00000105443.17:t:48478066-48478145;48477719-48478069). The identified gene may include DAZAP1 (ENSG00000071626.17:s:1422348-1422396;1422396-1425878). The identified gene may include DCTD (ENSG00000129187.15:s:182917288-182917477;182915575-182917311). The identified gene may include DCTD (ENSG00000129187.15:t:182915461-182915575;182915575-182917288). The identified gene may include DECR1 (ENSG00000104325.7:s:90001405-90001561;90001561-90017124). The identified gene may include DECR1 (ENSG00000104325.7:s:90001405-90001561;90001561-90018909). The identified gene may include DECR1 (ENSG00000104325.7:t:90018564-90018966;90001561-90018909). The identified gene may include DEF8 (ENSG00000140995.17:t:89954172-89954376;89949513-89954243). The identified gene may include DGKD (ENSG00000077044.11:s:233390403-233390483;233390483-233392070). The identified gene may include DGKZ (ENSG00000149091.15:t:46367291-46367399;46345585-46367291). The identified gene may include DNM2 (ENSG00000079805.19:s:10795372-10795439;10795439-10797380). The identified gene may include DOCK8 (ENSG00000107099.18:t:271627-271729;215029-271627). The identified gene may include DUT (ENSG00000128951.14:t:48332103-48332737;48331479-48332268). The identified gene may include DYRK1A (ENSG00000157540.22:s:37418905-37420384;37420384-37472684). The identified gene may include DYSF (ENSG00000135636.16:t:71480883-71480938;71454086-71480883). The identified gene may include EIF4G1 (ENSG00000114867.22:t:184317321-184317497;184314674-184317321). The identified gene may include EIF4G3 (ENSG00000075151.24:t:20981048-20981227;20981227-20997601). The identified gene may include EMC8 (ENSG00000131148.9:s:85781211-85781760;85779867-85781211). The identified gene may include EPSTI1 (ENSG00000133106.15:s:42991978-42992271;42969177-42991978). The identified gene may include EXOSC1 (ENSG00000171311.13:s:97438663-97438703;97437750-97438663). The identified gene may include EXOSC1 (ENSG00000171311.13:t:97437700-97437750;97437750-97438663). The identified gene may include F2RL3 (ENSG00000127533.4:s:16888999-16889298;16889298-16889577). The identified gene may include FAM219A (ENSG00000164970.15:s:34405865-34405964;34402807-34405916). The identified gene may include FAM3A (ENSG00000071889.17:s:154512823-154512936;154511871-154512823). The identified gene may include FBXO44 (ENSG00000132879.14:t:11658736-11658871;11658628-11658736). The identified gene may include FKBP1B (ENSG00000119782.14:s:24060814-24060926;24060926-24063019). The identified gene may include FMNL3 (ENSG00000161791.14:t:49657082-49657190;49657190-49658442). The identified gene may include FOXP1 (ENSG00000114861.24:t:71112536-71112637;71112637-71130498). The identified gene may include G3BP1 (ENSG00000145907.16:t:151786572-151786715;151772036-151786603). The identified gene may include GET1 (ENSG00000182093.16:t:39390698-39390803;39380556-39390698). The identified gene may include GLUL (ENSG00000135821.19:t:182388572-182390304;182388750-182391175). The identified gene may include GORASP1 (ENSG00000114745.15:s:39107479-39108063;39103553-39107479). The identified gene may include GORASP1 (ENSG00000114745.15:t:39101016-39101102;39103553-39107479). The identified gene may include GSE1 (ENSG00000131149.19:t:85648552-85648751;85556363-85648552). The identified gene may include GSTT1 (ENSG00000277656.3:t:270997-271173;271173-278295). The identified gene may include GUCD1 (ENSG00000138867.17:t:24548917-24549001;24549001-24555615). The identified gene may include HDAC7 (ENSG00000061273.18:s:47796207-47796298;47796016-47796207). The identified gene may include HES6 (ENSG00000144485.11:t:238239487-238239568;238239568-238239825) The identified gene may include HUS1 (ENSG00000136273.13:s:47979410-47979615;47978816-47979410). The identified gene may include IFI27 (ENSG00000275214.4:t:1230073-1230504;1229442-1230343). The identified gene may include IGF2BP3 (ENSG00000136231.14:t:23342064-23342189;23342189-23343718). The identified gene may include IKBKB (ENSG00000104365.16:t:42290156-42290273;42288728-42290156). The identified gene may include IKBKG (ENSG00000269335.7:t:154551988-154552189;154542452-154551988). The identified gene may include IKZF3 (ENSG00000161405.17:t:39777651-39777767;39777767-39788258). The identified gene may include IL10RA (ENSG00000110324.12:t:117988382-117988502;117986534-117988382). The identified gene may include IL15RA (ENSG00000134470.21:t:5960367-5960567;5960567-5963743). The identified gene may include IMPDH1 (ENSG00000106348.18:t:128405767-128405948;128405865-128409289). The identified gene may include INO80E (ENSG00000169592.15:s:30001414-30001575;30001530-30005221). The identified gene may include INPP5D (ENSG00000281614.3:s:67445-67595;67595-71086). The identified gene may include IP6K2 (ENSG00000068745.15:t:48694872-48695421;48695421-48717157). The identified gene may include IRF7 (ENSG00000276561.4:s:144369-144427;144292-144369). The identified gene may include IRF7 (ENSG00000276561.4:s:144676-144900;144424-144690). The identified gene may include ITSN2 (ENSG00000198399.16:s:24246129-24246320;24242206-24246129). The identified gene may include JAML (ENSG00000160593.19:t:118212407-118212561;118212561-118214824). The identified gene may include JOSD2 (ENSG00000161677.12:t:50506378-50506572;50506572-50507574). The identified gene may include KANK2 (ENSG00000197256.11:s:11195556-11195881;11194590-11195556). The identified gene may include KANK2 (ENSG00000197256.11:t:11194553-11194590;11194590-11195556). The identified gene may include KANSL3 (ENSG00000114982.19:s:96636739-96637228;96631543-96636921). The identified gene may include KANSL3 (ENSG00000114982.19:t:96631312-96631543;96631543-96636921). The identified gene may include KAT5 (ENSG00000172977.13:s:65712922-65713319;65713058-65713348). The identified gene may include KATNB1 (ENSG00000140854.13:s:57755345-57755494;57755494-57755841). The identified gene may include KDM5C (ENSG00000126012.13:s:53217796-53217966;53217274-53217796). The identified gene may include KIAA1671 (ENSG00000197077.14:t:25170820-25170938;25049364-25170820). The identified gene may include KIFC3 (ENSG00000140859.16:t:57772223-57772288;57772288-57785464). The identified gene may include KIFC3 (ENSG00000140859.16:t:57798072-57798282;57798282-57802370). The identified gene may include KMT2B (ENSG00000272333.8:t:35719784-35720940;35719541-35719784). The identified gene may include LDB1 (ENSG00000198728.11:s:102109029-102109177;102108323-102109029). The identified gene may include LDB2 (ENSG00000169744.13:s:16511981-16512104;16508686-16511981). The identified gene may include LETMD1 (ENSG00000050426.16:s:51048301-51048518;51048478-51056350). The identified gene may include LST1 (ENSG00000204482.11:s:31587117-31587318;31587318-31587641). The identified gene may include LST1 (ENSG00000204482.11:t:31587944-31587966;31587318-31587944). The identified gene may include LST1 (ENSG00000206433.10:s:2842938-2843143;2843139-2844386). The identified gene may include LST1 (ENSG00000223465.8:s:2834852-2835057;2835053-2835376). The identified gene may include LST1 (ENSG00000223465.8:t:2835679-2835701;2835053-2835679). The identified gene may include LST1 (ENSG00000226182.8:t:3065231-3065253;3064605-3065231). The identified gene may include LST1 (ENSG00000230791.8:s:2886832-2887014;2887014-2887225). The identified gene may include LST1 (ENSG00000230791.8:t:2887225-2887247;2886599-2887225). The identified gene may include LST1 (ENSG00000231048.8:s:2892158-2892363;2892359-2892682). The identified gene may include LST1 (ENSG00000231048.8:t:2892985-2893007;2892359-2892985). The identified gene may include LTB (ENSG00000204487.8:s:3059543-3059714;3058917-3059543). The identified gene may include LTB (ENSG00000223448.7:s:2887297-2887468;2886671-2887297). The identified gene may include LTB (ENSG00000238114.7:s:2881536-2881707;2880910-2881536). The identified gene may include LTBP4 (ENSG00000090006.18:s:40619347-40619493;40619493-40622401). The identified gene may include LTBP4 (ENSG00000090006.18:t:40619347-40619493;40614446-40619347). The identified gene may include LTBP4 (ENSG00000090006.18:t:40623604-40623732;40623021-40623604). The identified gene may include MAPK9 (ENSG00000050748.18:t:180269280-180269409;180269409-180279796). The identified gene may include MARK2 (ENSG00000072518.22:s:63904786-63905043;63905043-63908260). The identified gene may include MAST3 (ENSG00000099308.13:s:18146881-18147044;18147017-18147443). The identified gene may include MAX (ENSG00000125952.20:t:65077193-65077428;65077428-65077913). The identified gene may include MBNL1 (ENSG00000152601.18:t:152414941-152415111;152269092-152414941. The identified gene may include METTL9 (ENSG00000197006.15:s:21624931-21625233;21625115-21655227). The identified gene may include MGLL (ENSG00000074416.16:t:127821694-127821838;127821838-127822309). The identified gene may include MGRN1 (ENSG00000102858.13:s:4677463-4677572;4677572-4681550). The identified gene may include MIEF1 (ENSG00000100335.15:s:39511849-39512026;39512026-39512232). The identified gene may include MLX (ENSG00000108788.12:s:42567619-42567655;42567655-42568837). The identified gene may include MMAB (ENSG00000139428.12:s:109561420-109561517;109561329-109561420). The identified gene may include MPI (ENSG00000178802.18:s:74890005-74890201;74890089-74890527). The identified gene may include MPRIP (ENSG00000133030.22:t:17142627-17142765;17138429-17142627). The identified gene may include MRPL33 (ENSG00000243147.8:s:27772674-27772855;27772692-27779433). The identified gene may include MSRB3 (ENSG00000174099.12:s:65278643-65278865;65278865-65326826). The identified gene may include MTA1 (ENSG00000182979.18:s:105445418-105445511;105445511-105450058). The identified gene may include MTA1 (ENSG00000182979.18:t:105450058-105450184;105445511-105450058). The identified gene may include MTHFD2 (ENSG00000065911.13:t:74207704-74207826;74205889-74207704). The identified gene may include MTMR12 (ENSG00000150712.11:s:32312758-32312987;32274122-32312758). The identified gene may include MTMR12 (ENSG00000150712.11:t:32273980-32274122;32274122-32312758). The identified gene may include NAP1L4 (ENSG00000273562.4:t:175479-176694;176694-182308). The identified gene may include NCOA2 (ENSG00000140396.13:t:70141179-70141399;70141399-70148273). The identified gene may include NCOR2 (ENSG00000196498.14:s:124330845-124330898;124326370-124330845). The identified gene may include NCOR2 (ENSG00000196498.14:s:124378237-124378384;124372610-124378237). The identified gene may include NCOR2 (ENSG00000196498.14:t:124372022-124372610;124372610-124378237). The identified gene may include NDRG2 (ENSG00000165795.25:t:21022392-21022820;21022497-21022864). The identified gene may include NDUFV3 (ENSG00000160194.18:t:42908864-42913304;42897047-42908864). The identified gene may include NFE2L1 (ENSG00000082641.16:s:48056386-48056598;48056598-48057032). The identified gene may include NFIA (ENSG00000162599.18:t:61455303-61462788;61406727-61455303). The identified gene may include NKIRAS2 (ENSG00000168256.18:t:42023654-42025644;42022640-42023654). The identified gene may include NOC2L (ENSG00000188976.11:s:945042-945146;944800-945057). The identified gene may include NTAN1 (ENSG00000275779.4:s:613600-613788;613788-621580). The identified gene may include NTPCR (ENSG00000135778.12:t:232956347-232956443;232950744-232956347). The identified gene may include NUDT22 (ENSG00000149761.9:t:64229131-64229344;64227132-64229247). The identified gene may include NUP214 (ENSG00000126883.19:s:131146129-131146304;131146304-131147490). The identified gene may include NXT2 (ENSG00000101888.12:t:109538045-109538131;109537270-109538045). The identified gene may include OCEL1 (ENSG00000099330.9:s:17226213-17226353;17226353-17226693). The identified gene may include P2RX4 (ENSG00000135124.16:s:121210065-121210298;121210298-121217134). The identified gene may include PABIR2 (ENSG00000156504.17:s:134781821-134781917;134772283-134781821). The identified gene may include PALM2AKAP2 (ENSG00000157654.19:s:110138171-110138539;110138539-110168399). The identified gene may include PANK4 (ENSG00000157881.16:t:2521101-2521315;2521315-2526464). The identified gene may include PAPOLA (ENSG00000090060.19:s:96556175-96556413;96556413-96560649). The identified gene may include PARL (ENSG00000175193.14:s:183862607-183862801;183844326-183862697). The identified gene may include PARL (ENSG00000175193.14:t:183844231-183844774;183844326-183862753). The identified gene may include PARVB (ENSG00000188677.15:t:44093928-44094017;44069162-44093928). The identified gene may include PCYT1B (ENSG00000102230.14:t:24618985-24619084;24619084-24646989). The identified gene may include PDE7A (ENSG00000205268.11:t:65782783-65782843;65782843-65841371). The identified gene may include PEX26 (ENSG00000215193.14:s:18083437-18083732;18083732-18087972). The identified gene may include PFKFB3 (ENSG00000170525.21:t:6213623-6213748;6203336-6213623). The identified gene may include PKN1 (ENSG00000123143.13:t:14441143-14441443;14433542-14441143). The identified gene may include PLEKHM1 (ENSG00000225190.12:s:45481603-45482612;45478147-45482433). The identified gene may include PLS3 (ENSG00000102024.19:t:115610243-115610323;115561260-115610243). The identified gene may include PML (ENSG00000140464.20:s:74034478-74034558;74034530-74042989). The identified gene may include PML (ENSG00000140464.20:t:74033156-74033422;74032715-74033156). The identified gene may include POLR2J3 (ENSG00000168255.20:t:102566997-102567083;102567083-102568010). The identified gene may include POLR2J3 (ENSG00000285437.2:t:102566966-102567083;102567083-102568010). The identified gene may include PPIE (ENSG00000084072.17:s:39752910-39753266;39753052-39753287). The identified gene may include PPM1N (ENSG00000213889.11:t:45499949-45500066;45497343-45499949). The identified gene may include PPP6R2 (ENSG00000100239.16:s:50436367-50436452;50436452-50436988) The identified gene may include PPP6R2 (ENSG00000100239.16:t:50437506-50437603;50437068-50437506). The identified gene may include PRKAR1B (ENSG00000188191.16:t:596146-596304;596304-602553). The identified gene may include PRKCD (ENSG00000163932.16:t:53178404-53178537;53165215-53178404). The identified gene may include PSEN1 (ENSG00000080815.20:t:73170797-73171923;73148094-73170797). The identified gene may include PSMB8 (ENSG00000230034.8:t:4086512-4086659;4086659-4087849). The identified gene may include PSMG4 (ENSG00000180822.12:s:3263684-3263759;3263759-3264209). The identified gene may include PTK2B (ENSG00000120899.18:t:27450749-27450895;27445919-27450749). The identified gene may include PTPN12 (ENSG00000127947.16:s:77600664-77600965;77600806-77607235). The identified gene may include PTPN18 (ENSG00000072135.13:t:130368897-130369201;130356200-130369133). The identified gene may include PUF60 (ENSG00000179950.15:s:143821118-143821743;143818534-143821597). The identified gene may include PUF60 (ENSG00000179950.15:s:143821118-143821743;143820716-143821597). The identified gene may include PUM1 (ENSG00000134644.16:s:30967099-30967310;30966278-30967167). The identified gene may include PYM1 (ENSG00000170473.17:t:55903387-55903480;55903480-55927076). The identified gene may include RAB11FIP1 (ENSG00000156675.16:t:37870387-37870528;37870528-37871278). The identified gene may include RAB11FIP5 (ENSG00000135631.17:t:73075993-73076182;73076182-73088050). The identified gene may include RAB4A (ENSG00000168118.12:t:229295848-229295910;229271370-229295848). The identified gene may include RABGAP1L (ENSG00000152061.24:t:174969277-174969387;174957549-174969277). The identified gene may include RAP1B (ENSG00000127314.18:t:68650244-68650882;68648781-68650400). The identified gene may include RARA (ENSG00000131759.18:t:40348316-40348464;40331396-40348316). The identified gene may include RBCK1 (ENSG00000125826.22:s:409830-410025;410025-417526). The identified gene may include RBM10 (ENSG00000182872.16:t:47173128-47173197;47169498-47173128). The identified gene may include RBM39 (ENSG00000131051.24:s:35713019-35713203;35709256-35713019). The identified gene may include RCOR3 (ENSG00000117625.14:t:211313424-211316385;211312961-211313424). The identified gene may include REPS2 (ENSG00000169891.18:s:17022123-17022371;17022271-17025059). The identified gene may include RFFL (ENSG00000092871.17:s:35026374-35026568;35021781-35026374). The identified gene may include RHD (ENSG00000187010.21:s:25303322-25303459;25303459-25328898). The identified gene may include RMND5B (ENSG00000145916.19:t:178138108-178138396;178131054-178138108). The identified gene may include RPUSD1 (ENSG00000007376.8:t:786826-786928;786928-787077). The identified gene may include SEC16A (ENSG00000148396.19:s:136447227-136447364;136446949-136447227). The identified gene may include SIGLEC10 (ENSG00000142512.15:t:51414422-51414515;51414515-51415181). The identified gene may include SIRT3 (ENSG00000142082.15:s:218778-219041;216718-218778). The identified gene may include SLA2 (ENSG00000101082.14:s:36615225-36615374;36614387-36615225). The identified gene may include SLC66A2 (ENSG00000122490.19:t:79903446-79904183;79904183-79919184). The identified gene may include SMARCA4 (ENSG00000127616.21:s:11034914-11035132;11035132-11041298). The identified gene may include SMARCA4 (ENSG00000127616.21:t:11040634-11041560;11035132-11041298). The identified gene may include SMARCC2 (ENSG00000139613.12:s:56173696-56173849;56173029-56173696). The identified gene may include SNX20 (ENSG00000167208.16:s:50675770-50675921;50669148-50675770). The identified gene may include SPIB (ENSG00000269404.7:s:50423605-50423755;50423751-50428038). The identified gene may include SRSF6 (ENSG00000124193.16:s:43458361-43458509;43458509-43459153). The identified gene may include SSX2IP (ENSG00000117155.17:t:84670646-84670815;84670815-84690371). The identified gene may include STAMBP (ENSG00000124356.17:t:73830845-73831059;73829070-73830845). The identified gene may include STAT3 (ENSG00000168610.16:t:42317182-42317224;42317224-42319856). The identified gene may include SWI5 (ENSG00000175854.15:t:128276587-128276755;128276402-128276707). The identified gene may include SYNE1 (ENSG00000131018.25:s:152301869-152302269;152300781-152301869). The identified gene may include TANGO2 (ENSG00000183597.16:t:20061530-20061802;20056013-20061530). The identified gene may include TCF3 (ENSG00000071564.19:s:1615686-1615804;1612433-1615686). The identified gene may include TCF7L2 (ENSG00000148737.18:s:113146011-113146097;113146097-113150998). The identified gene may include TECR (ENSG00000099797.15:s:14562356-14562575;14562575-14563174). The identified gene may include TJP2 (ENSG00000119139.21:t:69212548-69212601;69151771-69212548). The identified gene may include TLE4 (ENSG00000106829.21:s:79627374-79627752;79627448-79652593). The identified gene may include TLE4 (ENSG00000106829.21:t:79652590-79652629;79627448-79652593). The identified gene may include TMBIM1 (ENSG00000135926.15:s:218292466-218292586;218282181-218292466). The identified gene may include TMBIM4 (ENSG00000155957.19:s:66169855-66170027;66161172-66169855). The identified gene may include TMEM11 (ENSG00000178307.10:s:21214091-21214176;21211227-21214091). The identified gene may include TMEM126B (ENSG00000171204.13:t:85631687-85631808;85628688-85631687). The identified gene may include TMEM219 (ENSG000000149932.17:t:29962677-29963308;29962132-29963107). The identified gene may include TNFSF12 (ENSG00000239697.12:t:7549466-7549618;7549312-7549474). The identified gene may include TNK2 (ENSG00000061938.21:t:195888426-195888606;195888606-195908485). The identified gene may include TNRC18 (ENSG00000182095.15:t:5325096-5325574;5325248-5329937). The identified gene may include TRAPPC2 (ENSG00000196459.15:s:13734525-13734635;13719982-13734525). The identified gene may include TRAPPC6A (ENSG00000007255.10:t:45164725-45164970;45164970-45165127). The identified gene may include TSC22D3 (ENSG00000157514.18:t:107715773-107715950;107715950-107775100). The identified gene may include TSPAN32 (ENSG00000064201.16:s:2314485-2314666;2314571-2316229). The identified gene may include UBAC2 (ENSG00000134882.16:t:99238427-99238554;99200939-99238427). The identified gene may include UFD1 (ENSG00000070010.19:t:19471687-19471808;19471808-19479083). The identified gene may include URI1 (ENSG00000105176.18:s:29942239-29942664;29942664-29985223). The identified gene may include URI1 (ENSG00000105176.18:t:29985223-29985301;29942664-29985223). The identified gene may include USP15 (ENSG000000135655.16:s:62325872-62325933;62325933-62349221). The identified gene may include USP22 (ENSG00000124422.12:t:21028542-21028674;21028674-21043321). The identified gene may include VGLL4 (ENSG00000144560.16:t:11601833-11602022;11602022-11702971). The identified gene may include WWP2 (ENSG00000198373.13:t:69786996-69787080;69762391-69786996). The identified gene may include YAF2 (ENSG00000015153.15:s:42237599-42237800;42199235-42237599). The identified gene may include YIPF6 (ENSG00000181704.12:s:68498562-68499157;68499123-68511849). The identified gene may include YIPF6 (ENSG00000181704.12:t:68513327-68515340;68511977-68513327). The identified gene may include ZBTB7B (ENSG00000160685.14:t:155014448-155015814;155002943-155014655). The identified gene may include ZEB2 (ENSG00000169554.23:t:144517278-144517557;144517419-144517612). The identified gene may include ZFAND1 (ENSG00000104231.11:s:81718182-81718224;81715114-81718182). The identified gene may include ZFAND1 (ENSG00000104231.11:t:81714987-81715114;81715114-81718182). The identified gene may include ZFAND2B (ENSG00000158552.13:s:219207648-219207779;219207774-219207887). The identified gene may include ZMIZ2 (ENSG00000122515.16:s:44759281-44759460;44759460-44760151). The identified gene may include ZMYND8 (ENSG00000101040.20:t:47236326-47236516;47236516-47238758). The identified gene may include ZNF185 (ENSG00000147394.20:s:152922720-152922809;152922809-152928575). The identified gene may include ZNF451 (ENSG00000112200.17:t:57099061-57099141;57090274-57099061). The identified gene may comprise any gene provided in Tables 1-3. In some cases, the expressed splice junctions may comprise expressed genes.

The methods may comprise assessing the effect of a compound. In some cases, the assessing the effect of the compound is based at least in part on computer processing.

The methods may further include identifying a tissue of a disease state. The methods may comprise analyzing the cf-mRNA in the biological sample and determining a tissue that the cf-mRNA originated from. The tissue may be identified to be under duress. The tissue may be identified to be impacted by the disease state. The tissue may be identified to be the origin of the disease state. The tissue may be nervous tissue, such as tissue of the brain, spinal cord, or nerves. The tissue may comprise circulating immune cells. The tissue may be muscle tissue, such as cardiac muscle tissue, smooth muscle tissue, or skeletal muscle tissue. The muscle tissue may originate from muscles in the body. The tissue may be epithelial tissue, such as lining of the gastrointestinal tract of organs or the skin surface (epidermis). The tissue may be connective tissue, such as tissue from fat (or other soft padding tissue), bone, or tendons. The tissue may be any tissue in the body.

The methods may further comprise identifying an organ of a disease state. One or more organs may be identified of the disease state. The methods may comprise analyzing the cf-mRNA in the biological sample and determining an organ that the cf-mRNA originated from. The organ may be identified to be under duress. The organ may be identified to be impacted by the disease state. The organ may be identified to be the origin of the disease state. The organ may be the lungs. The organ may be the liver. The organ may be the bladder. The organ may be the kidneys. The organ may be the heart. The organ may be the stomach. The organ may be the intestines, such as the small intestine or the large intestine. The organ may be the brain. The organ may be the pancreas. The organ may be the gallbladder. The organ may be any organ in the body.

The methods may further comprise identifying one or more biological pathways of the disease state. The biological pathways may be identified to be under duress. The biological pathways may be identified to be impacted by the disease state. The biological pathways may be identified to be the origin of the disease state. The biological pathways may include neurological pathways, digestive pathways, muscular pathways, respiratory pathways, endocrine pathways, reproductive pathways, skeletal pathways, lymphatic pathways, immune pathways, immunological pathways, gastrointestinal pathways, nervous system pathways, or any combination thereof. The biological pathways may relate to the disease state. For example, the biological pathways may relate to a neurodegenerative disease. In some cases, the neurodegenerative disease is Alzheimer's disease.

### Systems

### Computer Systems

Disclosed herein are computer systems for detecting a disease state in a subject, comprising: a non-transitory memory; and a processor in communication with the non-transitory memory, the processor configured to execute the following operations in order to effectuate a method comprising the operations of: assaying cell-free messenger RNA (cf-mRNA) in a biological sample to determine a level of the cf-mRNA that contains a non-contiguous junction relative to genomic DNA, thereby detecting one or more splice junctions; processing the detected one or more splice junctions; and detecting the disease state of the subject based at least in part on the processing.

Further disclosed herein are non-transitory computer-readable memories storing one or more instructions executable by one or more processors, that when executed by the one or more processors cause the one or more processors to perform processing comprising: assaying cell-free messenger RNA (cf-mRNA) in a biological sample to determine a level of the cf-mRNA that contains a non-contiguous junction relative to genomic DNA, thereby detecting one or more splice junctions; processing the detected one or more splice junctions; and detecting the disease state of the subject based at least in part on the processing.

Further disclosed herein are computer systems for determining a risk of a disease state in a subject, the system comprising: a non-transitory memory; and a processor in communication with the non-transitory memory, the processor configured to execute the following operations in order to effectuate a method comprising the operations of: assaying cell-free messenger RNA (cf-mRNA) in a biological sample to detect one or more splice junctions in the cf-mRNA, wherein the one or more splice junctions correspond to one or more genes.

Further disclosed herein are non-transitory computer-readable memories storing one or more instructions executable by one or more processors, that when executed by the one or more processors cause the one or more processors to perform processing comprising: assaying cell-free messenger RNA (cf-mRNA) in a biological sample to detect one or more splice junctions in the cf-mRNA, wherein the one or more splice junctions correspond to one or more genes.

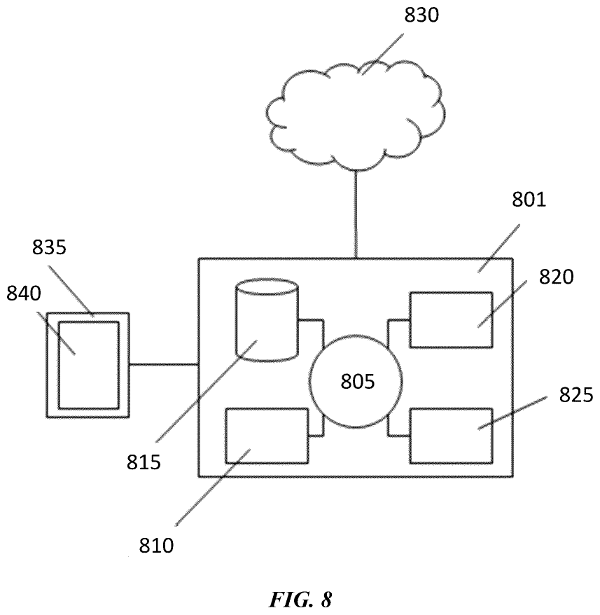

The present disclosure provides computer control systems that are programmed to implement methods of the disclosure. FIG. 8 shows a computer system 801. The computer system 801 may be programmed to detect or determine a risk of a disease state in a subject. The computer system 801 can regulate various aspects of the present disclosure, such as, for example, obtaining a biological sample from the subject; assaying cell-free messenger RNA (cf-mRNA) in the biological sample to determine a level of the cf-mRNA that contains a non-contiguous junction relative to genomic DNA, thereby detecting one or more splice junctions in the cf-mRNA; computer processing the detected one or more splice junctions; and detecting the disease state of the subject based at least in part on the computer processing. As another example, the computer system 801 can regulate additional aspects of the disclosure, such as, for example, obtaining a biological sample from the subject; assaying cell-free messenger RNA (cf-mRNA) in the biological sample to detect one or more splice junctions in the cf-mRNA, wherein the one or more splice junctions correspond to one or more genes. In some embodiments, the biological sample comprises a blood sample, a plasma sample, a serum sample, a urine sample, or a saliva sample. In some embodiments, the biological sample comprises the plasma sample. As yet another example, the computer system 801 can regulate additional aspects of the disclosure, such as for example, assaying a first expression profile of a first cell-free biological sample obtained or derived from a subject at a first time point, to detect a first set of splice junctions; administering the compound to the subject; assaying a second expression profile of a second cell-free biological sample obtained or derived from the subject at a second time point subsequent to the administering, to detect a second set of splice junctions; computer processing the detected first and second sets of splice junctions; and assessing the effect of the compound based at least in part on the computer processing. The computer system 801 can be an electronic device of a user or a computer system that is remotely located with respect to the electronic device. The electronic device can be a mobile electronic device.

The computer system 801 may include a central processing unit (CPU, also “processor” and “computer processor” herein) 805, which can be a single core or multi core processor, or a plurality of processors for parallel processing. The computer system 801 may also include memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 may be in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases may be a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries, and saved programs. The storage unit 815 can store user data, e.g., user preferences, user programs, or the like. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., a medical worker that is inquiring a risk score). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as disclosed herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, a report based on the risk score containing information direct to monitoring and/or treating AD progression. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, be used to generate the classifier to calculate a risk score of having AD or cognitive impairment.

### Machine Learning Models

Machine learning models provide benefit to many applications due to their ability to perform calculations and give outputs. A machine learning model may be, for example, a support vector machine, a logistic regression, a random forest, a clustering algorithm, a naïve bayes, a hidden Markov model, a reinforcement learning model, a q-star (Q*) model, a neural network (NN), a convolutional neural network (CNN), a deep neural network (DNN), a multilayer perceptron, an ensemble method, an unsupervised method, a supervised method, a multi-input method, a multi-output method, a regularization (e.g., Elastic Net Regularization) or the like.

A machine learning model may include many methods working in parallel or in tandem. A machine learning model may perform classification, regression, clustering, dimensionality reduction, or the like. A machine learning method may generally take an input and perform a series of predefined steps on data to transform it into an output. A machine learning model may have two phases, such as for example, training and inference. The machine learning model may have parameters and hyper parameters. A parameter may be tuned during a training phase. Non-limiting examples of parameters may include, for example, coefficients of a logistic regression or the weights of a neural network. The training phase may include a method to evaluate learned information. When a machine learning model is trained, the machine learning model may infer.

Non-limiting examples of machine learning models include scale-invariant feature transform (SIFT), speeded up robust features (SURF), oriented FAST and rotated BRIEF (ORB), binary robust invariant scalable keypoints (BRISK), fast retina keypoint (FREAK), Viola-Jones algorithm, Eigenfaces approach, Lucas-Kanade algorithm, Hom-Schunk algorithm, Mean-shift algorithm, visual simultaneous location and mapping (vSLAM) techniques, a sequential Bayesian estimator (e.g., Kalman filter, extended Kalman filter, or the like), bundle adjustment, adaptive thresholding, Iterative Closest Point (ICP), Semi Global Matching (SGM), Semi Global Block Matching (SGBM), Feature Point Histograms, and various other machine learning algorithms (e.g., support vector machine, k-nearest neighbors algorithm, Naive Bayes, neural network (including deep neural networks), or other supervised/unsupervised machine learning models).

Additional non-limiting examples of machine learning models can include supervised, semi-supervised, or non-supervised machine learning models, including regression models (e.g., Ordinary Least Squares Regression), instance-based models (e.g., Learning Vector Quantization), decision tree models (e.g., classification and regression trees), Bayesian models (e.g., Naive Bayes), clustering models (e.g., k-means clustering), association rule learning models (e.g., a-priori models), artificial neural network models (e.g., Perceptron), deep learning models (e.g., Deep Boltzmann Machine, deep neural network, or the like), dimensionality reduction models (e.g., Principal Component Analysis), ensemble models (e.g., Stacked Generalization or decision trees), or various other machine learning models.

A neural network machine learning model may have multiple layers one or more of which may be input and one or more of which may be output. Layers of the machine learning model may use different activation functions. Layers of the machine learning model may also have different methods of input and processing such as those associated with fully connected layers, recurrent layers, long short-term memory (LSTM) layers, bidirection recurrent layers, bidirectional LSTM layers, convolutional layers, pooling layers, attention layers and transformer layers, or the like.

### Classifiers

The present disclosure provides classifiers for processing or analyzing data generated from a biological sample to yield an output. Such an output may result in an assessment of the splice junctions from the cf-mRNA of a subject for detecting a disease state or determining a risk of a disease state in the subject.

A classifier may be a machine learning algorithm. The machine learning algorithm may be a trained machine learning algorithm. The machine learning algorithm may be trained via supervised or unsupervised learning, for example. For example, the machine learning algorithm may comprise generative modeling (e.g., a statistical model of a joint probability distribution on an observable variable X on a target variable Y; such as a naïve Bayes classifier and linear discriminant analysis), discriminative modeling (e.g., a model of a conditional probability of a target variable Y, given an observation x of an observable variable X; such as a logistic regression, a perceptron, or a support vector machine), or reinforcement learning (RL).

As used herein, the terms “machine learning,” “machine learning model,” “machine learning procedure,” “machine learning operation,” and “machine learning algorithm” generally refer to any system or analytical and/or statistical procedure that may progressively (e.g., iteratively) improve computer performance of a task. Machine learning (ML) may comprise one or more supervised, semi-supervised, or unsupervised machine learning techniques. For example, a ML algorithm may be a trained algorithm that may be trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. Non-limiting examples of ML include: k-means, k-means clustering, k-nearest neighbors, learning vector quantization, linear regression, non-linear regression, least squares regression, partial least squares regression, logistic regression, stepwise regression, multivariate adaptive regression splines, ridge regression, principle component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, generative adversarial networks, and the like.

As used herein, the terms “reinforcement learning,” “reinforcement learning procedure,” “reinforcement learning operation,” and “reinforcement learning algorithm” generally refer to any system or computational procedure that may take one or more actions to enhance or maximize some notion of a cumulative reward to its interaction with an environment. The agent performing the reinforcement learning (RL) procedure may receive positive or negative reinforcements, called an “instantaneous reward,” from taking one or more actions in the environment and therefore placing itself and the environment in various new states.

A goal of the agent may be to enhance or maximize some notion of cumulative reward. For instance, the goal of the agent may be to enhance or maximize a “discounted reward function” or an “average reward function.” A “Q-function” may represent the maximum cumulative reward obtainable from a state and an action taken at that state. A “value function” and a “generalized advantage estimator” may represent the maximum cumulative reward obtainable from a state given an optimal or best choice of actions. RL may utilize any one of more of such notions of cumulative reward. As used herein, any such function may be referred to as a “cumulative reward function.” Therefore, computing a best or optimal cumulative reward function may be equivalent to finding a best or optimal policy for the agent.

The agent and its interaction with the environment may be formulated as one or more Markov Decision Processes (MDPs), for example. The RL procedure may not assume knowledge of an exact mathematical model of the MDPs. The MDPs may be completely unknown, partially known, or completely known to the agent. The RL procedure may sit in a spectrum between the two extents of “model-based” or “model-free” with respect to prior knowledge of the MDPs. As such, the RL procedure may target large MDPs where exact methods may be infeasible or unavailable due to an unknown or stochastic nature of the MDPs.

The RL procedure may be implemented using one or more computer processors disclosed herein. The digital processing unit may utilize an agent that trains, stores, and later on deploys a “policy” to enhance or maximize the cumulative reward. The policy may be sought (for instance, searched for) for a period of time that may be as long as possible or desired. Such an optimization problem may be solved by storing an approximation of an optimal policy, by storing an approximation of the cumulative reward function, or both. In some cases, RL procedures may store one or more tables of approximate values for such functions. In other cases, RL procedure may utilize one or more “function approximators.”

Non-limiting examples of function approximators include neural networks (e.g., deep neural networks) and probabilistic graphical models (e.g., Boltzmann machines, Helmholtz machines, Hopfield networks, or the like). A function approximator may create a parameterization of an approximation of the cumulative reward function. Optimization of the function approximator with respect to its parameterization may consist of perturbing the parameters in a direction that enhances or maximizes the cumulative rewards and therefore enhances or optimizes the policy (such as in a policy gradient method), or by perturbing the function approximator to get closer to satisfy Bellman's optimality criteria (such as in a temporal difference method).

During training, the agent may take actions in the environment to obtain more information about the environment and about good or best choices of policies for survival or better utility. The actions of the agent may be randomly generated (for instance, especially in early stages of training) or may be prescribed by another machine learning paradigm (such as supervised learning, imitation learning, or any other machine learning procedure disclosed herein). The actions of the agent may be refined by selecting actions closer to the agent's perception of what an enhanced or optimal policy is. Various training strategies may sit in a spectrum between the two extents of off-policy and on-policy methods with respect to choices between exploration and exploitation.

The trained algorithm may be configured to accept a plurality of input variables and to produce one or more output values based on the plurality of input variables. The plurality of input variables may comprise a presence or abundance of a splice junction or a cf-mRNA transcript corresponding to one or more genes. The plurality of input variables may also include clinical health data of a subject. The one or more output values may comprise a state or condition of a subject. For example, the state or condition of the subject may include whether the subject has a disease state (e.g., Alzheimer's disease) or a risk that the subject has the disease state (e.g., Alzheimer's disease).

The trained algorithm may comprise a classifier, such that each of the one or more output values comprises one of a fixed number of possible values (e.g., a linear classifier, a logistic regression classifier, or the like) indicating a classification of a state or condition of the subject by the classifier. The trained algorithm may comprise a binary classifier, such that each of the one or more output values comprises one of two values (e.g., {0, 1}, {positive, negative}, {present, absent}, or {high-risk, low-risk}) indicating a classification of the state of the subject (e.g., disease state). The trained algorithm may be another type of classifier, such that each of the one or more output values comprises one of more than two values (e.g., {0, 1, 2}, {positive, negative, indeterminate}, {present, absent, or indeterminate}, or {high-risk, intermediate-risk, low-risk}) indicating a classification of the state of the subject (e.g., disease state).

The output values may comprise descriptive labels, numerical values, or a combination thereof. Some of the output values may comprise descriptive labels. Such descriptive labels may provide an identification or indication of a state of the subject, and may comprise, for example, positive, negative, present, absent, high-risk, intermediate-risk, low-risk, or indeterminate. Such descriptive labels may provide an identification of a treatment for the state of the subject, and may comprise, for example, a therapeutic intervention, a duration of the therapeutic intervention, and/or a dosage of the therapeutic intervention suitable to treat the state or condition of the subject. Such descriptive labels may provide an identification of secondary clinical tests that may be appropriate to perform on the subject, and may comprise, for example, a blood test, a genetic test, or a medical imaging. As another example, such descriptive labels may provide a prognosis of the state of the subject. As another example, such descriptive labels may provide a relative assessment of the state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, {present, absent}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the state or condition of the subject. Some numerical values may be mapped to descriptive labels, for example, by mapping 1 to “positive” or “present,” and 0 to “negative” or “absent.”

Some of the output values may be assigned based on one or more cutoff values. For example, a binary classification of subjects may assign an output value of “positive,” “present,” or 1 if the subject has at least a 50% probability of having the state or condition. For example, a binary classification of subjects may assign an output value of “negative,” “absent,” or 0 if the subject has less than a 50% probability of having the state or condition. In this case, a single cutoff value of 50% is used to classify subjects into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of subjects may assign an output value of “positive,” “present, or 1 if the subject has a probability of having the state of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of subjects may assign an output value of “positive” or 1 if the subject has a probability of having the state or condition of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of subjects may assign an output value of “negative,” absent, or 0 if the subject has a probability of having the state of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of subjects may assign an output value of “negative” or 0 if the subject has a probability of the state or condition of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of subjects may assign an output value of “indeterminate” or 2 if the subject is not classified as “positive,” “negative,” “present,” “absent,” 1, or 0. In this case, a set of two cutoff values is used to classify subjects into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify subjects into one of n+1 possible output values, where n is any positive integer.

The trained algorithm may be trained with a plurality of independent training samples. Each of the independent training samples may comprise a dataset of input variables (e.g., a presence or abundance of at least one splice junction in a cf-mRNA corresponding to a gene that is organ/tissue specific collected from a subject at a given time point, and one or more known output values (e.g., a state) corresponding to the subject. Independent training samples may comprise datasets of input variables and associated output values obtained or derived from a plurality of different subjects. Independent training samples may comprise datasets of input variables and associated output values obtained at a plurality of different time points from the same subject (e.g., on a regular basis such as weekly, biweekly, or monthly). Independent training samples may be associated with presence of the state or condition (e.g., training samples comprising datasets of input variables and associated output values obtained or derived from a plurality of subjects known to have the state or condition). Independent training samples may be associated with absence of the state or condition (e.g., training samples comprising datasets of input variables and associated output values obtained or derived from a plurality of subjects who are known to not have a previous diagnosis of the state or condition or who have received a negative test result for the state or condition). A plurality of different trained algorithms may be trained, such that each of the plurality of trained algorithms is trained using a different set of independent training samples (e.g., sets of independent training samples corresponding to presence or absence of different states).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise datasets of input variables associated with presence of the state or condition and/or datasets of input variables associated with absence of the state or condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the state or condition. In some embodiments, the dataset of input variables is independent of samples used to train the trained algorithm.

The trained algorithm may be trained with a first number of independent training samples associated with presence of the state and a second number of independent training samples associated with absence of the state. The first number of independent training samples associated with presence of the state may be no more than the second number of independent training samples associated with absence of the state. The first number of independent training samples associated with presence of the state may be equal to the second number of independent training samples associated with absence of the state. The first number of independent training samples associated with presence of the state may be greater than the second number of independent training samples associated with absence of the state.

A machine learning algorithm may be trained with a training set of samples from subjects with identified or diagnosed disease states, such as subject with Alzheimer's disease. The machine learning algorithm may be trained with at least about 5, 10, 20, 30, 40, 50, 100, 200, 300, 400, 500, 1000, or more samples. Once trained, the machine learning algorithm may be used to process data generated from one or more samples independent of samples from the training set to identify one or more features in the one or more samples (e.g., one or more splice junctions in cf-mRNA corresponding to a gene) at an accuracy of at least about 60%, 70%, 80%, 85%, 90%, 95%, or more. The machine learning algorithm may be used to process the data to identify the one or more features at a sensitivity of at least about 60%, 70%, 80%, 85%, 90%, 95%, or more. The machine learning algorithm may be used to process the data to identify the one or more features at a specificity of at least about 60%, 70%, 80%, 85%, 90%, 95%, or more.

The trained algorithm may be configured to identify the state or condition as disclosed herein at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the state or condition by the trained algorithm may be calculated as the percentage of independent test samples (e.g., subjects known to have the state or subjects with negative clinical test results for the state) that are correctly identified or classified as having or not having the state.

The trained algorithm may be configured to identify the state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the state or condition using the trained algorithm may be calculated as the percentage of datasets of input variables identified or classified as having the state that correspond to subjects that truly have the state (e.g., disease state of Alzheimer's disease).

The trained algorithm may be configured to identify the state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the state or condition using the trained algorithm may be calculated as the percentage of datasets of input variables identified or classified as not having the state that correspond to subjects that truly do not have the state.

The trained algorithm may be configured to identify the state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the state using the trained algorithm may be calculated as the percentage of independent test samples associated with presence of the state (e.g., subjects known to have the state (e.g., disease state)) that are correctly identified or classified as having the state.

The trained algorithm may be configured to identify the state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the state using the trained algorithm may be calculated as the percentage of independent test samples associated with absence of the state (e.g., subjects with negative clinical test results for the state) that are correctly identified or classified as not having the state.

The trained algorithm may be configured to identify the state with an Area Under Curve (AUC) value of the Receiver Operating Characteristic (ROC) curve of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC value may be calculated as an integral of the Receiver Operating Characteristic (ROC) curve (e.g., the area under the ROC curve) associated with the trained algorithm in classifying datasets of input variables as having or not having the state.

The trained algorithm may be adjusted or tuned to improve one or more of the performance, accuracy, PPV, NPV, clinical sensitivity, clinical specificity, or AUC of identifying the state. The trained algorithm may be adjusted or tuned by adjusting parameters of the trained algorithm (e.g., a set of cutoff values used to classify a dataset of input variables as disclosed elsewhere herein, or parameters or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of features (e.g., of the input variables) may be identified as most influential or most important to be included for making high-quality classifications or identifications of the state. The plurality of features or a subset thereof may be ranked based on classification metrics indicative of each feature's influence or importance toward making high-quality classifications or identifications of the state. Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUROC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Compositions

Provided herein are compositions of any one of the methods disclosed herein.

In some cases, the composition comprises a treatment for a disease state. In some cases, the composition comprises a treatment for Alzheimer's disease. In some cases, the composition is used to alleviate symptoms such as memory loss, misplacement of items, difficulty in decision making, reduced ability to understand visual images, confusion with time, mood swings, repetitive speech, difficulty in problem solving, social withdrawal, sleep problems, or the like. In some cases, the composition comprises a cholinesterase inhibitor. In some cases, the composition comprises a N-methyl-D-aspartate (NMDA) antagonist. In some cases, the composition is a tablet or a capsule.

### Kits

Provided herein are kits comprising any of the compositions disclosed herein and instructions for use of the composition according to any of the methods disclosed herein.

In some cases, a kit disclosed herein comprises one or more compositions and/or reagents for detecting a disease state in a subject or determining a risk of a disease state in a subject. A kit as disclosed herein can further comprise instructions for practicing any of the methods disclosed herein. The kits disclosed herein can further comprise reagents to enable detection of cf-mRNAs by various assay types such as by reverse transcription, polynucleotide amplification, sequencing, probe hybridization, microarray hybridization, or the like. The kids disclosed herein can further comprise a computer readable medium comprising computer executable code for implementing a method disclosed herein.

In some cases, the kits disclosed herein comprises oligonucleotide primers that may hybridize to cDNA sequences transcribed from cf-mRNAs corresponding to one or more genes disclosed herein.

In some cases, the kits disclosed herein may include a packaging material. As used herein, the term “packaging material” can refer to a physical structure housing the components of the kit. The packaging material can maintain sterility of the kit components, and can be made of material commonly used for such purposes (e.g., paper, corrugated fiber, glass, plastic, foil, ampules, or the like). The kits disclosed herein can further comprise a buffering agent, a preservative, or a protein/nucleic acid stabilizing agent. The kids disclosed herein can further comprise components for obtaining a biological sample from a subject. Non-limiting examples of such components include gloves, hypodermic needles or syringes, tubing, tubes or vessels to hold a sample (e.g., the biological sample), sterilization components (e.g., isopropyl alcohol wipes, sterile gauze, or the like), and/or cooling material (e.g., freezer pack, dry ice, ice, or the like). In some cases, the kits disclosed herein are used in accordance of any of the disclosed methods

The kits disclosed herein may be used to detect a disease state in a subject. Additionally or alternatively, the kits disclosed herein may be used to determine a risk of a disease state in a subject. Additionally or alternatively, the kids disclosed herein may be used to assess an effect of a compound.

The kits disclosed herein may comprise at least one reagent. The kits disclosed herein may comprise greater than or equal to one reagent, two reagents, three reagents, five reagents, 10 reagents, 15 reagents, 20 reagents, 25 reagents, or 50 reagents.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in the context of a number refers to a range spanning from 10% greater than the number to 10% less than the number.

As used herein, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone; B alone; C alone; A and B together; A and C together; B and C together; or A, B, and C together.

The terms “determining,” “measuring,” “evaluating,” “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

The terms “panel,” “biomarker panel,” “protein panel,” “classifier model,” and “model” are used interchangeably herein to refer to a set of biomarkers, wherein the set of biomarkers comprises at least two biomarkers. Exemplary biomarkers are cf-mRNAs mapped to a list of differentially expressed genes disclosed herein. However, additional biomarkers are also contemplated, for example, age or gender of the individual providing a sample. The biomarker panel is often predictive and/or informative of a subject's health status, disease, or condition.

The “level” of a biomarker panel refers to the absolute and relative levels of the panel's constituent markers and the relative pattern of the panel's constituent biomarkers.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be cognitive impairment. The cognitive impairment can be a symptom for AD. In some cases, the subject is not necessarily diagnosed or suspected of being at high risk for the disease.

The term sensitivity, or true positive rate, can refer to a test's ability to identify a condition correctly. For example, in a diagnostic test, the sensitivity of a test is the proportion of patients known to have the disease, who will test positive for it. In some cases, this is calculated by determining the proportion of true positives (i.e., patients who test positive who have the disease) to the total number of individuals in the population with the condition (i.e., the sum of patients who test positive and have the condition and patients who test negative and have the condition).

The quantitative relationship between sensitivity and specificity can change as different diagnostic cut-offs are chosen. This variation can be represented using ROC curves. The x-axis of a ROC curve shows the false-positive rate of an assay, which can be calculated as (1−specificity). The y-axis of a ROC curve reports the sensitivity for an assay. This allows one to easily determine a sensitivity of an assay for a given specificity, and vice versa.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “machine learning,” “machine learning procedure,” “machine learning operation,” “machine learning model,” and “machine learning algorithm” generally refer to any system or analytical and/or statistical procedure that may progressively improve computer performance of a task. Machine learning may include a machine learning algorithm. The machine learning algorithm may be a trained algorithm. Machine learning (ML) may comprise one or more supervised, semi-supervised, or unsupervised machine learning techniques. For example, an ML algorithm may be a trained algorithm that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principle component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

### EXAMPLES

### Example 1: Alzheimer's Disease

Protein coding cf-mRNA was identified from 50 subjects with Alzheimer's disease and 23 non-cognitively impaired subjects. Splicing junctions were identified from cf-mRNA using the software, MAJIQ (v2.2).

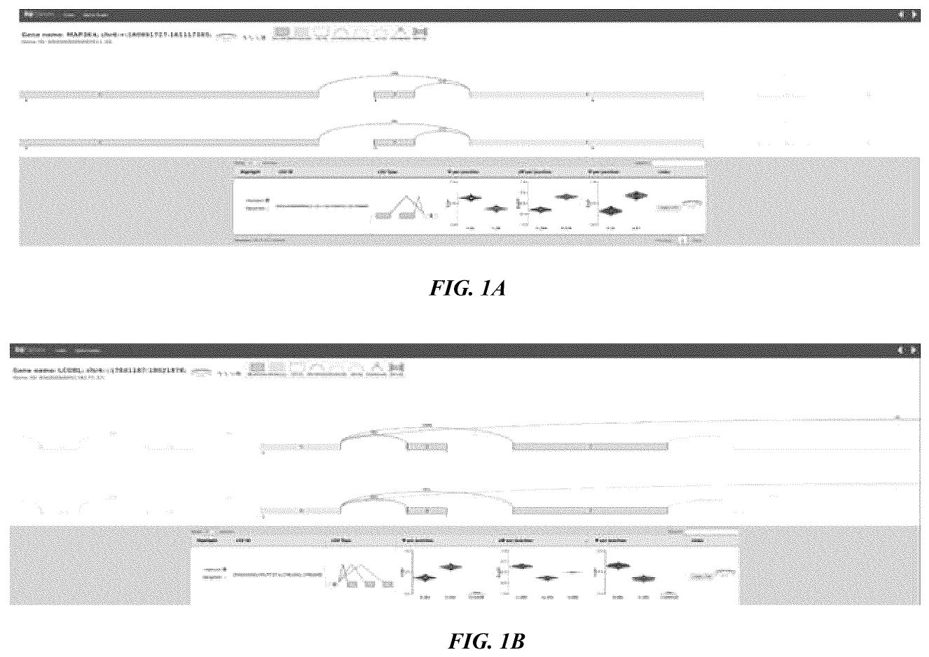

FIG. 1A, MAP3K4—This MAJIQ figure demonstrates exon skipping, with double the percent spliced in (PSI) expression in Alzheimer's disease of exon 1-3 (RED) in comparison to Non-cognitively impaired, at a Delta PSI (dPSI) of ˜30 percent, with a confidence level of 100 percent changing. In the example of FIG. 1B, LCORL—This MAJIQ figure demonstrates exon skipping and an inverse PSI expression from the source exon 5, with up to 3 target exons, and only 2 target exons being expressed. Double the percent spliced in (PSI) expression from exons 5-6 (RED) was observed for non-cognitively impaired as compared to Alzheimer's disease. In exons 5-7 (BLUE) double the PSI expression was observed in Alzheimer's disease compared to non-cognitively impaired, which demonstrated an inverse expression at a dPSI of ˜30 percent.

Disclosed herein is an example workflow of the splice junction feature detection pipeline for cf-mRNA isolated from human clinical samples.

FastQC (v0.11.9) provided a summary quality assessment of Forward and Reverse sequencing of cf-mRNA reads, pre-alignment.

HISAT2 (v2.2.1) was utilized with custom configuration settings for cf-mRNA sequence alignment and mapping to the human genome.

PICARD MergeSamFiles (v2.6.1) merged the cf-mRNA sequencing data files (e.g., sequencing lanes or alignment files from technical replicates). Samtools view (v1.15) retained cf-mRNA sequencing reads with a score of MAPQ60.

MAPQ60 was the HISAT2 scoring scheme for uniquely mapped reads. To mark cf-mRNA sequencing reads which are possible PCR duplicates, Picard MarkDuplicates (v2.6.1) was performed.

Post-sequencing quality alignment metrics and read coverage of individual genes was determined using the Quality of RNA-Seq Tool-Set (v1.3) and CoverageView. Bedtools2 collapsed and counted cf-mRNA sequencing alignments, which passed quality control.

A splice junction catalogue was designed, customized and created in RStudio (2022.02.1+461). This catalogue was populated using Regtools Extract and Annotate (v0.0.1) for cf-mRNA exon-exon splice junction identification, extraction & annotation alongside MAJIQ (v2.2) for detecting, quantifying, and visualizing local splicing variations from cf-mRNA sequencing reads.

This pipeline provides information on the absence or presence of a splice junction and its gene across study subjects and conditions in cf-mRNA, and builds a library of junction counts and aggregate confidence across samples exhibiting lowly expressed splice junctions. Settings can be customized according to the pipeline established, and inherent parameter differences include maximum intron length, number of allowed mismatches, exon reads versus junction reads, tissue specific splicing events, and the like.

One example of a software tool that performs splicing based analysis to qualify and quantify the disclosed pipeline is MAJIQ.

### Example 2: Bioinformatics Workflow

A bioinformatics workflow used in the methods disclosed herein is provided.

### Quality Control and Trimming

FastQC (v0.11.9) provided a summary quality assessment of Forward and Reverse sequencing of cell free mRNA reads pre-alignment.

TrimmomaticPE (v0.39) was used to remove adaptor and poly-G bases.

Read sequences greater than 74 base pairs were retained.

### Two Pass Alignment and Filtering

A HISAT2 (v2.2.1) two-pass alignment approach was used.

In pass 1, paired end alignment was used.

SAMTOOLS view (v1.15 & v1.19) was used to filter MAPQ0 unmapped reads.

SAMTOOLS fastq was used to convert read sequences to FASTQ Forward and Reverse read files.

FASTP (v0.23.4) was used to trim 12 base pairs off both read tails. A 4 base pair read tail sliding window was set with a minimum quality score of 30. Read sequences greater than 74 base pairs were retained.

In pass 2, paired end and singleton read alignment were used.

SAMTOOLS view was used to filter MAPQ60 unique alignments and MAPQ1 primary multi-mapping alignments (highest score). Orphan reads were filtered and logged from all BAM files.

SAMTOOLS addreplacerg tags were appended to each read (e.g., passes 1 and 2).

### UMI Deduplication

UMI-Tools (v1.1.4) were used to deduplicate singleton and paired BAM files.

### Clipping

QoRTs (v1.3) was used to quantify overlapping paired-end reads in deduplicated BAM files.

BamUtil:clipOverlap (v1.0.14) was used to remove completely overlapping paired-end reads and soft-clips partial overlapping read pairs to obviate duplicate counting of splice junction features.

### Metrics

QoRTs was used to produce metrics on deduplicated and clipped BAM files.

R (2022-11-10 r83330), R-Studio (2022.07.2+576), MultiQC (v1.12) and RSeQC (v5.0.1) functions were used to analyze metrics.

Files of low quality, inadequate annotated splices, and low total known splice events were filtered.

### Differential Splicing Analysis

Modelling Alternative Junction Inclusion Quantification (MAJIQ, v2.5) was used to identify differentially spliced cf-mRNA transcripts.

MAJIQ Build used cohort BAM files and a transcriptome annotation file to define splice graphs and known Local Splicing Variations (LSVs), with settings of group 1 (non-cognitively impaired subjects), group 2 (Alzheimer's disease subjects), minimum experiments 0.1, minimum reads 3, and minimum positions 1.

Files were split into quartiles based on total known splice events.

MAJIQ Heterogen was used to quantify Percentage Spliced In (PSI) of LSVs between groups, with settings of minimum experiments 0.5, minimum reads 15, and minimum positions 1.

Linux command suite was used to identify and parse the highest statistically significant LSVs into the junction coordinate ID.

### Classifier Development

R-Studio imported PSI values of junction coordinate IDs, PSI distributions, and correlation were determined across the cohort.

MissForest (v1.5) was used to impute missing junction coordinate ID values using a random forest non-parametric approach.

PSI distributions following NA imputation were determined and compared.

Bayesian Model Averaging (v3.18.17) and glmulti (v1.0.8) identified junction coordinate ID variables of importance.

Glmnet (v4.1-8) implemented junction coordinate values of interest into a classifier model using Elastic Net regularization, iterated 20× for optimal settings of alpha. A 3-fold cross validation nested within a 5-fold test and train shuffle, stratified by outcome, was used to tune for the optimal lambda and final coefficient averages, respectively.

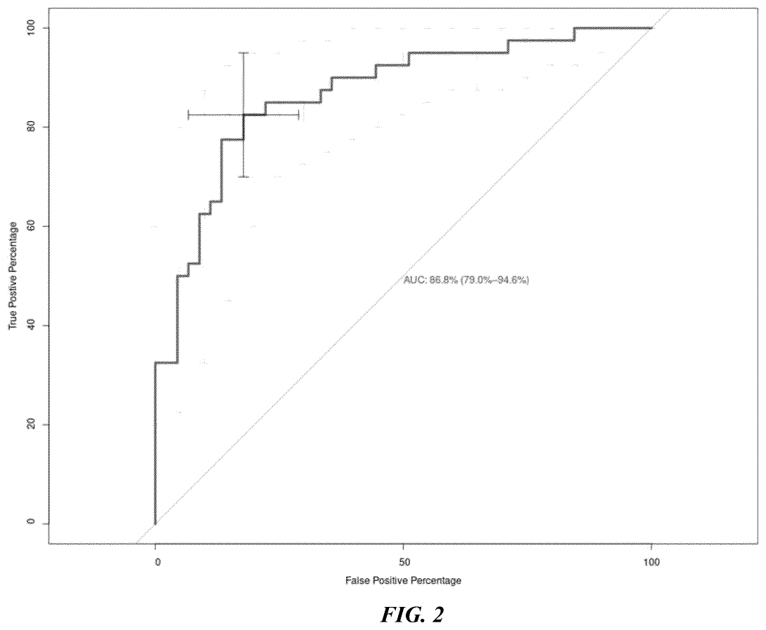

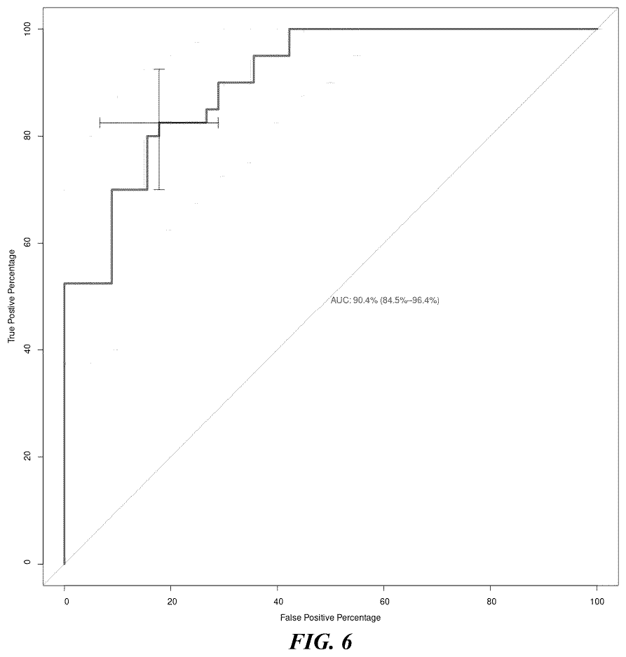

pROC (v.1.18.4) was used to generate ROC curves (see, for example, FIG. 2, FIG. 6, and FIG. 6).

Caret (v6.0-94) was used to generate confusion matrices.

### Example 3: Splicing Identification

Biological samples comprising cf-mRNA were obtained from subjects with Alzheimer's disease and non-cognitively impaired subjects. Splice junctions were identified in the cf-mRNA using methods disclosed herein.

Table 1 provides a list of 278 junctions (e.g., intron junctions) that were identified to be differentially present in Alzheimer's disease subjects as compared to non-cognitively impaired subjects. Each of the 278 junctions provided in Table 1 includes the gene name and LSV ID (LSV identifier).

TABLE 1
240 Differentially Present Splice Junctions in Alzheimer's
Disease subjects vs. non-cognitively impaired subjects.

LSV ID (Gene; LSV ID)
ABLIM1; ENSG00000099204.21:t:114491791-114491878; 114491878-114545005
ACAA1; ENSG00000060971.19:s:38126493-38126700; 38126341-38126510
ACAP1; ENSG00000072818.12:t:7341948-7342067; 7336787-7341948
ACOT8; ENSG00000101473.17:t:45848450-45849110; 45848675-45857188
ACP1; ENSG00000143727.16:t:272192-273155; 272065-272192
ADD3; ENSG00000148700.15:t:110100625-110100848; 110008299-110100625
ADGRE5; ENSG00000123146.20:t:14397414-14397804; 14391079-14397658
ADK; ENSG00000156110.15:t:74200764-74200838; 74151343-74200764
AGAP3; ENSG00000133612.19:s:151119987-151120145; 151120145-151122725
AKAP13; ENSG00000170776.22:t:85575131-85575329; 85543955-85575131
ALAD; ENSG00000148218.16:s:113393161-113393634; 113392169-113393447
AMPD2; ENSG00000116337.20:t:109625303-109625433; 109621266-109625303
ANAPC11; ENSG00000141552.18:t:81894298-81894624; 81891833-81894467
AP1B1; ENSG00000100280.17:s:29330378-29330532; 29329720-29330378
AP1B1; ENSG00000100280.17:t:29327680-29328895; 29328895-29329712
APP; ENSG00000142192.21:s:26000015-26000182; 25982477-26000015
APP; ENSG00000142192.21:t:25897573-25897983; 25897673-25911741
ARAP1; ENSG00000186635.15:s:72693325-72693470; 72688537-72693325
ARFRP1; ENSG00000101246.20:t:63706999-63707658; 63707097-63707867
ARHGAP17; ENSG00000140750.17:t:24935470-24936827; 24935639-24941987
ARHGAP17; ENSG00000288353.1:t:188953-190310; 189122-195470
ARHGEF10; ENSG00000274726.4:s:43270-43560; 43560-47749
ARHGEF1; ENSG00000076928.19:s:41896377-41896878; 41896482-41897315
ARHGEF7; ENSG00000102606.20:t:111217679-111217885; 111210002-111217679
ARMC10; ENSG00000170632.14:s:103074881-103075411; 103075411-103075777
ARRDC2; ENSG00000105643.11:t:18008711-18008861; 18001573-18008711
ATE1; ENSG00000107669.19:t:121924266-121924329; 121924329-121928347
ATP6V1D; ENSG00000100554.12:t:67350611-67350690; 67350690-67359658
ATXN2L; ENSG00000168488.19:s:28835933-28836176; 28836122-28836748
ATXN2L; ENSG00000168488.19:t:28836728-28837237; 28836122-28836748
ATXN2L; ENSG00000168488.19:t:28836728-28837237; 28836485-28836728
AURKB; ENSG00000178999.13:t:8207738-8208225; 8207840-8210530
BLOC1S6; ENSG00000104164.12:s:45592135-45592276; 45592276-45605428
C12orf76; ENSG00000174456.16:s:110048363-110048870; 110042459-110048363
CAMTA1; ENSG00000171735.20:t:6825092-6825210; 6820250-6825092
CBFA2T3; ENSG00000129993.15:t:88892244-88892485; 88892485-88898078
CBFB; ENSG00000067955.15:t:67066682-67066962; 67036755-67066682
CCDC28B; ENSG00000160050.16:t:32204598-32204620; 32204379-32204598
CCDC92; ENSG00000119242.9:s:123972529-123972831; 123943493-123972529
CCDC92; ENSG00000119242.9:t:123943347-123943495; 123943493-123972529
CD300H; ENSG00000284690.3:s:74563919-74564275; 74560824-74563919
CD34; ENSG00000174059.17:s:207888682-207888846; 207887923-207888682
CDAN1; ENSG00000140326.13:s:42736989-42737128; 42736780-42737013
CDC14B; ENSG00000081377.17:t:96565393-96565512; 96565483-96619219
CDK5RAP2; ENSG00000136861.19:t:120402806-120403071; 120403071-120403316
CELF2; ENSG00000048740.19:t:11249153-11249201; 11165682-11249153
CEP164; ENSG00000110274.16:t:117409618-117410701; 117409028-117409618
CLEC1B; ENSG00000165682.15:s:9995140-9995246; 9986158-9995140
COX20; ENSG00000203667.10:s:244835616-244835756; 244835756-244842195
CPNE1; ENSG00000214078.13:s:35627280-35627531; 35626800-35627280
CYTH2; ENSG00000105443.17:s:48474838-48476276; 48474949-48477717
CYTH2; ENSG00000105443.17:t:48478066-48478145; 48477719-48478069
DAZAP1; ENSG00000071626.17:s:1422348-1422396; 1422396-1425878
DCTD; ENSG00000129187.15:s:182917288-182917477; 182915575-182917311
DCTD; ENSG00000129187.15:t:182915461-182915575; 182915575-182917288
DECR1; ENSG00000104325.7:s:90001405-90001561; 90001561-90017124
DECR1; ENSG00000104325.7:s:90001405-90001561; 90001561-90018909
DECR1; ENSG00000104325.7:t:90018564-90018966; 90001561-90018909
DEF8; ENSG00000140995.17:t:89954172-89954376; 89949513-89954243
DGKD; ENSG00000077044.11:s:233390403-233390483; 233390483-233392070
DGKZ; ENSG00000149091.15:t:46367291-46367399; 46345585-46367291
DNM2; ENSG00000079805.19:s:10795372-10795439; 10795439-10797380
DOCK8; ENSG00000107099.18:t:271627-271729; 215029-271627
DUT; ENSG00000128951.14:t:48332103-48332737; 48331479-48332268
DYRK1A; ENSG00000157540.22:s:37418905-37420384; 37420384-37472684
DYSF; ENSG00000135636.16:t:71480883-71480938; 71454086-71480883
EIF4G1; ENSG00000114867.22:t:184317321-184317497; 184314674-184317321
EIF4G3; ENSG00000075151.24:t:20981048-20981227; 20981227-20997601
EMC8; ENSG00000131148.9:s:85781211-85781760; 85779867-85781211
EPSTI1; ENSG00000133106.15:s:42991978-42992271; 42969177-42991978
EXOSC1; ENSG00000171311.13:s:97438663-97438703; 97437750-97438663
EXOSC1; ENSG00000171311.13:t:97437700-97437750; 97437750-97438663
F2RL3; ENSG00000127533.4:s:16888999-16889298; 16889298-16889577
FAM219A; ENSG00000164970.15:s:34405865-34405964; 34402807-34405916
FAM3A; ENSG00000071889.17:s:154512823-154512936; 154511871-154512823
FBXO44; ENSG00000132879.14:t:11658736-11658871; 11658628-11658736
FKBP1B; ENSG00000119782.14:s:24060814-24060926; 24060926-24063019
FMNL3; ENSG00000161791.14:t:49657082-49657190; 49657190-49658442
FOXP1; ENSG00000114861.24:t:71112536-71112637; 71112637-71130498
G3BP1; ENSG00000145907.16:t:151786572-151786715; 151772036-151786603
GET1; ENSG00000182093.16:t:39390698-39390803; 39380556-39390698
GLUL; ENSG00000135821.19:t:182388572-182390304; 182388750-182391175
GORASP1; ENSG00000114745.15:s:39107479-39108063; 39103553-39107479
GORASP1; ENSG00000114745.15:t:39101016-39101102; 39103553-39107479
GSE1; ENSG00000131149.19:t:85648552-85648751; 85556363-85648552
GSTT1; ENSG00000277656.3:t:270997-271173; 271173-278295
GUCD1; ENSG00000138867.17:t:24548917-24549001; 24549001-24555615
HDAC7; ENSG00000061273.18:s:47796207-47796298; 47796016-47796207
HES6; ENSG00000144485.11:t:238239487-238239568; 238239568-238239825
HUS1; ENSG00000136273.13:s:47979410-47979615; 47978816-47979410
IFI27; ENSG00000275214.4:t:1230073-1230504; 1229442-1230343
IGF2BP3; ENSG00000136231.14:t:23342064-23342189; 23342189-23343718
IKBKB; ENSG00000104365.16:t:42290156-42290273; 42288728-42290156
IKBKG; ENSG00000269335.7:t:154551988-154552189; 154542452-154551988
IKZF3; ENSG00000161405.17:t:39777651-39777767; 39777767-39788258
IL10RA; ENSG00000110324.12:t:117988382-117988502; 117986534-117988382
IL15RA; ENSG00000134470.21:t:5960367-5960567; 5960567-5963743
IMPDH1; ENSG00000106348.18:t:128405767-128405948; 128405865-128409289
INO80E; ENSG00000169592.15:s:30001414-30001575; 30001530-30005221
INPP5D; ENSG00000281614.3:s:67445-67595; 67595-71086
IP6K2; ENSG00000068745.15:t:48694872-48695421; 48695421-48717157
IRF7; ENSG00000276561.4:s:144369-144427; 144292-144369
IRF7; ENSG00000276561.4:s:144676-144900; 144424-144690
ITSN2; ENSG00000198399.16:s:24246129-24246320; 24242206-24246129
JAML; ENSG00000160593.19:t:118212407-118212561; 118212561-118214824
JOSD2; ENSG00000161677.12:t:50506378-50506572; 50506572-50507574
KANK2; ENSG00000197256.11:s:11195556-11195881; 11194590-11195556
KANK2; ENSG00000197256.11:t:11194553-11194590; 11194590-11195556
KANSL3; ENSG00000114982.19:s:96636739-96637228; 96631543-96636921
KANSL3; ENSG00000114982.19:t:96631312-96631543; 96631543-96636921
KAT5; ENSG00000172977.13:s:65712922-65713319; 65713058-65713348
KATNB1; ENSG00000140854.13:s:57755345-57755494; 57755494-57755841
KDM5C; ENSG00000126012.13:s:53217796-53217966; 53217274-53217796
KIAA1671; ENSG00000197077.14:t:25170820-25170938; 25049364-25170820
KIFC3; ENSG00000140859.16:t:57772223-57772288; 57772288-57785464
KIFC3; ENSG00000140859.16:t:57798072-57798282; 57798282-57802370
KMT2B; ENSG00000272333.8:t:35719784-35720940; 35719541-35719784
LDB1; ENSG00000198728.11:s:102109029-102109177; 102108323-102109029
LDB2; ENSG00000169744.13:s:16511981-16512104; 16508686-16511981
LETMD1; ENSG00000050426.16:s:51048301-51048518; 51048478-51056350
LST1; ENSG00000204482.11:s:31587117-31587318; 31587318-31587641
LST1; ENSG00000204482.11:t:31587944-31587966; 31587318-31587944
LST1; ENSG00000206433.10:s:2842938-2843143; 2843139-2844386
LST1; ENSG00000223465.8:s:2834852-2835057; 2835053-2835376
LST1; ENSG00000223465.8:t:2835679-2835701; 2835053-2835679
LST1; ENSG00000226182.8:t:3065231-3065253; 3064605-3065231
LST1; ENSG00000230791.8:s:2886832-2887014; 2887014-2887225
LST1; ENSG00000230791.8:t:2887225-2887247; 2886599-2887225
LST1; ENSG00000231048.8:s:2892158-2892363; 2892359-2892682
LST1; ENSG00000231048.8:t:2892985-2893007; 2892359-2892985
LTB; ENSG00000204487.8:s:3059543-3059714; 3058917-3059543
LTB; ENSG00000223448.7:s:2887297-2887468; 2886671-2887297
LTB; ENSG00000238114.7:s:2881536-2881707; 2880910-2881536
LTBP4; ENSG00000090006.18:s:40619347-40619493; 40619493-40622401
LTBP4; ENSG00000090006.18:t:40619347-40619493; 40614446-40619347
LTBP4; ENSG00000090006.18:t:40623604-40623732; 40623021-40623604
MAPK9; ENSG00000050748.18:t:180269280-180269409; 180269409-180279796
MARK2; ENSG00000072518.22:s:63904786-63905043; 63905043-63908260
MAST3; ENSG00000099308.13:s:18146881-18147044; 18147017-18147443
MAX; ENSG00000125952.20:t:65077193-65077428; 65077428-65077913
MBNL1; ENSG00000152601.18:t:152414941-152415111; 152269092-152414941
METTL9; ENSG00000197006.15:s:21624931-21625233; 21625115-21655227
MGLL; ENSG00000074416.16:t:127821694-127821838; 127821838-127822309
MGRN1; ENSG00000102858.13:s:4677463-4677572; 4677572-4681550
MIEF1; ENSG00000100335.15:s:39511849-39512026; 39512026-39512232
MLX; ENSG00000108788.12:s:42567619-42567655; 42567655-42568837
MMAB; ENSG00000139428.12:s:109561420-109561517; 109561329-109561420
MPI; ENSG00000178802.18:s:74890005-74890201; 74890089-74890527
MPRIP; ENSG00000133030.22:t:17142627-17142765; 17138429-17142627
MRPL33; ENSG00000243147.8:s:27772674-27772855; 27772692-27779433
MSRB3; ENSG00000174099.12:s:65278643-65278865; 65278865-65326826
MTA1; ENSG00000182979.18:s:105445418-105445511; 105445511-105450058
MTA1; ENSG00000182979.18:t:105450058-105450184; 105445511-105450058
MTHFD2; ENSG00000065911.13:t:74207704-74207826; 74205889-74207704
MTMR12; ENSG00000150712.11:s:32312758-32312987; 32274122-32312758
MTMR12; ENSG00000150712.11:t:32273980-32274122; 32274122-32312758
NAP1L4; ENSG00000273562.4:t:175479-176694; 176694-182308
NCOA2; ENSG00000140396.13:t:70141179-70141399; 70141399-70148273
NCOR2; ENSG00000196498.14:s:124330845-124330898; 124326370-124330845
NCOR2; ENSG00000196498.14:s:124378237-124378384; 124372610-124378237
NCOR2; ENSG00000196498.14:t:124372022-124372610; 124372610-124378237
NDRG2; ENSG00000165795.25:t:21022392-21022820; 21022497-21022864
NDUFV3; ENSG00000160194.18:t:42908864-42913304; 42897047-42908864
NFE2L1; ENSG00000082641.16:s:48056386-48056598; 48056598-48057032
NFIA; ENSG00000162599.18:t:61455303-61462788; 61406727-61455303
NKIRAS2; ENSG00000168256.18:t:42023654-42025644; 42022640-42023654
NOC2L; ENSG00000188976.11:s:945042-945146; 944800-945057
NTAN1; ENSG00000275779.4:s:613600-613788; 613788-621580
NTPCR; ENSG00000135778.12:t:232956347-232956443; 232950744-232956347
NUDT22; ENSG00000149761.9:t:64229131-64229344; 64227132-64229247
NUP214; ENSG00000126883.19:s:131146129-131146304; 131146304-131147490
NXT2; ENSG00000101888.12:t:109538045-109538131; 109537270-109538045
OCEL1; ENSG00000099330.9:s:17226213-17226353; 17226353-17226693
P2RX4; ENSG00000135124.16:s:121210065-121210298; 121210298-121217134
PABIR2; ENSG00000156504.17:s:134781821-134781917; 134772283-134781821
PALM2AKAP2; ENSG00000157654.19:s:110138171-110138539; 110138539-110168399
PANK4; ENSG00000157881.16:t:2521101-2521315; 2521315-2526464
PAPOLA; ENSG00000090060.19:s:96556175-96556413; 96556413-96560649
PARL; ENSG00000175193.14:s:183862607-183862801; 183844326-183862697
PARL; ENSG00000175193.14:t:183844231-183844774; 183844326-183862753
PARVB; ENSG00000188677.15:t:44093928-44094017; 44069162-44093928
PCYT1B; ENSG00000102230.14:t:24618985-24619084; 24619084-24646989
PDE7A; ENSG00000205268.11:t:65782783-65782843; 65782843-65841371
PEX26; ENSG00000215193.14:s:18083437-18083732; 18083732-18087972
PFKFB3; ENSG00000170525.21:t:6213623-6213748; 6203336-6213623
PKN1; ENSG00000123143.13:t:14441143-14441443; 14433542-14441143
PLEKHM1; ENSG00000225190.12:s:45481603-45482612; 45478147-45482433
PLS3; ENSG00000102024.19:t:115610243-115610323; 115561260-115610243
PML; ENSG00000140464.20:s:74034478-74034558; 74034530-74042989
PML; ENSG00000140464.20:t:74033156-74033422; 74032715-74033156
POLR2J3; ENSG00000168255.20:t:102566997-102567083; 102567083-102568010
POLR2J3; ENSG00000285437.2:t:102566966-102567083; 102567083-102568010
PPIE; ENSG00000084072.17:s:39752910-39753266; 39753052-39753287
PPM1N; ENSG00000213889.11:t:45499949-45500066; 45497343-45499949
PPP6R2; ENSG00000100239.16:s:50436367-50436452; 50436452-50436988
PPP6R2; ENSG00000100239.16:t:50437506-50437603; 50437068-50437506
PRKAR1B; ENSG00000188191.16:t:596146-596304; 596304-602553
PRKCD; ENSG00000163932.16:t:53178404-53178537; 53165215-53178404
PSEN1; ENSG00000080815.20:t:73170797-73171923; 73148094-73170797
PSMB8; ENSG00000230034.8:t:4086512-4086659; 4086659-4087849
PSMG4; ENSG00000180822.12:s:3263684-3263759; 3263759-3264209
PTK2B; ENSG00000120899.18:t:27450749-27450895; 27445919-27450749
PTPN12; ENSG00000127947.16:s:77600664-77600965; 77600806-77607235
PTPN18; ENSG00000072135.13:t:130368897-130369201; 130356200-130369133
PUF60; ENSG00000179950.15:s:143821118-143821743; 143818534-143821597
PUF60; ENSG00000179950.15:s:143821118-143821743; 143820716-143821597
PUM1; ENSG00000134644.16:s:30967099-30967310; 30966278-30967167
PYM1; ENSG00000170473.17:t:55903387-55903480; 55903480-55927076
RAB11FIP1; ENSG00000156675.16:t:37870387-37870528; 37870528-37871278
RAB11FIP5; ENSG00000135631.17:t:73075993-73076182; 73076182-73088050
RAB4A; ENSG00000168118.12:t:229295848-229295910; 229271370-229295848
RABGAP1L; ENSG00000152061.24:t:174969277-174969387; 174957549-174969277
RAP1B; ENSG00000127314.18:t:68650244-68650882; 68648781-68650400
RARA; ENSG00000131759.18:t:40348316-40348464; 40331396-40348316
RBCK1; ENSG00000125826.22:s:409830-410025; 410025-417526
RBM10; ENSG00000182872.16:t:47173128-47173197; 47169498-47173128
RBM39; ENSG00000131051.24:s:35713019-35713203; 35709256-35713019
RCOR3; ENSG00000117625.14:t:211313424-211316385; 211312961-211313424
REPS2; ENSG00000169891.18:s:17022123-17022371; 17022271-17025059
RFFL; ENSG00000092871.17:s:35026374-35026568; 35021781-35026374
RHD; ENSG00000187010.21:s:25303322-25303459; 25303459-25328898
RMND5B; ENSG00000145916.19:t:178138108-178138396; 178131054-178138108
RPUSD1; ENSG00000007376.8:t:786826-786928; 786928-787077
SEC16A; ENSG00000148396.19:s:136447227-136447364; 136446949-136447227
SIGLEC10; ENSG00000142512.15:t:51414422-51414515; 51414515-51415181
SIRT3; ENSG00000142082.15:s:218778-219041; 216718-218778
SLA2; ENSG00000101082.14:s:36615225-36615374; 36614387-36615225
SLC66A2; ENSG00000122490.19:t:79903446-79904183; 79904183-79919184
SMARCA4; ENSG00000127616.21:s:11034914-11035132; 11035132-11041298
SMARCA4; ENSG00000127616.21:t:11040634-11041560; 11035132-11041298
SMARCC2; ENSG00000139613.12:s:56173696-56173849; 56173029-56173696
SNX20; ENSG00000167208.16:s:50675770-50675921; 50669148-50675770
SPIB; ENSG00000269404.7:s:50423605-50423755; 50423751-50428038
SRSF6; ENSG00000124193.16:s:43458361-43458509; 43458509-43459153
SSX2IP; ENSG00000117155.17:t:84670646-84670815; 84670815-84690371
STAMBP; ENSG00000124356.17:t:73830845-73831059; 73829070-73830845
STAT3; ENSG00000168610.16:t:42317182-42317224; 42317224-42319856
SWI5; ENSG00000175854.15:t:128276587-128276755; 128276402-128276707
SYNE1; ENSG00000131018.25:s:152301869-152302269; 152300781-152301869
TANGO2; ENSG00000183597.16:t:20061530-20061802; 20056013-20061530
TCF3; ENSG00000071564.19:s:1615686-1615804; 1612433-1615686
TCF7L2; ENSG00000148737.18:s:113146011-113146097; 113146097-113150998
TECR; ENSG00000099797.15:s:14562356-14562575; 14562575-14563174
TJP2; ENSG00000119139.21:t:69212548-69212601; 69151771-69212548
TLE4; ENSG00000106829.21:s:79627374-79627752; 79627448-79652593
TLE4; ENSG00000106829.21:t:79652590-79652629; 79627448-79652593
TMBIM1; ENSG00000135926.15:s:218292466-218292586; 218282181-218292466
TMBIM4; ENSG00000155957.19:s:66169855-66170027; 66161172-66169855
TMEM11; ENSG00000178307.10:s:21214091-21214176; 21211227-21214091
TMEM126B; ENSG00000171204.13:t:85631687-85631808; 85628688-85631687
TMEM219; ENSG00000149932.17:t:29962677-29963308; 29962132-29963107
TNFSF12; ENSG00000239697.12:t:7549466-7549618; 7549312-7549474
TNK2; ENSG00000061938.21:t:195888426-195888606; 195888606-195908485
TNRC18; ENSG00000182095.15:t:5325096-5325574; 5325248-5329937
TRAPPC2; ENSG00000196459.15:s:13734525-13734635; 13719982-13734525
TRAPPC6A; ENSG00000007255.10:t:45164725-45164970; 45164970-45165127
TSC22D3; ENSG00000157514.18:t:107715773-107715950; 107715950-107775100
TSPAN32; ENSG00000064201.16:s:2314485-2314666; 2314571-2316229
UBAC2; ENSG00000134882.16:t:99238427-99238554; 99200939-99238427
UFD1; ENSG00000070010.19:t:19471687-19471808; 19471808-19479083
URI1; ENSG00000105176.18:s:29942239-29942664; 29942664-29985223
URI1; ENSG00000105176.18:t:29985223-29985301; 29942664-29985223
USP15; ENSG00000135655.16:s:62325872-62325933; 62325933-62349221
USP22; ENSG00000124422.12:t:21028542-21028674; 21028674-21043321
VGLL4; ENSG00000144560.16:t:11601833-11602022; 11602022-11702971
WWP2; ENSG00000198373.13:t:69786996-69787080; 69762391-69786996
YAF2; ENSG00000015153.15:s:42237599-42237800; 42199235-42237599
YIPF6; ENSG00000181704.12:s:68498562-68499157; 68499123-68511849
YIPF6; ENSG00000181704.12:t:68513327-68515340; 68511977-68513327
ZBTB7B; ENSG00000160685.14:t:155014448-155015814; 155002943-155014655
ZEB2; ENSG00000169554.23:t:144517278-144517557; 144517419-144517612
ZFAND1; ENSG00000104231.11:s:81718182-81718224; 81715114-81718182
ZFAND1; ENSG00000104231.11:t:81714987-81715114; 81715114-81718182
ZFAND2B; ENSG00000158552.13:s:219207648-219207779; 219207774-219207887
ZMIZ2; ENSG00000122515.16:s:44759281-44759460; 44759460-44760151
ZMYND8; ENSG00000101040.20:t:47236326-47236516; 47236516-47238758
ZNF185; ENSG00000147394.20:s:152922720-152922809; 152922809-152928575
ZNF451; ENSG00000112200.17:t:57099061-57099141; 57090274-57099061

The list of genes and LSV ID's present in Table 1 were further analyzed to allow for heterogenous data that allows for a complex disease classification tool. A list of 69 genes from Table 1 were determined. The list of 69 genes, including their LSV ID (LSV identifier) and Uniprot ID (Uniprot identifier), are provided in Table 2.

TABLE 2
69 Differentially Present Splice Junctions in Alzheimer's
Disease subjects vs. non-cognitively impaired subjects.

| Gene | LSV ID | Uniprot ID |
|---|---|---|
| UFD1 | ENSG00000070010.19:t:19471687-19471808 | Q92890 |
| LST1 | ENSG00000204482.11:t:31587944-31587966 | O00453 |
| LST1 | ENSG00000204482.11:s:31587117-31587318 | O00453 |
| METTL9 | ENSG00000197006.15:s:21624931-21625233 | Q9H1A3 |
| MSRB3 | ENSG00000174099.12:s:65278643-65278865 | Q8IXL7 |
| EXOSC1 | ENSG00000171311.13:t:97437700-97437750 | Q9Y3B2 |
| EXOSC1 | ENSG00000171311.13:s:97438663-97438703 | Q9Y3B2 |
| JOSD2 | ENSG00000161677.12:t:50506378-50506572 | Q8TAC2 |
| NCOA2 | ENSG00000140396.13:t:70141179-70141399 | Q15596 |
| CBFA2T3 | ENSG00000129993.15:t:88892244-88892485 | O75081 |
| ABLIM1 | ENSG00000099204.21:t:114491791-114491878 | O14639 |
| PAPOLA | ENSG00000090060.19:s:96556175-96556413 | P51003 |
| PPIE | ENSG00000084072.17:s:39752910-39753266 | Q9UNP9 |
| ARHGEF1 | ENSG00000076928.19:s:41896377-41896878 | Q92888 |
| POLR2J3 | ENSG00000285437.2:t:102566966-102567083 | Q9H1A7 |
| IFI27 | ENSG00000275214.4:t:1230073-1230504 | P40305 |
| LTB | ENSG00000238114.7:s:2881536-2881707 | Q06643 |
| LST1 | ENSG00000231048.8:t:2892985-2893007 | O00453 |
| LST1 | ENSG00000231048.8:s:2892158-2892363 | O00453 |
| LST1 | ENSG00000223465.8:t:2835679-2835701 | O00453 |
| LST1 | ENSG00000223465.8:s:2834852-2835057 | O00453 |
| LTB | ENSG00000223448.7:s:2887297-2887468 | Q06643 |
| PPM1N | ENSG00000213889.11:t:45499949-45500066 | Q8N819 |
| LST1 | ENSG00000206433.10:s:2842938-2843143 | O00453 |
| LTB | ENSG00000204487.8:s:3059543-3059714 | Q06643 |
| COX20 | ENSG00000203667.10:s:244835616-244835756 | Q5RI15 |
| KANK2 | ENSG00000197256.11:t:11194553-11194590 | Q63ZY3 |
| KANK2 | ENSG00000197256.11:s:11195556-11195881 | Q63ZY3 |
| NCOR2 | ENSG00000196498.14:s:124378237-124378384 | Q9Y618 |
| NOC2L | ENSG00000188976.11:s:945042-945146 | Q9Y3T9 |
| PARVB | ENSG00000188677.15:t:44093928-44094017 | Q9HBI1 |
| PRKAR1B | ENSG00000188191.16:t:596146-596304 | P31321 |
| ARAP1 | ENSG00000186635.15:s:72693325-72693470 | Q96P48 |
| MTA1 | ENSG00000182979.18:t:105450058-105450184 | Q13330 |
| MTA1 | ENSG00000182979.18:s:105445418-105445511 | Q13330 |
| GET1 | ENSG00000182093.16:t:39390698-39390803 | O00258 |
| PUF60 | ENSG00000179950.15:s:143821118-143821743 | Q9UHX1 |
| PFKFB3 | ENSG00000170525.21:t:6213623-6213748 | Q16875 |
| REPS2 | ENSG00000169891.18:s:17022123-17022371 | Q8NFH8 |
| LDB2 | ENSG00000169744.13:s:16511981-16512104 | O43679 |
| POLR2J3 | ENSG00000168255.20:t:102566997-102567083 | Q9H1A7 |
| CCDC28B | ENSG00000160050.16:t:32204598-32204620 | Q9BUN5 |
| PALM2AKAP2 | ENSG00000157654.19:s:110138171-110138539 | Q8IXS6 |
| MBNL1 | ENSG00000152601.18:t:152414941-152415111 | Q9NR56 |
| MTMR12 | ENSG00000150712.11:t:32273980-32274122 | Q9C0I1 |
| MTMR12 | ENSG00000150712.11:s:32312758-32312987 | Q9C0I1 |
| DGKZ | ENSG00000149091.15:t:46367291-46367399 | Q13574 |
| TCF7L2 | ENSG00000148737.18:s:113146011-113146097 | Q9NQB0 |
| G3BP1 | ENSG00000145907.16:t:151786572-151786715 | Q13283 |
| APP | ENSG00000142192.21:t:25897573-25897983 | P05067 |
| APP | ENSG00000142192.21:s:26000015-26000182 | P05067 |
| KIFC3 | ENSG00000140859.16:t:57798072-57798282 | Q9BVG8 |
| RAB11FIP5 | ENSG00000135631.17:t:73075993-73076182 | Q9BXF6 |
| DCTD | ENSG00000129187.15:t:182915461-182915575 | P32321 |
| RAP1B | ENSG00000127314.18:t:68650244-68650882 | P61224 |
| RBCK1 | ENSG00000125826.22:s:409830-410025 | Q9BYM8 |
| ATE1 | ENSG00000107669.19:t:121924266-121924329 | 095260 |
| DOCK8 | ENSG00000107099.18:t:271627-271729 | Q8NF50 |
| TLE4 | ENSG00000106829.21:s:79627374-79627752 | Q04727 |
| IKBKB | ENSG00000104365.16:t:42290156-42290273 | O14920 |
| DECR1 | ENSG00000104325.7:t:90018564-90018966 | Q16698 |
| DECR1 | ENSG00000104325.7:s:90001405-90001561 | Q16698 |
| ZFAND1 | ENSG00000104231.11:s:81718182-81718224 | Q8TCF1 |
| ARFRP1 | ENSG00000101246.20:t:63706999-63707658 | Q13795 |
| AP1B1 | ENSG00000100280.17:s:29330378-29330532 | Q10567 |
| DNM2 | ENSG00000079805.19:s:10795372-10795439 | P50570 |
| EIF4G3 | ENSG00000075151.24:t:20981048-20981227 | O43432 |
| DAZAP1 | ENSG00000071626.17:s:1422348-1422396 | Q96EP5 |
| TRAPPC6A | ENSG00000007255.10:t:45164725-45164970 | O75865 |

A list of 16 junctions were selected to be integrated into a machine learning algorithm disclosed herein. The 16 genes, as well as their LSV ID (LSV identifiers) are provided in Table 3.

TABLE 3
16 Differentially Present Splice Junctions in Alzheimer's
Disease subjects vs. non-cognitively impaired subjects.

LSV ID (Gene; LSV ID)
LDB2; ENSG00000169744.13:s:16511981-16512104; 16508686-16511981
JOSD2; ENSG00000161677.12:t:50506378-50506572; 50506572-50507574
KIFC3; ENSG00000140859.16:t:57798072-57798282; 57798282-57802370
DOCK8; ENSG00000107099.18:t:271627-271729; 215029-271627
METTL9; ENSG00000197006.15:s:21624931-21625233; 21625115-21655227
CCDC28B; ENSG00000160050.16:t:32204598-32204620; 32204379-32204598
EXOSC1; ENSG00000171311.13:s:97438663-97438703; 97437750-97438663
CBFA2T3; ENSG00000129993.15:t:88892244-88892485; 88892485-88898078
PUF60; ENSG00000179950.15:s:143821118-143821743; 143818534-143821597
NOC2L; ENSG00000188976.11:s:945042-945146; 944800-945057
AP1B1; ENSG00000100280.17:s:29330378-29330532; 29329720-29330378
PPIE; ENSG00000084072.17:s:39752910-39753266; 39753052-39753287
ARAP1; ENSG00000186635.15:s:72693325-72693470; 72688537-72693325
POLR2J3; ENSG00000285437.2:t:102566966-102567083; 102567083-102568010
DAZAP1; ENSG00000071626.17:s:1422348-1422396; 1422396-1425878
ARHGEF1; ENSG00000076928.19:s:41896377-41896878; 41896482-41897315

### Example 4: 16-Junction Classifier

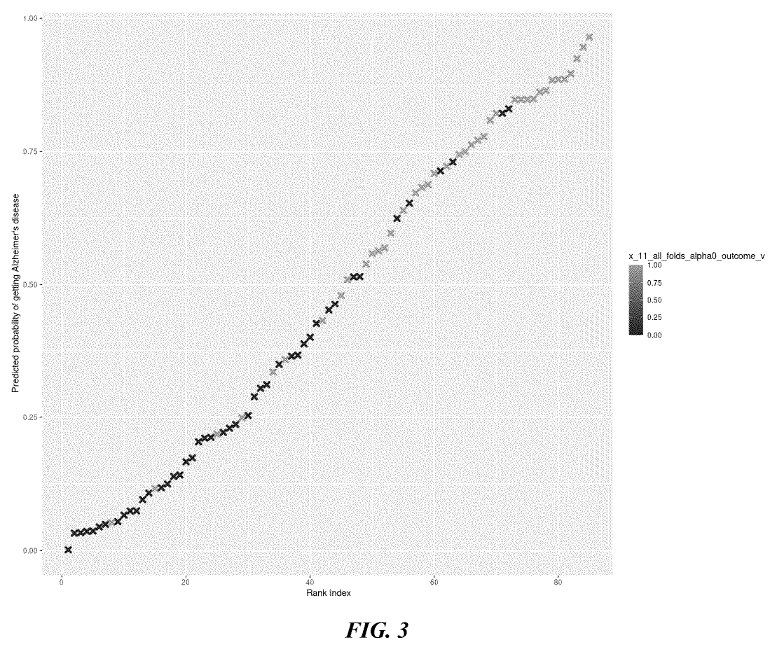

FIG. 2 shows a receiver operating characteristic (ROC) curve for a 16-junction classifier having an area under the curve (AUC) value of 0.868. FIG. 3 shows a sigmoid curve for a 16-junction classifier. The genes and LSV IDs used for the 16-junction classifier were SPIB (ENSG00000269404.7:s:50423605-50423755;50423751-50428038), KIFC3 (ENSG00000140859.16:t:57798072-57798282;57798282-57802370), LDB2 (ENSG00000169744.13:s:16511981-16512104;16508686-16511981), JOSD2 (ENSG00000161677.12:t:50506378-50506572;50506572-50507574), LST1 (ENSG00000230791.8:t:2887225-2887247;2886599-2887225), CCDC28B (ENSG00000160050.16:t:32204598-32204620;32204379-32204598), METTL9 (ENSG00000197006.15:s:21624931-21625233;21625115-21655227), PPIE (ENSG00000084072.17:s:39752910-39753266;39753052-39753287), CBFA2T3 (ENSG00000129993.15:t:88892244-88892485;88892485-88898078), TCF3 (ENSG00000071564.19:s:1615686-1615804;1612433-1615686), DCTD (ENSG00000129187.15:s:182917288-182917477;182915575-182917311), IL10RA (ENSG00000110324.12:t:117988382-117988502;117986534-117988382), ZNF451 (ENSG00000112200.17:t:57099061-57099141;57090274-57099061), AP1B1 (ENSG00000100280.17:s:29330378-29330532;29329720-29330378), EXOSC1 (ENSG00000171311.13:s:97438663-97438703;97437750-97438663), PUF60 (ENSG00000179950.15:s:143821118-143821743;143818534-143821597), POLR2J3 (ENSG00000285437.2:t:102566966-102567083;102567083-102568010), DOCK8 (ENSG00000107099.18:t:271627-271729;215029-271627), NOC2L (ENSG00000188976.11:s:945042-945146;944800-945057), NAP1L4 (ENSG00000273562.4:t:175479-176694;176694-182308), DAZAP1 (ENSG00000071626.17:s:1422348-1422396;1422396-1425878), ARAP1 (ENSG00000186635.15:s:72693325-72693470;72688537-72693325), RBM39 (ENSG00000131051.24:s:35713019-35713203;35709256-35713019), MSRB3 (ENSG00000174099.12:s:65278643-65278865;65278865-65326826), ZBTB7B (ENSG00000160685.14:t:155014448-155015814;155002943-155014655), NUP214 (ENSG00000126883.19:s:131146129-131146304;131146304-131147490), and ARHGEF1 (ENSG00000076928.19:s:41896377-41896878;41896482-41897315).

### Example 5: 27-Junction Classifier

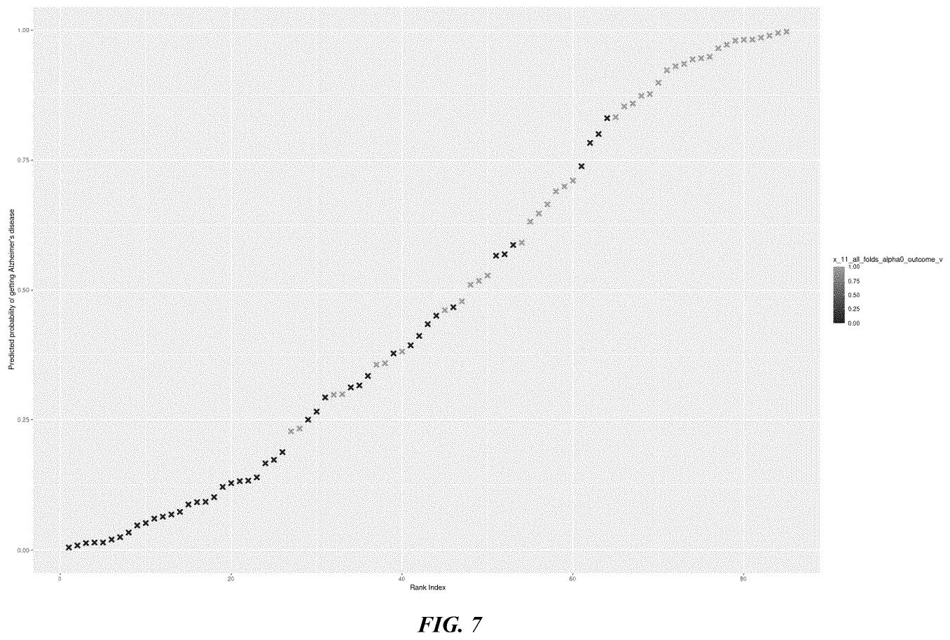

FIG. 6 shows a receiver operating characteristic (ROC) curve for a 27-junction classifier having an area under the curve (AUC) value of 0.904. FIG. 7 shows a sigmoid curve for a 27-junction classifier. The genes, as well as their LSV IDs, used for the 27-junction classifier were SPIB (ENSG00000269404.7:s:50423605-50423755;50423751-50428038), KIFC3 (ENSG00000140859.16:t:57798072-57798282;57798282-57802370), LDB2 (ENSG00000169744.13:s:16511981-16512104;16508686-16511981), JOSD2 (ENSG00000161677.12:t:50506378-50506572;50506572-50507574), LST1 (ENSG00000230791.8:t:2887225-2887247;2886599-2887225), CCDC28B (ENSG00000160050.16:t:32204598-32204620;32204379-32204598), METTL9 (ENSG00000197006.15:s:21624931-21625233;21625115-21655227), PPIE (ENSG00000084072.17:s:39752910-39753266;39753052-39753287), CBFA2T3 (ENSG00000129993.15:t:88892244-88892485;88892485-88898078), TCF3 (ENSG00000071564.19:s:1615686-1615804;1612433-1615686), DCTD (ENSG00000129187.15:s:182917288-182917477;182915575-182917311), IL10RA (ENSG00000110324.12:t:117988382-117988502;117986534-117988382), ZNF451 (ENSG00000112200.17:t:57099061-57099141;57090274-57099061), AP1B1 (ENSG00000100280.17:s:29330378-29330532;29329720-29330378), EXOSC1 (ENSG00000171311.13:s:97438663-97438703;97437750-97438663), PUF60 (ENSG00000179950.15:s:143821118-143821743;143818534-143821597), POLR2J3 (ENSG00000285437.2:t:102566966-102567083;102567083-102568010), DOCK8 (ENSG00000107099.18:t:271627-271729;215029-271627), NOC2L (ENSG00000188976.11:s:945042-945146;944800-945057), NAP1L4 (ENSG00000273562.4:t:175479-176694;176694-182308), DAZAP1 (ENSG00000071626.17:s:1422348-1422396;1422396-1425878), ARAP1 (ENSG00000186635.15:s:72693325-72693470;72688537-72693325), RBM39 (ENSG00000131051.24:s:35713019-35713203;35709256-35713019), MSRB3 (ENSG00000174099.12:s:65278643-65278865;65278865-65326826), ZBTB7B (ENSG00000160685.14:t:155014448-155015814;155002943-155014655), NUP214 (ENSG00000126883.19:s:131146129-131146304;131146304-131147490), and ARHGEF1 (ENSG00000076928.19:s:41896377-41896878;41896482-41897315).

### Example 6: 42-Junction Classifier

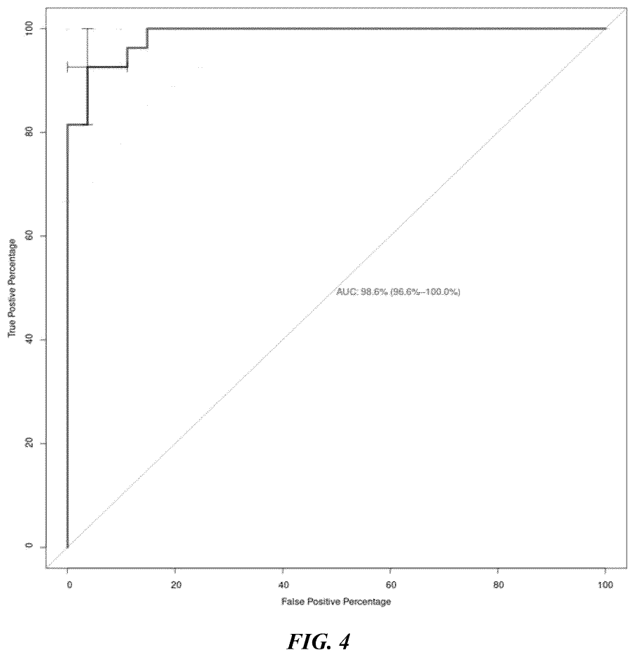

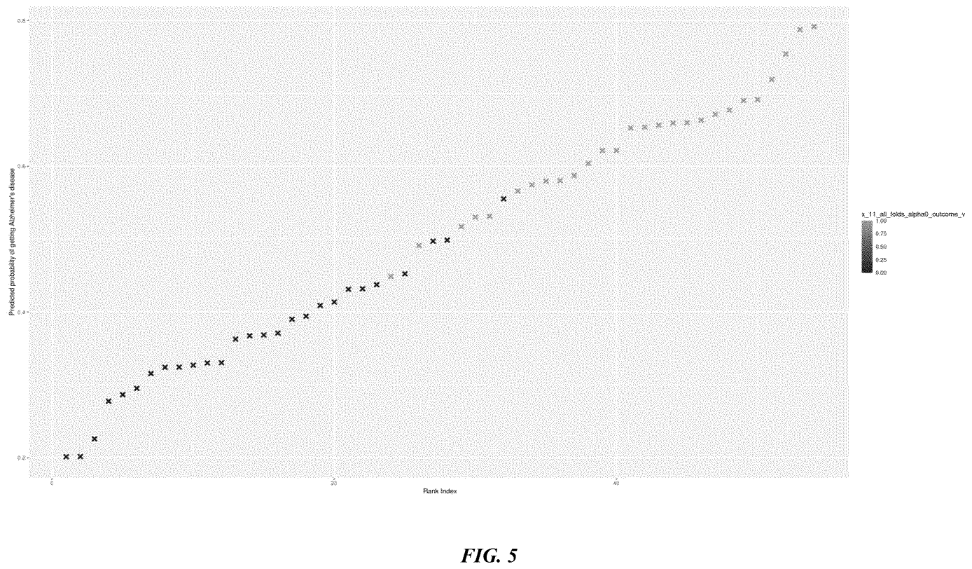

FIG. 4 shows a receiver operating characteristic (ROC) curve for a 42-junction classifier having an area under the curve (AUC) value of 0.986. FIG. 5 shows a sigmoid curve for a 42-junction classifier. The genes, as well as their LSV IDs, used for the 42-junction classifier were ACAP1 (ENSG00000072818.12:t:7341948-7342067;7336787-7341948), ADGRE5 (ENSG00000123146.20:t:14397414-14397804;14391079-14397658), ADK (ENSG00000156110.15:t:74200764-74200838;74151343-74200764), ARHGEF1 (ENSG00000076928.19:s:41896377-41896878;41896482-41897315), ATXN2L (ENSG00000168488.19:t:28836728-28837237;28836485-28836728), CBFB (ENSG00000067955.15:t:67066682-67066962;67036755-67066682), CCDC92 (ENSG00000119242.9:s:123972529-123972831;123943493-123972529), DCTD (ENSG00000129187.15:s:182917288-182917477;182915575-182917311), DECR1 (ENSG00000104325.7:s:90001405-90001561;90001561-90017124), DOCK8 (ENSG00000107099.18:t:271627-271729;215029-271627), DYRK1A (ENSG00000157540.22:s:37418905-37420384;37420384-37472684), EXOSC1 (ENSG00000171311.13:s:97438663-97438703;97437750-97438663), FAM219A (ENSG00000164970.15:s:34405865-34405964;34402807-34405916), GLUL (ENSG00000135821.19:t:182388572-182390304;182388750-182391175), GSE1 (ENSG00000131149.19:t:85648552-85648751;85556363-85648552), GUCD1 (ENSG00000138867.17:t:24548917-24549001;24549001-24555615), LST1 (ENSG00000230791.8:t:2887225-2887247;2886599-2887225), MAST3 (ENSG00000099308.13:s:18146881-18147044;18147017-18147443), METTL9 (ENSG00000197006.15:s:21624931-21625233;21625115-21655227), MPRIP (ENSG00000133030.22:t:17142627-17142765;17138429-17142627), MRPL33 (ENSG00000243147.8:s:27772674-27772855;27772692-27779433), NAP1L4 (ENSG00000273562.4:t:175479-176694;176694-182308), NCOR2 (ENSG00000196498.14:s:124330845-124330898;124326370-124330845), NDRG2 (ENSG00000165795.25:t:21022392-21022820;21022497-21022864), NKIRAS2 (ENSG00000168256.18:t:42023654-42025644;42022640-42023654), PDE7A (ENSG00000205268.11:t:65782783-65782843;65782843-65841371), PKN1 (ENSG00000123143.13:t:14441143-14441443;14433542-14441143), PLEKHM1 (ENSG00000225190.12:s:45481603-45482612;45478147-45482433), PTPN12 (ENSG00000127947.16:s:77600664-77600965;77600806-77607235), RAB11FIP1 (ENSG00000156675.16:t:37870387-37870528;37870528-37871278), RCOR3 (ENSG00000117625.14:t:211313424-211316385;211312961-211313424), RFFL (ENSG00000092871.17:s:35026374-35026568;35021781-35026374), SPIB (ENSG00000269404.7:s:50423605-50423755;50423751-50428038), SSX2IP (ENSG00000117155.17:t:84670646-84670815;84670815-84690371), TMEM219 (ENSG00000149932.17:t:29962677-29963308;29962132-29963107), TNFSF12 (ENSG00000239697.12:t:7549466-7549618;7549312-7549474), TRAPPC2 (ENSG00000196459.15:s:13734525-13734635;13719982-13734525), USP15 (ENSG00000135655.16:s:62325872-62325933;62325933-62349221), YIPF6 (ENSG00000181704.12:t:68513327-68515340;68511977-68513327), ZFAND2B (ENSG00000158552.13:s:219207648-219207779;219207774-219207887), ZMYND8 (ENSG00000101040.20:t:47236326-47236516;47236516-47238758), and ZNF451 (ENSG00000112200.17:t:57099061-57099141;57090274-57099061).

While preferred embodiments of the present inventive concepts have been shown and disclosed herein, it will be obvious to those skilled in the art that such embodiments are provided by way of example only. Numerous variations, changes, and substitutions will now occur to those skilled in the art without departing from the inventive concepts. It should be understood that various alternatives to the embodiments of the inventive concepts disclosed herein may be employed in practicing the inventive concepts. It is intended that the following claims define the scope of the inventive concepts and that methods and structures within the scope of these claims and their equivalents be covered thereby.

1.-93. (canceled)

94. A method comprising:
(a) obtaining a biological sample from the subject; and
(b) assaying cell-free messenger ribonucleic acid (cf-mRNA) in the biological sample to determine a level of the cf-mRNA that comprises a non-contiguous junction relative to genomic deoxyribonucleic acid (DNA), thereby detecting one or more splice junctions in the cf-mRNA, wherein the one or more splice junctions are associated with the disease state.

95. The method of claim 94, wherein the biological sample comprises a blood sample, a plasma sample, or a serum sample.

96. The method of claim 95, wherein the biological sample comprises the plasma sample.

97.-98. (canceled)

99. The method of claim 94, wherein the assaying comprises sequencing, array hybridization, amplification, or a combination thereof.

100. The method of claim 99, wherein the sequencing comprises next generation sequencing.

101. The method of claim 94, wherein the assaying comprises converting the cf-mRNA to complementary deoxyribonucleic acid (cDNA), thereby producing sample cDNA.

102. The method of claim 94, wherein the assaying comprises determining a relative abundance of the one or more splice junctions.

103.-108. (canceled)

109. The method of claim 94, wherein the one or more splice junctions correspond to one or more genes expressed in a first population of subjects with Alzheimer's disease as compared to a second population of subjects without Alzheimer's disease.

110. The method of claim 94, wherein the one or more splice junctions comprise one or more isoforms.

111. The method of claim 94, wherein the one or more splice junctions comprise exon-exon junctions, exon-intron junctions, intron-intron junctions, or a combination thereof.

112. (canceled)

113. A method of assessing an effect of a compound, the method comprising:
(a) assaying a first expression profile of a first cell-free biological sample obtained or derived from a subject at a first time point, to detect a first set of splice junctions;
(b) administering the compound to the subject;
(c) assaying a second expression profile of a second cell-free biological sample obtained or derived from the subject at a second time point subsequent to the administering of (b), to detect a second set of splice junctions;
(d) computer processing the detected first and second sets of splice junctions; and
(e) assessing the effect of the compound based at least in part on the computer processing.

114. The method of claim 94, further comprising computer processing the one or more splice junctions detected in (b).

115. The method of claim 114, further comprising detecting the disease state of the subject based at least in part on the computer processing.

116. The method of claim 115, wherein the disease state comprises a presence or an absence of a neurodegenerative disorder.

117. The method of claim 116, wherein the neurodegenerative disorder is Alzheimer's disease.

118. The method of claim 102, further comprising comparing the relative abundance to a reference sample.

119. The method of claim 115, further comprising administering a treatment to the subject, thereby treating the disease state of the subject.

120. The method of claim 119, wherein the treatment comprises a medicinal therapy, a behavioral therapy, a sleep therapy, or a combination thereof.

121. The method of claim 120, wherein the medicinal therapy comprises a cholinesterase inhibitor, a N-methyl-D-aspartate (NMDA) antagonist, or an amyloid monoclonal antibody (mab) therapy.

122. The method of claim 114, wherein the computer processing comprises use of machine learning.

123. The method of claim 114, wherein the computer processing comprises use of prediction, classification, or a combination thereof.
